(19) United States
(12) Patent Application Publication
(10) Pub. No.: US 2012/0277165 A1
(43) Pub. Date: Nov. 1, 2012

(54) METHODS OF MODULATING FUCOSYLATION OF GLYCOPROTEINS

(76) Inventors: Brian E. Collins, Arlington, MA (US); Lakshmanan Thiruneelakantapillai, Boston, MA (US); Dorota A. Bulik, Malden, MA (US); Kevin Millea, Saugus, MA (US)

(21) Appl. No.: 13/375,577

(22) PCT Filed: Jun. 4, 2010
(86) PCT No.: PCT/US10/37454

§ 371 Date: Jul. 13, 2012

Related U.S. Application Data
(60) Provisional application No. 61/184,493, filed on Jun. 5, 2009.

(51) Int. Cl. C12P 21/00; C12N 5/071; C12M 3/00; A61K 38/14; G01N 27/62; G01N 30/02; G01N 24/08; C12Q 3/00; C12P 21/08; C12Q 1/02

(52) U.S. Cl. 514/20.9; 435/70.3; 435/325; 435/289.1; 435/29; 435/3

(57) ABSTRACT

The present invention provides methods and materials useful for monitoring and regulating the glycosylation of glycoproteins that are recombinantly produced from cells. In particular, methods are provided for monitoring and regulating levels of cellular indicators which affect the level of fucosylation produced by cells.

### CLAIM OF PRIORITY

This application claims priority under 35 USC §119(e) to U.S. Patent Application Ser. No. 61/184,493, filed on Jun. 5, 2009, the entire contents of which is hereby incorporated by reference.

### FIELD OF THE INVENTION

The invention relates to glycoproteins and glycoprotein preparations having reduced core fucosylation and methods related thereto, e.g., methods of making and using the glycoproteins and glycoprotein preparations.

### BACKGROUND OF INVENTION

A typical glycoprotein consists not only of an amino acid backbone but also includes one or more glycan moieties. The glycan moieties attached to the amino acid backbone of a glycoprotein can vary structurally in many ways including, sequence, branching, sugar content, and heterogeneity. Glycosylation adds not only to the structural complexity of the molecules, but also affects or conditions many of a glycoprotein's biological and clinical attributes.

### SUMMARY OF INVENTION

As is disclosed herein, the relationship between GDP-fucose levels in a cell and the level of fucosylation of proteins produced by a cell is not linear. A relatively modest reduction in GDP-fucose levels in the cell can result in a much lower level of fucosylation on proteins produced by the cell. Thus, when levels of GDP-fucose taught herein are used, the reduction of fucose on proteins produced by the cells can be maximized with minimal reduction in GDP-fucose levels and minimal disruption of other aspects of metabolism. E.g., one or more manipulations described herein can be used to achieve a minimal reduction of GDP-fucose levels but still provide a relatively great reduction in fucosylation. Thus, methods described herein allow optimization of the levels of GDP-fucose reduction with reduction in the fucosylation of proteins made by the cell.

The inventors have shown that the relationship between the level of GDP-fucose in a cell and the level of fucosylation on proteins made by the cell is non-linear. In embodiments the curve which describes the relationship between level of GDP-fucose in a cell and level of fucosylation of proteins made by the cell includes three phases. In embodiments the three phase are as follows: a first phase, beginning at relatively high concentrations of GDP-fucose, and continuing through declining levels of GDP-fucose, wherein the level of fucosylation on proteins made by the cell is, compared to the other two phases, relatively constant; a second phase, beginning at levels of GDP-fucose that are lower than the levels seen in the first phase, wherein the level of fucosylation on proteins made by the cell, compared to the other two phases, drops rapidly in response to a decrease in GDP-fucose level; and a third phase, beginning at levels of GDP-fucose that are lower than levels in the second phase, and continuing through declining levels of GDP-fucose, wherein the level of fucosylation on proteins made by the cell is, compared to the other two phases, relatively constant.

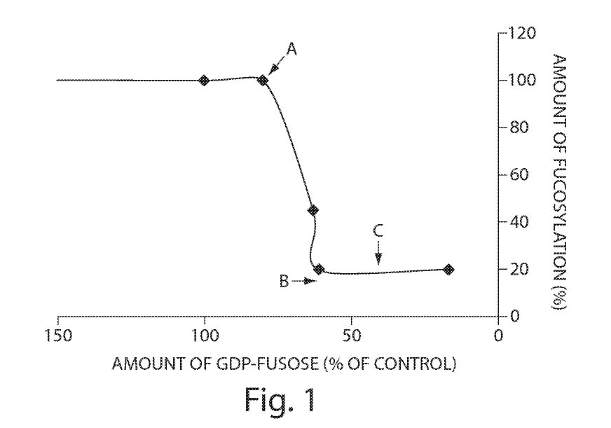

In embodiments the curve which describes the relationship between level of GDP-fucose in a cell and level of fucosylation of proteins made by the cell has three phases: a phase having a high relatively constant (relatively independent of the amount of GDP-fucose) level of fucosylation (points to the left of point A in FIG. 1), a phase of rapid decrease in fucosylation (points between A and B in FIG. 1, wherein the level of fucosylation is relatively sensitive to the amount of GDP-fucose), and phase having a lower, relatively constant, level of fucosylation (relatively independent of the amount of GDP-fucose) (points to the right of point B in FIG. 1). (FIG. 1 and the contents therein are typical. Of course analogous plots may also be used. In embodiments the curve plotting the relationship between level of GDP-fucose in a cell and level of fucosylation of proteins made by the cell may look different from that in FIG. 1, but it will still have the three phases described.)

The appreciation of this relationship can be used to guide selection of the level of GDP-fucose, e.g., to allow minimization of the level of fucosylation with minimal reduction in the level of GDP-fucose in the cell. The balance between low fucose and undesirable contributions of low GDP-fucose levels can be optimized. This can allow minimizing the negative effects of very low concentrations of GDP-fucose.

For example, in some embodiments a decrease in GDP-mannose concentrations can be an undesirable side effect of very low GDP-fucose levels. In some instances a loss of GDP-fucose can lead to higher levels of conversion of GDP-mannose to GDP-fucose, leading to an undesirable decrease in intracellular levels of GDP-mannose. A decrease in GDP-mannose can result in a decrease in high mannose structures on proteins produced by the cell. High mannose structures mediate effector function, and particularly ADCC activity, of an antibody. Thus, if ADCC activity is a desirable property, a decrease in high mannose structures can be undesirable. Alternatively, if less ADCC activity is desired decreased GDP-mannose can be desirable.

Optimal levels can be determined by monitoring the levels of GDP-mannose in the cell; as needed the levels of GDP-fucose can be elevated if the levels of GDP-mannose begin to drop. In particular embodiments, GDP-fucose is increased, e.g., added, if GDP-mannose levels are less than about 90%, 80%, 70%, 60%, 50%, 40%, 30%, 25%, 20%, 15% or 10% of a reference GDP-mannose level, e.g., the level seen in an otherwise similar cell that does not have a reduction in GDP-mannose.

In other embodiments an increase in GDP-mannose concentrations is can be an undesirable side effect of very low GDP-fucose levels. In some instances a loss of GDP-fucose may lead to decreased conversion of GDP-mannose to GDP-fucose, leading to an undesirable increase in the levels of GDP-mannose (in some embodiments this might be observed when a cell is largely or completely deficient in the enzymes involved in the conversion of GDP-mannose to GDP-fucose). Optimal levels can be determined by monitoring the levels of GDP-mannose in the cell; as needed the levels of GDP-fucose or the level of the converting enzyme responsible for the GDP-fucose can be elevated if the levels of GDP-mannose begin to rise. In particular embodiments, GDP-fucose or the level of the converting enzyme is increased if GDP-mannose levels are more than about 2×, 3×, 4×, 5×, 6×, 7×, 8×, 9×, or 10× of a reference GDP-mannose level, e.g. the level seen in an otherwise similar cell that does not have reduction ion the GDP-mannose.

The invention features glycoproteins, e.g., antibodies, and preparations thereof having reduced fucosylation, e.g., reduced core fucosylation. Exemplary proteins include a peptide which comprises a human IgG constant region, e.g., one made in cultured cells, e.g., CHO cells, and having a glycan component attached in the CH2 region, e.g., at residue Asn 297. Preparations, e.g., pharmaceutically acceptable preparations, of these, and other proteins having reduced levels of fucosylation, e.g., core fucosylation, are provided. The presence of core fucosylation on an antibody significantly attenuates its ADCC activity. Reduction of core fucosylation increases ADCC activity.

The invention provides methods in which cells having a manipulation (defined below) can be used to provide proteins having reduced fucosylation. E.g, one or both of a genetically engineered alteration and culture conditions can be used to provide an optimized level of GDP-fucose and an optimized level of fucosylation on proteins made by a cell.

Accordingly, in one aspect, the invention features, a method of reducing fucosylation of a glycoprotein (or a preparation of glycoproteins). The method comprises:

providing a cell having or subject to a manipulation that results in a level of GDP-fucose in said cell that is below a first preselected level and, in embodiments, above a second preselected level and optionally memorializing one or both levels;

culturing said cell, e.g., to provide a batch of cultured cells;

optionally, measuring the level of GDP-fucose in said cell or batch of cultured cells;

optionally, separating the glycoprotein from at least one component with which said cell or batch of cultured cells was cultured; and

optionally, evaluating the glycoprotein (or a glycoprotein on the surface of the cell) for a parameter related to fucosylation;

thereby providing a glycoprotein with reduced fucosylation, e.g., wherein the level of fucosylation is reduced by a predetermined level in comparison with a reference.

In an embodiment the manipulation is or was selected on the basis of providing a level of GDP fucose below a first preselected level and optionally above a second preselected level.

In one embodiment, the method further comprises evaluating a glycan on the surface of said cell or batch of cultured cells in order to determine if the glycoprotein produced by said cell or batch of cultured cells has reduced fucosylation. In another embodiment, said evaluation comprises evaluating a glycan on the surface of said cell or batch of cultured cells, to determine a property of said glycan, comparing the property to a reference, to thereby determine if said glycan structure is present on the product.

In one embodiment, said first preselected level of GDP-fucose is selected from a level that is:

i.a) approximately equal to or less than 80%, 70% or 60% of a reference level, e.g., the level in said cell or batch of cultured cells, e.g., a cell or batch of cultured cells which is otherwise similar, without the manipulation;

ii.a) approximately equal to, or less than, the point of maximum curvature above the inflection point (e.g., the inflection point in the second phase) on a graph of the amount of fucosylation vs. decrease in GDP-fucose;

ii.1.a) approximately equal to, or less than, the lowest level that results in a normal (e.g., that seen in an un-manipuated cell) level of fucosylation;

iii.a) approximately equal to or less than the point of maximum curvature below the inflection point on a graph of the amount of fucosylation vs. decrease in GDP-fucose;

iii.1.a) approximately equal to, or less than, the highest level that results in no further reduction in fucosylation;

iv.a) approximately equal to or less than point A on the curve in FIG. 1, or less than or equal to an analogous point on a plot of the amount of fucosylation (%) vs. the amount of GDP fucose as a % of control;

v.a) approximately equal to or less than that corresponding to an amount between points A and B on the curve in FIG. 1, or less than or equal to an analogous point on a plot of the amount of fucosylation (%) vs. the amount of GDP fucose as a % of control; or

vi.a) approximately equal to or less than point B on the curve in FIG. 1, or less than or equal to an analogous point on a plot of the amount of fucosylation (%) vs. the amount of GDP fucose as a % of control.

In one embodiment, said second preselected level of GDP-fucose is selected from a level:

i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;

ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;

iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level

iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;

v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level;

vi.b) approximately equal to or greater than point C on the curve in FIG. 1, or greater than or equal to an analogous point on a plot of the amount of fucosylation (%) vs. the amount of GDP fucose as a % of control.

In an embodiment the first level is i.a and the second level is selected from i.b, ii.b, iii.b, iv.b, v.b, and vi.b.

In an embodiment the first level is ii.a and the second level is selected from i.b, ii.b, iii.b, iv.b, v.b, and vi.b.

In an embodiment the first level is ii.1.a and the second level is selected from i.b, ii.b, iii.b, iv.b, v.b, and vi.b.

In an embodiment the first level is iii.a and the second level is selected from i.b, ii.b, iii.b, iv.b, v.b, and vi.b.

In an embodiment the first level is iii.1.a and the second level is selected from i.b, ii.b, iii.b, iv.b, v.b, and vi.b.

In an embodiment the first level is iv.a and the second level is selected from i.b, ii.b, iii.b, iv.b, v.b, and vi.b.

In an embodiment the first level is v.a and the second level is selected from i.b, ii.b, iii.b, iv.b, v.b, and vi.b.

In an embodiment the first level is vi.a and the second level is selected from i.b, ii.b, iii.b, iv.b, v.b, and vi.b.

In an embodiment the first level is selected from i.a, ii.a, ii.1.a, iii.a, iii.1.a, iv.a, v.a, and vi.a and the second level is i.b.

In an embodiment the first level is selected from i.a, ii.a, ii.1.a, iii.a, iii.1.a, iv.a, v.a, and vi.a and the second level is ii.b.

In an embodiment the first level is selected from i.a, ii.a, ii.1.a, iii.a, iii.1.a, iv.a, v.a, and vi.a and the second level is iii.b.

In an embodiment the first level is selected from i.a, ii.a, ii.1.a, iii.a, iii.1.a, iv.a, v.a, and vi.a and the second level is iv.b.

In an embodiment the first level is selected from i.a, ii.a, ii.1.a, iii.a, iii.1.a, iv.a, v.a, and vi.a and the second level is v.b.

In an embodiment the first level is selected from i.a, ii.a, ii.1.a, iii.a, iii.1.a, iv.a, v.a, and vi.a and the second level is vi.b.

In an embodiment the level of GDP-fucose is between point B and C on the curve in FIG. 1 or in an analogous range on a plot of the amount of fucosylation (%) vs. the amount of GDP fucose as a % of control.

In an embodiment the level of GDP-fucose is between point A and C on the curve in FIG. 1 or in an analogous range on a plot of the amount of fucosylation (%) vs. the amount of GDP fucose as a % of control.

In one embodiment, the level of GDP-fucose is selected to be outside the range between A and B on the curve in FIG. 1 (as relatively small changes in GDP-fucose will result in relatively large changes in the amount of fucosylation. In an embodiment the level is also less than B.) In another embodiment, the level of GDP-fucose is reduced by a predetermined level, e.g., in comparison with a reference. In another embodiment, the reference is the amount present in a cell or batch of cultured cells, e.g., a CHO cell or batch of cultured cells, lacking the manipulation but otherwise the same or essentially the same as the cell having the manipulation. In another embodiment, the level of GDP-fucose is reduced by, as much as, or more than, 10, 15, 20, 25, 30, 35, 40, 45, 50, 60, 70, 80 or 90%, as compared to the reference.

In one embodiment, the method further comprises evaluating the glycoprotein for a parameter related to fucosylation, e.g., the amount of fucosylation in the glycan complement, the amount or fucosylation on a component of the glycan complement, or the amount of fucosylation on a glycan component, e.g., in a preparation of glycoproteins.

In one embodiment, the method further comprises evaluating the glycoprotein for a parameter related to fucosylation, e.g., the proportion of a preselected glycan component which bears a fucosyl moiety, e.g., at a selected position on the glycan component, e.g., in a preparation of glycoproteins.

In one embodiment, the level of fucosylation at one, two, three, or more preselected amino acid residues is evaluated. In another embodiment, the level of fucosylation is reduced by a predetermined level in comparison with a reference. In another embodiment, the reference is the amount present in a cell or batch of cultured cells, e.g., a CHO cell or batch of cultured cells, lacking the manipulation but otherwise the same or essentially the same as the cell or batch of cultured cells having the manipulation. In another embodiment, the level of fucosylation is reduced by, as much as, or more than, 10, 15, 20, 25, 30, 35, 40, 45, 50, 60, 70, 80 or 90%, as compared to the reference.

In one embodiment, X_{F} is greater than X_{G},

and wherein,

X_{F} is the % or proportion of reduction in the level of fucosylation (e.g., as compared to the level of fucosylation in a cell or batch of cultured cells lacking the manipulation); and

X_{G} is the % or proportion of reduction in the level of GDP fucose (as compared to the level of GDP fucose in a cell or batch of cultured cells lacking the manipulation).

In one embodiment, said manipulation is not a genetic lesion or the presence of an siRNA that reduces the level of an enzyme that promotes formation of GDP-fucose, or the attachment of a fucosyl moiety. For example, the manipulation is not a lesion that decreases the expression of GMD, FX, fucokinase, GFPP, GDP-synthetase, a fucosyltransferase or a GDP-Fucose transporter. In another embodiment, the cell or batch of cultured cells is wild-type for one or all of GMD, FX, fucokinase, GFPP, GDP-synthetase, a fucosyltransferase or a GDP-Fucose transporter. In another embodiment, the cell or batch of cultured cells does not include an siRNA that targets GMD, FX, fucokinase, GFPP, GDP-synthetase, a fucosyltransferase or a GDP-Fucose transporter. In another embodiment, absent the manipulation, the level of fucosylation is substantially the same as the level in a wild-type cell. In another embodiment, the manipulated cell carries no mutation that substantially lowers GDP-fucose levels. In another embodiment, the manipulated cell has no siRNA that substantially lowers GDP-fucose levels.

In one embodiment, the cell has a mutation (e.g., a genetically engineered change) that decreases the level of GDP-fucose. Exemplary mutations include those which alter the activity of GMD, FX, fucokinase, GFPP, GDP-synthetase, a fucosyltransferase or a GDP-Fucose transporter.

The mutation can be in the structural gene which encodes GMD, FX, fucokinase, GFPP, GDP-synthetase, a fucosyltransferase or a GDP-Fucose transporter. Such mutations can decrease the activity of the encoded protein. The decrease can be partial or complete. Such mutations can act, e.g., by altering the catalytic activity of the protein or by altering its half-life. Other exemplary mutations can be in a sequence that control expression of GMD, FX, fucokinase, GFPP, GDP-synthetase, a fucosyltransferase or a GDP-Fucose transporter. These can be mutations that completely, or partially, reduce the expression of the gene, at the RNA or protein level. Such mutations include deletion or other mutations in endogenous of control sequence. Such mutations also include the introduction of heterologous control sequence, e.g., the introduction of heterologous control regions, e.g., a sequence that will give a desired level of expression. (A heterologous control sequence is a sequence other than a sequence naturally associated with and operably linked to the structural gene.) In embodiments the manipulation comprises a mutation in the structural region or in a control sequence operably linked to the gene.

In an embodiment a cell having a mutation that that decreases the level of GDP-fucose, e.g., a mutation that decreases the activity of GMD, FX, fucokinase, GFPP, GDP-synthetase, a fucosyltransferase or a GDP-Fucose transporter is cultured in the presence of a substance, e.g., fucose, that results in a GDP-fucose level and/or a fucosylation level described herein. In an embodiment the cell includes a mutation that, in the absence of fucose in the culture medium, would result in a cell having an unacceptably low level of GDP-fucose. When, however, cultured under the appropriate conditions, e.g., media supplemented, e.g., with fucose, that cell can exhibit a desired level of GDP-fucose, e.g., a level of GDP-fucose described herein. Thus, fucose or another substance is present in the culture medium at a level that results in a level of GDP-fucose recited above.

In another embodiment, the manipulation is the presence of an siRNA that reduces the level of an enzyme that promotes formation of GDP-fucose, or the attachment of a fucosyl moiety, e.g., an siRNA that targets GMD, FX, fucokinase, GFPP, GDP-synthetase, a fucosyltransferase or a GDP-Fucose transporter, and fucose or another substance is present in the culture medium at a level that results in a level of GDP-fucose recited above.

In one embodiment, said culturing comprises culturing the cell in a medium that results in said level of GDP-fucose.

In one embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the cell is a Chinese Hamster Ovary (CHO) cell. In another embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the glycoprotein is selected from Table 1.

In one embodiment, the method further comprises culturing a plurality of the cells and separating as much as, or at least, 1, 10, 100, 1,000, or 10,000 grams of the glycoprotein from the cells. In another embodiment, the method further comprises combining the glycoprotein having reduced fucosylation with a pharmaceutically acceptable component and, e.g., formulating the glycoprotein having reduced fucosylation into a pharmaceutically acceptable formulation.

In one embodiment, the glycoprotein is analyzed by one or more of HPLC, CE, MALDI-MS and NMR.

In one embodiment, the method further comprises memorializing the result of the evaluation.

In one embodiment, the manipulation is, or is the product of, a selection for reduced levels of GDP-fucose. In another embodiment, the manipulation is, or is the product of, a selection for reduced fucosylation of a glycoprotein. In another embodiment, the manipulation comprises contact with, or inclusion in or on the cell or batch of cultured cells, of an exogenous inhibitor of an enzyme involved in GDP-fucose biosynthesis, e.g., a specific or non-specific inhibitor.

In one embodiment, the level of fucosylation at one, two, three, or more preselected amino acid residues is evaluated.

In one embodiment, one or more of said cell or said batch of cultured cells, said manipulation, and said glycoprotein, is selected on the basis that it or the combination will provide a glycoprotein having reduced fucosylation.

In one embodiment, one or more of said cell or said batch of cultured cells, said manipulation (or manipulations), and said glycoprotein, is selected on the basis that it or the combination will provide a level of GDP-fucose described herein, e.g., a level which gives a minimal level of fucosylation (e.g., with reference to a curve analogous to that in FIG. 1, the level is to the right of point B) but which is above a preselected level In some embodiments the level is above a level that gives an unwanted decrease in the level of GDP-mannose, e.g., a decrease in GDP-mannose that is equal to, or more than, 10%, 20%, 30%, 40% or 50% as compared to a reference level, e.g., the level of GDP-mannose in a cell or batch of cultured cells, e.g., a cell or batch of cultured cells which is otherwise similar, without the manipulation.

In some embodiments the level is above a level that gives an unwanted increase in the level of GDP-mannose, e.g., an increase in GDP-mannose that is equal to, or more than, about 2×, 3×, 4×, 5×, ×, 7×, 8×, 9×, or 10× of a reference level, e.g., the level of GDP-mannose in a cell or batch of cultured cells, e.g., a cell or batch of cultured cells which is otherwise similar, without the manipulation.

In one embodiment, the method further comprises providing a value for a parameter associated with a compound other than GDP-fucose, wherein a parameter for the compound, e.g., the level of the compound, is correlated to the level of GDP-fucose.

In another embodiment, the method further comprises providing a comparison of the value with a reference value, wherein optionally, a preselected relationship of the value to the reference value, e.g., greater than, equal to, or less than, is indicative of whether the level of GDP fucose is above, at or below the second level. In another embodiment, the method further comprises, responsive to the result of the comparison, increasing the level of GDP-fucose, decreasing the level of GDP-fucose or continuing cell culture without intervening to change the level of GDP-fucose. In one embodiment, the compound other than GDP-fucose is GDP-mannose. In one embodiment, the compound other than GDP-fucose is GDP-mannose and the parameter is the level of GDP-mannose.

In one embodiment, the method further comprises providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose. In one embodiment, the method comprises continuing to culture said cells, and repeating the steps above.

In an embodiment, an inhibitor, e.g., an inhibitor of GMD, FX, fucokinase, GFPP, GDP-fucose synthetase, or enzymes involved in the biosynthesis of GDP-mannose, is used, e.g., in the culture medium, to lower the levels of the GDP-fucose. In an embodiment the inhibitor can be guanosine-5′-O-(2-thiodiphosphate)-fucose, guanosine-5′-O-(2-thiodiphosphate)-mannose, pyridoxal-5′-phosphate, GDP-4-dehydro-6-L-deoxygalactose, GDP-L-fucose, guanosine diphosphate (GDP), guanosine monophosphate (GMP), GDP-D-glucose, or p-chloromercuriphenylsulfonate EDTA. The inhibitor can be used with a cell which is mutant or wildtype for one or more GMD, FX, fucokinase, GFPP, GDP-synthetase, a fucosyltransferase or a GDP-Fucose transporter.

In an embodiment the media contains a substance that can increase the level of GDP-fucose, e.g., butyrate or fucose. Such media can be used, e.g., with a cell having a mutation that eliminates or decreased the activity of one or more of GMD, FX, fucokinase, GFPP, GDP-synthetase, a fucosyltransferase or a GDP-Fucose transporter.

While some methods described herein rely at least in part on mutations in a gene that conditions the level of GDP-fucose other methods described herein do not. Thus, cells that are not mutant at key genes involved in maintaining GDP-fucose levels can be used to provide proteins having reduced fucosylation. Levels of GDP-fucose can, e.g., be manipulated by culture conditions.

Thus, in another aspect, the invention features, a method of reducing fucosylation of a glycoprotein or a preparation of glycoproteins, the method comprising:

providing a cell that expresses said glycoprotein and that is wild-type for one or more (or all) of GMD, FX, fucokinase, GFPP, GDP-Fucose synthetase, a fucosyltransferase or a GDP-Fucose transporter;

culturing said cell under conditions that result in a level of GDP-fucose in said cell that is below a first preselected level and, in embodiments, above a second preselected level, and results in a preselected level of fucosylation, which is less than in a reference cell cultured under reference conditions, e.g., to provide a batch of cultured cells;

optionally, measuring the level of GDP-fucose in said cell or batch of cultured cells; and

optionally, separating the glycoprotein from at least one component with which said cell or batch of cultured cells was cultured,

optionally, evaluating the glycoprotein (or a glycoprotein on the surface of the cell or batch of cultured cells) for a parameter related to fucosylation;

thereby providing a glycoprotein with reduced fucosylation, e.g., wherein the level of fucosylation is reduced by a predetermined level in comparison with a reference.

In one embodiment, the method further comprises evaluating a glycan on the surface of said cell or batch of cultured cells in order to determine if the glycoprotein produced by said cell or batch of cultured cells has reduced fucosylation. In another embodiment, said evaluation comprises evaluating a glycan on the surface of said cell or batch of cultured cells, to determine a property of said glycan, comparing the property to a reference, to thereby determine if said glycan structure is present on the product.

In one embodiment, said first preselected level of GDP-fucose is selected from a level that is:

i.a) approximately equal to or less than 80%, 70% or 60% of a reference level, e.g., the level in said cell or batch of cultured cells, e.g., a cell or batch of cultured cells which is otherwise similar, without the manipulation;

ii.a) approximately equal to, or less than, the point of maximum curvature above the inflection point (e.g., the inflection point in the second phase) on a graph of the amount of fucosylation vs. decrease in GDP-fucose;

ii.1.a) approximately equal to, or less than, the lowest level that results in a normal (e.g., that seen in an un-manipuated cell) level of fucosylation;

iii.a) approximately equal to or less than the point of maximum curvature below the inflection point on a graph of the amount of fucosylation vs. decrease in GDP-fucose;

iii.1.a) approximately equal to, or less than, the highest level that results in no further reduction in fucosylation;

iv.a) approximately equal to or less than point A on the curve in FIG. 1, or less than or equal to an analogous point on a plot of the amount of fucosylation (%) vs. the amount of GDP fucose as a % of control;

v.a) approximately equal to or less than that corresponding to an amount between points A and B on the curve in FIG. 1, or less than or equal to an analogous point on a plot of the amount of fucosylation (%) vs. the amount of GDP fucose as a % of control; or

vi.a) approximately equal to or less than point B on the curve in FIG. 1, or less than or equal to an analogous point on a plot of the amount of fucosylation (%) vs. the amount of GDP fucose as a % of control.

In one embodiment, said second preselected level of GDP-fucose is selected from a level:

i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;

ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;

iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level;

iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;

v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level; or

vi.b) approximately equal to or greater than point C on the curve in FIG. 1, or greater than or equal to an analogous point on a plot of the amount of fucosylation (%) vs. the amount of GDP fucose as a % of control.

In an embodiment the first level is i.a and the second level is selected from i.b, ii.b, iii.b, iv.b, v.b, and vi.b.

In an embodiment the first level is ii.a and the second level is selected from i.b, ii.b, iii.b, iv.b, v.b, and vi.b.

In an embodiment the first level is ii.1.a and the second level is selected from i.b, ii.b, iii.b, iv.b, v.b, and vi.b.

In an embodiment the first level is iii.a and the second level is selected from i.b, ii.b, iii.b, iv.b, v.b, and vi.b.

In an embodiment the first level is iii.1.a and the second level is selected from i.b, ii.b, iii.b, iv.b, v.b, and vi.b.

In an embodiment the first level is iv.a and the second level is selected from i.b, ii.b, iii.b, iv.b, v.b, and vi.b.

In an embodiment the first level is v.a and the second level is selected from i.b, ii.b, iii.b, iv.b, v.b, and vi.b.

In an embodiment the first level is vi.a and the second level is selected from i.b, ii.b, iii.b, iv.b, v.b, and vi.b.

In an embodiment the first level is selected from i.a, ii.a, ii.1.a, iii.a, iii.1.a, iv.a, v.a, and vi.a and the second level is i.b.

In an embodiment the first level is selected from i.a, ii.a, ii.1.a, iii.a, iii.1.a, iv.a, v.a, and vi.a and the second level is ii.b.

In an embodiment the first level is selected from i.a, ii.a, ii.1.a, iii.a, iii.1.a, iv.a, v.a, and vi.a and the second level is iii.b.

In an embodiment the first level is selected from i.a, ii.a, ii.1.a, iii.a, iii.1.a, iv.a, v.a, and vi.a and the second level is iv.b.

In an embodiment the first level is selected from i.a, ii.a, ii.1.a, iii.a, iii.1.a, iv.a, v.a, and vi.a and the second level is v.b.

In an embodiment the first level is selected from i.a, ii.a, ii.1.a, iii.a, iii.1.a, iv.a, v.a, and vi.a and the second level is vi.b.

In an embodiment the level of GDP-fucose is between point B and C on the curve in FIG. 1 or in an analogous range on a plot of the amount of fucosylation (%) vs. the amount of GDP fucose as a % of control.

In an embodiment the level of GDP-fucose is between point A and C on the curve in FIG. 1 or in an analogous range on a plot of the amount of fucosylation (%) vs. the amount of GDP fucose as a % of control.

In one embodiment, the level of GDP-fucose is selected to be outside the range between A and B on the curve in FIG. 1 (as relatively small changes in GDP-fucose will result in relatively large changes in the amount of fucosylation. In an embodiment the level is also less than B.). In another embodiment, the level of GDP-fucose is reduced by a predetermined level, e.g., in comparison with a reference. In another embodiment, the reference is the amount present in a cell or batch of cultured cells, e.g., a CHO cell or batch of cultured cells, cultured under reference conditions but otherwise the same or essentially the same as the cell cultured under conditions that result in said level of GDP-fucose. In another embodiment, the level of GDP-fucose is reduced by, as much as, or more than, 10, 15, 20, 25, 30, 35, 40, 45, 50, 60, 70, 80 or 90%, as compared to the reference.

In one embodiment, the method further comprises evaluating the glycoprotein for a parameter related to fucosylation, e.g., the amount of fucosylation in the glycan complement, the amount or fucosylation on a component of the glycan complement, or the amount of fucosylation on a glycan component, e.g., in a preparation of glycoproteins.

In one embodiment, the method further comprises evaluating the glycoprotein for a parameter related to fucosylation, e.g., the proportion of a preselected glycan component which bears a fucosyl moiety, e.g., at a selected position on the glycan component, e.g., in a preparation of glycoproteins.

In one embodiment, the level of fucosylation at one, two, three, or more preselected amino acid residues is evaluated. In another embodiment, the level of fucosylation is reduced by a predetermined level in comparison with a reference. In another embodiment, the reference is the amount present in a cell or batch of cultured cells, e.g., a CHO cell or batch of cultured cells, cultured under reference conditions but otherwise the same or essentially the same as the cell cultured under conditions that result in said level of GDP-fucose. In another embodiment, the level of fucosylation is reduced by, as much as, or more than, 10, 15, 20, 25, 30, 35, 40, 45, 50, 60, 70, 80 or 90%, as compared to the reference.

In one embodiment, wherein X_{F} is greater than X_{G},

and wherein,

X_{F} is the % or proportion of reduction in the level of fucosylation (e.g., as compared to the level of fucosylation in a cell or batch of cultured cells cultured under reference conditions); and

X_{G} is the % or proportion of reduction in the level of GDP fucose (as compared to the level of GDP fucose in a cell or batch of cultured cells cultured under reference conditions).

In one embodiment, the cell or batch of cultured cells does not include an siRNA that targets GMD, FX, fucokinase, GFPP, GDP-synthetase, a fucosyltransferase or a GDP-Fucose transporter.

In one embodiment, the cell or batch of cultured cells does includes an siRNA that targets GMD, FX, fucokinase, GFPP, GDP-synthetase, a fucosyltransferase or a GDP-Fucose transporter.

In an embodiments, an inhibitor, e.g., an inhibitor of GMD, FX, fucokinase, GFPP, GDP-fucose synthetase, or enzymes involved in the biosynthesis of GDP-mannose, is used, e.g., in the culture medium, to lower the levels of the GDP-fucose.

In an embodiment the inhibitor can be guanosine-5′-O-(2-thiodiphosphate)-fucose, guanosine-5′-O-(2-thiodiphosphate)-mannose, pyridoxal-5′-phosphate, GDP-4-dehydro-6-L-deoxygalactose, GDP-L-fucose, guanosine diphosphate (GDP), guanosine monophosphate (GMP), GDP-D-glucose, or p-chloromercuriphenylsulfonate EDTA.

In an embodiment the media contains a substance that can increase the level of GDP-fucose, e.g., butyrate or fucose.

In one embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the cell is a Chinese Hamster Ovary (CHO) cell. In another embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the glycoprotein is selected from Table 1.

In one embodiment, the method further comprises culturing a plurality of the cells and separating as much as, or at least, 1, 10, 100, 1,000, or 10,000 grams of the glycoprotein from the cells. In another embodiment, the method further comprises combining the glycoprotein having reduced fucosylation with a pharmaceutically acceptable component and, e.g., formulating the glycoprotein having reduced fucosylation into a pharmaceutically acceptable formulation.

In one embodiment, the glycoprotein is analyzed by one or more of HPLC, CE, MALDI-MS and NMR.

In one embodiment, the method further comprises memorializing the result of the evaluation.

In one embodiment, the level of fucosylation at one, two, three, or more preselected amino acid residues is evaluated.

In one embodiment, the method further comprises providing a value for a parameter associated with a compound other than GDP-fucose, wherein a parameter for the compound, e.g., the level of the compound, is correlated to the level of GDP-fucose.

In another embodiment, the method further comprises providing a comparison of the value with a reference value, wherein optionally, a preselected relationship of the value to the reference value, e.g., greater than, equal to, or less than, is indicative of whether the level of GDP fucose is above, at or below the second level. In another embodiment, the method further comprises, responsive to the result of the comparison, increasing the level of GDP-fucose, decreasing the level of GDP-fucose or continuing cell culture without intervening to change the level of GDP-fucose. In one embodiment, the compound other than GDP-fucose is GDP-mannose. In one embodiment, the compound other than GDP-fucose is GDP-mannose and the parameter is the level of GDP-mannose.

In one embodiment, the method further comprises providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose. In one embodiment, the method comprises continuing to culture said cells, and repeating the steps above.

Methods described herein allow the production of proteins having reduced fucosylation from a cell line that is not genetically altered to reduce fucosylation. Such methods allow the use of a cell line that produces a reference glycoprotein, e.g., an approved product, by culturing that cell line to provide the reference glycoprotein with optimized levels of fucosylation. E.g., a cell line that has been optimized or otherwise selected for use in producing a protein, e.g., an FDA approved therapeutic protein, can be used to produce a protein having reduced fucosylation according to the invention, without genetically engineering the production line cell.

Accordingly, in another aspect, the invention features, a method of providing a glycoprotein (or preparation thereof) having fucosylation that is reduced compared to a reference glycoprotein, e.g., an FDA approved glycoprotein. The method comprises:

providing a cell that expresses said reference glycoprotein, which optionally, is wild-type for one or more (or all) of GMD, FX, fucokinase, GFPP, GDP-Fucose synthetase, a fucosyltransferase or a GDP-Fucose transporter;

culturing said cell (without inducing a mutation in, or adding an siRNA that targets one or more of GMD, FX, fucokinase, GFPP, GDP-Fuc synthetase, a fucosyltransferase or a GDP-Fucose transporter) under culture conditions that result in a level of GDP-fucose in said cell that is below a first preselected level and, in embodiments, above a second preselected level, and results in a preselected level of fucosylation, which is less than in a reference cell cultured under reference conditions, e.g., to provide a batch of cultured cells;

optionally, measuring the level of GDP-fucose in said cell or batch of cultured cells; and

optionally, separating the glycoprotein from at least one component with which said cell or batch of cultured cells was cultured;

optionally, evaluating the glycoprotein (or a glycoprotein on the surface of the cell or batch of cultured cells) for a parameter related to fucosylation;

thereby providing a glycoprotein having fucosylation that is reduced compared to a reference glycoprotein, e.g., an FDA approved glycoprotein.

In one embodiment, the method further comprises evaluating a glycan on the surface of said cell or batch of cultured cells in order to determine if the glycoprotein produced by said cell or batch of cultured cells has reduced fucosylation. In another embodiment, said evaluation comprises evaluating a glycan on the surface of said cell or batch of cultured cells, to determine a property of said glycan, comparing the property to a reference, to thereby determine if said glycan structure is present on the product.

In one embodiment, the method further comprises evaluating a glycan on the surface of said cell or batch of cultured cells in order to determine if the glycoprotein produced by said cell or batch of cultured cells has reduced fucosylation. In another embodiment, said evaluation comprises evaluating a glycan on the surface of said cell or batch of cultured cells, to determine a property of said glycan, comparing the property to a reference, to thereby determine if said glycan structure is present on the product.

In one embodiment, said first preselected level of GDP-fucose is selected from a level that is:

i.a) approximately equal to or less than 80%, 70% or 60% of a reference level, e.g., the level in said cell or batch of cultured cells, e.g., a cell or batch of cultured cells which is otherwise similar, without the manipulation;

ii.a) approximately equal to, or less than, the point of maximum curvature above the inflection point (e.g., the inflection point in the second phase) on a graph of the amount of fucosylation vs. decrease in GDP-fucose;

ii.1.a) approximately equal to, or less than, the lowest level that results in a normal (e.g., that seen in an un-manipuated cell) level of fucosylation;

iii.a) approximately equal to or less than the point of maximum curvature below the inflection point on a graph of the amount of fucosylation vs. decrease in GDP-fucose;

iii.1.a) approximately equal to, or less than, the highest level that results in no further reduction in fucosylation;

iv.a) approximately equal to or less than point A on the curve in FIG. 1, or less than or equal to an analogous point on a plot of the amount of fucosylation (%) vs. the amount of GDP fucose as a % of control;

v.a) approximately equal to or less than that corresponding to an amount between points A and B on the curve in FIG. 1, or less than or equal to an analogous point on a plot of the amount of fucosylation (%) vs. the amount of GDP fucose as a % of control; or

vi.a) approximately equal to or less than point B on the curve in FIG. 1, or less than or equal to an analogous point on a plot of the amount of fucosylation (%) vs. the amount of GDP fucose as a % of control.

In one embodiment, said second preselected level of GDP-fucose is selected from a level:

i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;

ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;

iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level;

iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;

v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level; or

vi.b) approximately equal to or greater than point C on the curve in FIG. 1, or greater than or equal to an analogous point on a plot of the amount of fucosylation (%) vs. the amount of GDP fucose as a % of control.

In an embodiment the first level is i.a and the second level is selected from i.b, ii.b, iii.b, iv.b, v.b, and vi.b.

In an embodiment the first level is ii.a and the second level is selected from i.b, ii.b, iii.b, iv.b, v.b, and vi.b.

In an embodiment the first level is ii.1.a and the second level is selected from i.b, ii.b, iii.b, iv.b, v.b, and vi.b.

In an embodiment the first level is iii.a and the second level is selected from i.b, ii.b, iii.b, iv.b, v.b, and vi.b.

In an embodiment the first level is iii.1.a and the second level is selected from i.b, ii.b, iii.b, iv.b, v.b, and vi.b.

In an embodiment the first level is iv.a and the second level is selected from i.b, ii.b, iii.b, iv.b, v.b, and vi.b.

In an embodiment the first level is v.a and the second level is selected from i.b, ii.b, iii.b, iv.b, v.b, and vi.b.

In an embodiment the first level is vi.a and the second level is selected from i.b, ii.b, iii.b, iv.b, v.b, and vi.b.

In an embodiment the first level is selected from i.a, ii.a, ii.1.a, iii.a, iii.1.a, iv.a, v.a, and vi.a and the second level is i.b.

In an embodiment the first level is selected from i.a, ii.a, ii.1.a, iii.a, iii.1.a, iv.a, v.a, and vi.a and the second level is ii.b.

In an embodiment the first level is selected from i.a, ii.a, ii.1.a, iii.a, iii.1.a, iv.a, v.a, and vi.a and the second level is iii.b.

In an embodiment the first level is selected from i.a, ii.a, ii.1.a, iii.a, iii.1.a, iv.a, v.a, and vi.a and the second level is iv.b.

In an embodiment the first level is selected from i.a, ii.a, ii.1.a, iii.a, iii.1.a, iv.a, v.a, and vi.a and the second level is v.b.

In an embodiment the first level is selected from i.a, ii.a, ii.1.a, iii.a, iii.1.a, iv.a, v.a, and vi.a and the second level is vi.b.

In one embodiment, the level of GDP-fucose is selected to be outside the range between A and B on the curve in FIG. 1 (as relatively small changes in GDP-fucose will result in relatively large changes in the amount of fucosylation. In an embodiment the level is also less than B.). In another embodiment, the level of GDP-fucose is reduced by a predetermined level, e.g., in comparison with a reference. In another embodiment, the reference is the amount present in a cell or batch of cultured cells, e.g., a CHO cell or batch of cultured cells, cultured under reference conditions but otherwise the same or essentially the same as the cell cultured under conditions that result in said level of GDP-fucose. In another embodiment, the level of GDP-fucose is reduced by, as much as, or more than, 10, 15, 20, 25, 30, 35, 40, 45, 50, 60, 70, 80 or 90%, as compared to the reference.

In one embodiment, the method further comprises evaluating the glycoprotein for a parameter related to fucosylation, e.g., the amount of fucosylation in the glycan complement, the amount or fucosylation on a component of the glycan complement, or the amount of fucosylation on a glycan component, e.g., in a preparation of glycoproteins.

In one embodiment, the method further comprises evaluating the glycoprotein for a parameter related to fucosylation, e.g., the proportion of a preselected glycan component which bears a fucosyl moiety, e.g., at a selected position on the glycan component, e.g., in a preparation of glycoproteins.

In one embodiment, the level of fucosylation at one, two, three, or more preselected amino acid residues is evaluated. In another embodiment, the level of fucosylation is reduced by a predetermined level in comparison with a reference. In another embodiment, the reference is the amount present in a cell or batch of cultured cells, e e.g., a CHO cell or batch of cultured cells, cultured under reference conditions but otherwise the same or essentially the same as the cell cultured under conditions that result in said level of GDP-fucose. In another embodiment, the level of fucosylation is reduced by, as much as, or more than, 10, 15, 20, 25, 30, 35, 40, 45, 50, 60, 70, 80 or 90%, as compared to the reference.

In one embodiment, wherein X_{F} is greater than X_{G},

and wherein,

X_{F} is the % or proportion of reduction in the level of fucosylation (e.g., as compared to the level of fucosylation in a cell or batch of cultured cells cultured under reference conditions); and

X_{G} is the % or proportion of reduction in the level of GDP fucose (as compared to the level of GDP fucose in a cell or batch of cultured cells cultured under reference conditions).

In an embodiments, an inhibitor, e.g., an inhibitor of GMD, FX, fucokinase, GFPP, GDP-fucose synthetase, or enzymes involved in the biosynthesis of GDP-mannose, is used, e.g., in the culture medium, to lower the levels of the GDP-fucose. In an embodiment the inhibitor can be guanosine-5′-O-(2-thiodiphosphate)-fucose, guanosine-5′-O-(2-thiodiphosphate)-mannose, pyridoxal-5′-phosphate, GDP-4-dehydro-6-L-deoxygalactose, GDP-L-fucose, guanosine diphosphate (GDP), guanosine monophosphate (GMP), GDP-D-glucose, or p-chloromercuriphenylsulfonate EDTA.

In an embodiment the media contains a substance that can increase the level of GDP-fucose, e.g., butyrate or fucose.

In one embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the cell is a Chinese Hamster Ovary (CHO) cell. In another embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the glycoprotein is selected from Table 1.

In one embodiment, the method further comprises culturing a plurality of the cells and separating as much as, or at least, 1, 10, 100, 1,000, or 10,000 grams of the glycoprotein from the cells. In another embodiment, the method further comprises combining the glycoprotein having reduced fucosylation with a pharmaceutically acceptable component and, e.g., formulating the glycoprotein having reduced fucosylation into a pharmaceutically acceptable formulation.

In one embodiment, the glycoprotein is analyzed by one or more of HPLC, CE, MALDI-MS and NMR.

In one embodiment, the method further comprises memorializing the result of the evaluation.

In one embodiment, the level of fucosylation at one, two, three, or more preselected amino acid residues is evaluated.

In one embodiment, the method further comprises providing a value for a parameter associated with a compound other than GDP-fucose, wherein a parameter for the compound, e.g., the level of the compound, is correlated to the level of GDP-fucose.

In another embodiment, the method further comprises providing a comparison of the value with a reference value, wherein optionally, a preselected relationship of the value to the reference value, e.g., greater than, equal to, or less than, is indicative of whether the level of GDP fucose is above, at or below the second level. In another embodiment, the method further comprises, responsive to the result of the comparison, increasing the level of GDP-fucose, decreasing the level of GDP-fucose or continuing cell culture without intervening to change the level of GDP-fucose. In one embodiment, the compound other than GDP-fucose is GDP-mannose. In one embodiment, the compound other than GDP-fucose is GDP-mannose and the parameter is the level of GDP-mannose.

In one embodiment, the method further comprises providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose. In one embodiment, the method comprises continuing to culture said cells, and repeating the steps above.

In another aspect, the invention features, a reaction mixture containing one or more of a cell or batch of cultured cells having a manipulation, culture medium, and a glycoprotein having reduced fucosylation produced by the cell.

In another aspect, the invention features, a device for the culture of cells comprising one or more of a cell having a manipulation, culture medium, and a glycoprotein having reduced fucosylation produced by the cell.

When reduced fucosylation is desired, methods described herein allow selecting a cell which makes a desired protein, selecting a manipulation(s) that gives reduced fucosylation according to the invention, providing the manipulations to a cell, and optionally, using the cell for making the protein. Although useful in other applications, this method can be used to use and/or further modify an existing cell line that has been used to make a protein not having reduced fucosylation.

Accordingly, in another aspect, the invention features, a method of making, or providing, a glycoprotein, or preparation thereof, having a glycan structure having reduced fucosylation, comprising:

optionally, selecting a glycan structure having reduced fucosylation, e.g., from a list comprising a plurality of glycan structures having reduced fucosylation (in embodiments the list is provided), and optionally memorializing said selected glycan structure;

selecting a cell, preferably on the basis that it produces a protein having the primary amino acid sequence of said glycoprotein but which protein lacks said glycan structure having reduced fucosylation;

optionally, selecting a manipulation, e.g., selecting the manipulation on the basis that the manipulation decreases fucosylation and which manipulation thereby promotes the formation of said glycan structure having reduced fucosylation (in embodiments the manipulation is from a list comprising a plurality of manipulations, and in embodiments the list is provided);

providing said manipulation to said cell to provide a cell having or subject to a manipulation that decreases the level of fucosylation and which manipulation thereby promotes the formation of said glycan structure having reduced fucosylation;

culturing said selected cell, e.g., to provide a batch of cultured cells;

optionally, separating the glycoprotein having a glycan structure from at least one component with which the cell or batch of cultured cells was cultured;

optionally, analyzing said glycoprotein to confirm the presence of the glycan structure having reduced fucosylation;

thereby making, or providing, a glycoprotein having a glycan structure having reduced fucosylation, e.g., by inhibiting or promoting the addition of a fucose moiety to a protein or glycoprotein.

In one embodiment, the method further comprises evaluating a glycan on the surface of said cell or batch of cultured cells in order to determine if the glycoprotein produced by said cell or batch of cultured cells has reduced fucosylation. In another embodiment, said evaluation comprises evaluating a glycan on the surface of said cell or batch of cultured cells, to determine a property of said glycan, comparing the property to a reference, to thereby determine if said glycan structure is present on the product.

In one embodiment, the manipulation results in a level of GDP-fucose in said cell that is below a first preselected level and, in embodiments, above a second preselected level. In embodiment said first preselected level of GDP-fucose is selected from a level that is:

i.a) approximately equal to or less than 80%, 70% or 60% of a reference level, e.g., the level in said cell or batch of cultured cells, e.g., a cell or batch of cultured cells which is otherwise similar, without the manipulation;

ii.a) approximately equal to, or less than, the point of maximum curvature above the inflection point (e.g., the inflection point in the second phase) on a graph of the amount of fucosylation vs. decrease in GDP-fucose;

ii.1.a) approximately equal to, or less than, the lowest level that results in a normal (e.g., that seen in an un-manipuated cell) level of fucosylation;

iii.a) approximately equal to or less than the point of maximum curvature below the inflection point on a graph of the amount of fucosylation vs. decrease in GDP-fucose;

iii.1.a) approximately equal to, or less than, the highest level that results in no further reduction in fucosylation;

iv.a) approximately equal to or less than point A on the curve in FIG. 1, or less than or equal to an analogous point on a plot of the amount of fucosylation (%) vs. the amount of GDP fucose as a % of control;

v.a) approximately equal to or less than that corresponding to an amount between points A and B on the curve in FIG. 1, or less than or equal to an analogous point on a plot of the amount of fucosylation (%) vs. the amount of GDP fucose as a % of control; or

vi.a) approximately equal to or less than point B on the curve in FIG. 1, or less than or equal to an analogous point on a plot of the amount of fucosylation (%) vs. the amount of GDP fucose as a % of control.

In one embodiment, said second preselected level of GDP-fucose is selected from a level:

i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;

ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;

iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level;

iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;

v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level; or

vi.b) approximately equal to or greater than point C on the curve in FIG. 1, or greater than or equal to an analogous point on a plot of the amount of fucosylation (%) vs. the amount of GDP fucose as a % of control.

In an embodiment the first level is i.a and the second level is selected from i.b, ii.b, iii.b, iv.b, v.b, and vi.b.

In an embodiment the first level is ii.a and the second level is selected from i.b, ii.b, iii.b, iv.b, v.b, and vi.b.

In an embodiment the first level is ii.1.a and the second level is selected from i.b, ii.b, iii.b, iv.b, v.b, and vi.b.

In an embodiment the first level is iii.a and the second level is selected from i.b, ii.b, iii.b, iv.b, v.b, and vi.b.

In an embodiment the first level is iii.1.a and the second level is selected from i.b, ii.b, iii.b, iv.b, v.b, and vi.b.

In an embodiment the first level is iv.a and the second level is selected from i.b, ii.b, iii.b, iv.b, v.b, and vi.b.

In an embodiment the first level is v.a and the second level is selected from i.b, ii.b, iii.b, iv.b, v.b, and vi.b.

In an embodiment the first level is vi.a and the second level is selected from i.b, ii.b, iii.b, iv.b, v.b, and vi.b.

In an embodiment the first level is selected from i.a, ii.a, ii.1.a, iii.a, iii.1.a, iv.a, v.a, and vi.a and the second level is i.b.

In an embodiment the first level is selected from i.a, ii.a, ii.1.a, iii.a, iii.1.a, iv.a, v.a, and vi.a and the second level is ii.b.

In an embodiment the first level is selected from i.a, ii.a, ii.1.a, iii.a, iii.1.a, iv.a, v.a, and vi.a and the second level is iii.b.

In an embodiment the first level is selected from i.a, ii.a, ii.1.a, iii.a, iii.1.a, iv.a, v.a, and vi.a and the second level is iv.b.

In an embodiment the first level is selected from i.a, ii.a, ii.1.a, iii.a, iii.1.a, iv.a, v.a, and vi.a and the second level is v.b.

In an embodiment the first level is selected from i.a, ii.a, ii.1.a, iii.a, iii.1.a, iv.a, v.a, and vi.a and the second level is vi.b.

In an embodiment the level of GDP-fucose is between point B and C on the curve in FIG. 1 or in an analogous range on a plot of the amount of fucosylation (%) vs. the amount of GDP fucose as a % of control.

In an embodiment the level of GDP-fucose is between point A and C on the curve in FIG. 1 or in an analogous range on a plot of the amount of fucosylation (%) vs. the amount of GDP fucose as a % of control.

In one embodiment, the level of GDP-fucose is selected to be outside the range between A and B on the curve in FIG. 1 (as relatively small changes in GDP-fucose will result in relatively large changes in the amount of fucosylation. In an embodiment the level is also less than B.) In another embodiment, the level of GDP-fucose is reduced by a predetermined level, e.g., in comparison with a reference. In another embodiment, the reference is the amount present in a cell or batch of cultured cells, e.g., a CHO cell or batch of cultured cells, lacking the manipulation but otherwise the same or essentially the same as the cell having the manipulation. In another embodiment, the level of GDP-fucose is reduced by, as much as, or more than, 10, 15, 20, 25, 30, 35, 40, 45, 50, 60, 70, 80 or 90%, as compared to the reference.

In one embodiment, the method further comprises evaluating the glycoprotein for a parameter related to fucosylation, e.g., the amount of fucosylation in the glycan complement, the amount or fucosylation on a component of the glycan complement, or the amount of fucosylation on a glycan component, e.g., in a preparation of glycoproteins.

In one embodiment, the method further comprises evaluating the glycoprotein for a parameter related to fucosylation, e.g., the proportion of a preselected glycan component which bears a fucosyl moiety, e.g., at a selected position on the glycan component, e.g., in a preparation of glycoproteins.

In one embodiment, the level of fucosylation at one, two, three, or more preselected amino acid residues is evaluated. In another embodiment, the level of fucosylation is reduced by a predetermined level in comparison with a reference. In another embodiment, the reference is the amount present in a cell or batch of cultured cells, e.g., a CHO cell or batch of cultured cells, lacking the manipulation but otherwise the same or essentially the same as the cell or batch of cultured cells having the manipulation. In another embodiment, the level of fucosylation is reduced by, as much as, or more than, 10, 15, 20, 25, 30, 35, 40, 45, 50, 60, 70, 80 or 90%, as compared to the reference.

In one embodiment, X_{F} is greater than X_{G},

and wherein,

X_{F} is the % or proportion of reduction in the level of fucosylation (e.g., as compared to the level of fucosylation in a cell or batch of cultured cells lacking the manipulation); and

X_{G} is the % or proportion of reduction in the level of GDP fucose (as compared to the level of GDP fucose in a cell or batch of cultured cells lacking the manipulation).

In one embodiment, said manipulation is not a genetic lesion or the presence of an siRNA that reduces the level of an enzyme that promotes formation of GDP-fucose, or the attachment of a fucosyl moiety. For example, the manipulation is not a lesion that decreases the expression of GMD, FX, fucokinase, GFPP, GDP-synthetase, a fucosyltransferase or a GDP-Fucose transporter. In another embodiment, the cell or batch of cultured cells is wild-type for one or all of GMD, FX, fucokinase, GFPP, GDP-synthetase, a fucosyltransferase or a GDP-Fucose transporter. In another embodiment, the cell or batch of cultured cells does not include an siRNA that targets GMD, FX, fucokinase, GFPP, GDP-synthetase, a fucosyltransferase or a GDP-Fucose transporter. In another embodiment, absent the manipulation, the level of fucosylation is substantially the same as the level in a wild-type cell. In another embodiment, the manipulated cell carries no mutation that substantially lowers GDP-fucose levels. In another embodiment, the manipulated cell has no siRNA that substantially lowers GDP-fucose levels.

In one embodiment, the cell has a mutation (e.g., a genetically engineered change) that decreases the level of GDP-fucose. Exemplary mutations include those which alter the activity of GMD, FX, fucokinase, GFPP, GDP-synthetase, a fucosyltransferase or a GDP-Fucose transporter. The mutation can be in the structural gene which encodes GMD, FX, fucokinase, GFPP, GDP-synthetase, a fucosyltransferase or a GDP-Fucose transporter. Such mutations can decrease the activity of the encoded protein. The decrease can be partial or complete. Such mutations can act, e.g., by altering the catalytic activity of the protein or by altering its half-life. Other exemplary mutations can be in a sequence that control expression of GMD, FX, fucokinase, GFPP, GDP-synthetase, a fucosyltransferase or a GDP-Fucose transporter. These can be mutations that completely, or partially, reduce the expression of the gene, at the RNA or protein level. Such mutations include deletion or other mutations in endogenous of control sequence. Such mutations also include the introduction of heterologous control sequence, e.g., the introduction of heterologous control regions, e.g., a sequence that will give a desired level of expression. (A heterologous control sequence is a sequence other than a sequence naturally associated with and operably linked to the structural gene.) In embodiments the manipulation comprises a mutation in the structural region or in a control sequence operably linked to the gene.

In an embodiment a cell having a mutation that that decreases the level of GDP-fucose, e.g., a mutation that decreases the activity of GMD, FX, fucokinase, GFPP, GDP-synthetase, a fucosyltransferase or a GDP-Fucose transporter is cultured in the presence of a substance, e.g., fucose, that results in a GDP-fucose level and/or a fucosylation level described herein. In an embodiment the cell includes a mutation that, in the absence of fucose in the culture medium, would result in a cell having an unacceptably low level of GDP-fucose. When, however, cultured under the appropriate conditions, e.g., media supplemented, e.g., with fucose, that cell can exhibit a desired level of GDP-fucose, e.g., a level of GDP-fucose described herein. Thus, fucose or another substance is present in the culture medium at a level that results in a level of GDP-fucose recited above.

In another embodiment, the manipulation is the presence of an siRNA that reduces the level of an enzyme that promotes formation of GDP-fucose, or the attachment of a fucosyl moiety, e.g., an siRNA that targets GMD, FX, fucokinase, GFPP, GDP-synthetase, a fucosyltransferase or a GDP-Fucose transporter, and fucose or another substance is present in the culture medium at a level that results in formation of said glycoprotein having a glycan structure having reduced fucosylation.

In one embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the cell is a Chinese Hamster Ovary (CHO) cell. In another embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the glycoprotein is selected from Table 1.

In one embodiment, the method further comprises culturing a plurality of the cells and separating as much as, or at least, 1, 10, 100, 1,000, or 10,000 grams of the glycoprotein from the cells. In another embodiment, the method further comprises combining the glycoprotein having reduced fucosylation with a pharmaceutically acceptable component and, e.g., formulating the glycoprotein having reduced fucosylation into a pharmaceutically acceptable formulation.

In one embodiment, the glycoprotein is analyzed by one or more of HPLC, CE, MALDI-MS and NMR.

In one embodiment, the method further comprises memorializing the result of the evaluation.

In one embodiment, the manipulation is, or is the product of, a selection for reduced levels of GDP-fucose. In another embodiment, the manipulation is, or is the product of, a selection for reduced fucosylation of a glycoprotein. In another embodiment, the manipulation comprises contact with, or inclusion in or on the cell or batch of cultured cells, of an exogenous inhibitor of an enzyme involved in GDP-fucose biosynthesis, e.g., a specific or non-specific inhibitor.

In one embodiment, the level of fucosylation at one, two, three, or more preselected amino acid residues is evaluated.

In one embodiment, one or more of said cell or said batch of cultured cells, said manipulation, and said glycoprotein, is selected on the basis that it or the combination will provide a glycoprotein having reduced fucosylation.

In one embodiment, one or more of said cell or said batch of cultured cells, said manipulation (or manipulations), and said glycoprotein, is selected on the basis that it or the combination will provide a level of GDP-fucose described herein, e.g., a level which gives a minimal level of fucosylation (e.g., with reference to a curve analogous to that in FIG. 1, the level is to the right of point B) but which is above a preselected level. E.g., in a an embodiment the level is above a level that gives an unwanted decrease in the level of GDP-mannose, e.g., a decrease in GDP-mannose that is equal to, or more than, 10%, 20%, 30%, 40% or 50% as compared to a reference level, e.g., the level of GDP-mannose in a cell or batch of cultured cells, e.g., a cell or batch of cultured cells which is otherwise similar, without the manipulation.

In some embodiments the level is above a level that gives an unwanted increase in the level of GDP-mannose, e.g., an increase in GDP-mannose that is equal to, or more than, about 2×, 3×, 4×, 5×, ×, 7×, 8×, 9×, or 10× of a reference level, e.g., the level of GDP-mannose in a cell or batch of cultured cells, e.g., a cell or batch of cultured cells which is otherwise similar, without the manipulation.

In one embodiment, the method further comprises providing a value for a parameter associated with a compound other than GDP-fucose, wherein a parameter for the compound, e.g., the level of the compound, is correlated to the level of GDP-fucose.

In another embodiment, the method further comprises providing a comparison of the value with a reference value, wherein optionally, a preselected relationship of the value to the reference value, e.g., greater than, equal to, or less than, is indicative of whether the level of GDP fucose is above, at or below the second level. In another embodiment, the method further comprises, responsive to the result of the comparison, increasing the level of GDP-fucose, decreasing the level of GDP-fucose or continuing cell culture without intervening to change the level of GDP-fucose. In one embodiment, the compound other than GDP-fucose is GDP-mannose. In one embodiment, the compound other than GDP-fucose is GDP-mannose and the parameter is the level of GDP-mannose.

In one embodiment, the method further comprises providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose. In one embodiment, the method comprises continuing to culture said cells, and repeating the steps above.

In an embodiments, an inhibitor, e.g., an inhibitor of GMD, FX, fucokinase, GFPP, GDP-fucose synthetase, or enzymes involved in the biosynthesis of GDP-mannose, is used, e.g., in the culture medium, to lower the levels of the GDP-fucose. In an embodiment the inhibitor can be guanosine-5′-O-(2-thiodiphosphate)-fucose, guanosine-5′-O-(2-thiodiphosphate)-mannose, pyridoxal-5′-phosphate, GDP-4-dehydro-6-L-deoxygalactose, GDP-L-fucose, guanosine diphosphate (GDP), guanosine monophosphate (GMP), GDP-D-glucose, or p-chloromercuriphenylsulfonate EDTA. The inhibitor can be used with a cell which is mutant or wildtype for one or more GMD, FX, fucokinase, GFPP, GDP-synthetase, a fucosyltransferase or a GDP-Fucose transporter.

In an embodiment the media contains a substance that can increase the level of GDP-fucose, e.g., butyrate or fucose. Such media can be used, e.g., with a cell having a mutation that eliminates or decreased the activity of one or more of GMD, FX, fucokinase, GFPP, GDP-synthetase, a fucosyltransferase or a GDP-Fucose transporter.

When reduced fucosylation is desired, methods described herein allow selecting a cell which makes the desired protein. Although useful in other applications, this method can be used to use and/or further modify an existing cell line that has been used to make a protein not having reduced fucosylation.

In one aspect, the invention features a method of providing a cell that makes a glycoprotein having a glycan structure having reduced fucosylation, comprising:

optionally, selecting a glycan structure having reduced fucosylation, e.g., from a list comprising a plurality of glycan structures having reduced fucosylation (in embodiments the list is provided), and optionally memorializing said selected glycan structure;

selecting a cell, preferably on the basis that it produces a protein having the primary amino acid sequence of said glycoprotein but which protein lacks said glycan structure having reduced fucosylation;

optionally, selecting a manipulation, e.g., selecting the manipulation on the basis that the manipulation decreases the level of fucosylation, and which manipulation thereby promotes the formation of said glycan structure having reduced fucosylation (in embodiments the manipulation is from a list comprising a plurality of manipulations, and in embodiments the list is provided);

providing said manipulation to said cell to provide a cell having or subject to a manipulation that decreases fucosylation, and which manipulation thereby promotes the formation of said glycan structure having reduced fucosylation;

optionally producing glycoprotein from said cell and determining if said glycoprotein has said glycan structure having reduced fucosylation, thereby providing a cell that makes a glycoprotein having a glycan structure.

In one embodiment, the method further comprises evaluating a glycan on the surface of said cell or batch of cultured cells in order to determine if the glycoprotein produced by said cell or batch of cultured cells has reduced fucosylation. In another embodiment, said evaluation comprises evaluating a glycan on the surface of said cell or batch of cultured cells, to determine a property of said glycan, comparing the property to a reference, to thereby determine if said glycan structure is present on the product.

In one embodiment, the manipulation results in a level of GDP-fucose in said cell that is below a first preselected level and, in embodiments, above a second preselected level. In one embodiment, said first preselected level of GDP-fucose is selected from a level that is:

i.a) approximately equal to or less than 80%, 70% or 60% of a reference level, e.g., the level in said cell or batch of cultured cells, e.g., a cell or batch of cultured cells which is otherwise similar, without the manipulation;

ii.a) approximately equal to, or less than, the point of maximum curvature above the inflection point (e.g., the inflection point in the second phase) on a graph of the amount of fucosylation vs. decrease in GDP-fucose;

ii.1.a) approximately equal to, or less than, the lowest level that results in a normal (e.g., that seen in an un-manipuated cell) level of fucosylation;

iii.a) approximately equal to or less than the point of maximum curvature below the inflection point on a graph of the amount of fucosylation vs. decrease in GDP-fucose;

iii.1.a) approximately equal to, or less than, the highest level that results in no further reduction in fucosylation;

iv.a) approximately equal to or less than point A on the curve in FIG. 1, or less than or equal to an analogous point on a plot of the amount of fucosylation (%) vs. the amount of GDP fucose as a % of control;

v.a) approximately equal to or less than that corresponding to an amount between points A and B on the curve in FIG. 1, or less than or equal to an analogous point on a plot of the amount of fucosylation (%) vs. the amount of GDP fucose as a % of control; or

vi.a) approximately equal to or less than point B on the curve in FIG. 1, or less than or equal to an analogous point on a plot of the amount of fucosylation (%) vs. the amount of GDP fucose as a % of control.

In one embodiment, said second preselected level of GDP-fucose is selected from a level:

i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;

ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;

iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level;

iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;

v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level; or

vi.b) approximately equal to or greater than point C on the curve in FIG. 1, or greater than or equal to an analogous point on a plot of the amount of fucosylation (%) vs. the amount of GDP fucose as a % of control.

In an embodiment the first level is i.a and the second level is selected from i.b, ii.b, iii.b, iv.b, v.b, and vi.b.

In an embodiment the first level is ii.a and the second level is selected from i.b, ii.b, iii.b, iv.b, v.b, and vi.b.

In an embodiment the first level is ii.1.a and the second level is selected from i.b, ii.b, iii.b, iv.b, v.b, and vi.b.

In an embodiment the first level is iii.a and the second level is selected from i.b, ii.b, iii.b, iv.b, v.b, and vi.b.

In an embodiment the first level is iii.1.a and the second level is selected from i.b, ii.b, iii.b, iv.b, v.b, and vi.b.

In an embodiment the first level is iv.a and the second level is selected from i.b, ii.b, iii.b, iv.b, v.b, and vi.b.

In an embodiment the first level is v.a and the second level is selected from i.b, ii.b, iii.b, iv.b, v.b, and vi.b.

In an embodiment the first level is vi.a and the second level is selected from i.b, ii.b, iii.b, iv.b, v.b, and vi.b.

In an embodiment the first level is selected from i.a, ii.a, ii.1.a, iii.a, iii.1.a, iv.a, v.a, and vi.a and the second level is i.b.

In an embodiment the first level is selected from i.a, ii.a, ii.1.a, iii.a, iii.1.a, iv.a, v.a, and vi.a and the second level is ii.b.

In an embodiment the first level is selected from i.a, ii.a, ii.1.a, iii.a, iii.1.a, iv.a, v.a, and vi.a and the second level is iii.b.

In an embodiment the first level is selected from i.a, ii.a, ii.1.a, iii.a, iii.1.a, iv.a, v.a, and vi.a and the second level is iv.b.

In an embodiment the first level is selected from i.a, ii.a, ii.1.a, iii.a, iii.1.a, iv.a, v.a, and vi.a and the second level is v.b.

In an embodiment the first level is selected from i.a, ii.a, ii.1.a, iii.a, iii.1.a, iv.a, v.a, and vi.a and the second level is vi.b.

In an embodiment the level of GDP-fucose is between point B and C on the curve in FIG. 1 or in an analogous range on a plot of the amount of fucosylation (%) vs. the amount of GDP fucose as a % of control.

In an embodiment the level of GDP-fucose is between point A and C on the curve in FIG. 1 or in an analogous range on a plot of the amount of fucosylation (%) vs. the amount of GDP fucose as a % of control.

In one embodiment, the level of GDP-fucose is selected to be outside the range between A and B on the curve in FIG. 1 (as relatively small changes in GDP-fucose will result in relatively large changes in the amount of fucosylation. In an embodiment the level is also less than B.) In another embodiment, the level of GDP-fucose is reduced by a predetermined level, e.g., in comparison with a reference. In another embodiment, the reference is the amount present in a cell or batch of cultured cells, e.g., a CHO cell or batch of cultured cells, lacking the manipulation but otherwise the same or essentially the same as the cell having the manipulation. In another embodiment, the level of GDP-fucose is reduced by, as much as, or more than, 10, 15, 20, 25, 30, 35, 40, 45, 50, 60, 70, 80 or 90%, as compared to the reference.

In one embodiment, the method further comprises evaluating the glycoprotein for a parameter related to fucosylation, e.g., the amount of fucosylation in the glycan complement, the amount or fucosylation on a component of the glycan complement, or the amount of fucosylation on a glycan component, e.g., in a preparation of glycoproteins.

In one embodiment, the method further comprises evaluating the glycoprotein for a parameter related to fucosylation, e.g., the proportion of a preselected glycan component which bears a fucosyl moiety, e.g., at a selected position on the glycan component, e.g., in a preparation of glycoproteins.

In one embodiment, the level of fucosylation at one, two, three, or more preselected amino acid residues is evaluated. In another embodiment, the level of fucosylation is reduced by a predetermined level in comparison with a reference. In another embodiment, the reference is the amount present in a cell or batch of cultured cells, e.g., a CHO cell or batch of cultured cells, lacking the manipulation but otherwise the same or essentially the same as the cell or batch of cultured cells having the manipulation. In another embodiment, the level of fucosylation is reduced by, as much as, or more than, 10, 15, 20, 25, 30, 35, 40, 45, 50, 60, 70, 80 or 90%, as compared to the reference.

In one embodiment, X_{F} is greater than X_{G},

and wherein,

X_{F} is the % or proportion of reduction in the level of fucosylation (e.g., as compared to the level of fucosylation in a cell or batch of cultured cells lacking the manipulation); and

X_{G} is the % or proportion of reduction in the level of GDP fucose (as compared to the level of GDP fucose in a cell or batch of cultured cells lacking the manipulation).

In one embodiment, said manipulation is not a genetic lesion or the presence of an siRNA that reduces the level of an enzyme that promotes formation of GDP-fucose, or the attachment of a fucosyl moiety. For example, the manipulation is not a lesion that decreases the expression of GMD, FX, fucokinase, GFPP, GDP-synthetase, a fucosyltransferase or a GDP-Fucose transporter. In another embodiment, the cell or batch of cultured cells is wild-type for one or all of GMD, FX, fucokinase, GFPP, GDP-synthetase, a fucosyltransferase or a GDP-Fucose transporter. In another embodiment, the cell or batch of cultured cells does not include an siRNA that targets GMD, FX, fucokinase, GFPP, GDP-synthetase, a fucosyltransferase or a GDP-Fucose transporter. In another embodiment, absent the manipulation, the level of fucosylation is substantially the same as the level in a wild-type cell. In another embodiment, the manipulated cell carries no mutation that substantially lowers GDP-fucose levels. In another embodiment, the manipulated cell has no siRNA that substantially lowers GDP-fucose levels.

In one embodiment, the cell has a mutation (e.g., a genetically engineered change) that decreases the level of GDP-fucose. Exemplary mutations include those which alter the activity of GMD, FX, fucokinase, GFPP, GDP-synthetase, a fucosyltransferase or a GDP-Fucose transporter.

The mutation can be in the structural gene which encodes GMD, FX, fucokinase, GFPP, GDP-synthetase, a fucosyltransferase or a GDP-Fucose transporter. Such mutations can decrease the activity of the encoded protein. The decrease can be partial or complete. Such mutations can act, e.g., by altering the catalytic activity of the protein or by altering its half-life. Other exemplary mutations can be in a sequence that control expression of GMD, FX, fucokinase, GFPP, GDP-synthetase, a fucosyltransferase or a GDP-Fucose transporter. These can be mutations that completely, or partially, reduce the expression of the gene, at the RNA or protein level. Such mutations include deletion or other mutations in endogenous of control sequence. Such mutations also include the introduction of heterologous control sequence, e.g., the introduction of heterologous control regions, e.g., a sequence that will give a desired level of expression. (A heterologous control sequence is a sequence other than a sequence naturally associated with and operably linked to the structural gene.) In embodiments the manipulation comprises a mutation in the structural region or in a control sequence operably linked to the gene.

In an embodiment a cell having a mutation that that decreases the level of GDP-fucose, e.g., a mutation that decreases the activity of GMD, FX, fucokinase, GFPP, GDP-synthetase, a fucosyltransferase or a GDP-Fucose transporter is cultured in the presence of a substance, e.g., fucose, that results in a GDP-fucose level and/or a fucosylation level described herein. In an embodiment the cell includes a mutation that, in the absence of fucose in the culture medium, would result in a cell having an unacceptably low level of GDP-fucose. When, however, cultured under the appropriate conditions, e.g., media supplemented, e.g., with fucose, that cell can exhibit a desired level of GDP-fucose, e.g., a level of GDP-fucose described herein. Thus, fucose or another substance is present in the culture medium at a level that results in a level of GDP-fucose recited above.

In another embodiment, the manipulation is the presence of an siRNA that reduces the level of an enzyme that promotes formation of GDP-fucose, or the attachment of a fucosyl moiety, e.g., an siRNA that targets GMD, FX, fucokinase, GFPP, GDP-synthetase, a fucosyltransferase or a GDP-Fucose transporter, and fucose or another substance is present in the culture medium at a level that results in formation of said glycoprotein having a glycan structure having reduced fucosylation.

In one embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the cell is a Chinese Hamster Ovary (CHO) cell. In another embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the glycoprotein is selected from Table 1.

In one embodiment, the method further comprises culturing a plurality of the cells and separating as much as, or at least, 1, 10, 100, 1,000, or 10,000 grams of the glycoprotein from the cells. In another embodiment, the method further comprises combining the glycoprotein having reduced fucosylation with a pharmaceutically acceptable component and, e.g., formulating the glycoprotein having reduced fucosylation into a pharmaceutically acceptable formulation.

In one embodiment, the glycoprotein is analyzed by one or more of HPLC, CE, MALDI-MS and NMR.

In one embodiment, the method further comprises memorializing the result of the evaluation.

In one embodiment, the manipulation is, or is the product of, a selection for reduced levels of GDP-fucose. In another embodiment, the manipulation is, or is the product of, a selection for reduced fucosylation of a glycoprotein. In another embodiment, the manipulation comprises contact with, or inclusion in or on the cell or batch of cultured cells, of an exogenous inhibitor of an enzyme involved in GDP-fucose biosynthesis, e.g., a specific or non-specific inhibitor.

In one embodiment, the level of fucosylation at one, two, three, or more preselected amino acid residues is evaluated.

In one embodiment, one or more of said cell or said batch of cultured cells, said manipulation, and said glycoprotein, is selected on the basis that it or the combination will provide a glycoprotein having reduced fucosylation.

In one embodiment, one or more of said cell or said batch of cultured cells, said manipulation (or manipulations), and said glycoprotein, is selected on the basis that it or the combination will provide a level of GDP-fucose described herein, e.g., a level which gives a minimal level of fucosylation (e.g., with reference to a curve analogous to that in FIG. 1, the level is to the right of point B) but which is above a preselected level, e.g., above a level that gives an unwanted decrease in the level of GDP-mannose. E.g., the level is above a level that gives a decrease in GDP-mannose that is equal to, or more than, 10%, 20%, 30%, 40% or 50% as compared to a reference level, e.g., the level of GDP-mannose in a cell or batch of cultured cells, e.g., a cell or batch of cultured cells which is otherwise similar, without the manipulation.

In some embodiments the level is above a level that gives an unwanted increase in the level of GDP-mannose, e.g., an increase in GDP-mannose that is equal to, or more than, about 2×, 3×, 4×, 5×, ×, 7×, 8×, 9×, or 10× of a reference level, e.g., the level of GDP-mannose in a cell or batch of cultured cells, e.g., a cell or batch of cultured cells which is otherwise similar, without the manipulation.

In one embodiment, the method further comprises providing a value for a parameter associated with a compound other than GDP-fucose, wherein a parameter for the compound, e.g., the level of the compound, is correlated to the level of GDP-fucose.

In another embodiment, the method further comprises providing a comparison of the value with a reference value, wherein optionally, a preselected relationship of the value to the reference value, e.g., greater than, equal to, or less than, is indicative of whether the level of GDP fucose is above, at or below the second level. In another embodiment, the method further comprises, responsive to the result of the comparison, increasing the level of GDP-fucose, decreasing the level of GDP-fucose or continuing cell culture without intervening to change the level of GDP-fucose. In one embodiment, the compound other than GDP-fucose is GDP-mannose. In one embodiment, the compound other than GDP-fucose is GDP-mannose and the parameter is the level of GDP-mannose.

In one embodiment, the method further comprises providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose. In one embodiment, the method comprises continuing to culture said cells, and repeating the steps above.

In an embodiments, an inhibitor, e.g., an inhibitor of GMD, FX, fucokinase, GFPP, GDP-fucose synthetase, or enzymes involved in the biosynthesis of GDP-mannose, is used, e.g., in the culture medium, to lower the levels of the GDP-fucose. In an embodiment the inhibitor can be guanosine-5′-O-(2-thiodiphosphate)-fucose, guanosine-5′-O-(2-thiodiphosphate)-mannose, pyridoxal-5′-phosphate, GDP-4-dehydro-6-L-deoxygalactose, GDP-L-fucose, guanosine diphosphate (GDP), guanosine monophosphate (GMP), GDP-D-glucose, or p-chloromercuriphenylsulfonate EDTA. The inhibitor can be used with a cell which is mutant or wildtype for one or more GMD, FX, fucokinase, GFPP, GDP-synthetase, a fucosyltransferase or a GDP-Fucose transporter.

In an embodiment the media contains a substance that can increase the level of GDP-fucose, e.g., butyrate or fucose. Such media can be used, e.g., with a cell having a mutation that eliminates or decreased the activity of one or more of GMD, FX, fucokinase, GFPP, GDP-synthetase, a fucosyltransferase or a GDP-Fucose transporter.

Methods described herein allow monitoring a process of making a protein, e.g., to insure that the process is in compliance with parameters set out herein.

Thus, in another aspect, the invention features, a method of monitoring a process, e.g., a process of culturing cells, e.g., of a selected type, to produce a product, comprising:

optionally, selecting a glycan structure having reduced fucosylation, e.g., from a list comprising a plurality of glycan structures having reduced fucosylation (in embodiments the list is provided), and optionally memorializing said selected glycan structure;

optionally, selecting a cell on the basis of the cell having or subject to a manipulation that decreases the level of fucosylation or GDP-fucose, and which manipulation decreases the level of fucosylation or GDP-fucose (in embodiments the manipulation is from a list comprising a plurality of manipulations, and in embodiments the list is provided);

providing a cell having or subject to a manipulation that decreases the level of fucosylation or GDP-fucose, e.g., a cell having a manipulation described herein or a cell a cell selected by a method described herein;

culturing said cell, e.g., to provide a batch of cultured cells; and

evaluating (directly or indirectly) the level of GDP-fucose of, or a glycan complement, glycan component or glycan structure produced by, the cell or the batch of cultured cells,

to thereby monitor the process.

In one embodiment, the evaluating step comprises any of:

(a) isolating glycoproteins produced from the cell or the batch of cultured cells and evaluating the glycans containing on the glycoproteins,

(b) isolating a specific glycoprotein composition produced from the cell or the batch of cultured cells and evaluating the glycans from the isolated glycoprotein composition,

(c) obtaining a glycan preparation from a glycoprotein preparation or isolated glycoprotein produced from the cell or the batch of cultured cells and evaluating the glycans in the glycan preparation,

(d) cleaving monosaccharides from glycans present on a glycoprotein produced from the cell or the batch of cultured cells or from glycans on the surface of the cell or the batch of cultured cells, and detecting the cleaved monosaccharides,

(e) providing at least one peptide from a glycoprotein preparation produced from the cell or the batch of cultured cells, and evaluating the glycans on the at least one peptide, and

(f) evaluating glycans from glycans on the cell surface of the cell or the batch of cultured cells.

In another embodiment, the evaluating step comprises isolating glycoproteins produced from the cell or the batch of cultured cells and evaluating the glycans containing on the glycoproteins. In another embodiment, the evaluating step comprises isolating a specific glycoprotein composition produced from the cell or the batch of cultured cells and evaluating the glycans from the isolated glycoprotein composition. In another embodiment, the evaluating step comprises obtaining a glycan preparation from a glycoprotein preparation or isolated glycoprotein produced from the cell or the batch of cultured cells and evaluating the glycans in the glycan preparation. In another embodiment, the evaluating step comprises cleaving monosaccharides from glycans present on a glycoprotein produced from the cell or the batch of cultured cells or from glycans on the surface of the cell or the batch of cultured cells, and detecting the cleaved monosaccharides. In another embodiment, the evaluating step comprises providing at least one peptide from a glycoprotein preparation produced from the cell or the batch of cultured cells, and evaluating the glycans on the at least one peptide. In another embodiment, the evaluating step comprises evaluating glycans from glycans on the cell surface of the cell or the batch of cultured cells.

In another embodiment, the method further comprises, if an observed value from an evaluation step does not meet a reference value, discarding said cell, continuing culture of said cell, or altering a culture condition and further culturing said cell. In another embodiment, the method further comprises, if an observed value from an evaluation step meets said reference value, continuing culture of said cell or said batch of cultured cells, altering a culture condition and further culturing said cell or said batch of cultured cells, or discarding said cell or said batch of cultured cells. In another embodiment, the method further comprises continuing culture of the cell or the batch of cultured cells. In another embodiment, the method further comprises altering a culture condition and further culturing said cell or said batch of cultured cells and optionally repeating the evaluation.

In one embodiment, the evaluation comprises determining if the level of GDP-fucose in said cell that is below a first preselected level and, in embodiments, above a second preselected level. In one embodiment, said first preselected level of GDP-fucose is selected from a level that is:

i.a) approximately equal to or less than 80%, 70% or 60% of a reference level, e.g., the level in said cell or batch of cultured cells, e.g., a cell or batch of cultured cells which is otherwise similar, without the manipulation;

ii.a) approximately equal to, or less than, the point of maximum curvature above the inflection point (e.g., the inflection point in the second phase) on a graph of the amount of fucosylation vs. decrease in GDP-fucose;

ii.1.a) approximately equal to, or less than, the lowest level that results in a normal (e.g., that seen in an un-manipuated cell) level of fucosylation;

iii.a) approximately equal to or less than the point of maximum curvature below the inflection point on a graph of the amount of fucosylation vs. decrease in GDP-fucose;

iii.1.a) approximately equal to, or less than, the highest level that results in no further reduction in fucosylation;

iv.a) approximately equal to or less than point A on the curve in FIG. 1, or less than or equal to an analogous point on a plot of the amount of fucosylation (%) vs. the amount of GDP fucose as a % of control;

v.a) approximately equal to or less than that corresponding to an amount between points A and B on the curve in FIG. 1, or less than or equal to an analogous point on a plot of the amount of fucosylation (%) vs. the amount of GDP fucose as a % of control; or

vi.a) approximately equal to or less than point B on the curve in FIG. 1, or less than or equal to an analogous point on a plot of the amount of fucosylation (%) vs. the amount of GDP fucose as a % of control.

In one embodiment, said second preselected level of GDP-fucose is selected from a level:

i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;

ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;

iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level;

iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;

v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level; or

vi.b) approximately equal to or greater than point C on the curve in FIG. 1, or greater than or equal to an analogous point on a plot of the amount of fucosylation (%) vs. the amount of GDP fucose as a % of control.

In an embodiment the first level is i.a and the second level is selected from i.b, ii.b, iii.b, iv.b, v.b, and vi.b.

In an embodiment the first level is ii.a and the second level is selected from i.b, ii.b, iii.b, iv.b, v.b, and vi.b.

In an embodiment the first level is ii.1.a and the second level is selected from i.b, ii.b, iii.b, iv.b, v.b, and vi.b.

In an embodiment the first level is iii.a and the second level is selected from i.b, ii.b, iii.b, iv.b, v.b, and vi.b.

In an embodiment the first level is iii.1.a and the second level is selected from i.b, ii.b, iii.b, iv.b, v.b, and vi.b.

In an embodiment the first level is iv.a and the second level is selected from i.b, ii.b, iii.b, iv.b, v.b, and vi.b.

In an embodiment the first level is v.a and the second level is selected from i.b, ii.b, iii.b, iv.b, v.b, and vi.b.

In an embodiment the first level is vi.a and the second level is selected from i.b, ii.b, iii.b, iv.b, v.b, and vi.b.

In an embodiment the first level is selected from i.a, ii.a, ii.1.a, iii.a, iii.1.a, iv.a, v.a, and vi.a and the second level is i.b.

In an embodiment the first level is selected from i.a, ii.a, ii.1.a, iii.a, iii.1.a, iv.a, v.a, and vi.a and the second level is ii.b.

In an embodiment the first level is selected from i.a, ii.a, ii.1.a, iii.a, iii.1.a, iv.a, v.a, and vi.a and the second level is iii.b.

In an embodiment the first level is selected from i.a, ii.a, ii.1.a, iii.a, iii.1.a, iv.a, v.a, and vi.a and the second level is iv.b.

In an embodiment the first level is selected from i.a, ii.a, ii.1.a, iii.a, iii.1.a, iv.a, v.a, and vi.a and the second level is v.b.

In an embodiment the first level is selected from i.a, ii.a, ii.1.a, iii.a, iii.1.a, iv.a, v.a, and vi.a and the second level is vi.b.

In an embodiment the level of GDP-fucose is between point B and C on the curve in FIG. 1 or in an analogous range on a plot of the amount of fucosylation (%) vs. the amount of GDP fucose as a % of control.

In an embodiment the level of GDP-fucose is between point A and C on the curve in FIG. 1 or in an analogous range on a plot of the amount of fucosylation (%) vs. the amount of GDP fucose as a % of control.

In one embodiment, the level of GDP-fucose is selected to be outside the range between A and B on the curve in FIG. 1 (as relatively small changes in GDP-fucose will result in relatively large changes in the amount of fucosylation. In an embodiment the level is also less than B.) In another embodiment, the level of GDP-fucose is reduced by a predetermined level, e.g., in comparison with a reference. In another embodiment, the reference is the amount present in a cell or batch of cultured cells, e.g., a CHO cell or batch of cultured cells, lacking the manipulation but otherwise the same or essentially the same as the cell having the manipulation. In another embodiment, the level of GDP-fucose is reduced by, as much as, or more than, 10, 15, 20, 25, 30, 35, 40, 45, 50, 60, 70, 80 or 90%, as compared to the reference.

In one embodiment, said manipulation is not a genetic lesion or the presence of an siRNA that reduces the level of an enzyme that promotes formation of GDP-fucose, or the attachment of a fucosyl moiety. For example, the manipulation is not a lesion that decreases the expression of GMD, FX, fucokinase, GFPP, GDP-synthetase, a fucosyltransferase or a GDP-Fucose transporter. In another embodiment, the cell or batch of cultured cells is wild-type for one or all of GMD, FX, fucokinase, GFPP, GDP-synthetase, a fucosyltransferase or a GDP-Fucose transporter. In another embodiment, the cell or batch of cultured cells does not include an siRNA that targets GMD, FX, fucokinase, GFPP, GDP-synthetase, a fucosyltransferase or a GDP-Fucose transporter. In another embodiment, absent the manipulation, the level of fucosylation is substantially the same as the level in a wild-type cell. In another embodiment, the manipulated cell carries no mutation that substantially lowers GDP-fucose levels. In another embodiment, the manipulated cell has no siRNA that substantially lowers GDP-fucose levels.

In one embodiment, the cell has a mutation (e.g., a genetically engineered change) that decreases the level of GDP-fucose. Exemplary mutations include those which alter the activity of GMD, FX, fucokinase, GFPP, GDP-synthetase, a fucosyltransferase or a GDP-Fucose transporter. The mutation can be in the structural gene which encodes GMD, FX, fucokinase, GFPP, GDP-synthetase, a fucosyltransferase or a GDP-Fucose transporter. Such mutations can decrease the activity of the encoded protein. The decrease can be partial or complete. Such mutations can act, e.g., by altering the catalytic activity of the protein or by altering its half-life. Other exemplary mutations can be in a sequences that control expression of GMD, FX, fucokinase, GFPP, GDP-synthetase, a fucosyltransferase or a GDP-Fucose transporter. These can be mutations that completely, or partially, reduce the expression of the gene, at the RNA or protein level. Such mutations include deletion or other mutations in endogenous of control sequence. Such mutations also include the introduction of heterologous control sequence, e.g., the introduction of heterologous control regions, e.g., a sequence that will give a desired level of expression. (A heterologous control sequence is a sequence other than a sequence naturally associated with and operably linked to the structural gene.) In embodiments the manipulation comprises a mutation in the structural region or in a control sequence operably linked to the gene.

In an embodiment a cell having a mutation that that decreases the level of GDP-fucose, e.g., a mutation that decreases the activity of GMD, FX, fucokinase, GFPP, GDP-synthetase, a fucosyltransferase or a GDP-Fucose transporter is cultured in the presence of a substance, e.g., fucose, that results in a GDP-fucose level and/or a fucosylation level described herein. In an embodiment the cell includes a mutation that, in the absence of fucose in the culture medium, would result in a cell having an unacceptably low level of GDP-fucose. When, however, cultured under the appropriate conditions, e.g., media supplemented, e.g., with fucose, that cell can exhibit a desired level of GDP-fucose, e.g., a level of GDP-fucose described herein. Thus, fucose or another substance is present in the culture medium at a level that results in a level of GDP-fucose recited above.

In another embodiment, the manipulation is the presence of an siRNA that reduces the level of an enzyme that promotes formation of GDP-fucose, or the attachment of a fucosyl moiety, e.g., an siRNA that targets GMD, FX, fucokinase, GFPP, GDP-synthetase, a fucosyltransferase or a GDP-Fucose transporter, and fucose or another substance is present in the culture medium at a level that results in formation of said glycan structure having reduced fucosylation.

In one embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the cell is a Chinese Hamster Ovary (CHO) cell. In another embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the glycoprotein is selected from Table 1.

In one embodiment, the method further comprises culturing a plurality of the cells and separating as much as, or at least, 1, 10, 100, 1,000, or 10,000 grams of the glycoprotein from the cells. In another embodiment, the method further comprises combining the glycoprotein having reduced fucosylation with a pharmaceutically acceptable component and, e.g., formulating the glycoprotein having reduced fucosylation into a pharmaceutically acceptable formulation.

In one embodiment, the glycoprotein is analyzed by one or more of HPLC, CE, MALDI-MS and NMR.

In one embodiment, the method further comprises memorializing the result of the evaluation.

In one embodiment, the manipulation is, or is the product of, a selection for reduced levels of GDP-fucose. In another embodiment, the manipulation is, or is the product of, a selection for reduced fucosylation of a glycoprotein. In another embodiment, the manipulation comprises contact with, or inclusion in or on the cell or batch of cultured cells, of an exogenous inhibitor of an enzyme involved in GDP-fucose biosynthesis, e.g., a specific or non-specific inhibitor.

In one embodiment, the level of fucosylation at one, two, three, or more preselected amino acid residues is evaluated.

In one embodiment, the method further comprises providing a value for a parameter associated with a compound other than GDP-fucose, wherein a parameter for the compound, e.g., the level of the compound, is correlated to the level of GDP-fucose.

In another embodiment, the method further comprises providing a comparison of the value with a reference value, wherein optionally, a preselected relationship of the value to the reference value, e.g., greater than, equal to, or less than, is indicative of whether the level of GDP fucose is above, at or below the second level. In another embodiment, the method further comprises, responsive to the result of the comparison, increasing the level of GDP-fucose, decreasing the level of GDP-fucose or continuing cell culture without intervening to change the level of GDP-fucose. In one embodiment, the compound other than GDP-fucose is GDP-mannose. In one embodiment, the compound other than GDP-fucose is GDP-mannose and the parameter is the level of GDP-mannose.

In one embodiment, the method further comprises providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose. In one embodiment, the method comprises continuing to culture said cells, and repeating the steps above.

In an embodiments, an inhibitor, e.g., an inhibitor of GMD, FX, fucokinase, GFPP, GDP-fucose synthetase, or enzymes involved in the biosynthesis of GDP-mannose, is used, e.g., in the culture medium, to lower the levels of the GDP-fucose. In an embodiment the inhibitor can be guanosine-5′-O-(2-thiodiphosphate)-fucose, guanosine-5′-O-(2-thiodiphosphate)-mannose, pyridoxal-5′-phosphate, GDP-4-dehydro-6-L-deoxygalactose, GDP-L-fucose, guanosine diphosphate (GDP), guanosine monophosphate (GMP), GDP-D-glucose, or p-chloromercuriphenylsulfonate EDTA. The inhibitor can be used with a cell which is mutant or wildtype for one or more GMD, FX, fucokinase, GFPP, GDP-synthetase, a fucosyltransferase or a GDP-Fucose transporter.

In an embodiment the media contains a substance that can increase the level of GDP-fucose, e.g., butyrate or fucose. Such media can be used, e.g., with a cell having a mutation that eliminates or decreased the activity of one or more of GMD, FX, fucokinase, GFPP, GDP-synthetase, a fucosyltransferase or a GDP-Fucose transporter.

Methods described herein allow monitoring a process of making a protein, e.g., to insure that the process is in compliance with parameters set out herein.

In one aspect, the invention features a method of controlling a process for making a glycoprotein having a glycan structure with reduced fucosylation, comprising:
(1) providing a glycoprotein made by the process of

optionally, selecting a glycan structure having reduced fucosylation, e.g., from a list comprising a plurality of glycan structures having reduced fucosylation (in embodiments the list is provided);

optionally, selecting a cell on the basis of the cell having or subject to a manipulation that decreases the level of fucosylation or GDP-fucose, and which manipulation decreases the level of fucosylation or GDP-fucose (in embodiments the manipulation is from a list comprising a plurality of manipulations, and in embodiments the list is provided);

providing a cell having or subject to a manipulation that decreases the level of decreases the level of fucosylation or GDP-fucose; and

culturing the cell to provide a glycoprotein and, e.g., form a batch of cultured cells;
(2) evaluating (directly or indirectly) the level of GDP-fucose in the cells or the glycan structure of the glycoprotein,
(3) responsive to said evaluation, selecting a production parameter, e.g., a culture
   condition, e.g., a level of a nutrient or other component in the culture medium, e.g., to provide a selected level of GDP-fucose in the cells or the selected glycan structure of the glycoprotein,

to thereby control the process for making a glycoprotein having a glycan structure.

In one embodiment, the method comprises continuing culture of the cell or batch of cultured cells under conditions that differ from those used prior to the evaluation. In another embodiment, the method comprises continuing culture of the cell or batch of cultured cells under the same conditions used prior to the evaluation.

In one embodiment, the evaluation comprises determining if the level of GDP-fucose in said cell that is below a first preselected level and, in embodiments, above a second preselected level. In one embodiment, said first preselected level of GDP-fucose is selected from a level that is:

i.a) approximately equal to or less than 80%, 70% or 60% of a reference level, e.g., the level in said cell or batch of cultured cells, e.g., a cell or batch of cultured cells which is otherwise similar, without the manipulation;

ii.a) approximately equal to, or less than, the point of maximum curvature above the inflection point (e.g., the inflection point in the second phase) on a graph of the amount of fucosylation vs. decrease in GDP-fucose;

ii.1.a) approximately equal to, or less than, the lowest level that results in a normal (e.g., that seen in an un-manipuated cell) level of fucosylation;

iii.a) approximately equal to or less than the point of maximum curvature below the inflection point on a graph of the amount of fucosylation vs. decrease in GDP-fucose;

iii.1.a) approximately equal to, or less than, the highest level that results in no further reduction in fucosylation;

iv.a) approximately equal to or less than point A on the curve in FIG. 1, or less than or equal to an analogous point on a plot of the amount of fucosylation (%) vs. the amount of GDP fucose as a % of control;

v.a) approximately equal to or less than that corresponding to an amount between points A and B on the curve in FIG. 1, or less than or equal to an analogous point on a plot of the amount of fucosylation (%) vs. the amount of GDP fucose as a % of control; or

vi.a) approximately equal to or less than point B on the curve in FIG. 1, or less than or equal to an analogous point on a plot of the amount of fucosylation (%) vs. the amount of GDP fucose as a % of control.

In one embodiment, said second preselected level of GDP-fucose is selected from a level:

i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;

ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;

iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level;

iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;

v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level; or

vi.b) approximately equal to or greater than point C on the curve in FIG. 1, or greater than or equal to an analogous point on a plot of the amount of fucosylation (%) vs. the amount of GDP fucose as a % of control.

In an embodiment the first level is i.a and the second level is selected from i.b, ii.b, iii.b, iv.b, v.b, and vi.b.

In an embodiment the first level is ii.a and the second level is selected from i.b, ii.b, iii.b, iv.b, v.b, and vi.b.

In an embodiment the first level is ii.1.a and the second level is selected from i.b, ii.b, iii.b, iv.b, v.b, and vi.b.

In an embodiment the first level is iii.a and the second level is selected from i.b, ii.b, iii.b, iv.b, v.b, and vi.b.

In an embodiment the first level is iii.1.a and the second level is selected from i.b, ii.b, iii.b, iv.b, v.b, and vi.b.

In an embodiment the first level is iv.a and the second level is selected from i.b, ii.b, iii.b, iv.b, v.b, and vi.b.

In an embodiment the first level is v.a and the second level is selected from i.b, ii.b, iii.b, iv.b, v.b, and vi.b.

In an embodiment the first level is vi.a and the second level is selected from i.b, ii.b, iii.b, iv.b, v.b, and vi.b.

In an embodiment the first level is selected from i.a, ii.a, ii.1.a, iii.a, iii.1.a, iv.a, v.a, and vi.a and the second level is i.b.

In an embodiment the first level is selected from i.a, ii.a, ii.1.a, iii.a, iii.1.a, iv.a, v.a, and vi.a and the second level is ii.b.

In an embodiment the first level is selected from i.a, ii.a, ii.1.a, iii.a, iii.1.a, iv.a, v.a, and vi.a and the second level is iii.b.

In an embodiment the first level is selected from i.a, ii.a, ii.1.a, iii.a, iii.1.a, iv.a, v.a, and vi.a and the second level is iv.b.

In an embodiment the first level is selected from i.a, ii.a, ii.1.a, iii.a, iii.1.a, iv.a, v.a, and vi.a and the second level is v.b.

In an embodiment the first level is selected from i.a, ii.a, ii.1.a, iii.a, iii.1.a, iv.a, v.a, and vi.a and the second level is vi.b.

In an embodiment the level of GDP-fucose is between point B and C on the curve in FIG. 1 or in an analogous range on a plot of the amount of fucosylation (%) vs. the amount of GDP fucose as a % of control.

In an embodiment the level of GDP-fucose is between point A and C on the curve in FIG. 1 or in an analogous range on a plot of the amount of fucosylation (%) vs. the amount of GDP fucose as a % of control.

In one embodiment, the level of GDP-fucose is selected to be outside the range between A and B on the curve in FIG. 1 (as relatively small changes in GDP-fucose will result in relatively large changes in the amount of fucosylation. In an embodiment the level is also less than B.) In another embodiment, the level of GDP-fucose is reduced by a predetermined level, e.g., in comparison with a reference. In another embodiment, the reference is the amount present in a cell or batch of cultured cells, e.g., a CHO cell or batch of cultured cells, lacking the manipulation but otherwise the same or essentially the same as the cell having the manipulation. In another embodiment, the level of GDP-fucose is reduced by, as much as, or more than, 10, 15, 20, 25, 30, 35, 40, 45, 50, 60, 70, 80 or 90%, as compared to the reference.

In one embodiment, said evaluation step comprises comparing the structure of said glycan structure having reduced fucosylation present on a glycoprotein from said cultured cell or batch of cultured cells to a reference, and determining if said glycan structure having reduced fucosylation present on a glycoprotein from said cultured cell or batch of cultured cells differs from the corresponding glycan structure formed by a cell or batch of cultured cells that lacks the manipulation.

In one embodiment, the method further comprises evaluating the glycoprotein for a parameter related to fucosylation, e.g., the amount of fucosylation in the glycan complement, the amount or fucosylation on a component of the glycan complement, or the amount of fucosylation on a glycan component, e.g., in a preparation of glycoproteins. In another embodiment, the method further comprises evaluating the glycoprotein for a parameter related to fucosylation, e.g., the proportion of a preselected glycan component which bears a fucosyl moiety, e.g., at a selected position on the glycan component, e.g., in a preparation of glycoproteins.

In one embodiment, the level of fucosylation at one, two, three, or more preselected amino acid residues is evaluated. In another embodiment, the level of fucosylation is reduced by a predetermined level in comparison with a reference. In another embodiment, the reference is the amount present in a cell or batch of cultured cells, e.g., a CHO cell or batch of cultured cells, lacking the manipulation but otherwise the same or essentially the same as the cell or batch of cultured cells having the manipulation. In another embodiment, the level of fucosylation is reduced by, as much as, or more than, 10, 20, 30, 40, 50, 60, 70, 80 or 90%, as compared to the reference.

In one embodiment, said manipulation is not a genetic lesion or the presence of an siRNA that reduces the level of an enzyme that promotes formation of GDP-fucose, or the attachment of a fucosyl moiety. For example, the manipulation is not a lesion that decreases the expression of GMD, FX, fucokinase, GFPP, GDP-synthetase, a fucosyltransferase or a GDP-Fucose transporter. In another embodiment, the cell or batch of cultured cells is wild-type for one or all of GMD, FX, fucokinase, GFPP, GDP-synthetase, a fucosyltransferase or a GDP-Fucose transporter. In another embodiment, the cell or batch of cultured cells does not include an siRNA that targets GMD, FX, fucokinase, GFPP, GDP-synthetase, a fucosyltransferase or a GDP-Fucose transporter. In another embodiment, absent the manipulation, the level of fucosylation is substantially the same as the level in a wild-type cell. In another embodiment, the manipulated cell carries no mutation that substantially lowers GDP-fucose levels. In another embodiment, the manipulated cell has no siRNA that substantially lowers GDP-fucose levels.

In one embodiment, the cell has a mutation (e.g., a genetically engineered change) that decreases the level of GDP-fucose. Exemplary mutations include those which alter the activity of GMD, FX, fucokinase, GFPP, GDP-synthetase, a fucosyltransferase or a GDP-Fucose transporter.

The mutation can be in the structural gene which encodes GMD, FX, fucokinase, GFPP, GDP-synthetase, a fucosyltransferase or a GDP-Fucose transporter. Such mutations can decrease the activity of the encoded protein. The decrease can be partial or complete. Such mutations can act, e.g., by altering the catalytic activity of the protein or by altering its half-life. Other exemplary mutations can be in a sequences that control expression of GMD, FX, fucokinase, GFPP, GDP-synthetase, a fucosyltransferase or a GDP-Fucose transporter. These can be mutations that completely, or partially, reduce the expression of the gene, at the RNA or protein level. Such mutations include deletion or other mutations in endogenous of control sequence. Such mutations also include the introduction of heterologous control sequence, e.g., the introduction of heterologous control regions, e.g., a sequence that will give a desired level of expression. (A heterologous control sequence is a sequence other than a sequence naturally associated with and operably linked to the structural gene.) In embodiments the manipulation comprises a mutation in the structural region or in a control sequence operably linked to the gene.

In an embodiment a cell having a mutation that that decreases the level of GDP-fucose, e.g., a mutation that decreases the activity of GMD, FX, fucokinase, GFPP, GDP-synthetase, a fucosyltransferase or a GDP-Fucose transporter is cultured in the presence of a substance, e.g., fucose, that results in a GDP-fucose level and/or a fucosylation level described herein. In an embodiment the cell includes a mutation that, in the absence of fucose in the culture medium, would result in a cell having an unacceptably low level of GDP-fucose. When, however, cultured under the appropriate conditions, e.g., media supplemented, e.g., with fucose, that cell can exhibit a desired level of GDP-fucose, e.g., a level of GDP-fucose described herein. Thus, fucose or another substance is present in the culture medium at a level that results in a level of GDP-fucose recited above.

In another embodiment, the manipulation is the presence of an siRNA that reduces the level of an enzyme that promotes formation of GDP-fucose, or the attachment of a fucosyl moiety, e.g., an siRNA that targets GMD, FX, fucokinase, GFPP, GDP-synthetase, a fucosyltransferase or a GDP-Fucose transporter, and fucose or another substance is present in the culture medium at a level that results in formation of said glycan structure having reduced fucosylation.

In one embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the cell is a Chinese Hamster Ovary (CHO) cell. In another embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the glycoprotein is selected from Table 1.

In one embodiment, the method further comprises culturing a plurality of the cells and separating as much as, or at least, 1, 10, 100, 1,000, or 10,000 grams of the glycoprotein from the cells. In another embodiment, the method further comprises combining the glycoprotein having reduced fucosylation with a pharmaceutically acceptable component and, e.g., formulating the glycoprotein having reduced fucosylation into a pharmaceutically acceptable formulation.

In one embodiment, the glycoprotein is analyzed by one or more of HPLC, CE, MALDI-MS and NMR.

In one embodiment, the method further comprises memorializing the result of the evaluation.

In one embodiment, the manipulation is, or is the product of, a selection for reduced levels of GDP-fucose. In another embodiment, the manipulation is, or is the product of, a selection for reduced fucosylation of a glycoprotein. In another embodiment, the manipulation comprises contact with, or inclusion in or on the cell or batch of cultured cells, of an exogenous inhibitor of an enzyme involved in GDP-fucose biosynthesis, e.g., a specific or non-specific inhibitor.

In one embodiment, the level of fucosylation at one, two, three, or more preselected amino acid residues is evaluated.

In one embodiment, one or more of said cell or said batch of cultured cells, said manipulation, and said glycoprotein, is selected on the basis that it or the combination will provide a glycoprotein having reduced fucosylation.

In one embodiment, the method further comprises providing a value for a parameter associated with a compound other than GDP-fucose, wherein a parameter for the compound, e.g., the level of the compound, is correlated to the level of GDP-fucose.

In another embodiment, the method further comprises providing a comparison of the value with a reference value, wherein optionally, a preselected relationship of the value to the reference value, e.g., greater than, equal to, or less than, is indicative of whether the level of GDP fucose is above, at or below the second level. In another embodiment, the method further comprises, responsive to the result of the comparison, increasing the level of GDP-fucose, decreasing the level of GDP-fucose or continuing cell culture without intervening to change the level of GDP-fucose. In one embodiment, the compound other than GDP-fucose is GDP-mannose. In one embodiment, the compound other than GDP-fucose is GDP-mannose and the parameter is the level of GDP-mannose.

In one embodiment, the method further comprises providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose. In one embodiment, the method comprises continuing to culture said cells, and repeating the steps above.

In an embodiments, an inhibitor, e.g., an inhibitor of GMD, FX, fucokinase, GFPP, GDP-fucose synthetase, or enzymes involved in the biosynthesis of GDP-mannose, is used, e.g., in the culture medium, to lower the levels of the GDP-fucose. In an embodiment the inhibitor can be guanosine-5′-O-(2-thiodiphosphate)-fucose, guanosine-5′-O-(2-thiodiphosphate)-mannose, pyridoxal-5′-phosphate, GDP-4-dehydro-6-L-deoxygalactose, GDP-L-fucose, guanosine diphosphate (GDP), guanosine monophosphate (GMP), GDP-D-glucose, or p-chloromercuriphenylsulfonate EDTA. The inhibitor can be used with a cell which is mutant or wildtype for one or more GMD, FX, fucokinase, GFPP, GDP-synthetase, a fucosyltransferase or a GDP-Fucose transporter.

In an embodiment the media contains a substance that can increase the level of GDP-fucose, e.g., butyrate or fucose. Such media can be used, e.g., with a cell having a mutation that eliminates or decreased the activity of one or more of GMD, FX, fucokinase, GFPP, GDP-synthetase, a fucosyltransferase or a GDP-Fucose transporter.

Methods described herein allow monitoring a process of making a protein, e.g., to insure that the process is in compliance with parameters set out herein.

In one aspect, the invention features method of controlling a process for making a glycoprotein having a glycan structure with reduced fucosylation, comprising:

(1) providing a glycoprotein made by the process of:

optionally, selecting a glycan structure having reduced fucosylation, e.g., from a list comprising a plurality of glycan structures having reduced fucosylation (in embodiments the list is provided);

optionally, selecting a cell on the basis of the cell having or subject to a manipulation that decreases the level of fucosylation or GDP-fucose, and which manipulation decreases the level of fucosylation or GDP-fucose (in embodiments the manipulation is from a list comprising a plurality of manipulations, and in embodiments the list is provided);

providing a cell having or subject to a manipulation that decreases the level of decreases the level of fucosylation or GDP-fucose; and

culturing the cell to provide a glycoprotein and, e.g., form a batch of cultured cells;

(2) providing a value for a parameter associated with a compound other than GDP-fucose, wherein a parameter for the compound, e.g., the level of the compound, is correlated to the level of GDP-fucose,

(3) providing a comparison of the value with a reference value, wherein optionally, a preselected relationship of the value to the reference value, e.g., greater than, equal to, or less than, is indicative of whether the level of GDP fucose is above, at or below a preselected level

(4) responsive to said comparison, selecting a production parameter, e.g., a culture condition, e.g., a level of a nutrient or other component in the culture medium, to thereby control the process for making a glycoprotein having a glycan structure.

In one embodiment, the method further comprises, responsive to the result of the comparison, increasing the level of GDP-fucose, decreasing the level of GDP-fucose or continuing cell culture without intervening to change the level of GDP-fucose. In another embodiment, the compound other than GDP-fucose is GDP-mannose. In another embodiment, the compound other than GDP-fucose is GDP-mannose and the parameter is the level of GDP-mannose.

In one embodiment, the method further comprises providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose. In another embodiment, the method comprises continuing to culture said cells, and repeating the steps above.

In one embodiment, said manipulation is not a genetic lesion or the presence of an siRNA that reduces the level of an enzyme that promotes formation of GDP-fucose, or the attachment of a fucosyl moiety. For example, the manipulation is not a lesion that decreases the expression of GMD, FX, fucokinase, GFPP, GDP-synthetase, a fucosyltransferase or a GDP-Fucose transporter. In another embodiment, the cell or batch of cultured cells is wild-type for one or all of GMD, FX, fucokinase, GFPP, GDP-synthetase, a fucosyltransferase or a GDP-Fucose transporter. In another embodiment, the cell or batch of cultured cells does not include an siRNA that targets GMD, FX, fucokinase, GFPP, GDP-synthetase, a fucosyltransferase or a GDP-Fucose transporter. In another embodiment, absent the manipulation, the level of fucosylation is substantially the same as the level in a wild-type cell. In another embodiment, the manipulated cell carries no mutation that substantially lowers GDP-fucose levels. In another embodiment, the manipulated cell has no siRNA that substantially lowers GDP-fucose levels.

In one embodiment, the cell has a mutation (e.g., a genetically engineered change) that decreases the level of GDP-fucose. Exemplary mutations include those which alter the activity of GMD, FX, fucokinase, GFPP, GDP-synthetase, a fucosyltransferase or a GDP-Fucose transporter. The mutation can be in the structural gene which encodes GMD, FX, fucokinase, GFPP, GDP-synthetase, a fucosyltransferase or a GDP-Fucose transporter. Such mutations can decrease the activity of the encoded protein. The decrease can be partial or complete. Such mutations can act, e.g., by altering the catalytic activity of the protein or by altering its half-life. Other exemplary mutations can be in a sequences that control expression of GMD, FX, fucokinase, GFPP, GDP-synthetase, a fucosyltransferase or a GDP-Fucose transporter. These can be mutations that completely, or partially, reduce the expression of the gene, at the RNA or protein level. Such mutations include deletion or other mutations in endogenous of control sequence. Such mutations also include the introduction of heterologous control sequence, e.g., the introduction of heterologous control regions, e.g., a sequence that will give a desired level of expression. (A heterologous control sequence is a sequence other than a sequence naturally associated with and operably linked to the structural gene.) In embodiments the manipulation comprises a mutation in the structural region or in a control sequence operably linked to the gene.

In an embodiment a cell having a mutation that that decreases the level of GDP-fucose, e.g., a mutation that decreases the activity of GMD, FX, fucokinase, GFPP, GDP-synthetase, a fucosyltransferase or a GDP-Fucose transporter is cultured in the presence of a substance, e.g., fucose, that results in a GDP-fucose level and/or a fucosylation level described herein. In an embodiment the cell includes a mutation that, in the absence of fucose in the culture medium, would result in a cell having an unacceptably low level of GDP-fucose. When, however, cultured under the appropriate conditions, e.g., media supplemented, e.g., with fucose, that cell can exhibit a desired level of GDP-fucose, e.g., a level of GDP-fucose described herein. Thus, fucose or another substance is present in the culture medium at a level that results in a level of GDP-fucose recited above.

In another embodiment, the manipulation is the presence of an siRNA that reduces the level of an enzyme that promotes formation of GDP-fucose, or the attachment of a fucosyl moiety, e.g., an siRNA that targets GMD, FX, fucokinase, GFPP, GDP-synthetase, a fucosyltransferase or a GDP-Fucose transporter, and fucose or another substance is present in the culture medium at a level that results in formation of said glycan structure having reduced fucosylation.

In one embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the cell is a Chinese Hamster Ovary (CHO) cell. In another embodiment, the glycoprotein is an antibody. In another embodiment, the antibody has reduced core fucosylation. In another embodiment, the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

In one embodiment, the glycoprotein is selected from Table 1.

In one embodiment, the method further comprises culturing a plurality of the cells and separating as much as, or at least, 1, 10, 100, 1,000, or 10,000 grams of the glycoprotein from the cells. In another embodiment, the method further comprises combining the glycoprotein having reduced fucosylation with a pharmaceutically acceptable component and, e.g., formulating the glycoprotein having reduced fucosylation into a pharmaceutically acceptable formulation.

In one embodiment, the glycoprotein is analyzed by one or more of HPLC, CE, MALDI-MS and NMR.

In one embodiment, the method further comprises memorializing the result of the evaluation.

In one embodiment, the manipulation is, or is the product of, a selection for reduced levels of GDP-fucose. In another embodiment, the manipulation is, or is the product of, a selection for reduced fucosylation of a glycoprotein. In another embodiment, the manipulation comprises contact with, or inclusion in or on the cell or batch of cultured cells, of an exogenous inhibitor of an enzyme involved in GDP-fucose biosynthesis, e.g., a specific or non-specific inhibitor.

In an embodiments, an inhibitor, e.g., an inhibitor of GMD, FX, fucokinase, GFPP, GDP-fucose synthetase, or enzymes involved in the biosynthesis of GDP-mannose, is used, e.g., in the culture medium, to lower the levels of the GDP-fucose. In an embodiment the inhibitor can be guanosine-5′-O-(2-thiodiphosphate)-fucose, guanosine-5′-O-(2-thiodiphosphate)-mannose, pyridoxal-5′-phosphate, GDP-4-dehydro-6-L-deoxygalactose, GDP-L-fucose, guanosine diphosphate (GDP), guanosine monophosphate (GMP), GDP-D-glucose, or p-chloromercuriphenylsulfonate EDTA. The inhibitor can be used with a cell which is mutant or wildtype for one or more GMD, FX, fucokinase, GFPP, GDP-synthetase, a fucosyltransferase or a GDP-Fucose transporter.

In an embodiment the media contains a substance that can increase the level of GDP-fucose, e.g., butyrate or fucose. Such media can be used, e.g., with a cell having a mutation that eliminates or decreased the activity of one or more of GMD, FX, fucokinase, GFPP, GDP-synthetase, a fucosyltransferase or a GDP-Fucose transporter.

In one aspect, the invention features a method of making a glycoprotein having reduced fucosylation, comprising:
(a) providing, acknowledging, selecting, accepting, or memorializing a defined, desired or preselected glycan structure having reduced fucosylation for the glycoprotein,
(b) optionally providing a cell manipulated to decrease the level of fucosylation or fucose-GDP,
(c) culturing a cell manipulated to decrease the level of fucosylation or fucose-GDP, e.g., to form a batch of cultured cells, and
(d) isolating from the cell or batch of cultured cells a glycoprotein having the desired glycan structure,
   thereby making a glycoprotein.

In one aspect, the invention features method of making a glycoprotein, comprising:

providing, acknowledging, selecting, accepting, or memorializing a defined, desired or preselected glycan structure having reduced fucosylation for the glycoprotein, chosen, e.g., from Table 1;

optionally, providing, acknowledging, selecting, accepting, or memorializing a manipulation described herein;

culturing a cell having the manipulation, e.g., to form a batch of cultured cells;
isolating from the cell or batch of cultured cells a glycoprotein having the desired glycan structure,
   thereby making a glycoprotein.

In one aspect, the invention features method of formulating a pharmaceutical composition comprising:

contacting a glycoprotein made by a method described herein with a pharmaceutically acceptable substance, e.g., an excipient or diluent.

In one aspect, the invention features pharmaceutical preparation of a glycoprotein described herein or made by a method described herein, wherein the glycoprotein is selected from Table 1.

Any step that generates information in a method described herein, e.g., a selection, analysis, comparison with a reference, or other evaluation or determination, can be memorialized, for example, by entry into a computer database. Such information can further be compared to a reference, or itself serve as a reference, for an evaluation made in the process.

### DETAILED DESCRIPTION

The drawings are first described.

FIG. 1 is a plot of increasing amount of fucosylation on a glycoprotein produced by a cell (as a percentage of a cell without manipulation) (Y axis) against decreasing cellular GDP-fucose in the cell (as a percentage of a cell without manipulation). The plot shows a non-linear relationship indicative of a threshold relationship. E.g., reducing parental GDP-fucose levels by 20% gives little reduction in the amount of fucosylation. Reduction of more than 20% in GDP-fucose levels produced significant further reduction in glycosylation. Point A on the plot shows the point at which reduction in GDP-fucose begins to result in a significant reduction in fucosylation. Point B on the plot shows the point at which further reduction in GDP-fucose fails to result in further significant reduction in fucosylation. The region between points B and C is an optimal range. [>20% and <80% of parental GDP-fucose levels, e.g., >40% and <65% of parental GDP-fucose levels.]

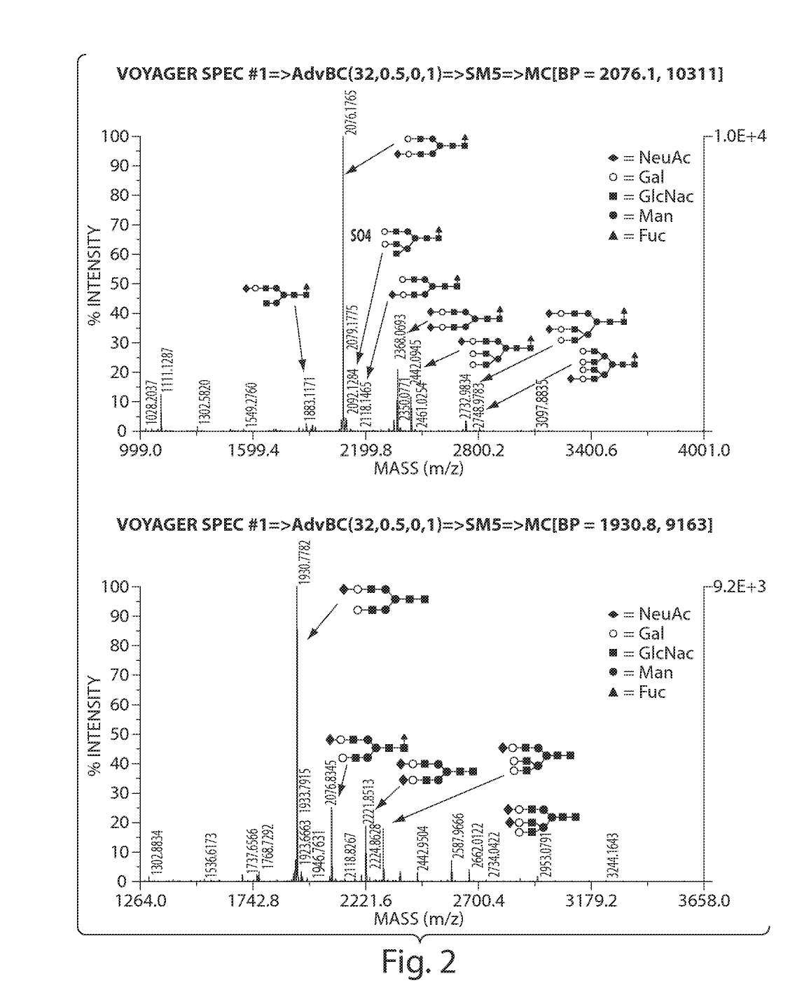

FIG. 2 is a depiction of glycan profiles from glycoproteins expressed from wild type CHO cells (top) and Lec 13.6 A cells (bottom). Data are negative mode MALDI spectra with the most abundant glycans indicated by structure. As indicated, glycans from the Lec 13.6 A cells have very low levels of fucosylation.

### DEFINITIONS

“Branched fucose” as used herein refers to a fucose moiety that is attached via an cd-3 or cd-4 linkage to an N-acetylglucosamine sugar of an N-linked or O-linked glycan component.

“Core fucose” as used herein refers to a fucose moiety that is attached via an cd-6 linkage to the N-acetylglucosamine sugar that is directly attached to the asparagine amino acid in an N-linked glycan component.

“Culturing” as used herein refers to placing a cell, e.g., a vertebrate, mammalian or rodent cell, under conditions that allow for at least some of the steps for the production of a glycoprotein to proceed. In embodiments, the conditions are sufficient to allow the glycosylation process to be completed. In embodiments, the conditions are sufficient to allow all of the steps, e.g., through secretion, to occur. Culturing refers to cultures of cells, cell lines, and populations of cells. The cells can be eukaryotic or a prokaryotic cells, e.g., animal, plant, yeast, fungal, insect or bacterial cells. In embodiments, culturing refers to in vitro culture of cells, e.g., primary or secondary cell lines.

“Glycan complement” as used herein refers to all of the glycan components of a glycoprotein. In the case of a protein having a single glycosylation site, the glycan component attached thereto forms the glycan complement. In the case of a protein having more than one glycosylation site, the glycan complement is made up of the glycan components attached at all of the sites. The N-linked glycan complement refers to all of the N-linked glycan components of a protein. The O-linked glycan complement refers to all of the O-linked glycan components of a protein. A “component of the glycan complement” refers to a subset of the glycan components making up the glycan complement, e.g., one or more glycan components attached to its or their respective glycosylation site or sites.

“Glycan component” as used herein refers to a sugar moiety, e.g., a monosaccharide, oligosaccharide or polysaccharide (e.g., a disaccharide, trisaccharide, tetrasaccharide, etc.) attached to a protein at one site. In embodiments the attachment is covalent and the glycan component is N- or O-linked to the protein. Glycan components can be chains of monosaccharides attached to one another via glycosidic linkages. Glycan components can be linear or branched. Fucose moieties are typically attached to an N-acetylglucosamine sugar of an N-linked or O-linked glycan component via an cd-3, cd-4 or cd-6 linkage.

“Glycan structure” as used herein refers to the structure of a glycan complement, component of a glycan complement, or glycan component. In embodiments it refers to one or more of the placement and number of fucosyl moieties.

A glycan structure can be described in terms of a comparison of the presence, absence or amount of a first glycan structure to a second glycan structure, for example, the presence, absence or amount of fucose relative to the presence, absence or amount of some other component. In other examples, the presence, absence or amount of fucose can be compared, e.g., to the presence, absence or amount of a sialic acid derivative such as N-glycolylneuraminic acid.

Glycan structures can be described, identified or assayed in a number of ways. A glycan structure can be described, e.g., in defined structural terms, e.g., by chemical name, or by a functional or physical property, e.g., by molecular weight or by a parameter related to purification or separation, e.g., retention time of a peak in a column or other separation device. In embodiments a glycan structure can, by way of example, be a peak or other fraction (representing one or more species) from glycan structures derived from a glycoprotein, e.g., from an enzymatic digest.

“Manipulation” as used herein can be any of a cell/activity-based manipulation, an envirocultural manipulation, or a selected functional manipulation. In general a manipulation is induced, selected, isolated, engineered, or is otherwise the product of the “hand of man.”

A “cell/activity-based manipulation” as used herein refers to a property of a cell that decreases the level of GDP-fucose activity in a cell, e.g., which decreases the level of activity of an enzyme involved in GDP-fucose biosynthesis. Decreased means by comparison with a cell that is not subject to the cell/activity-based manipulation.

Examples of cell/activity-based manipulations include:

the presence in or on the cell of an exogenous inhibitor (e.g., an siRNA or a chemical inhibitor) of the activity of an enzyme involved in GDP-fucose biosynthesis; or

a mutation or other genetic event that inhibits the activity of an enzyme involved in GDP-fucose biosynthesis. In some embodiments a cell/activity-based manipulation excludes genetic lesions, e.g., genetic knock-outs, discussed elsewhere herein.

An “envirocultural manipulation” as used herein refers to a property of the culture conditions, e.g., of the culture medium, that lowers GDP-fucose level and results in a decrease in transfer of a fucose moiety to a glycoprotein. Examples include the modulation of salt or ion concentrations in the culture medium. Specific examples of media conditions that will lead to altered levels of GDP-fucose include but are not limited to altering the levels of cobalt, butyrate, fucose, guanosine, and manganese.

A selected functional manipulation is a physical characteristic or property characterized, e.g., by the process that gave rise to it, e.g., a cell that was placed under selective conditions that result in the cell being able to produce a glycoprotein having a glycan structure characterized by a reduced GDP-fucose level, wherein the underlying basis for the ability to produce said glycoprotein having a glycan structure may or may not be known or characterized.

“Reduced fucosylation” relates to the amount or frequency of fucosylation. With regard to a single molecule, it means fewer fucose moieties, e.g., as compared to a reference, e.g., a protein made by a cell without the manipulation that gave rise to reduced fucosylation. With regard to a plurality of molecules, e.g., a pharmaceutically acceptable preparation, it can mean fewer fucose moieties on the molecules of the plurality (e.g., as compared to a reference, e.g., the plurality made by cells without the manipulation that gave rise to reduced fucosylation). The comparison can be with regard to all fucosylation sites on the subject molecule or with regard to the fucosylation at one or more specific sites. Reduced fucosylation can mean reduced occupancy by, or presence of, a fucosyl moiety at a selected site, e.g., as compared to a reference preparation, e.g., a reference preparation made by cells without the manipulation that gave rise to reduced fucosylation.

### Regulation of Glycosylation

Glycosylation is a nonlinear non-template driven process. To this end, regulation of a particular glycan structure may be due to a number of orthogonal inputs such as precursor levels, donor levels, and transferase levels to name a few. Glycosylation of proteins can have dramatic effect on their activities, such as regulating receptor affinity, regulating bioavailability, or altering immunogenicity. For example, the presence of core fucosylation on an antibody may significantly attenuate antibody-dependent cell-mediated cytotoxicity (ADCC).

Eukaryotic glycosylation occurs in the endoplasmic reticulum (ER) and Golgi through a stepwise process in which one monosaccharide is added through the activity of a glycosyltransferase, utilizing an activated sugar nucleotide as the donor molecule. The graphic below illustrates this with GDP-fucose.

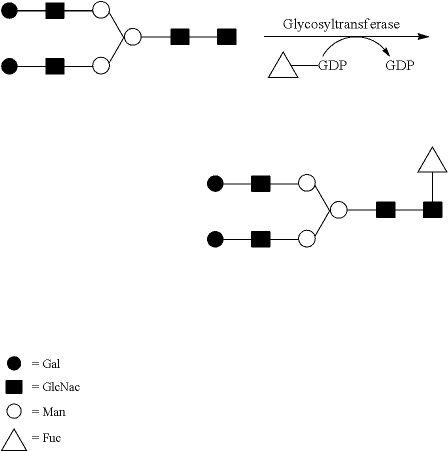

It should be noted that fucose can be added to a glycan structure at various points during the diversification process. This is one example of a glycan structure that may be fucosylated.

### GDP-Fucose Biosynthesis

Two pathways have been described for synthesis of GDP-fucose in the cytosol of essentially all mammalian cells, the de novo pathway and the salvage pathway. The de novo pathway transforms GDP-mannose to GDP-fucose via three enzymatic reactions carried out by two proteins, GDP-mannose 4,6-dehydratase (GMD) and GDP-keto-6-deoxymannose-3,5-epimerase-4-reductase (also known as the FX protein or tissue specific transplantation antigen P35B) (Scheme 1). The salvage pathway synthesizes GDP-fucose from free fucose derived from extracellular or lysosomal sources via the reactions of two proteins, a fucose kinase (fucokinase) followed by either GDP-fucose pyrophosphorylase (GFPP) (also known as fucose-1-phosphate guanylyltransferase) or GDP-fucose synthetase (Scheme 2). Quantitative studies of fucose metabolism in HeLa cells indicate that greater than 90% of GDP-fucose is derived from the de novo pathway (Yurchenco and Atkinson, Biochemistry 14(14):3107-14, 1975; Yurchenco and Atkinson, Biochemistry 16(5):944-53, 1977).

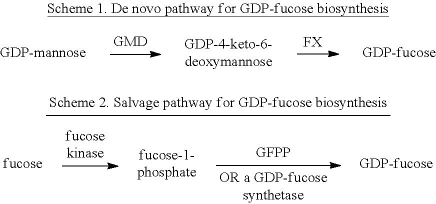

Methods of regulating fucosylation by modulating levels of GDP-fucose, e.g., lowering GDP-fucose levels below a threshold level, are disclosed herein. In some embodiments this may involve the use of inhibitors of enzymes critical for GDP-fucose biosynthesis, such as GMD, FX, fucose kinase, GFPP and/or GDP-fucose synthetase.

Exemplary proteins involved in GDP-fucose biosynthesis include the following:

Protein sequence of human GDP-mannose 4,6-
dehydratase
(SEQ ID NO: 1)
MAHAPARCPSARGSGDGEMGKPRNVALITGITGQDGSYLAEFLLEKGYEV
HGIVRRSSSFNTGRIEHLYKNPQAHIEGNMKLHYGDLTDSTCLVKIINEV
KPTEIYNLGAQSHVKISFDLAEYTADVDGVGTLRLLDAVKTCGLINSVKF
YQASTSELYGKVQEIPQKETTPFYPRSPYGAAKLYAYWIVVNFREAYNLF
AVNGILFNHESPRRGANFVTRKISRSVAKIYLGQLECFSLGNLDAKRDWG
HAKDYVEAMWLMLQNDEPEDFVIATGEVHSVREFVEKSFLHIGKTIVWEG
KNENEVGRCKETGKVHVTVDLKYYRPTEVDFLQGDCTKAKQKLNWKPRVA
FDELVREMVHADVELMRTNPNA
GenBank Accession No. NP_001491 (GenBank version
dated 10-DEC-2008)
mRNA sequence of human GDP-mannose 4,6-dehydratase
(SEQ ID NO: 2)
ATGGCACACGCACCGGCACGCTGCCCCAGCGCCCGGGGCTCCGGGGACGG
CGAGATGGGCAAGCCCAGGAACGTGGCGCTCATCACCGGTATCACAGGCC
AGGATGGTTCCTACCTGGCTGAGTTCCTGCTGGAGAAAGGCTATGAGGTC
CATGGAATTGTACGGCGGTCCAGTTCATTTAATACGGGTCGAATTGAGCA
TCTGTATAAGAATCCCCAGGCTCACATTGAAGGAAACATGAAGTTGCACT
ATGGCGATCTCACTGACAGTACCTGCCTTGTGAAGATCATTAATGAAGTA
AAGCCCACAGAGATCTACAACCTTGGAGCCCAGAGCCACGTCAAAATTTC
CTTTGACCTCGCTGAGTACACTGCGGACGTTGACGGAGTTGGCACTCTAC
GACTTCTAGATGCAGTTAAGACTTGTGGCCTTATCAACTCTGTGAAGTTC
TACCAAGCCTCAACAAGTGAACTTTATGGGAAAGTGCAGGAAATACCCCA
GAAGGAGACCACCCCTTTCTATCCCCGGTCACCCTATGGGGCAGCAAAAC
TCTATGCCTATTGGATTGTGGTGAACTTCCGTGAGGCGTATAATCTCTTT
GCAGTGAACGGCATTCTCTTCAATCATGAGAGTCCCAGAAGAGGAGCTAA
TTTCGTTACTCGAAAAATTAGCCGGTCAGTAGCTAAGATTTACCTTGGAC
AACTGGAATGTTTCAGTTTGGGAAATCTGGATGCCAAACGAGATTGGGGC
CATGCCAAGGACTATGTGGAGGCTATGTGGTTGATGTTGCAGAATGATGA
GCCGGAGGACTTCGTTATAGCTACTGGGGAGGTCCATAGTGTCCGGGAAT
TTGTCGAGAAATCATTCTTGCACATTGGAAAAACCATTGTGTGGGAAGGA
AAGAATGAAAATGAAGTGGGCAGATGTAAAGAGACCGGCAAAGTTCACGT
GACTGTGGATCTCAAGTACTACCGGCCAACTGAAGTGGACTTTCTGCAGG
GCGACTGCACCAAAGCGAAACAGAAGCTGAACTGGAAGCCCCGGGTCGCT
TTCGATGAGCTGGTGAGGGAGATGGTGCACGCCGACGTGGAGCTCATGAG
GACAAACCCCAATGCCTGA
GenBank Accession No. NM_001500 (GenBank version
dated 10-DEC-2008)
Protein sequence of mouse GDP-mannose 4,6-
dehydratase
(SEQ ID NO: 3)
MAQAPAKCPSYPGSGDGEMGKLRKVALITGITGQDGSYLAEFLLEKGYEV
HGIVRRSSSFNTGRIEHLYKNPQAHIEGNMKLHYGDLTDSTCLVKIINEV
KPTEIYNLGAQSHVKISFDLAEYTADVDGVGTLRLLDAIKTCGLINSVKF
YQASTSELYGKVQEIPQKETTPFYPRSPYGAAKLYAYWIVVNFREAYNLF
AVNGILFNHESPRRGANFVTRKISRSVAKIYLGQLECFSLGNLDAKRDWG
HAKDYVEAMWLMLQNDEPEDFVIATGEVHSVREFVEKSFMHIGKTIVWEG
KNENEVGRCKETGKVHVTVDLKYYRPTEVDFLQGDCSKAQQKLNWKPRVA
FDELVREMVQADVELMRTNPNA
GenBank Accession No. NP_666153 (GenBank version
dated 18-APR-2009)
mRNA sequence of mouse GDP-mannose 4,6-dehydratase
(SEQ ID NO: 4)
ATGGCTCAAGCTCCCGCTAAGTGCCCGAGCTACCCGGGCTCCGGGGATGG
CGAGATGGGCAAGCTCAGGAAGGTGGCTCTCATCACTGGCATCACCGGAC
AGGATGGTTCGTACTTGGCAGAATTCCTGTTGGAGAAAGGGTACGAGGTC
CATGGAATAGTACGGCGATCTAGTTCATTTAATACAGGTCGAATTGAACA
TTTATATAAGAATCCTCAGGCTCATATTGAAGGAAACATGAAGTTGCACT
ATGGTGACCTCACTGACAGCACCTGCCTAGTGAAAATCATCAATGAAGTC
AAGCCTACAGAGATCTATAATCTTGGAGCCCAGAGCCATGTCAAGATCTC
CTTTGACTTAGCTGAGTACACCGCAGATGTTGATGGCGTTGGCACCTTGC
GGCTTCTGGATGCAATTAAAACTTGTGGCCTTATAAATTCTGTGAAGTTC
TACCAGGCCTCAACAAGTGAACTTTATGGAAAAGTGCAGGAAATACCCCA
GAAGGAGACCACACCTTTCTATCCGAGGTCACCCTATGGAGCAGCCAAAC
TCTATGCCTATTGGATTGTGGTGAATTTCCGTGAAGCTTATAATCTCTTT
GCAGTGAATGGAATTCTCTTCAATCATGAGAGTCCCAGAAGAGGAGCTAA
TTTTGTTACTCGAAAAATTAGCCGGTCAGTAGCTAAGATTTACCTTGGAC
AACTGGAATGTTTCAGCTTGGGAAATCTGGATGCCAAACGAGACTGGGGC
CATGCCAAGGACTATGTAGAGGCTATGTGGCTCATGTTGCAGAATGATGA
GCCAGAGGACTTTGTCATAGCTACTGGGGAAGTTCACAGTGTCCGTGAAT
TTGTTGAAAAGTCATTCATGCACATCGGAAAAACCATTGTGTGGGAAGGA
AAGAATGAAAATGAAGTGGGCAGATGTAAAGAGACCGGCAAAGTTCACGT
GACTGTGGATCTGAAATACTACCGACCGACTGAAGTGGACTTTCTGCAGG
GAGACTGCTCCAAGGCTCAGCAGAAGCTAAACTGGAAGCCCCGCGTTGCC
TTTGACGAGCTGGTGAGGGAGATGGTGCAGGCCGACGTGGAGCTCATGAG
GACCAACCCCAACGCTTGA
GenBank Accession No. NM_146041 (GenBank version
dated 18-APR-2009)
Protein sequence of rat GDP-mannose 4,6-
dehydratase
(SEQ ID NO: 5)
MAHAPASCRRYPGSGDGEMGKLRKVALITGITGQDGSYLAEFLLEKGYEV
HGIVRRSSSFNTGRIEHLYKNPQAHIEGNMKLHYGDLTDSTCLVKIINEV
KPTEIYNLGAQSHVKISFDLAEYTADVDGVGTLRLLDAIKTCGLINSVKF
YQASTSELYGKVQEIPQKETTPFYPRSPYGAAKLYAYWIVVNFREAYNLF
AVNGILFNHESPRRGANFVTRKISRSVAKIYLGQLECFSLGNLDAKRDWG
HAKDYVEAMWLMLQNDEPEDFVIATGEVHSVREFVEKSFMHIGKTIVWEG
KNENEVGRCKETGKIHVTVDLKYYRPTEVDFLQGDCSKAQQKLNWKPRVA
FDELVREMVQADVELMRTNPNA
GenBank Accession No. NP_001034695 (GenBank
version dated 18-APR-2009)
mRNA sequence of rat GDP-mannose 4,6-dehydratase
(SEQ ID NO: 6)
ATGGCCCACGCTCCCGCTAGCTGCCGGAGATACCCGGGCTCCGGGGATGG
CGAGATGGGCAAGCTCAGGAAGGTAGCTCTCATCACCGGCATCACTGGCC
AGGATGGTTCATACTTGGCAGAATTCCTGCTGGAGAAAGGATACGAGGTC
CATGGAATAGTACGGCGATCTAGTTCATTTAATACAGGTCGAATTGAACA
TTTATATAAGAATCCTCAGGCTCATATTGAAGGAAACATGAAGTTGCACT
ATGGCGACCTGACTGACAGCACCTGCCTGGTGAAAATCATCAATGAAGTG
AAGCCTACAGAGATCTACAATCTTGGCGCTCAGAGCCATGTCAAGATCTC
CTTTGACTTAGCTGAATACACCGCAGACGTTGATGGAGTTGGCACCTTGC
GGCTTCTGGATGCAATTAAAACTTGCGGCCTTATAAATTCTGTGAAGTTC
TACCAGGCCTCGACAAGTGAACTTTATGGAAAAGTTCAGGAAATACCCCA
GAAAGAGACCACACCTTTCTATCCGAGGTCACCCTATGGAGCCGCCAAGC
TCTATGCCTATTGGATTGTGGTGAATTTCCGTGAAGCTTATAATCTCTTT
GCAGTGAATGGCATTCTCTTCAATCACGAGAGCCCCAGAAGAGGAGCTAA
TTTTGTTACTCGAAAAATTAGCCGGTCAGTAGCTAAGATTTACCTTGGAC
AACTGGAATGTTTCAGTTTGGGAAATCTGGATGCCAAACGAGACTGGGGC
CATGCCAAGGACTATGTAGAGGCTATGTGGCTGATGTTGCAAAATGATGA
GCCGGAGGACTTTGTCATAGCTACTGGGGAAGTTCACAGTGTCCGTGAAT
TTGTTGAAAAATCATTCATGCACATTGGAAAAACCATTGTGTGGGAAGGA
AAGAATGAAAATGAAGTAGGCAGATGTAAGGAGACCGGCAAAATTCACGT
GACTGTGGATCTGAAATACTACCGACCGACTGAAGTGGACTTTCTACAGG
GAGACTGCTCCAAGGCTCAGCAGAAACTGAACTGGAAACCCCGCGTTGCC
TTCGATGAGCTGGTGAGAGAGATGGTGCAGGCCGACGTGGAGCTCATGAG
GACCAACCCCAACGCTTGA
GenBank Accession No. NM_001039606 (GenBank
version dated 18-APR-2009)
Protein sequence of Chinese hamster GDP-mannose
4,6-dehydratase
(SEQ ID NO: 7)
MAHAPARCPSARGSGDGEMGKPRNVALITGITGQDGSYLAEFLLEKGYEV
HGIVRRSSSFNTGRIEHLYKNPQAHIEGNMKLHYGDLTDSTCLVKIINEV
KPTEIYNLGAQSHVKISFDLAEYTADVDGVGTLRLLDAVKTCGLINSVKF
YQASTSELYGKVQEIPQKETTPFYPRSPYGAAKLYAYWIVVNFREAYNLF
AVNGILFNHESPRRGANFVTRKISRSVAKIYLGQLECFSLGNLDAKRDWG
HAKDYVEAMWLMLQNDEPEDFVIATGEVHSVREFVEKSFLHIGKTIVWEG
KNENEVGRCKETGKVHVTVDLKYYRPTEVDFLQGDCTKAKQKLNWKPRVA
FDELVREMVHADVELMRTNPNA
GenBank Accession No. Q8K3X3 (GenBank version
dated 20-JAN-2009)
mRNA sequence of Chinese hamster GDP-mannose
4,6-dehydratase
(SEQ ID NO: 8)
agactgtggcggccgctgcagctccgtgaggcgactggcgcgcgcaccca
cgtctctgtcggcccgctgccggttccacggttccactcctccttccact
cggctgcacgctcacccgcccgcggcgacATGGCTCACGCTCCCGCTAGC
TGCCCGAGCTCCAGGAACTCTGGGGACGGCGATAAGGGCAAGCCCAGGAA
GGTGGCGCTCATCACGGGCATCACCGGCCAGGATGGCTCATACTTGGCAG
AATTCCTGCTGGAGAAAGGATACGAGGTTCATGGAATTGTACGGCGATCC
AGTTCATTTAATACAGGTCGAATTGAACATTTATATAAGAATCCACAGGC
TCATATTGAAGGAAACATGAAGTTGCACTATGGTGACCTCACCGACAGCA
CCTGCCTAGTAAAAATCATCAATGAAGTCAAACCTACAGAGATCTACAAT
CTTGGTGCCCAGAGCCATGTCAAGATTTCCTTTGACTTAGCAGAGTACAC
TGCAGATGTTGATGGAGTTGGCACCTTGCGGCTTCTGGATGCAATTAAGA
CTTGTGGCCTTATAAATTCTGTGAAGTTCTACCAGGCCTCAACTAGTGAA
CTGTATGGAAAAGTGCAAGAAATACCCCAGAAAGAGACCACCCCTTTCTA
TCCAAGGTCGCCCTATGGAGCAGCCAAACTTTATGCCTATTGGATTGTAG
TGAACTTTCGAGAGGCTTATAATCTCTTTGCGGTGAACGGCATTCTCTTC
AATCATGAGAGTCCTAGAAGAGGAGCTAATTTTGTTACTCGAAAAATTAG
CCGGTCAGTAGCTAAGATTTACCTTGGACAACTGGAATGTTTCAGTTTGG
GAAATCTGGACGCCAAACGAGACTGGGGCCATGCCAAGGACTATGTCGAG
GCTATGTGGCTGATGTTACAAAATGATGAACCAGAGGACTTTGTCATAGC
TACTGGGGAAGTTCATAGTGTCCGTGAATTTGTTGAGAAATCATTCATGC
ACATTGGAAAGACCATTGTGTGGGAAGGAAAGAATGAAAATGAAGTGGGC
AGATGTAAAGAGACCGGCAAAATTCATGTGACTGTGGATCTGAAATACTA
CCGACCAACTGAAGTGGACTTCCTGCAGGGAGACTGCTCCAAGGCGCAGC
AGAAACTGAACTGGAAGCCCCGCGTTGCCTTTGACGAGCTGGTGAGGGAG
ATGGTGCAAGCCGATGTGGAGCTCATGAGAACCAACCCCAACGCCTGAgc
acctctacaaaaaattcgcgagacatggactatggtgcagagccagccaa
ccagagtccagccactcctgagaccatcgaccataaaccctcgactgcct
gtgtcgtccccacagctaagagctgggccacaggtttgtgggcaccagga
cggggacactccagagctaaggccacttcgcttttgtcaaaggctcctct
gaatgattttgggaaatcaagaagtttaaaatcacatactcattttactt
gaaattatgtcactagacaacttaaatttttgagtcttgagattgttttt
ctcttttcttattaaatgatctttctatgaaccagcaaaaaaaaaaaaaa
aaaaaa
GenBank Accession No. AF525364 (GenBank version
dated 04-AUG-2002)
Protein sequence of human GDP-keto-6-deoxymannose
3,5-epimerase, 4-reductase (FX protein, tissue
specific transplantation antigen P35B)
(SEQ ID NO: 9)
MGEPQGSMRILVTGGSGLVGKAIQKVVADGAGLPGEDWVFVSSKDADLTD
TAQTRALFEKVQPTHVIHLAAMVGGLFRNIKYNLDFWRKNVHMNDNVLHS
AFEVGARKVVSCLSTCIFPDKTTYPIDETMIHNGPPHNSNFGYSYAKRMI
DVQNRAYFQQYGCTFTAVIPTNVFGPHDNFNIEDGHVLPGLIHKVHLAKS
SGSALTVWGTGNPRRQFIYSLDLAQLFIWVLREYNEVEPIILSVGEEDEV
SIKEAAEAVVEAMDFHGEVTFDTTKSDGQFKKTASNSKLRTYLPDFRFTP
FKQAVKETCAWFTDNYEQARK
GenBank Accession No. NP_003304 (GenBank version
dated 10-DEC-2008)
mRNA sequence of human GDP-keto-6-deoxymannose
3,5-epimerase, 4-reductase (FX protein, tissue
specific transplantation antigen P35B)
(SEQ ID NO: 10)
ATGGGTGAACCCCAGGGATCCATGCGGATTCTAGTGACAGGGGGCTCTGG
GCTGGTAGGCAAAGCCATCCAGAAGGTGGTAGCAGATGGAGCTGGACTTC
CTGGAGAGGACTGGGTGTTTGTCTCCTCTAAAGACGCCGATCTCACGGAT
ACAGCACAGACCCGCGCCCTGTTTGAGAAGGTCCAACCCACACACGTCAT
CCATCTTGCTGCAATGGTGGGGGGCCTGTTCCGGAATATCAAATACAATT
TGGACTTCTGGAGGAAAAACGTGCACATGAACGACAACGTCCTGCACTCG
GCCTTTGAGGTGGGCGCCCGCAAGGTGGTGTCCTGCCTGTCCACCTGTAT
CTTCCCTGACAAGACGACCTACCCGATAGATGAGACCATGATCCACAATG
GGCCTCCCCACAACAGCAATTTTGGGTACTCGTATGCCAAGAGGATGATC
GACGTGCAGAACAGGGCCTACTTCCAGCAGTACGGCTGCACCTTCACCGC
TGTCATCCCCACCAACGTCTTCGGGCCCCACGACAACTTCAACATCGAGG
ATGGCCACGTGCTGCCTGGCCTCATCCACAAGGTGCACCTGGCCAAGAGC
AGCGGCTCGGCCCTGACGGTGTGGGGTACAGGGAATCCGCGGAGGCAGTT
CATATACTCGCTGGACCTGGCCCAGCTCTTTATCTGGGTCCTGCGGGAGT
ACAATGAAGTGGAGCCCATCATCCTCTCCGTGGGCGAGGAAGATGAGGTC
TCCATCAAGGAGGCAGCCGAGGCGGTGGTGGAGGCCATGGACTTCCATGG
GGAAGTCACCTTTGATACAACCAAGTCGGATGGGCAGTTTAAGAAGACAG
CCAGTAACAGCAAGCTGAGGACCTACCTGCCCGACTTCCGGTTCACACCC
TTCAAGCAGGCGGTGAAGGAGACCTGTGCTTGGTTCACTGACAACTACGA
GCAGGCCCGGAAGTGA
GenBank Accession No. NM_003313 (GenBank version
dated 10-DEC-2008)
Protein sequence of mouse GDP-keto-6-deoxymannose
3,5-epimerase, 4-reductase (FX protein, tissue
specific transplantation antigen P35B)
(SEQ ID NO: 11)
MGEPHGSMRILVTGGSGLVGRAIQKVVADGAGLPGEEWVFVSSKDADLTD
AAQTQALFQKVQPTHVIHLAAMVGGLFRNIKYNLDFWRKNVHINDNVLHS
AFEVGARKVVSCLSTCIFPDKTTYPIDETMIHNGPPHSSNFGYSYAKRMI
DVQNRAYFQQHGCTFTAVIPTNVFGPYDNFNIEDGHVLPGLIHKVHLAKS
SDSALTVWGTGKPRRQFIYSLDLARLFIWVLREYSEVEPIILSVGEEDEV
SIKEAAEAVVEAMDFNGEVTFDSTKSDGQYKKTASNGKLRSYLPDFRFTP
FKQAVKETCTWFTDNYEQARK
GenBank Accession No. NP_112478 (GenBank version
dated 10-MAY-2009)
mRNA sequence of mouse GDP-keto-6-deoxymannose
3,5-epimerase, 4-reductase (FX protein, tissue
specific transplantation antigen P35B)
(SEQ ID NO: 12)
ATGGGCGAACCCCATGGATCCATGAGGATCCTAGTGACAGGGGGCTCTGG
ACTGGTGGGTAGAGCCATCCAGAAGGTGGTTGCAGATGGGGCCGGCTTAC
CTGGAGAGGAATGGGTGTTTGTCTCCTCCAAAGATGCAGATCTGACGGAT
GCAGCCCAAACCCAAGCACTCTTCCAGAAAGTACAGCCCACCCACGTCAT
CCATCTCGCTGCAATGGTAGGCGGCCTTTTCCGGAATATCAAATACAACT
TGGATTTCTGGCGGAAAAACGTGCACATCAATGACAACGTCCTGCATTCG
GCCTTCGAGGTGGGCGCTCGCAAGGTGGTCTCCTGCCTGTCCACCTGCAT
CTTCCCTGACAAGACCACCTATCCTATTGACGAGACAATGATCCACAACG
GGCCGCCTCACAGCAGCAATTTCGGGTACTCATACGCCAAGAGGATGATT
GACGTGCAGAACAGAGCCTACTTCCAGCAGCACGGCTGTACCTTCACCGC
CGTCATCCCTACCAATGTCTTTGGGCCTTATGACAACTTCAACATCGAAG
ATGGCCACGTGCTACCCGGCCTCATCCATAAGGTGCACCTGGCCAAGAGT
AGTGACTCGGCCCTGACGGTGTGGGGTACAGGGAAGCCGCGGAGGCAGTT
CATCTACTCACTGGACCTCGCCCGGCTCTTCATCTGGGTCCTACGGGAGT
ACAGTGAGGTGGAGCCCATCATCCTCTCAGTGGGTGAGGAAGATGAAGTG
TCCATCAAGGAGGCAGCTGAGGCTGTAGTGGAGGCCATGGACTTCAATGG
GGAAGTCACTTTTGATTCAACAAAGTCAGATGGGCAATATAAGAAGACAG
CCAGCAATGGCAAGTTGCGGTCCTACTTGCCCGACTTCCGTTTCACACCC
TTCAAGCAGGCTGTGAAGGAAACCTGCACTTGGTTCACCGACAACTATGA
GCAGGCCCGGAAGTAA
GenBank Accession No. NM_031201 (GenBank version
dated 10-MAY-2009)
Protein sequence of rat GDP-keto-6-deoxymannose
3,5-epimerase, 4-reductase (FX protein, tissue
specific transplantation antigen P35B)
(SEQ ID NO: 13)
MGEPHGSMRILVTGGSGLVGRAIQKVVADGAGLPGEEWVFVSSKDADLTD
AAQTQALFQKVQPTHVIHLAAMVGGLFRNIKYNLDFWRKNVHINDNVLHS
AFEVGTRKVVSCLSTCIFPDKTTYPIDETMIHNGPPHSSNFGYSYAKRMI
DVQNRAYFQQHGCTFTSVIPTNVFGPYDNFNIEDGHVLPGLIHKVHLAKS
SGSALTVWGTGKPRRQFIYSLDLARLFIWVLREYNEVEPIILSVGEEDEV
SIKEAAEAVVEAMDFSGEVTFDSTKSDGQYKKTASNGKLRSYLPDFCFTP
FKQAVKETCAWFTENYEQARK
GenBank Accession No. NP_001120927 (GenBank
version dated 24-AUG-2008)
mRNA sequence of rat GDP-keto-6-deoxymannose 3,5-
epimerase, 4-reductase (FX protein, tissue
specific transplantation antigen P35B)
(SEQ ID NO: 14)
ATGGGTGAACCCCACGGATCCATGAGGATCCTAGTAACAGGGGGCTCTGG
ACTGGTGGGCAGAGCCATCCAGAAGGTGGTCGCAGATGGGGCCGGCTTGC
CTGGAGAGGAATGGGTGTTTGTCTCCTCCAAAGATGCAGATCTGACGGAT
GCAGCGCAAACCCAAGCTCTGTTCCAGAAGGTACAGCCCACCCACGTCAT
CCATCTTGCTGCAATGGTAGGCGGCCTTTTCCGGAATATTAAATACAACT
TGGATTTCTGGAGGAAGAACGTGCACATCAATGACAACGTCCTACATTCA
GCCTTCGAGGTGGGCACACGCAAGGTGGTCTCCTGCCTGTCCACCTGCAT
CTTCCCTGACAAGACCACCTATCCTATTGATGAGACCATGATCCACAACG
GGCCGCCTCACAGCAGCAATTTTGGGTACTCATATGCCAAGAGGATGATT
GACGTGCAGAACAGGGCCTACTTCCAGCAGCATGGCTGTACCTTCACCTC
TGTCATCCCTACCAATGTCTTTGGGCCTTACGACAACTTCAACATCGAAG
ATGGCCACGTGCTGCCGGGCCTCATCCATAAGGTGCACCTGGCCAAGAGC
AGTGGTTCAGCCTTGACTGTGTGGGGTACGGGGAAGCCGCGGAGACAGTT
CATCTACTCACTGGACCTAGCCCGGCTCTTCATCTGGGTCCTTCGGGAGT
ACAATGAGGTGGAGCCCATCATCCTCTCAGTGGGCGAGGAAGATGAAGTG
TCTATCAAGGAGGCAGCTGAGGCTGTGGTGGAGGCCATGGACTTCTCTGG
GGAAGTCACTTTTGATTCAACAAAGTCAGATGGGCAGTATAAGAAGACAG
CCAGCAATGGCAAGTTGCGGTCCTACTTGCCTGACTTCTGTTTCACACCC
TTCAAGCAGGCTGTGAAGGAAACTTGTGCTTGGTTCACTGAAAACTACGA
GCAGGCCCGGAAGTAA
GenBank Accession No. NM_001127455 (GenBank
version dated 24-AUG-2008)
Protein sequence of Chinese hamster GDP-keto-6-
deoxymannose 3,5-epimerase, 4-reductase
(SEQ ID NO: 15)
MGEPQGSRRILVTGGSGLVGRAIQKVVADGAGLPGEEWVFVSSKDADLTD
AAQTQALFQKVQPTHVIHLAAMVGGLFRNIKYNLDFWRKNVHINDNVLHS
AFEVGTRKVVSCLSTCIFPDKTTYPIDETMIHNGPPHSSNFGYSYAKRMI
DVQNRAYFQQHGCTFTAVIPTNVFGPHDNFNIEDGHVLPGLIHKVHLAKS
NGSALTVWGTGKPRRQFIYSLDLARLFIWVLREYNEVEPIILSVGEEDEV
SIKEAAEAVVEAMDFCGEVTFDSTKSDGQYKKTASNGKLRAYLPDFRFTP
FKQAVKETCAWFTDNYEQARK
GenBank Accession No. Q8K3X2 (GenBank version
dated 20-JAN-2009)
mRNA sequence of Chinese hamster GDP-keto-6-
deoxymannose 3,5-epimerase, 4-reductase (FX
protein)
(SEQ ID NO: 16)
ccggaagtagctcttggactggtggaaccctgcgcaggtgcagcaacaAT
GGGTGAGCCCCAGGGATCCAGGAGGATCCTAGTGACAGGGGGCTCTGGAC
TGGTGGGCAGAGCTATCCAGAAGGTGGTCGCAGATGGCGCTGGCTTACCC
GGAGAGGAATGGGTGTTTGTCTCCTCCAAAGATGCAGATCTGACGGATGC
AGCACAAACCCAAGCCCTGTTCCAGAAGGTACAGCCCACCCATGTCATCC
ATCTTGCTGCAATGGTAGGAGGCCTTTTCCGGAATATCAAATACAACTTG
GATTTCTGGAGGAAGAATGTGCACATCAATGACAACGTCCTGCACTCAGC
TTTCGAGGTGGGCACTCGCAAGGTGGTCTCCTGCCTGTCCACCTGTATCT
TCCCTGACAAGACCACCTATCCTATTGATGAAACAATGATCCACAATGGT
CCACCCCACAGCAGCAATTTTGGGTACTCGTATGCCAAGAGGATGATTGA
CGTGCAGAACAGGGCCTACTTCCAGCAGCATGGCTGCACCTTCACTGCTG
TCATCCCTACCAATGTCTTTGGACCTCATGACAACTTCAACATTGAAGAT
GGCCATGTGCTGCCTGGCCTCATCCATAAGGTGCATCTGGCCAAGAGTAA
TGGTTCAGCCTTGACTGTTTGGGGTACAGGGAAACCACGGAGGCAGTTCA
TCTACTCACTGGACCTAGCCCGGCTCTTCATCTGGGTCCTGCGGGAGTAC
AATGAAGTTGAGCCCATCATCCTCTCAGTGGGCGAGGAAGATGAAGTCTC
CATTAAGGAGGCAGCTGAGGCTGTAGTGGAGGCCATGGACTTCTGTGGGG
AAGTCACTTTTGATTCAACAAAGTCAGATGGGCAGTATAAGAAGACAGCC
AGCAATGGCAAGCTTCGGGCCTACTTGCCTGATTTCCGTTTCACACCCTT
CAAGCAGGCTGTGAAGGAGACCTGTGCCTGGTTCACCGACAACTATGAGC
AGGCCCGGAAGTGAagcatgggacaagcgggtgctcagctggcaatgccc
agtcagtaggctgcagtctcatcatttgcttgtcaagaactgaggacagt
atccagcaacctgagccacatgctggtctctctgccagggggcttcatgc
agccatccagtagggcccatgtttgtccatcctcgggggaaggccagacc
aacaccttgtttgtctgcttctgccccaacctcagtgcatccatgctggt
cctgctgtcccttgtctagaaaccaataaaatggattttcataaaaaaaa
aaaaaaaaaaa
GenBank Accession No. AF525365 (GenBank version
dated 04-AUG-2002)
Protein sequence of human GDP fucose pyrophos-
phorylase
(SEQ ID NO: 17)
MAAARDPPEVSLREATQRKLRRFSELRGKLVARGEFWDIVAITAADEKQE
LAYNQQLSEKLKRKELPLGVQYHVFVDPAGAKIGNGGSTLCALQCLEKLY
GDKWNSFTILLIHSGGYSQRLPNASALGKIFTALPLGNPIYQMLELKLAM
YIDFPLNMNPGILVTCADDIELYSIGEFEFIRFDKPGFTALAHPSSLTIG
TTHGVFVLDPFDDLKHRDLEYRSCHRFLHKPSIEKMYQFNAVCRPGNFCQ
QDFAGGDIADLKLDSDYVYTDSLFYMDHKSAKMLLAFYEKIGTLSCEIDA
YGDFLQALGPGATVEYTRNTSHVIKEESELVEMRQRIFHLLKGTSLNVVV
LNNSKFYHIGTTEEYLFYFTSDNSLKSELGLQSITFSIFPDIPECSGKTS
CIIQSILDSRCSVAPGSVVEYSRLGPDVSVGENCIISGSYILTKAALPAH
SFVCSLSLKMNRCLKYATMAFGVQDNLKKSVKTLSDIKLLQFFGVCFLSC
LDVWNLKVTEELFSGNKTCLSLWTARIFPVCSSLSDSVITSLKMLNAVKN
KSAFSLNSYKLLSIEEMLIYKDVEDMITYREQIFLEISLKSSLM
GenBank Accession No. AAC73005 (GenBank version
dated 12-NOV-1998)
mRNA sequence of human GDP fucose pyrophos-
phorylase
(SEQ ID NO: 18)
ATGGCAGCTGCTAGGGACCCTCCGGAAGTATCGCTGCGAGAAGCCACCCA
GCGAAAATTGCGGAGGTTTTCCGAGCTAAGAGGCAAACTTGTAGCACGTG
GAGAATTCTGGGACATAGTTGCAATAACAGCGGCTGATGAAAAACAGGA
ACTTGCTTACAACCAACAGCTGTCAGAAAAGCTGAAAAGAAAGGAGTTAC
CCCTTGGAGTTCAATATCACGTTTTTGTGGATCCTGCTGGAGCCAAAATT
GGAAATGGAGGATCAACACTTTGTGCCCTTCAATGTTTGGAAAAGCTATA
TGGAGATAAATGGAATTCTTTTACCATCTTATTAATTCACTCTGGTGGCT
ACAGTCAACGACTTCCAAATGCAAGTGCTCTGGGAAAAATTTTCACTGCT
TTACCTCTTGGTAACCCCATTTATCAGATGCTAGAATTAAAGCTAGCCAT
GTACATTGATTTCCCCTTAAATATGAATCCTGGAATTCTGGTTACCTGTG
CAGATGATATTGAACTTTATAGTATTGGAGAATTTGAGTTTATTAGGTTT
GACAAACCTGGCTTTACTGCTTTAGCTCATCCTTCTAGTTTGACGATAGG
TACCACACATGGAGTATTTGTCTTAGATCCTTTTGATGATTTAAAACATA
GAGACCTTGAATACAGGTCTTGCCATCGTTTCCTTCATAAGCCCAGCATA
GAAAAGATGTATCAGTTTAATGCTGTGTGTAGACCTGGAAATTTTTGTCA
ACAGGACTTTGCTGGGGGTGACATTGCCGATCTTAAATTAGACTCTGACT
ATGTCTACACAGATAGCCTATTTTATATGGATCATAAATCAGCAAAAATG
TTACTTGCTTTTTATGAAAAAATAGGCACACTGAGCTGTGAAATAGATGC
CTATGGTGACTTTCTGCAGGCTTTGGGACCTGGAGCAACTGTGGAGTACA
CCAGAAACACATCACATGTCATTAAAGAAGAGTCAGAGTTGGTAGAAATG
AGGCAGAGAATATTTCATCTTCTTAAAGGAACATCACTAAATGTTGTTGT
TCTTAATAACTCCAAATTTTATCACATTGGAACAACCGAAGAATATTTGT
TTTACTTTACCTCAGATAACAGTTTAAAGTCAGAGCTCGGCTTACAGTCC
ATAACTTTTAGTATCTTTCCAGATATACCAGAATGCTCTGGCAAAACATC
CTGTATCATTCAAAGCATACTGGATTCAAGATGTTCTGTGGCACCTGGCT
CAGTTGTGGAGTATTCCAGATTGGGGCCTGATGTTTCAGTTGGGGAAAAC
TGCATTATTAGTGGTTCTTACATCCTAACAAAAGCTGCCCTCCCCGCACA
TTCTTTTGTATGTTCCTTAAGCTTAAAGATGAATAGATGCTTAAAGTATG
CAACTATGGCATTTGGAGTGCAAGACAACTTGAAAAAGAGTGTGAAAACA
TTGTCAGATATAAAGTTACTTCAATTCTTTGGAGTCTGTTTCCTGTCATG
CTTAGATGTTTGGAATCTTAAAGTTACAGAGGAACTGTTCTCTGGTAACA
AGACATGTCTGAGTTTGTGGACTGCACGCATTTTCCCAGTTTGTTCTTCT
TTGAGTGACTCAGTTATAACATCCCTAAAGATGTTAAATGCTGTTAAGAA
CAAGTCAGCATTCAGCCTGAATAGCTATAAGTTGCTGTCCATTGAAGAAA
TGCTTATCTACAAAGATGTAGAAGATATGATAACTTACAGGGAACAAATT
TTTCTAGAAATCAGTTTAAAAAGCAGTTTGATGTAG
GenBank Accession No. AF017445 (GenBank version
dated 12-NOV-1998)
Protein sequence of mouse GDP fucose pyrophos-
phorylase (fucose-1-phosphate guanylyltransferase)
(SEQ ID NO: 19)
MASLREATLRKLRRFSELRGKPVAAGEFWDVVAITAADEKQELAYKQQLS
EKLKKRELPLGVQYHVFPDPAGTKIGNGGSTLCSLECLESLCGDKWNSLK
VLLIHSGGYSQRLPNASALGKIFTALPLGEPIYQMLELKLAMYVDFPSNM
RPGVLVTCADDIELYSVGDSEYIAFDQPGFTALAHPSSLAVGTTHGVFVL
HSDSSLQHGDLEYRQCYQFLHKPTIENMHRFNAVHRQRSFGQQNLSGGDT
DCLPLHTEYVYTDSLFYMDHKSAKKLLDFYKSEGPLNCEIDAYGDFLQAL
GPGATAEYTRNTSHVTKEESQLLDMRQKIFHLLKGTPLNVVVLNNSRFYH
IGTLQEYLLHFTSDSALKTELGLQSIAFSVSPSVPERSSGTACVIHSIVD
SGCCVAPGSVVEYSRLGPEVSIGENCIISSSVIAKTVVPAYSFLCSLSVK
INGHLKYSTMVFGMQDNLKNSVKTLEDIKALQFFGVCFLSCLDIWNLKAT
EKLFSGNKMNLSLWTACIFPVCSSLSESATASLGMLSAVRNHSPFNLSDF
NLLSIQEMLVYKDVQDMLAYREHIFLEISSNKNQSDLEKS
GenBank Accession No. NP_083606 (GenBank version
dated 10-FEB-2008)
mRNA sequence of mouse GDP fucose pyrophos-
phorylase (fucose-1-phosphate guanylyltransferase)
(SEQ ID NO: 20)
agtgtgctcccggaagtcggccATGGCGTCTCTCCGCGAAGCCACCCTGC
GGAAACTGCGCAGATTTTCTGAGCTGAGAGGCAAACCCGTGGCAGCTGGA
GAATTCTGGGATGTGGTTGCAATAACAGCAGCTGATGAAAAGCAGGAGCT
CGCTTACAAGCAACAGTTGTCCGAGAAGCTGAAGAAAAGGGAATTGCCTC
TTGGAGTTCAATACCATGTTTTTCCAGATCCTGCTGGGACCAAAATTGGA
AATGGAGGATCAACACTTTGTTCCCTTGAGTGTTTGGAAAGCCTCTGTGG
AGACAAATGGAATTCTCTGAAGGTCCTGCTAATCCACTCTGGTGGCTACA
GCCAACGCCTTCCCAATGCGAGTGCTTTAGGAAAGATCTTCACAGCCTTA
CCACTTGGTGAACCCATTTATCAGATGTTGGAGTTAAAACTAGCCATGTA
CGTGGATTTCCCCTCAAACATGAGGCCTGGAGTCTTGGTCACCTGTGCAG
ATGATATCGAACTCTACAGTGTTGGGGACAGTGAGTACATTGCCTTTGAC
CAGCCTGGCTTTACTGCCTTAGCCCATCCGTCTAGTCTGGCTGTAGGCAC
TACTCATGGAGTATTTGTCTTGCACTCTGACAGTTCCTTACAACATGGTG
ACCTTGAGTACAGGCAATGCTACCAATTCCTCCACAAGCCCACCATTGAA
AACATGCACCGCTTTAATGCTGTGCATAGACAACGAAGCTTTGGTCAACA
GAACTTGTCTGGAGGTGACACTGACTGTCTTCCATTGCACACTGAGTATG
TCTACACAGATAGCCTGTTTTACATGGATCACAAATCAGCCAAAAAGTTA
CTTGATTTCTATAAAAGTGAAGGCCCACTGAACTGTGAAATAGATGCCTA
TGGAGACTTTCTTCAGGCACTGGGGCCTGGAGCAACTGCAGAGTACACCA
GGAACACATCTCATGTCACTAAAGAAGAGTCCCAGTTGTTGGACATGAGG
CAGAAAATATTCCACCTCCTCAAGGGAACACCACTGAATGTTGTTGTTCT
TAATAACTCCAGATTTTATCACATTGGAACACTGCAAGAGTATCTGCTTC
ATTTCACCTCTGATAGTGCATTAAAGACGGAGCTGGGCTTACAATCCATA
GCTTTCAGTGTCTCTCCAAGTGTTCCTGAGCGCTCCAGTGGAACAGCCTG
TGTCATTCACAGTATAGTGGATTCAGGATGCTGTGTGGCCCCTGGCTCAG
TGGTAGAGTATTCTAGATTGGGGCCTGAGGTGTCCATCGGGGAAAACTGC
ATTATCAGCAGTTCTGTCATAGCAAAAACTGTTGTGCCAGCATATTCTTT
TTTGTGTTCTTTAAGTGTGAAGATAAATGGACACTTAAAATATTCTACTA
TGGTGTTTGGCATGCAAGACAACTTGAAGAACAGTGTTAAAACACTGGAA
GACATAAAGGCACTTCAGTTCTTTGGAGTCTGTTTTCTGTCTTGTTTAGA
CATTTGGAATCTTAAAGCTACAGAGAAACTATTCTCTGGAAATAAGATGA
ATCTGAGCCTGTGGACTGCATGCATTTTCCCTGTCTGTTCATCTCTGAGT
GAGTCGGCTACAGCATCCCTTGGGATGTTAAGCGCTGTAAGGAACCATTC
ACCATTCAACCTAAGTGACTTTAACCTTTTGTCCATCCAGGAAATGCTTG
TCTACAAAGATGTACAAGACATGCTAGCTTATAGGGAACACATTTTTCTA
GAAATTAGTTCAAATAAAAATCAATCTGATTTAGAGAAATCTTGAatata
ttttggccataaacaaaattgcaaatacaggcattttctatagacctctg
acatttttgtttgttttaataaagtaatataataaaaattatgttaatat
aactgttgtagcttggtaatgagaatggtacaactgaccacttctgctag
aagtacgttccaggactagagtcaggaaaggtcggctgttttagatgttt
acaccatcttacaattgtgctctttggtaaagatccatttatgggacact
gtttcattcacaaaataaatatttctgttttataggatgattttctaaac
ataacatatctttaaagcttttctatcttcttttgaaatttggaccaata
aaattctaggtgatatggaggattgtattgctcaacttctcatagtgaga
caacacgtaacaaaacattgttataaattcttagaagaaatgtcattatt
tgaggttttctttgaggactttgttctagttttattttatgtgtataaat
gtgttacctgcatgtatgcatgtgcaccacttgcctgcggcacccataga
ggctagaacagctgttctcaacatttgggttgggaccttttgtgggctca
aacaatcctttgaggggtaacctaagtccattggaaaacaaaatatttac
attatgattcataacagtagggaaattacagttaagtagcaacaaaaata
attttatatttggggtcactacagcatggggactgtattgaaaggatagc
agcatcaggaaggttaaaaactgccggtctagaagaaagcattgggtctc
ttggaactagagttatagatgcttagaacctccgtgttgcttctgtaagt
caacctccttagtcctatgaaagtgctatataatgatgtttgtgcctcat
tggtcttgccaaaatgatataaaagtatgtatggatgattttgttcttat
acactagaacatgtgttgccatatcttataaactatgtctactgatatat
tacactggtagctatgtacacacagaactcagttgtctgctcaggaggtg
gtagggatagttgagagccagtactcactcactatggaccttacttaatc
ctctcctagttaatccttctccaaatctcttaacttgacagtggacattt
gccttgcatcattggtggtagtgatgctgtgaacaaacaataggcccaaa
gagaggaaattcaaataggcaatctgaagaactactcaaatcataaacaa
ctgcagggaaatgaaatgggtggaattcctggttatgcgtacctattatg
aaataaacacattagtggaatgtccttaggttgaactgtaatagagttaa
attttatcatacttgtgtttaaaataccttaagtacattgtaatatctgc
tgtggcaactttaattctgtgtaagttttcataaaaatatatgataaaca
agatatctgtcaaaactcctttatattatttatataagaatatttgcctt
tttgaggtactagataataaagcaaagaatgtacgatactatatgacaat
tattggtaaagttacagagaattcaatggatgttaaatgttattaaatac
tcaagactaaagtcctatcaacgatgagaattatgatttcatgttccaag
aaaaaaatatcattaataaagaataccatcacttccttgtaaaaaaaaaa
aaaaaaaaaaaaaaaaaaaaaaaaaa
GenBank Accession No. NM_029330 (GenBank version
dated 10-FEB-2008)
Protein sequence of rat GDP fucose pyrophos-
phorylase (fucose-1-phosphate guanylyltransferase)
(SEQ ID NO: 21)
METLREATLRKLRRFSELRGKPVAAGEFWDVVAITAADEKQELAYKQQLS
EKLRRKELPLGVQYHVFPDPAGTKIGNGGSTLCSLQCLKSLYGDEWNSFK
VLLIHSGGYSQRLPNASALGKIFTALPLGEPIYQMLELKLAMYVDFPSHM
KPGVLVTCADDIELYSVGDCQYIAFDQPGFTALAHPSSLAVGTTHGVFVL
HSASSLQHGDLQYRQCHRFLHKPTIENMHQFNAVQRQGSFAQQDFPGGDT
ACLPLHTEYVYTDSLFYMDHKSAKKLLDFYKNVNQLNCEIDAYGDFLQAL
GPGATAEYTRNTSHVTKEDSQLLDMRQKIFHLLKGTPLNVVVLNNSRFYH
IGTTQEYLLHFTSDSTLRSRARLTVHSFQVSLQVSLNPPMKQPVSFTVYW
DSGCCVAPGSVVEYSRLGPEVSIGENCIVSSSVLANTAVPAYSFVCSLSV
RTNGLLEYSTMVFSVQDNLKGSVKTLEDIKALQFFGVCFLSCLDIWNLKA
TEKLFSGSKRNLSLWTARIFPVCPSLSESVTASLGMLSAVRSHSPFSLSN
FKLMSIQEMLVYKDVQDMLAYREQIFLEINSNKKQSDLEKS
GenBank Accession No. NP_955788 (GenBank version
dated 11-FEB-2008)
Protein sequence of rat GDP fucose pyrophos-
phorylase (fucose-1-phosphate guanylyltransferase)
(SEQ ID NO: 22)
ATGGAGACTCTCCGGGAAGCCACCCTGCGGAAACTGCGCAGATTTTCGGA
GCTGAGAGGCAAACCTGTGGCAGCTGGAGAATTCTGGGATGTGGTTGCGA
TAACAGCAGCCGATGAAAAGCAGGAGCTCGCTTACAAGCAGCAGTTGTCA
GAAAAGCTGAGAAGAAAGGAATTGCCTCTTGGAGTTCAATACCATGTTTT
TCCTGATCCTGCTGGGACCAAAATTGGAAATGGAGGATCGACACTTTGTT
CCCTTCAGTGCCTAAAAAGCCTCTATGGAGATGAATGGAATTCTTTCAAG
GTCCTGTTAATTCACTCCGGTGGCTACAGTCAACGCCTTCCCAATGCAAG
TGCTTTAGGAAAGATCTTCACAGCCTTACCACTTGGTGAACCCATCTATC
AGATGTTGGAGTTAAAACTAGCCATGTACGTGGATTTCCCCTCACACATG
AAGCCTGGAGTCTTGGTCACCTGTGCAGATGACATTGAACTGTACAGTGT
TGGGGACTGTCAGTACATTGCCTTTGACCAGCCTGGCTTTACTGCCTTAG
CCCATCCTTCCAGTCTGGCTGTAGGCACCACACACGGAGTATTTGTCTTG
CACTCTGCCAGTTCCTTACAACATGGTGACCTTCAGTACAGACAATGCCA
CCGTTTCCTCCACAAGCCCACCATTGAAAACATGCATCAGTTTAATGCTG
TGCAAAGACAAGGAAGCTTTGCTCAACAGGACTTCCCTGGAGGTGACACC
GCGTGTCTTCCATTGCACACTGAGTATGTCTACACAGATAGCCTGTTTTA
CATGGACCACAAATCGGCCAAAAAGTTACTTGATTTCTATAAAAATGTAA
ACCAACTGAACTGTGAAATAGATGCCTATGGTGACTTTCTGCAGGCACTG
GGGCCTGGAGCAACTGCAGAGTATACCAGGAACACATCACATGTCACTAA
AGAAGACTCCCAGTTGTTGGACATGAGGCAGAAAATATTCCACCTCCTCA
AGGGGACACCACTGAATGTTGTTGTTCTTAATAACTCCAGATTTTATCAC
ATTGGAACAACACAAGAATATCTGCTTCATTTCACGTCTGATAGTACGTT
AAGGTCAAGAGCTAGGCTTACAGTCCATAGCTTTCAAGTGTCTCTCCAAG
TATCCCTGAATCCTCCAATGAAACAGCCTGTATCATTCACAGTATACTGG
GATTCAGGATGCTGTGTGGCACCTGGCTCAGTTGTAGAGTATTCTAGACT
GGGGCCTGAGGTGTCCATTGGGGAAAACTGCATTGTCAGCAGCTCTGTCC
TAGCAAACACTGCTGTGCCGGCATATTCTTTTGTGTGTTCTCTAAGTGTG
AGGACAAATGGACTCTTGGAATATTCTACCATGGTGTTTAGTGTGCAGGA
CAACTTGAAAGGCAGTGTTAAAACCCTGGAAGATATAAAGGCACTTCAGT
TCTTTGGAGTCTGTTTCTTGTCTTGTTTAGACATCTGGAACCTTAAAGCT
ACAGAGAAACTGTTCTCTGGAAGTAAGAGGAACCTGAGCCTGTGGACTGC
ACGGATTTTCCCTGTCTGTCCTTCTCTGAGTGAGTCAGTTACAGCATCCC
TTGGGATGTTAAGTGCTGTAAGGAGCCATTCACCATTCAGCCTAAGCAAC
TTTAAGCTGATGTCCATCCAGGAAATGCTTGTCTACAAAGATGTACAAGA
CATGCTAGCTTATAGGGAGCAAATTTTTCTAGAAATTAATTCAAATAAAA
AACAATCTGATTTAGAGAAATCTTAA
GenBank Accession No. NM_199494 (GenBank version
dated 11-FEB-2008)
Protein sequence of human fucose kinase
(fucokinase)
(SEQ ID NO: 23)
MEQPKGVDWTVIILTCQYKDSVQVFQRELEVRQKREQIPAGTLLLAVEDP
EKRVGSGGATLNALLVAAEHLSARAGFTVVTSDVLHSAWILILHMGRDFP
FDDCGRAFTCLPVENPEAPVEALVCNLDCLLDIMTYRLGPGSPPGVWVCS
TDMLLSVPANPGISWDSFRGARVIALPGSPAYAQNHGVYLTDPQGLVLDI
YYQGTEAEIQRCVRPDGRVPLVSGVVFFSVETAERLLATHVSPPLDACTY
LGLDSGARPVQLSLFFDILHCMAENVTREDFLVGRPPELGQGDADVAGYL
QSARAQLWRELRDQPLTMAYVSSGSYSYMTSSASEFLLSLTLPGAPGAQI
VHSQVEEQQLLAAGSSVVSCLLEGPVQLGPGSVLQHCHLQGPIHIGAGCL
VTGLDTAHSKALHGRELRDLVLQGHHTRLHGSPGHAFTLVGRLDSWERQG
AGTYLNVPWSEFFKRTGVRAWDLWDPETLPAEYCLPSARLFPVLHPSREL
GPQDLLWMLDHQEDGGEALRAWRASWRLSWEQLQPCLDRAATLASRRDLF
FRQALHKARHVLEARQDLSLRPLIWAAVREGCPGPLLATLDQVAAGAGDP
GVAARALACVADVLGCMAEGRGGLRSGPAANPEWMRPFSYLECGDLAAGV
EALAQERDKWLSRPALLVRAARHYEGAGQILIRQAVMSAQHFVSTEQVEL
PGPGQWVVAECPARVDFSGGWSDTPPLAYELGGAVLGLAVRVDGRRPIGA
RARRIPEPELWLAVGPRQDEMTVKIVCRCLADLRDYCQPHAPGALLKAAF
ICAGIVHVHSELQLSEQLLRTFGGGFELHTWSELPHGSGLGTSSILAGTA
LAALQRAAGRVVGTEALIHAVLHLEQVLTTGGGWQDQVGGLMPGIKVGRS
RAQLPLKVEVEEVTVPEGFVQKLNDHLLLVYTGKTRLARNLLQDVLRSWY
ARLPAVVQNAHSLVRQTEECAEGFRQGSLPLLGQCLTSYWEQKKLMAPGC
EPLTVRRMMDVLAPHVHGQSLAGAGGGGFLYLLTKEPQQKEALEAVLAKT
EGLGNYSIHLVEVDTQGLSLKLLGTEASTCCPFP
GenBank Accession No. NP_659496 (GenBank version
dated 22-OCT-2008)
mRNA sequence of human fucose kinase (fucokinase)
(SEQ ID NO: 24)
ATGGAGCAGCCGAAGGGAGTTGATTGGACAGTCATCATCCTGACCTGCCA
GTACAAGGACAGTGTCCAGGTCTTTCAGAGAGAACTGGAAGTGCGGCAGA
AGCGGGAGCAGATCCCTGCTGGGACGCTGTTACTGGCCGTGGAGGACCCA
GAGAAGCGTGTGGGCAGCGGAGGAGCCACCCTCAACGCCCTGCTGGTGGC
TGCTGAACACCTGAGTGCCCGGGCAGGCTTCACTGTGGTCACATCCGATG
TCCTGCACTCGGCCTGGATCCTCATTCTGCACATGGGTCGAGACTTCCCC
TTTGATGACTGTGGCAGGGCTTTCACCTGCCTCCCCGTGGAGAACCCCGA
GGCCCCCGTGGAAGCCTTGGTCTGCAACCTGGACTGCCTGCTGGACATCA
TGACCTATCGGCTGGGCCCGGGCTCCCCGCCAGGCGTGTGGGTCTGCAGC
ACCGACATGCTGCTGTCTGTTCCTGCAAATCCTGGTATCAGCTGGGACAG
CTTCCGGGGAGCCAGAGTGATCGCCCTCCCAGGGAGCCCGGCCTACGCTC
AGAATCATGGCGTCTACCTAACTGACCCCCAGGGCCTTGTTTTGGACATT
TACTACCAGGGCACTGAGGCAGAGATTCAGCGGTGTGTCAGGCCTGATGG
GCGGGTGCCACTGGTCTCTGGGGTTGTCTTCTTCTCTGTGGAGACTGCCG
AGCGCCTCCTAGCCACCCACGTGAGCCCGCCCCTGGATGCCTGCACCTAC
CTAGGCTTGGACTCCGGAGCCCGGCCTGTCCAGCTGTCTCTGTTTTTTGA
CATTCTCCACTGCATGGCTGAGAACGTGACCAGGGAGGACTTCCTGGTGG
GGAGGCCCCCAGAGTTGGGGCAAGGCGATGCAGATGTAGCGGGTTATCTG
CAGAGCGCCCGGGCCCAGCTGTGGAGGGAGCTTCGCGATCAGCCCCTTAC
CATGGCCTATGTCTCCAGCGGCAGCTACAGCTACATGACCTCCTCAGCCA
GTGAGTTCCTGCTCAGCCTCACACTCCCCGGGGCTCCTGGGGCCCAGATT
GTGCACTCCCAGGTGGAGGAGCAGCAGCTTCTGGCGGCCGGGAGCTCTGT
GGTCAGCTGCCTGCTGGAGGGCCCTGTCCAGCTGGGTCCTGGGAGCGTCC
TGCAGCACTGCCACCTGCAGGGCCCCATTCACATAGGCGCTGGCTGCTTG
GTGACTGGCCTGGATACAGCCCACTCCAAGGCCCTGCATGGCCGGGAGCT
GCGTGACCTTGTCCTGCAGGGACACCACACGCGGCTACACGGCTCCCCGG
GCCACGCCTTCACCCTCGTTGGCCGTCTGGACAGCTGGGAGAGACAGGGG
GCAGGCACATATCTCAACGTGCCCTGGAGTGAATTCTTCAAGAGGACAGG
TGTTCGAGCCTGGGACCTGTGGGACCCTGAGACGCTGCCCGCAGAGTACT
GCCTTCCCAGCGCCCGCCTCTTTCCTGTGCTCCACCCCTCGAGGGAGCTG
GGACCCCAGGACCTGCTGTGGATGCTGGACCACCAGGAGGATGGGGGCGA
GGCCCTGCGAGCCTGGCGGGCCTCCTGGCGCCTGTCCTGGGAGCAGCTGC
AGCCGTGCCTGGATCGGGCTGCCACGCTGGCCTCTCGCCGGGACCTGTTC
TTCCGCCAGGCCCTGCATAAGGCGCGGCACGTGCTGGAGGCCCGGCAGGA
CCTCAGCCTGCGCCCGCTGATCTGGGCTGCTGTCCGCGAGGGCTGCCCCG
GGCCCCTGCTGGCCACGCTGGACCAGGTTGCAGCTGGGGCAGGAGACCCT
GGTGTGGCGGCACGGGCACTGGCCTGTGTGGCGGACGTCCTGGGCTGCAT
GGCAGAGGGCCGTGGGGGCTTGCGGAGCGGGCCAGCTGCCAACCCTGAGT
GGATGCGGCCCTTCTCATACCTGGAGTGTGGAGACCTGGCAGCGGGCGTG
GAGGCGCTTGCCCAGGAGAGGGACAAGTGGCTAAGCAGGCCAGCCTTGCT
GGTGCGAGCGGCCCGCCACTATGAGGGGGCTGGTCAGATCCTGATCCGCC
AGGCTGTGATGTCAGCCCAGCACTTTGTCTCCACAGAGCAGGTGGAACTG
CCGGGACCTGGGCAGTGGGTGGTGGCTGAGTGCCCGGCCCGTGTGGATTT
CTCTGGGGGCTGGAGTGACACGCCACCCCTTGCCTATGAGCTTGGCGGGG
CTGTGCTGGGCCTGGCTGTGCGAGTGGACGGCCGCCGGCCCATCGGAGCC
AGGGCACGCCGCATCCCGGAGCCTGAGCTGTGGCTGGCGGTGGGGCCTCG
GCAGGATGAGATGACTGTGAAGATAGTGTGCCGGTGCCTGGCTGACCTGC
GGGACTACTGCCAGCCTCATGCCCCAGGGGCCCTGCTGAAGGCGGCCTTC
ATCTGTGCAGGGATCGTGCATGTCCACTCGGAACTCCAGCTGAGTGAGCA
GCTGCTCCGCACCTTCGGGGGCGGCTTTGAGCTGCACACCTGGTCTGAGC
TGCCCCACGGCTCTGGCCTGGGCACCAGCAGCATCCTGGCAGGCACTGCC
CTGGCTGCCTTGCAGCGAGCCGCAGGCCGGGTGGTGGGCACGGAAGCCCT
GATCCACGCAGTGCTGCACCTGGAGCAGGTGCTCACCACTGGAGGTGGCT
GGCAGGACCAAGTAGGTGGCCTAATGCCTGGCATCAAGGTGGGGCGCTCC
CGGGCTCAGCTGCCACTGAAGGTGGAGGTAGAAGAGGTCACGGTGCCTGA
GGGCTTTGTCCAGAAGCTCAATGACCACCTGCTCTTGGTGTACACTGGCA
AGACCCGCCTGGCTCGGAACCTGCTGCAGGATGTGCTGAGGAGCTGGTAT
GCCCGACTTCCTGCTGTGGTGCAGAATGCCCACAGCCTGGTACGGCAAAC
TGAGGAGTGTGCTGAAGGCTTCCGCCAAGGAAGCCTGCCTCTGCTGGGCC
AGTGCCTGACCTCGTACTGGGAGCAGAAGAAGCTCATGGCTCCAGGCTGT
GAGCCCCTGACTGTGCGGCGTATGATGGATGTCCTGGCCCCCCACGTGCA
TGGCCAGAGCCTGGCTGGGGCAGGCGGTGGAGGCTTTCTCTATCTGTTGA
CCAAGGAGCCACAGCAAAAGGAGGCCTTGGAGGCGGTGCTGGCCAAGACC
GAGGGCCTTGGGAATTACAGCATCCACCTGGTTGAAGTGGACACTCAGGG
CCTGAGCCTGAAGCTGCTGGGGACCGAGGCCTCAACCTGTTGCCCTTTCC
CATGA
GenBank Accession No. NM_145059 (GenBank version
dated 22-OCT-2008)
Protein sequence of mouse fucose kinase
(fucokinase)
(SEQ ID NO: 25)
MEQSEGVNWTVIILTCQYKDSVQVFQRELEVRQRREQIPAGTMLLAVEDP
QTRVGSGGATLNALLVAAEHLSARAGFTVVTSDVLHSAWILILHMGRDFP
FDDCGRAFTCLPVENPQAPVEALVCNLDCLLDIMTHRLGPGSPPGVWVCS
TDMLLSVPPNPGISWDGFRGARVIAFPGSLAYALNHGVYLTDSQGLVLDI
YYQGTKAEIQRCVGPDGLVPLVSGVVFFSVETAEHLLATHVSPPLDACTY
MGLDSGAQPVQLSLFFDILLCMARNMSRENFLAGRPPELGQGDMDVASYL
KGARAQLWRELRDQPLTMVYVPDGGYSYMTTDATEFLHRLTMPGVAVAQI
VHSQVEEPQLLEATCSVVSCLLEGPVHLGPRSVLQHCHLRGPIRIGAGCF
VSGLDTAHSEALHGLELHDVILQGHHVRLHGSLSRVFTLAGRLDSWERQG
AGMYLNMSWNEFFKKTGIRDWDLWDPDTPPSDRCLLTARLFPVLHPTRAL
GPQDVLWMLHPRKHRGEALRAWRASWRLSWEQLQPCVDRAATLDFRRDLF
FCQALQKARHVLEARQDLCLRPLIRAAVGEGCSGPLLATLDKVAAGAEDP
GVAARALACVADVLGCMAEGRGGLRSGPAANPEWIQPFSYLECGDLMRGV
EALAQEREKWLTRPALLVRAARHYEGAEQILIRQAVMTARHFVSTQPVEL
PAPGQWVVTECPARVDFSGGWSDTPPIAYELGGAVLGLAVRVDGRRPIGA
KARRIPEPELWLAVGPRQDEMTMRIVCRSLDDLRDYCQPHAPGALLKAAF
ICAGIVHLHSELPLLEQLLHSFNGGFELHTWSELPHGSGLGTSSILAGAA
LAALQRAAGRAVGTEALIHAVLHLEQVLTTGGGWQDQVSGLMPGIKVGRS
RAQLPLKVEVEEITVPEGFVQKINDHLLLVYTGKTRLARNLLQDVLRNWY
ARLPVVVQNARRLVRQTEKCAEAFRQGNLPLLGQYLTSYWEQKKLMAPGC
EPLAVQRMMDVLAPYAYGQSLAGAGGGGFLYLLTKEPRQKETLEAVLAKA
EGLGNYSVHLVEVDPQGLSLQLLGHDTRLCGAGPSEVGTT
GenBank Accession No. NP_758487 (GenBank version
dated 05-AUG-2008)
mRNA sequence of mouse fucose kinase (fucokinase)
(SEQ ID NO: 26)
ATGGAGCAGTCAGAGGGAGTCAATTGGACTGTCATTATCCTGACATGCCA
GTACAAGGACAGTGTCCAGGTCTTTCAGAGAGAGCTGGAGGTAAGGCAG
AGACGGGAGCAGATTCCTGCGGGGACGATGTTACTGGCTGTGGAGGATCC
CCAGACTCGAGTCGGCAGCGGAGGAGCCACCCTCAACGCACTGCTGGTGG
CTGCTGAACACTTGAGTGCCCGAGCTGGCTTCACTGTGGTCACGTCCGAT
GTCCTGCACTCTGCCTGGATCCTCATCTTGCACATGGGCCGAGACTTCCC
CTTCGATGACTGTGGCAGGGCCTTCACTTGCCTCCCTGTGGAGAACCCAC
AGGCCCCTGTGGAGGCCTTGGTATGCAACCTGGACTGCCTGTTGGATATC
ATGACCCACCGGCTGGGTCCAGGTTCCCCACCAGGTGTGTGGGTCTGCAG
CACCGACATGCTTCTGTCTGTTCCTCCAAACCCTGGGATCAGTTGGGATG
GCTTCCGGGGAGCCAGAGTGATCGCCTTTCCTGGGAGCCTGGCCTATGCG
TTGAACCACGGTGTCTACCTCACTGACTCACAGGGCTTGGTTTTGGACAT
TTACTACCAGGGCACTAAGGCGGAGATACAACGTTGTGTCGGACCTGATG
GGCTGGTACCATTGGTCTCCGGGGTCGTCTTCTTCTCTGTGGAGACTGCT
GAGCACCTCCTAGCCACCCATGTGAGCCCACCGCTGGATGCCTGCACCTA
TATGGGCTTGGACTCTGGAGCCCAGCCTGTGCAGCTGTCTCTGTTTTTCG
ACATCCTGCTCTGCATGGCTCGGAATATGAGCAGGGAGAACTTCCTGGCT
GGGCGGCCCCCGGAGTTGGGGCAAGGTGACATGGATGTAGCAAGTTACCT
GAAGGGAGCCCGGGCCCAGCTGTGGAGGGAGCTTCGAGATCAGCCCCTCA
CAATGGTGTATGTCCCTGACGGCGGCTACAGCTACATGACGACTGATGCC
ACCGAGTTCCTGCACAGACTCACGATGCCTGGAGTAGCTGTGGCACAGAT
TGTTCACTCCCAGGTGGAGGAGCCACAGCTGCTAGAGGCTACGTGCTCGG
TGGTCAGCTGCCTGCTCGAGGGCCCTGTGCACCTGGGGCCTCGAAGTGTC
CTGCAGCACTGTCACCTGAGGGGCCCCATTCGCATCGGCGCTGGCTGCTT
TGTGAGTGGTCTGGATACAGCCCACTCGGAGGCACTGCATGGCCTGGAGC
TCCATGATGTCATCCTGCAGGGACACCATGTGCGGCTGCATGGCTCCCTG
AGCCGTGTATTTACTCTTGCTGGCCGTCTGGACAGCTGGGAAAGACAGGG
GGCAGGCATGTATCTCAACATGTCCTGGAATGAGTTCTTCAAGAAGACAG
GCATTCGAGACTGGGACCTGTGGGACCCAGATACACCCCCCTCAGATCGA
TGCCTCCTCACTGCCCGCCTTTTCCCTGTGCTCCACCCCACGAGGGCCCT
GGGGCCCCAGGATGTGCTGTGGATGCTGCACCCCCGCAAACACAGAGGTG
AGGCCCTTCGGGCCTGGCGAGCCTCCTGGCGTCTGTCCTGGGAGCAGCTG
CAACCTTGTGTGGACCGGGCTGCCACACTGGACTTCCGCCGAGATCTGTT
CTTCTGCCAGGCCTTGCAGAAGGCAAGGCATGTGTTAGAGGCGCGGCAGG
ACCTCTGCCTACGTCCACTGATCCGGGCCGCTGTCGGGGAAGGTTGCTCT
GGGCCCCTGCTGGCCACACTTGACAAGGTTGCAGCTGGGGCAGAAGATCC
TGGCGTGGCAGCCCGGGCTCTGGCTTGTGTGGCCGATGTGCTGGGCTGCA
TGGCAGAGGGCCGAGGAGGCTTGCGCAGTGGGCCAGCTGCCAACCCTGAG
TGGATTCAGCCTTTCTCATACTTGGAGTGTGGAGACCTGATGAGGGGTGT
GGAGGCGCTTGCCCAGGAGAGAGAGAAGTGGCTGACCAGGCCTGCCTTGC
TGGTTCGAGCTGCCCGCCATTACGAGGGGGCCGAGCAGATCCTGATCCGC
CAGGCTGTGATGACAGCCCGGCACTTCGTCTCCACCCAGCCCGTGGAGCT
GCCCGCACCCGGGCAGTGGGTGGTGACTGAGTGCCCAGCCCGTGTGGATT
TCTCTGGGGGCTGGAGTGACACACCGCCCATTGCCTATGAGCTTGGTGGA
GCAGTGTTGGGCCTGGCTGTGCGGGTGGATGGCCGCCGGCCCATCGGGGC
CAAAGCACGCCGCATCCCGGAGCCTGAGCTCTGGCTGGCAGTGGGACCTC
GGCAGGATGAGATGACCATGAGGATAGTGTGCCGGAGCCTGGATGACCTG
CGGGATTACTGCCAGCCTCATGCCCCAGGGGCCTTGCTGAAGGCAGCCTT
TATCTGTGCTGGCATTGTGCATCTCCACTCAGAGCTCCCTCTGCTTGAAC
AGTTGTTACACTCCTTTAATGGTGGCTTTGAGCTGCACACGTGGTCAGAG
CTGCCGCACGGCTCTGGTCTTGGCACCAGCAGCATCCTGGCAGGGGCTGC
CCTGGCTGCCTTACAGCGGGCTGCAGGCCGGGCAGTGGGCACGGAGGCTC
TCATCCACGCAGTGCTGCACCTGGAGCAGGTGCTCACCACAGGAGGTGGC
TGGCAGGACCAAGTCAGTGGCCTAATGCCTGGCATCAAAGTGGGGCGCTC
CCGGGCCCAGCTGCCCCTCAAGGTGGAGGTGGAGGAAATCACTGTGCCTG
AGGGCTTTGTCCAGAAGATCAATGACCATCTGCTCCTGGTTTATACCGGC
AAGACCCGATTGGCCCGGAATCTGCTGCAGGACGTGCTGAGGAACTGGTA
CGCTCGGTTGCCCGTTGTGGTACAGAATGCCCGCAGACTGGTGCGACAGA
CCGAGAAGTGCGCTGAAGCTTTCCGCCAAGGAAACCTGCCTCTGCTGGGA
CAGTACCTGACCTCATACTGGGAGCAGAAGAAGCTTATGGCCCCAGGCTG
CGAGCCGCTGGCCGTGCAGCGAATGATGGATGTCCTGGCCCCGTATGCGT
ATGGCCAAAGCCTGGCAGGGGCAGGTGGTGGGGGCTTTCTCTATCTATTG
ACCAAGGAACCCCGGCAGAAAGAGACTCTGGAAGCTGTCCTGGCCAAGGC
TGAGGGCCTTGGCAACTACAGTGTCCACCTGGTGGAAGTGGATCCTCAGG
GCCTGAGCCTGCAGCTGCTGGGACACGACACCCGTCTTTGTGGGGCCGGG
CCCTCTGAAGTGGGCACCACCTAG
GenBank Accession No. NM_172283 (GenBank version
dated 05-AUG-2008)
Protein sequence of rat fucose kinase (fucokinase)
(SEQ ID NO: 27)
MDQPKGVNWTVIILTCQYKDSVQVFQRELEVRQKREQIPAGTMLLAVEDP
QTRVGSGGATLNALLVAAEHLSARAGFTVVTSDVLHSAWILILHMGRDFP
FDDCGRAFTCLPVENPQAPVEALVCNLDCLLDIMTHRLGPGSPPGVWVCS
TDMLLSVPPNPGISWDGFRGTRVIAFPGSLAYALNHGVYLTDSQGVVLDI
YYQGTKAEIQRCVRPDGLVPLVSGVVFFSVETAEHLLATHVSPPLDACTY
MGLDSGAQPVQLSLFFDILLCMARNMSRENFVAGRPPEMGQGDPDVARYL
KGARAQLWRELRDQPLTMVYVPDGGYSYMTTDATEFLHRLTMPGVAVAQI
VHSQVEEPQLLEATCSVVSCLLEGPVHLGPRSVLQHCHLRGPIHIGAGCF
VSGLDTAHSEALHGLELHDLILQGHHIRLHGSQSRVFTLAGRLDSWERQG
AGMYLNMSWNEFFKKTGIRDWDLWDPDTPLSDRCLLSARLFPVLHPTRAL
GPQDVLWMLHPHKDRGEALRAWRASWRLSWEQLQPRLDRAATLDFRRDLF
FRQALQKARHVLEARQDLCLHPLIRAAVGEGCSGPLLATLDKVAAGAEDP
GVAARALACVADVLGCMAEGQGGLRSGPAANPEWIQPFSYLERGDLMRGV
EALAQEREKWLTRPALLVRAARHYEGAEQILIRQAVMTARHFVSTQPVEL
PAPGQWVVTECPARVDFSGGWSDTPPIAYELGGAVLGLAVRVDGRRPIGA
KARRILEPELWLAVGPRQDEMTVKIVCRSLDDLQDYCQPHAPGALLKAAF
ICADIVHVNSEVPLHEQLLRSFNGGFELHTWSELPHGSGLGTSSILAGAA
LAALQRAAGRTVGTEALIHAVLHLEQVLTTGGGWQDQVSGLMPGIKVGRS
RAQLPLKVEVEEITVPENFVQRKLMAPGCEPLAVHRMMDVLAPYAFGQSL
AGAGGGGFLYLLTKEPRQKEVLEAVLAKVEGLGNYSVHLVQVDTQGLSLQ
LLGHDAHLCGAGPSEVGNT
GenBank Accession No. NP_001100899 (GenBank
version dated 05-AUG-2008)
mRNA sequence of rat fucose kinase (fucokinase)
(SEQ ID NO: 28)
ATGGACCAGCCAAAGGGGGTCAATTGGACGGTCATTATCCTGACATGCCA
GTACAAGGACAGTGTCCAGGTCTTTCAGAGAGAGCTGGAGGTAAGGCAG
AAGCGGGAGCAGATCCCTGCCGGGACGATGTTACTGGCTGTGGAGGACCC
CCAGACCCGAGTAGGCAGTGGAGGAGCTACTCTCAATGCACTGCTGGTGG
CTGCTGAGCACCTGAGTGCCCGAGCTGGCTTCACCGTGGTCACGTCAGAT
GTCCTGCACTCGGCTTGGATTCTCATCTTGCACATGGGCCGAGACTTCCC
CTTTGATGACTGTGGCAGGGCCTTCACTTGCCTCCCTGTGGAGAATCCAC
AGGCCCCTGTGGAGGCCTTGGTATGCAACCTGGACTGCCTGTTGGATATC
ATGACCCACCGGCTGGGTCCAGGATCCCCACCAGGTGTGTGGGTCTGCAG
CACCGACATGCTTCTGTCTGTTCCTCCAAACCCTGGGATCAGTTGGGATG
GCTTCCGGGGAACCAGAGTGATCGCCTTTCCTGGGAGCCTGGCCTACGCT
CTAAACCACGGGGTCTACCTCACTGACTCGCAGGGCGTGGTTTTGGACAT
TTACTACCAGGGCACTAAGGCAGAGATACAACGGTGTGTCAGGCCTGATG
GACTGGTACCACTGGTCTCTGGGGTTGTCTTCTTCTCTGTGGAGACTGCT
GAGCACCTCCTAGCCACCCACGTGAGCCCACCGCTGGACGCCTGCACCTA
TATGGGCTTGGACTCTGGAGCCCAGCCTGTGCAGCTGTCTCTGTTTTTCG
ACATCCTGCTCTGCATGGCTCGGAATATGAGCAGGGAGAACTTCGTGGCT
GGGCGGCCCCCGGAGATGGGGCAAGGTGACCCGGATGTAGCACGTTACCT
GAAGGGAGCCCGGGCCCAGCTGTGGAGGGAGCTTCGAGATCAGCCCCTCA
CTATGGTGTATGTCCCTGATGGCGGTTACAGTTACATGACAACTGATGCC
ACGGAGTTCCTGCACAGACTCACGATGCCTGGAGTAGCTGTGGCCCAGAT
TGTTCACTCTCAGGTGGAGGAGCCACAGCTGCTAGAGGCTACGTGCTCCG
TGGTCAGCTGCCTGCTGGAGGGTCCCGTGCACCTGGGGCCTCGAAGTGTC
CTGCAGCACTGTCACCTGAGGGGCCCCATTCATATTGGCGCTGGCTGCTT
TGTGAGTGGCCTGGATACCGCCCACTCCGAGGCACTGCATGGCCTGGAGC
TTCATGACCTCATCCTTCAGGGACACCACATACGGCTGCATGGCTCCCAG
AGTCGTGTATTCACTCTTGCTGGCCGTCTGGACAGCTGGGAAAGACAGGG
GGCAGGCATGTATCTCAACATGTCCTGGAATGAGTTCTTCAAGAAGACAG
GCATTCGAGACTGGGACCTGTGGGACCCAGATACACCCCTCTCAGATCGA
TGCCTTCTCAGTGCCCGCCTTTTCCCTGTGCTCCACCCCACGAGGGCTCT
GGGGCCCCAGGATGTGCTGTGGATGCTGCATCCTCATAAGGACAGAGGCG
AGGCCCTGCGTGCCTGGAGAGCCTCCTGGCGTCTGTCCTGGGAGCAGCTG
CAACCTCGCCTGGACCGGGCTGCCACACTGGACTTCCGTCGGGATCTGTT
CTTCCGCCAGGCCTTGCAGAAGGCGAGGCATGTGTTAGAGGCCCGGCAGG
ACCTCTGCCTACATCCACTGATCCGGGCTGCTGTCGGTGAAGGTTGCTCT
GGGCCCCTGCTGGCCACACTTGACAAGGTTGCAGCAGGGGCAGAAGATCC
TGGTGTGGCAGCCCGGGCTCTGGCTTGTGTGGCAGATGTACTCGGCTGCA
TGGCAGAGGGCCAAGGAGGCTTGCGCAGTGGGCCAGCTGCCAACCCTGAG
TGGATTCAGCCTTTCTCATACTTGGAACGTGGAGACCTCATGAGGGGTGT
GGAGGCACTTGCCCAGGAAAGAGAGAAGTGGCTGACCAGGCCTGCCTTGT
TGGTTCGAGCTGCCCGCCATTATGAGGGGGCTGAGCAGATCCTGATCCGA
CAGGCTGTGATGACAGCCCGGCACTTCGTCTCCACCCAGCCAGTGGAATT
GCCAGCACCTGGGCAGTGGGTGGTGACTGAGTGCCCAGCCCGTGTGGATT
TCTCTGGGGGCTGGAGTGACACACCACCCATTGCCTATGAGCTTGGTGGA
GCAGTATTGGGCCTGGCTGTTCGGGTGGATGGCCGCCGGCCCATCGGGGC
CAAGGCACGCCGCATCCTAGAGCCTGAGCTCTGGCTGGCAGTGGGACCTC
GACAGGATGAGATGACCGTGAAGATAGTGTGCCGGAGCCTTGATGACCTG
CAGGATTACTGCCAGCCTCATGCCCCAGGTGCCTTGCTGAAGGCAGCCTT
TATCTGTGCGGATATTGTGCATGTCAACTCAGAGGTCCCTCTGCATGAAC
AGTTGCTACGCTCGTTTAATGGTGGCTTTGAGCTGCACACATGGTCAGAG
CTGCCACACGGCTCTGGTCTTGGCACTAGCAGCATCTTGGCAGGGGCTGC
CCTGGCTGCTTTGCAGCGGGCTGCAGGCCGGACAGTGGGCACAGAGGCTC
TCATCCATGCAGTGTTGCACCTGGAGCAGGTGCTCACCACAGGAGGTGGC
TGGCAGGACCAAGTGAGTGGCCTAATGCCTGGCATCAAGGTGGGGCGCTC
TCGGGCACAGCTGCCCCTAAAGGTGGAGGTGGAGGAAATCACTGTGCCTG
AGAACTTTGTCCAGAGGAAGCTTATGGCCCCAGGCTGTGAGCCGCTGGCT
GTGCATCGGATGATGGATGTCCTGGCCCCTTATGCCTTCGGCCAAAGTCT
GGCAGGGGCAGGCGGTGGGGGCTTTCTCTATCTGTTGACCAAGGAACCCC
GGCAGAAAGAGGTCCTAGAAGCTGTGCTGGCCAAGGTGGAGGGCCTCGGC
AACTACAGCGTCCACCTGGTGCAAGTGGACACTCAGGGCCTGAGCCTGCA
GCTGCTAGGACATGACGCCCATCTTTGCGGGGCTGGGCCCTCTGAAGTGG
GCAACACCTAG
GenBank Accession No. NP_001100899 (GenBank
version dated 05-AUG-2008)

### Fucosyltransferases

Fucosylated glycans are synthesized by fucosyltransferases, using GDP-fucose as the activated sugar-nucleotide donor. Thirteen fucosyltransferase genes have thus far been identified in the human genome, and include FUT8, FUT4, FUT7, FUT3 and FUT9. FUT8 is an α(1,6)-fucosyltransferase that directs addition of fucose to asparagine-linked GlcNAc moieties, resulting in core fucosylation.

Protein sequence of human fucosyltransferase 8 (α(1,6)-fucosyltransferase)
(SEQ ID NO: 29)
MAITVSLVNNKRKIVVLAQPTTVKRKRITPYKSIMTDLYYLSQTDGAGDWRE
KEAKDLTELVQRRITYLQNPKDCSKAKKLVCNINKGCGYGCQLHHVVYCFM
IAYGTQRTLILESQNWRYATGGWETVFRPVSETCTDRSGISTGHWSGEVKDK
NVQVVELPIVDSLHPRPPYLPLAVPEDLADRLVRVHGDPAVWWVSQFVKYLI
RPQPWLEKEIEEATKKLGFKHPVIGVHVRRTDKVGTEAAFHPIEEYMVHVEE
HFQLLARRMQVDKKRVYLATDDPSLLKEAKTKYPNYEFISDNSISWSAGLHN
RYTENSLRGVILDIHFLSQADFLVCTFSSQVCRVAYEIMQTLHPDASANFHSL
DDIYYFGGQNAHNQIAIYAHQPRTADEIPMEPGDIIGVAGNHWDGYSKGVNR
KLGRTGLYPSYKVREKIETVKYPTYPEAEK
GenBank Accession No. NP_004480 (GenBank version dated 22 OCT. 2008)
mRNA sequence of human fucosyltransferase 8 (α1,6)-fucosyltransferase)
(SEQ ID NO: 30)
ggccgacccgagcagccggttccctcctctccaggccccctccccatcccacccccgccgcctggccccagccgaccc
gtcccttcgtctccccgcggaatggggccggcactgctcagggtcgcgcgccctggacccagctcgctctcggtctcgcg
ctgtcagcgactgcccggctcgcgccgcctcgcgctctgcctcagtcagtggcgccgaaggctccgttaagcggcggcg
gcggttcctgtttccgtttcttcctctccgttcggtcgggagtagcatcctccactcagccacccttcccactcccccatcgtgg
ggcagctgcggctgagggctgtggctttggcagctgcgacggggagcggcggagaccgcctctgctcccgcctggggt
tgctgcttttgctcagaggacatccatgaccctaatggtctttttgttcaagataaagtgattttttgcctttgttgattaactggac
aaattcaggataccagaaggccctattgatcaggggccagctataggaagagtacgcgttttagaagagcagcttgttaag
gccaaagaacagattgaaaattacaagaaacagaccagaaatggtctggggaaggatcatgaaatcctgaggaggagga
ttgaaaatggagctaaagagctctggtttttcctacagagtgaattgaagaaattaaagaacttagaaggaaatgaactccaa
agacatgcagatgaatttcttttggatttaggacatcatgaaaggattctgatggcaattactgtctcattagtgaacaataaaa
gaaaaattgttgtattagcacaacctactactgtgaagaggaaaagaattaccccatacaagtctataatgacggatctatact
acctcagtcagacagatggagcaggtgattggcgggaaaaagaggccaaagatctgacagaactggttcagcggagaat
aacatatcttcagaatcccaaggactgcagcaaagccaaaaagctggtgtgtaatatcaacaaaggctgtggctatggctgt
cagctccatcatgtggtctactgcttcatgattgcatatggcacccagcgaacactcatcttggaatctcagaattggcgctat
gctactggtggatgggagactgtatttaggcctgtaagtgagacatgcacagacagatctggcatctccactggacactggt
caggtgaagtgaaggacaaaaatgttcaagtggtcgagcttcccattgtagacagtcttcatccccgtcctccatatttaccct
tggctgtaccagaagacctcgcagatcgacttgtacgagtgcatggtgaccctgcagtgtggtgggtgtctcagtttgtcaa
atacttgatccgcccacagccttggctagaaaaagaaatagaagaagccaccaagaagcttggcttcaaacatccagttatt
ggagtccatgtcagacgcacagacaaagtgggaacagaagctgccttccatcccattgaagagtacatggtgcatgttgaa
gaacattttcagcttcttgcacgcagaatgcaagtggacaaaaaaagagtgtatttggccacagatgacccttctttattaaag
gaggcaaaaacaaagtaccccaattatgaatttattagtgataactctatttcctggtcagctggactgcacaatcgatacaca
gaaaattcacttcgtggagtgatcctggatatacattttctctctcaggcagacttcctagtgtgtactttttcatcccaggtctgt
cgagttgcttatgaaattatgcaaacactacatcctgatgcctctgcaaacttccattctttagatgacatctactattttggggg
ccagaatgcccacaatcaaattgccatttatgctcaccaaccccgaactgcagatgaaattcccatggaacctggagatatc
attggtgtggctggaaatcattgggatggctattctaaaggtgtcaacaggaaattgggaaggacgggcctatatccctccta
caaagttcgagagaagatagaaacggtcaagtaccccacatatcctgaggctgagaaataaagctcagatggaagagata
aacgaccaaactcagttcgaccaaactcagttcaaaccatttcagccaaactgtagatgaagagggctctgatctaacaaaa
taaggttatatgagtagatactctcagcaccaagagcagctgggaactgacataggcttcaattggtggaattcctctttaaca
agggctgcaatgccctcatacccatgcacagtacaataatgtactcacatataacatgcaaacaggttgttttctactttgccc
ctttcagtatgtccccataagacaaacactgccatattgtgtaatttaagtgacacagacattttgtgtgagacttaaaacatggt
gcctatatctgagagacctgtgtgaactattgagaagatcggaacagctccttactctgaggaagttgattcttatttgatggtg
gtattgtgaccactgaattcactccagtcaacagattcagaatgagaatggacgtttggtttttttttgtttttgtttttgttttttccttt
ataaggttgtctgtttttttttttttaaataattgcatcagttcattgacctcatcattaataagtgaagaatacatcagaaaataaaat
attcactctccattagaaaattttgtaaaacaatgccatgaacaaattctttagtactcaatgtttctggacattctctttgataaca
aaaaataaattttaaaaaggaattttgtaaagtttctagaattttatatcattggatgatatgttgatcagccttatgtggaagaact
gtgataaaaagaggagctttttagtttttcagcttaaaaaaa
GenBank Accession No. NP_004480 (GenBank version dated 22 OCT. 2008)
Protein sequence of rat fucosyltransferase 8 (α1,6)-fucosyltransferase)
(SEQ ID NO: 31)
MRAWTGSWRWIMLILFAWGTLLFYIGGHLVRDNDHPDHSSRELSKILAKLER
LKQQNEDLRRMAESLR1PEGPIDQGTATGRVRVLEEQLVKAKEQIENYKKQA
RNGLGKDHELLRRRIENGAKELWFFLQSELKKLKHLEGNELQRHADEILLDL
GHHERSIMTDLYYLSQTDGAGDWREKEAKDLTELVQRRITYLQNPKDCSKA
RKLVCNINKGCGYGCQLHHVVYCFMIAYGTQRTLILESQNWRYATGGWETV
FRPVSETCTDRSGLSTGHWSGEVNDKNIQVVELPIVDSLHPRPPYLPLAVPEDL
ADRLVRVHGDPAVWWVSQFVKYLIRPQPWLEKEIEEATKKLGFKHPVIGVH
VRRTDKVGTEAAFHPIEEYMVHVEEHFQLLARRMQVDKKRVYLATDDPALL
KEAKTKYSNYEFISDNSISWSAGLHNRYTENSLRGVILDIHFLSQADFLVCTFS
SQVCRVAYEIMQTLHPDASANFHSLDDIYYFGGQNAHNQIAVYPHKPRTDEEI
PMEPGDIIGVAGNHWDGYSKGVNRKLGKTGLYPSYKVREKIETVKYPTYPEA
EK
GenBank Accession No. NP_001002289 (GenBank version dated 5 OCT. 2008)
mRNA sequence of rat fucosyltransferase 8 (α1,6)-fucosyltransferase)
(SEQ ID NO: 32)
atgcgggcatggactggttcctggcgttggattatgctcattctttttgcctgggggaccttgttgttttatataggtggtcatttg
gttcgagataatgaccaccctgatcactctagcagagaactctccaagattcttgcaaagcttgaacgcttaaaacaacaaaa
tgaagacttgaggcgaatggctgagtctctacgaataccagaaggccccattgaccaggggacggctacgggaagagtc
cgtgttttagaagaacagcttgttaaggccaaagaacagattgaaaattacaagaaacaagccagaaatggtctggggaag
gatcatgaactcttaaggaggaggattgaaaatggagctaaagagctctggttttttctacaaagtgaactgaagaaattaaa
gcatctagaaggaaatgaactccaaagacatgcagatgaaattcttttggatttaggacaccatgaaaggtctatcatgacgg
atctatactacctcagtcaaacagatggagcaggggattggcgtgaaaaagaggccaaagatctgacagagctggtccag
cggagaataacttatctccagaatcccaaggactgcagcaaagccaggaagctggtgtgtaacatcaataagggctgtgg
ctatggttgccaactccatcacgtggtctactgtttcatgattgcttatggcacccagcgaacactcatcttggaatctcagaatt
ggcgctatgctactggtggatgggagactgtgtttagacctgtaagtgagacatgcacagacagatctggcctctccactgg
acactggtcaggtgaagtgaatgacaaaaatattcaagtggtggagctccccattgtagacagcctccatcctcggcctcctt
acttaccactggctgttccagaagaccttgcagatcgactcgtaagagtccatggtgatcctgcagtgtggtgggtgtccca
gttcgtcaaatatttgattcgtccacaaccttggctagaaaaggaaatagaagaagccaccaagaagcttggcttcaaacatc
cagtcattggagtccatgtcagacgcacagacaaagtgggaacagaggcagccttccatcccatcgaagagtacatggta
catgttgaagaacattttcagcttctcgcacgcagaatgcaagtggataaaaaaagagtatatctggctaccgatgaccctgc
tttgttaaaggaggcaaagacaaagtactccaattatgaatttattagtgataactctatttcttggtcagctggattacacaatc
ggtacacagaaaattcacttcggggcgtgatcctggatatacactttctctctcaggctgacttcctagtgtgtactttttcatcc
caggtctgtcgggttgcttatgaaatcatgcaaaccctgcatcctgatgcctctgcaaacttccactctttagatgacatctact
attttggaggccaaaatgcccacaaccagattgccgtttatcctcacaaacctcgaactgatgaggaaattccaatggaacct
ggagatatcattggtgtggctggaaaccattgggatggttattctaaaggtgtcaacagaaaacttggaaaaacaggcttata
tccctcctacaaagtccgagagaagatagaaacagtcaagtatcccacatatcctgaagctgaaaaatag
GenBank Accession No. NM_001002289 (GenBank version dated 5 OCT. 2008)
Protein sequence of mouse fucosyltransferase 8 (α1,6)-fucosyltransferase)
(SEQ ID NO: 33)
MRAWTGSWRWIMLILFAWGTLLFYIGGHLVRDNDHPDHSSRELSKILAKLER
LKQQNEDLRRMAESLR1PEGPIDQGTATGRVRVLEEQLVKAKEQIENYKKQA
RNGLGKDHEILRRRIENGAKELWFFLQSELKKLKHLEGNELQRHADEILLDLG
HHERSIMTDLYYLSQTDGAGDWREKEAKDLTELVQRRITYLQNPKDCSKAR
KLVCNINKGCGYGCQLHHVVYCFMIAYGTQRTLILESQNWRYATGGWETVF
RPVSETCTDRSGLSTGHWSGEVNDKNIQVVELPIVDSLHPRPPYLPLAVPEDL
ADRLLRVHGDPAVWWVSQFVKYLIRPQPWLEKEIEEATKKLGFKHPVIGVHV
RRTDKVGTEAAFHPIEEYMVHVEEHFQLLARRMQVDKKRVYLATDDPTLLK
EAKTKYSNYEFISDNSISWSAGLHNRYTENSLRGVILDIHFLSQADFLVCTFSS
QVCRVAYEIMQTLHPDASANFHSLDDIYYFGGQNAHNQIAVYPHKPRTEEEIP
MEPGDIIGVAGNHWDGYSKGINRKLGKTGLYPSYKVREKIETVKYPTYPEAE
K
GenBank Accession No. NP_058589 (GenBank version dated 04 JAN. 2009)
mRNA sequence of mouse fucosyltransferase 8 (A1,6)-fucosyltransferase)
(SEQ ID NO: 34)
atgcgggcatggactggttcctggcgttggattatgctcattctttttgcctgggggaccttgttattttatataggtggtcatttg
gttcgagataatgaccaccctgatcactccagcagagaactctccaagattcttgcaaagcttgaacgcttaaaacagcaaa
atgaagacttgaggcgaatggctgagtctctccgaataccagaaggccccattgaccaggggacagctacaggaagagt
ccgtgttttagaagaacagcttgttaaggccaaagaacagattgaaaattacaagaaacaagctagaaatggtctggggaa
ggatcatgaaatcttaagaaggaggattgaaaatggagctaaagagctctggttttttctacaaagcgaactgaagaaattaa
agcatttagaaggaaatgaactccaaagacatgcagatgaaattcttttggatttaggacaccatgaaaggtctatcatgaca
gatctatactacctcagtcaaacagatggagcaggggattggcgtgaaaaagaggccaaagatctgacagagctggtcca
gcggagaataacatatctccagaatcctaaggactgcagcaaagccaggaagctggtgtgtaacatcaataaaggctgtg
gctatggttgtcaactccatcacgtggtctactgtttcatgattgcttatggcacccagcgaacactcatcttggaatctcagaa
ttggcgctatgctactggtggatgggagactgtgtttagacctgtaagtgagacatgtacagacagatctggcctctccactg
gacactggtcaggtgaagtaaatgacaaaaacattcaagtggtcgagctccccattgtagacagcctccatcctcggcctcc
ttacttaccactggctgttccagaagaccttgcagaccgactcctaagagtccatggtgaccctgcagtgtggtgggtgtccc
agtttgtcaaatacttgattcgtccacaaccttggctggaaaaggaaatagaagaagccaccaagaagcttggcttcaaaca
tccagttattggagtccatgtcagacgcacagacaaagtgggaacagaagcagccttccaccccatcgaggagtacatgg
tacacgttgaagaacattttcagcttctcgcacgcagaatgcaagtggataaaaaaagagtatatctggctactgatgatccta
ctttgttaaaggaggcaaagacaaagtactccaattatgaatttattagtgataactctatttcttggtcagctggactacacaat
cggtacacagaaaattcacttcggggtgtgatcctggatatacactttctctcacaggctgactttctagtgtgtactttttcatcc
caggtctgtcgggttgcttatgaaatcatgcaaaccctgcatcctgatgcctctgcgaacttccattctttggatgacatctact
attttggaggccaaaatgcccacaatcagattgctgtttatcctcacaaacctcgaactgaagaggaaattccaatggaacct
ggagatatcattggtgtggctggaaaccattgggatggttattctaaaggtatcaacagaaaacttggaaaaacaggcttata
tccctcctacaaagtccgagagaagatagaaacagtcaagtatcccacatatcctgaagctgaaaaatag
GenBank Accession No. NM_016893 (GenBank version dated 04 JAN. 2009)

### GDP-Fucose Transporters

Fucosylated glycans are synthesized by fucosyltransferases in the Golgi apparatus, while GDP-fucose is synthesized in the cytosol. Thus, GDP-fucose must be translocated to the Golgi by a GDP-fucose transporter, such as GDP-fucose transporter 1 (FUCT1).

Protein sequence of human GDP-fucose transporter 1
(FUCT1)
(SEQ ID NO: 35)
MNRAPLKRSRILHMALTGASDPSAEAEANGEKPFLLRALQIALVVSLYW
VTSISMVFLNKYLLDSPSLRLDTPIFVTFYQCLVTTLLCKGLSALAACC
PGAVDFPSLRLDLRVARSVLPLSVVFIGMITFNNLCLKYVGVAFYNVGR
SLTTVFNVLLSYLLLKQTTSFYALLTCGIIIGGFWLGVDQEGAEGTLSW
LGTVFGVLASLCVSLNAIYTTKVLPAVDGSIWRLTFYNNVNACILFLPL
LLLLGELQALRDFAQLGSAHFWGMMTLGGLFGFAIGYVTGLQIKFTSPL
THNVSGTAKACAQTVLAVLYYEETKSFLWWTSNMMVLGGSSAYTWVRGW
EMKKTPEEPSPKDSEKSAMGV
GenBank Accession No. NP_060859 (GenBank version
dated 27 FEB. 2009)
mRNA sequence of human GDP-fucose transporter 1
(FUCT1)
(SEQ ID NO: 36)
ATGAATAGGGCCCCTCTGAAGCGGTCCAGGATCCTGCACATGGCGCTGA
CCGGGGCCTCAGACCCCTCTGCAGAGGCAGAGGCCAACGGGGAGAAGCC
CTTTCTGCTGCGGGCATTGCAGATCGCGCTGGTGGTCTCCCTCTACTGG
GTCACCTCCATCTCCATGGTGTTCCTTAATAAGTACCTGCTGGACAGCC
CCTCCCTGCGGCTGGACACCCCCATCTTCGTCACCTTCTACCAGTGCCT
GGTGACCACGCTGCTGTGCAAAGGCCTCAGCGCTCTGGCCGCCTGCTGC
CCTGGTGCCGTGGACTTCCCCAGCTTGCGCCTGGACCTCAGGGTGGCCC
GCAGCGTCCTGCCCCTGTCGGTGGTCTTCATCGGCATGATCACCTTCAA
TAACCTCTGCCTCAAGTACGTCGGTGTGGCCTTCTACAATGTGGGCCGC
TCACTCACCACCGTCTTCAACGTGCTGCTCTCCTACCTGCTGCTCAAGC
AGACCACCTCCTTCTATGCCCTGCTCACCTGCGGTATCATCATCGGGGG
CTTCTGGCTTGGTGTGGACCAGGAGGGGGCAGAAGGCACCCTGTCGTGG
CTGGGCACCGTCTTCGGCGTGCTGGCTAGCCTCTGTGTCTCGCTCAACG
CCATCTACACCACGAAGGTGCTCCCGGCGGTGGACGGCAGCATCTGGCG
CCTGACTTTCTACAACAACGTCAACGCCTGCATCCTCTTCCTGCCCCTG
CTCCTGCTGCTCGGGGAGCTTCAGGCCCTGCGTGACTTTGCCCAGCTGG
GCAGTGCCCACTTCTGGGGGATGATGACGCTGGGCGGCCTGTTTGGCTT
TGCCATCGGCTACGTGACAGGACTGCAGATCAAGTTCACCAGTCCGCTG
ACCCACAATGTGTCGGGCACGGCCAAGGCCTGTGCCCAGACAGTGCTGG
CCGTGCTCTACTACGAGGAGACCAAGAGCTTCCTCTGGTGGACGAGCAA
CATGATGGTGCTGGGCGGCTCCTCCGCCTACACCTGGGTCAGGGGCTGG
GAGATGAAGAAGACTCCGGAGGAGCCCAGCCCCAAAGACAGCGAGAAGA
GCGCCATGGGGGTGTGA
GenBank Accession No. NM_018389 (GenBank version
dated 27 FEB. 2009)
Protein sequence of mouse GDP-fucose transporter 1
(FUCT1)
(SEQ ID NO: 37)
MNRAPLKRSRILRMALTGVSAVSEESESGNKPFLLRALQIALVVSSLYW
VTSISMVFLNKYLLDSPSLQLDTPIFVTFYQCLVTSLLCKGLSTLATCC
PGMVDFPTLNLDLKVARSVLPLSVVFIGMITFNNLCLKYVGVPFYNVGR
SLTTVFNVLLSYLLLKQTTSFYALLTCGVIIGGFWLGIDQEGAEGTLSL
TGTIFGVLASLCVSLNAIYTKKVLPAVDHSIWRLTFYNNVNACVLFLPL
MIVLGELRALLAFTHLSSAHFWLMMTLGGLFGFAIGYVTGLQIKFTSPL
THNVSGTAKACAQTVLAVLYYEEIKSFLWWTSNLMVLGGSSAYTWVRGW
EMQKTQEDPSSKDGEKSAIRV
GenBank Accession No. NP_997597 (GenBank version
dated 21-SEP-2008)
mRNA sequence of mouse GDP-fucose transporter 1
(FUCT1)
(SEQ ID NO: 38)
ATGAACAGGGCGCCTCTGAAGCGGTCCAGGATCCTGCGCATGGCGCTGA
CTGGAGTCTCTGCTGTCTCCGAGGAGTCAGAGAGCGGGAACAAGCCATT
TCTGCTCCGGGCTCTGCAGATCGCGCTGGTGGTCTCTCTCTACTGGGTC
ACCTCCATTTCCATGGTATTCCTCAACAAGTACCTGCTGGACAGCCCCT
CCCTGCAGCTGGATACCCCCATTTTTGTCACCTTCTACCAATGCCTGGT
GACCTCACTGCTGTGCAAGGGCCTCAGCACTCTGGCCACCTGCTGCCCC
GGCATGGTAGACTTCCCCACCCTAAACCTGGACCTCAAGGTGGCCCGAA
GTGTGCTGCCGCTGTCAGTGGTCTTTATCGGCATGATAACCTTCAATAA
CCTCTGCCTCAAGTACGTAGGGGTGCCCTTCTACAACGTGGGACGCTCG
CTCACCACCGTGTTCAACGTTCTTCTCTCCTACCTGCTGCTCAAACAGA
CCACTTCCTTCTATGCCCTGCTCACCTGCGGCGTCATCATTGGTGGTTT
CTGGCTGGGTATAGACCAAGAAGGAGCTGAGGGAACCTTGTCCCTGACG
GGCACCATCTTCGGGGTGCTGGCCAGCCTCTGCGTCTCCCTCAATGCCA
TCTATACCAAGAAGGTGCTCCCTGCAGTAGACCACAGTATCTGGCGCCT
AACCTTCTATAACAATGTCAATGCCTGCGTGCTCTTCTTGCCCCTGATG
ATAGTGCTGGGCGAGCTCCGTGCCCTCCTGGCCTTCACTCATCTGAGCA
GTGCCCACTTCTGGCTCATGATGACGCTGGGTGGCCTGTTTGGCTTTGC
CATCGGCTATGTGACAGGACTGCAGATCAAATTCACCAGTCCCCTGACC
CATAACGTGTCAGGCACGGCCAAGGCCTGTGCACAGACAGTGCTGGCCG
TGCTCTACTACGAAGAGATTAAGAGCTTCCTGTGGTGGACAAGCAACCT
GATGGTGCTGGGTGGCTCCTCCGCCTACACCTGGGTCAGGGGCTGGGAG
ATGCAGAAGACCCAGGAGGACCCCAGCTCCAAAGATGGTGAGAAGAGTG
CTATCAGGGTGTGA
GenBank Accession No. NM_211358 (GenBank version
dated 21 SEP. 2008)
Protein sequence of rat GDP-fucose transporter 1
(FUCT1)
(SEQ ID NO: 39)
MNRVPLKRSRILRMALTGASAVSEEADSENKPFLLRALQIALVVSLYWV
TSISMVFLNKYLLDSPSLQLDTPIFVTFYQCLVTSLLCKGLSTLATCCP
GMVDFPTLNLDLKVARSVLPLSVVFIGMITFNNLCLKYVGVAFYNVGRS
LTTVFNVLLSYLLLKQTTSFYALLTCAIIIGGFWLGIDQEGAEGTLSLT
GTIFGVLASLCVSLNAIYTKKVLPAVDHSIWRLTFYNNVNACVLFLPLM
VVLGELHALLAFAHLNSAHFWVMMTLGGLFGFAIGYVTGLQIKFTSPLT
HNVSGTAKACAQTVLAVLYYEEIKSFLWWTSNLMVLGGSSAYTWVRGWE
MQKTQEDPSSKEGEKSAIGV
GenBank Accession No. NP_001101218 (GenBank
version dated 18 FEB. 2009)
mRNA sequence of rat GDP-fucose transporter 1
(FUCT1)
(SEQ ID NO: 40)
ATGAACAGGGTCCCTCTGAAGCGGTCCAGGATCCTGCGCATGGCGCTGA
CTGGAGCCTCTGCTGTCTCTGAGGAGGCAGACAGCGAGAACAAGCCATT
TCTGCTACGGGCTCTGCAGATCGCGCTGGTGGTTTCTCTCTACTGGGTC
ACCTCCATCTCCATGGTATTCCTCAACAAGTACCTGCTGGACAGCCCCT
CCCTGCAGCTGGATACCCCCATCTTCGTCACCTTCTACCAATGCCTGGT
GACCTCACTGCTGTGCAAGGGCCTCAGCACTCTGGCCACCTGCTGCCCT
GGCATGGTAGACTTCCCCACCCTAAACCTGGACCTCAAGGTGGCCCGAA
GTGTGCTGCCGCTGTCCGTGGTCTTTATCGGCATGATAACCTTCAATAA
CCTCTGCCTCAAGTACGTGGGGGTGGCCTTCTACAACGTGGGACGCTCG
CTCACTACCGTGTTCAATGTGCTTCTCTCCTACCTGCTGCTTAAACAGA
CCACTTCCTTTTATGCCCTGCTCACCTGTGCCATCATCATTGGTGGTTT
CTGGCTGGGAATAGATCAAGAGGGAGCTGAGGGCACCCTGTCCCTGACG
GGCACCATCTTCGGGGTGCTGGCCAGCCTCTGTGTCTCACTCAATGCCA
TCTACACCAAGAAGGTGCTCCCTGCCGTAGACCACAGTATCTGGCGCCT
AACCTTCTATAACAACGTCAACGCCTGTGTGCTCTTCTTGCCCCTGATG
GTAGTGCTGGGCGAGCTCCATGCTCTCCTGGCCTTCGCTCATCTGAACA
GCGCCCACTTCTGGGTCATGATGACGCTGGGTGGACTCTTCGGCTTTGC
CATTGGCTATGTGACAGGACTGCAGATCAAATTCACCAGTCCCCTGACC
CATAATGTGTCGGGCACAGCCAAGGCCTGTGCACAGACAGTGCTGGCTG
TGCTCTACTATGAAGAGATTAAGAGCTTCCTGTGGTGGACAAGCAACTT
GATGGTGCTGGGTGGCTCCTCTGCCTACACCTGGGTCAGGGGCTGGGAG
ATGCAGAAGACCCAGGAGGACCCCAGCTCCAAAGAGGGTGAGAAGAGTG
CTATCGGGGTGTGA
GenBank Accession No. NM_001107748 (GenBank
version dated 18 FEB. 2009)
Protein sequence of Chinese hamster GDP-fucose
transporter 1 (FUCT1)
(SEQ ID NO: 41)
MNRAPLKRSRILRMALTGGSTASEEADEDSRNKPFLLRALQIALVVSLY
WVTSISMVFLNKYLLDSPSLQLDTPIFVTFYQCLVTSLLCKGLSTLATC
CPGTVDFPTLNLDLKVARSVLPLSVVFIGMISFNNLCLKYVGVAFYNVG
RSLTTVFNVLLSYLLLKQTTSFYALLTCGIIIGGFWLGIDQEGAEGTLS
LIGTIFGVLASLCVSLNAIYTKKVLPAVDNSIWRLTFYNNVNACVLFLP
LMVLLGELRALLDFAHLYSAHFWLMMTLGGLFGFAIGYVTGLQIKFTSP
LTHNVSGTAKACAQTVLAVLYYEETKSFLWWTSNLMVLGGSSAYTWVRG
WEMQKTQEDPSSKEGEKSAIRV
GenBank Accession No. BAE16173 (GenBank version
dated 12 SEP. 2008)
mRNA sequence of Chinese hamster GDP-fucose
transporter 1 (FUCT1)
(SEQ ID NO: 42)
ATGAACAGGGCGCCTCTGAAGCGGTCCAGGATCCTGCGCATGGCGCTGA
CTGGAGGCTCCACTGCCTCTGAGGAGGCAGATGAGGACAGCAGGAACAA
GCCGTTTCTGCTGCGGGCGCTGCAGATCGCGCTGGTCGTCTCTCTCTAC
TGGGTCACCTCCATCTCCATGGTATTCCTCAACAAGTACCTGCTGGACA
GCCCCTCCCTGCAGCTGGATACCCCTATCTTCGTCACTTTCTACCAATG
CCTGGTGACCTCTCTGCTGTGCAAGGGCCTCAGCACTCTGGCCACCTGC
TGCCCTGGCACCGTTGACTTCCCCACCCTGAACCTGGACCTTAAGGTGG
CCCGCAGCGTGCTGCCACTGTCGGTAGTCTTCATTGGCATGATAAGTTT
CAATAACCTCTGCCTCAAGTACGTAGGGGTGGCCTTCTACAACGTGGGG
CGCTCGCTCACCACCGTGTTCAATGTGCTTCTGTCCTACCTGCTGCTCA
AACAGACCACTTCCTTCTATGCCCTGCTCACATGTGGCATCATCATTGG
TGGTTTCTGGCTGGGTATAGACCAAGAGGGAGCTGAGGGCACCCTGTCC
CTCATAGGCACCATCTTCGGGGTGCTGGCCAGCCTCTGCGTCTCCCTCA
ATGCCATCTATACCAAGAAGGTGCTCCCAGCAGTGGACAACAGCATCTG
GCGCCTAACCTTCTATAACAATGTCAATGCCTGTGTGCTCTTCTTGCCC
CTGATGGTTCTGCTGGGTGAGCTCCGTGCCCTCCTTGACTTTGCTCATC
TGTACAGTGCCCACTTCTGGCTCATGATGACGCTGGGTGGCCTCTTCGG
CTTTGCCATTGGCTATGTGACAGGACTGCAGATCAAATTCACCAGTCCC
CTGACCCACAATGTATCAGGCACAGCCAAGGCCTGTGCGCAGACAGTGC
TGGCCGTGCTCTACTATGAAGAGACTAAGAGCTTCCTGTGGTGGACAAG
CAACCTGATGGTGCTGGGTGGCTCCTCAGCCTATACCTGGGTCAGGGGC
TGGGAGATGCAGAAGACCCAAGAGGACCCCAGCTCCAAAGAGGGTGAGA
AGAGTGCTATCAGGGTGTGA
GenBank Accession No. AB222037 (GenBank version
dated 12 SEP. 2008)

Proteins or nucleic acids used in the methods and cells described herein (e.g., GMD, FX, GFPP, fucose kinase, GDP-fucose synthetase, a fucosyltransferase or a GDP-fucose transporter) include mammalian (e.g., human, mouse, rat or hamster) proteins. A protein, nucleic acid or cell can be a primate (e.g., human) protein, nucleic acid or cell. In other embodiments the protein, nucleic acid or cell is a rodent (e.g., a mouse, rat or hamster) protein, nucleic acid or cell.

A protein sequence, e.g., a protein encoding sequence, can be used to decrease the protein expression in a cell. For example, a decrease in protein expression can be achieved by inactivating the endogenous gene, e.g., in the control or structural regions. A cloned sequence can be used to make a construct that will insert a deletion or other event into an endogenous gene to decrease levels of the protein it expresses.

The expression of endogenous protein can be decreased by the use of a genetic construct from the same species as the endogenous protein, or from a different species. For example, the expression of an endogenous protein in a mouse cell can be modulated with a construct made from mouse protein or with one made from a protein sequence from another species, e.g., a different rodent species. The protein of a rodent, e.g., a hamster, such as a Chinese hamster, can be manipulated with an allogeneic sequence (from the same species) or a xenogeneic sequence (from a different species). For example, a CHO cell can be manipulated with a Chinese hamster, mouse or rat sequence.

A nucleic acid sequence from one of the proteins disclosed herein can be used to isolate a gene from a different species. For example, a mouse or rat sequence described herein can be used to make primers to isolate a sequence from another rodent, e.g., a hamster, e.g., a Chinese hamster. That sequence can them be used to modify protein expression in a cell, e.g., in a Chinese hamster cell, such as a CHO cell.

### Manipulations

As described above, a manipulation, as used herein, refers to a property of a cell. Examples of manipulations include the presence in or on the cell of an exogenous inhibitor of an enzyme involved in the biosynthesis of GDP-fucose, or a nucleic acid antagonist (e.g., an siRNA)

A manipulated cell can be, e.g., a vertebrate, mammalian or rodent cell. Primers or other nucleic acids used, e.g., to form or make manipulations, can be, e.g., vertebrate, mammalian or rodent sequences. For example, a rodent primer or other nucleic acid, e.g., a nucleic acid encoding an active or inactivate rodent GMD, FX, fucokinase, GFPP, GDP-fucose synthetase, a fucosyltransferase or GDP-fucose transporter protein, can be used to manipulate a rodent cell. Similarly, a mammalian cell having a manipulation can be made with mammalian nucleic acids, e.g., mammalian primers or a nucleic acid encoding a mammalian GMD, FX, fucokinase, GFPP, GDP-fucose synthetase, a fucosyltransferase or GDP-fucose transporter protein. A sequence from a first species can be used to manipulate a cell of a second species. E.g., a primer or nucleic acid from a first species, e.g., a first rodent species, e.g., a mouse or rat, can be used to manipulate a cell from a second species, e.g., a second rodent species, e.g., a hamster cell, e.g., a CHO cell.

### Nucleic Acid Antagonists

In some embodiments, nucleic acid antagonists are used to decrease expression of a target protein, e.g., a protein involved in regulating GDP-fucose levels, e.g., a protein involved in GDP-fucose biosynthesis, a fucosyltransferase or a GDP-fucose transporter. In one embodiment, the nucleic acid antagonist is an siRNA that targets mRNA encoding the target protein. Other types of antagonistic nucleic acids can also be used, e.g., a nucleic acid aptamer, a dsRNA, a ribozyme, a triple-helix former, or an antisense nucleic acid.

siRNAs can be used to inhibit expression of a protein involved in GDP-fucose biosynthesis, a fucosyltransferase or a GDP-fucose transporter. siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically the siRNA sequences are exactly complementary to the target mRNA. dsRNAs and siRNAs in particular can be used to silence gene expression in mammalian cells (e.g., human cells). See, e.g., Clemens, J. C. et al. (2000) Proc. Natl. Sci. USA 97, 6499-6503; Billy, E. et al. (2001) Proc. Natl. Sci. USA 98, 14428-14433; Elbashir et al. (2001) Nature 411(6836):494-8; Yang, D. et al. (2002) Proc. Natl. Acad. Sci. USA 99, 9942-9947, US 2003-0166282, 2003-0143204, 2004-0038278, and 2003-0224432.

Anti-sense agents can also be used to inhibit expression of a protein involved in GDP-fucose biosynthesis or a fucosyltransferase and include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides that hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired.

Hybridization of antisense oligonucleotides with mRNA can interfere with one or more of the normal functions of mRNA. The functions of mRNA to be interfered with include all vital functions such as, for example, translocation of the RNA to the site of protein translation, translation of protein from the RNA, splicing of the RNA to yield one or more mRNA species, and catalytic activity that may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N4-(C1-C12)alkylaminocytosines and N4,N4-(C1-C12)dialkylaminocytosines. Modified nucleobases may also include 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N6-(C1-C12)alkylaminopurines and N6,N6-(C1-C12)dialkylaminopurines, including N6-methylaminoadenine and N6,N6-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C₁-C₃₀ alkyl, C₂-C₃₀ alkenyl, C₂-C₃₀ alkynyl, aryl, aralkyl, heteroaryl, halo, amino, amido, nitro, thio, sulfonyl, carboxyl, alkoxy, alkylcarbonyl, alkoxycarbonyl, and the like.

Descriptions of other types of nucleic acid agents are also available. See, e.g., U.S. Pat. No. 4,987,071; U.S. Pat. No. 5,116,742; U.S. Pat. No. 5,093,246; Woolf et al. (1992) Proc Natl Acad Sci USA; Antisense RNA and DNA, D. A. Melton, Ed., Cold Spring Harbor Laboratory, Cold Spring Harbor, N.Y. (1988); 89:7305-9; Haselhoff and Gerlach (1988) Nature 334:585-59; Helene, C. (1991) Anticancer Drug Des. 6:569-84; Helene (1992) Ann. N.Y. Acad. Sci. 660:27-36; and Maher, L. J. (1992) Bioassays 14:807-15.

### Genetically Engineered Cells

In some embodiments, a cell can be selected that has been genetically engineered for permanent or regulated inactivation (complete or partial) of a gene encoding a gene involved in GDP-fucose biosynthesis or a fucosyltransferase, or a protein involved in regulating GDP-fucose levels. For example, genes described herein can be inactivated. Permanent or regulated inactivation of gene expression can be achieved by targeting to a gene locus with a transfected plasmid DNA construct or a synthetic oligonucleotide. The plasmid construct or oligonucleotide can be designed to several forms. These include the following: 1) insertion of selectable marker genes or other sequences within an exon of the gene being inactivated; 2) insertion of exogenous sequences in regulatory regions of non-coding sequence; 3) deletion or replacement of regulatory and/or coding sequences; and, 4) alteration of a protein coding sequence by site specific mutagenesis.

In the case of insertion of a selectable marker gene into a coding sequence, it is possible to create an in-frame fusion of an endogenous exon of the gene with the exon engineered to contain, for example, a selectable marker gene. In this way following successful targeting, the endogenous gene expresses a fusion mRNA (nucleic acid sequence plus selectable marker sequence). Moreover, the fusion mRNA would be unable to produce a functional translation product.

In the case of insertion of DNA sequences into regulatory regions, the transcription of a gene can be reduced or silenced by disrupting the endogenous promoter region or any other regions in the 5′ untranslated region (5′ UTR) that is needed for transcription. Such regions include, for example, translational control regions and splice donors of introns. Secondly, a new regulatory sequence can be inserted upstream of the gene that would alter expression, e.g., eliminate expression, reduce expression, or render the gene subject to the control of extracellular factors. It would thus be possible to down-regulate or extinguish gene expression as desired for glycoprotein production. Moreover, a sequence that includes a selectable marker and a promoter can be used to disrupt expression of the endogenous sequence. Finally, all or part of the endogenous gene could be deleted by appropriate design of targeting substrates.

Cells Genetically Engineered to Express a Component Involved in Regulating GDP-fucose levels

Cells can be genetically engineered to express a component involved in regulation of GDP-fucose levels, e.g., a cell can be genetically engineered to overexpress a GMD, FX, fucokinase, GFPP, GDP-fucose synthetase, a GDP-fucose transporter, and/or a fucosyltransferase. When cells are to be genetically modified for the purposes of expressing or overexpressing a component, the cells may be modified by conventional genetic engineering methods or by gene activation.

According to conventional methods, a DNA molecule that contains cDNA or genomic DNA sequence encoding desired protein may be contained within an expression construct and transfected into primary, secondary, or immortalized cells by standard methods including, but not limited to, liposome-, polybrene-, or DEAE dextran-mediated transfection, electroporation, calcium phosphate precipitation, microinjection, or velocity driven microprojectiles (see, e.g., U.S. Pat. No. 6,048,729).

Alternatively, one can use a system that delivers the genetic information by viral vector. Viruses known to be useful for gene transfer include adenoviruses, adeno associated virus, herpes virus, mumps virus, pollovirus, retroviruses, Sindbis virus, and vaccinia virus such as canary pox virus.

Alternatively, the cells may be modified using a gene activation approach, for example, as described in U.S. Pat. No. 5,641,670; U.S. Pat. No. 5,733,761; U.S. Pat. No. 5,968,502; U.S. Pat. No. 6,200,778; U.S. Pat. No. 6,214,622; U.S. Pat. No. 6,063,630; U.S. Pat. No. 6,187,305; U.S. Pat. No. 6,270,989; and U.S. Pat. No. 6,242,218.

Accordingly, the term “genetically engineered,” as used herein in reference to cells, is meant to encompass cells that express a particular gene product following introduction of a DNA molecule encoding the gene product and/or including regulatory elements that control expression of a coding sequence for the gene product. The DNA molecule may be introduced by gene targeting or homologous recombination, i.e., introduction of the DNA molecule at a particular genomic site.

Methods of transfecting cells, and reagents such as promoters, markers, signal sequences that can be used for recombinant expression are known.

A component involved in regulating levels of GDP-fucose, e.g., GMD, FX, fucokinase, GFPP, GDP-fucose synthetase, a fucosyltransferase or a GDP-fucose transporter, can be placed under a selected form of control, e.g., inducible control. For example, a sequence encoding GMD, FX, fucokinase, GFPP, GDP-fucose synthetase, a fucosyltransferase or a GDP-fucose transporter, can be placed under the control of a promoter or other control element that is responsive to an inducer (or inhibitor) of expression. Such systems allow the cell to be maintained under a variety of conditions, e.g., a condition wherein the gene, e.g., a gene encoding GMD, FX, fucokinase, GFPP, GDP-fucose synthetase, a fucosyltransferase or a GDP-fucose transporter, is expressed or not expressed. This allows culture of the cell under a first condition, which provides glycoproteins having a first glycosylation state (e.g., fucosylated), or under a second condition, which provides glycoproteins having a second glycosylation state (e.g., lacking fucosylation).

Cells can also be engineered to express a hybrid nucleic acid; that is, a nucleic acid comprising at least two segments which have been isolated from at least two different sources. As one example of manipulation of a cell with a hybrid nucleic acid, a mammalian cell having a manipulation may express a hybrid nucleic acid comprising a regulatory sequence, such as a promoter and/or terminator sequence, of mammalian cell origin, which is functionally linked to a coding sequence, which may be of origin from a different species, e.g., from a different mammal or non-mammalian. In this manner, for example, a cell may be manipulated so that it can be induced to express the coding sequence in response to a stimulus that does not naturally induce expression of the linked coding sequence. An example of such a system is the TET On/Off regulatory system. In the Tet-Off system, gene expression is turned on when tetracycline (Tc) or doxycycline (Dox; a Tc derivative) is removed from the culture medium. In contrast, expression is turned on in the Tet-On system by the addition of Dox. The Tet-On system is responsive only to Dox, not to Tc. Both systems permit gene expression to be tightly regulated in response to varying concentrations of Tc or Dox.

Generally, one of ordinary skill can select promoters for a desired level of gene expression and place a selected gene under the control of such a promoter. The term promoter as used herein refers to a polynucleotide sequence which allows and controls the transcription of the genes or sequences functionally connected therewith. The sequences of promoters are deposited in databases such as GeneBank, and may be obtained as separate elements or elements cloned within polynucleotide sequences from commercial or individual sources. Exemplary types of promoters that can be used to express a desired gene of interest in eukaryotic cells (e.g., animal cells) include, but not limited to, constitutive and inducible promoters.

The activity of promoters may vary from one another in their strength, for example, across different cell types. Promoters that are particularly suitable for high expression in eukaryotic cells (e.g., animal cells) include, but not limited to, cytomegalovirus (CMV) immediate-early promoter, simian virus 40 (SV40) immediate-early promoter, human elongation factor 1α (EF-1α) promoter, chicken β-Actin promoter coupled with CMV early enhancer (CAG promoter), adenovirus major late promoter, and Rous sarcoma virus (RSV) promoter. Promoters that are suitable for intermediate or weak expression in eukaryotic cells (e.g., animal cells) include, but not limited to, human Ubiquitin C (UbC) promoter, murine phosphoglycerate kinase-1 (PGK) promoter, and herpes simplex virus (HSV) thymidine kinase (TK) promoter. Comparisons of the strength of various constitutive and inducible promoters in ectopic gene expression are described in, e.g., Qin, J. Y. et al., PLoS ONE 2010, 5(5):e10611; Cheng, X. et al., Int. J. Radiat. Biol. 1995, 67(3):261-267; Foecking, M. K. et al., Gene 1986, 45(1):101-105; Davis, M. G. et al., Biotechnol. Biochem. 1988, 10(1):6-12; Liu, Z. et al., Anal. Biochem. 1997, 246(1):150-152; Wenger, R. H. et al., Anal. Biochem. 1994, 221(2):416-418; Kronman, C. et al, Gene 1992, 121(2):295-304; Thompson, T. A. et al., In Vitro Cell Dev. Biol. 1993, 29A (2):165-170; Thompson, E. M. et al., Gene 1990, 96(2):257-262). One of ordinary skill can evaluate a particular combination of promoter, gene, and cell line to obtain the desired level of expression.

As mentioned above, with inducible promoters the activity of the promoter may be regulated (e.g., reduced or increased) in response to a signal (e.g., chemical signal (e.g., tetracycline, steroids, metal) or physical signal (e.g., temperature)). One example of an inducible promoter is the tetracycline (tet) promoter. As mentioned above, the tet promoter contains tetracycline a operator sequence (tetO) which can be induced by a tetracycline-regulated transactivator protein (tTA). Exemplary tetracycline-regulated promoters are described in e.g., U.S. Pat. Nos. 5,851,796, 5,464,758, 5,650,298, 5,589,362, 5,654,168, 5,789,156, 5,814,618, 5,888,981, 6,004,941, 6,136,954 and 6,271,348. Exemplary steroid-regulated promoters are described in e.g., U.S. Pat. Nos. 5,512,483 and 6,379,945. Exemplary metal-regulated promoters are described in e.g., U.S. Pat. Nos. 4,579,821 and 4,601,978. Examples of other inducible promoters include the jun, fos and heat shock promoter (see also Sambrook, J. et al., Molecular Cloning: A Laboratory Manual, Cold Spring Harbor Laboratory, Cold Spring Harbor, N.Y., 1989; Gossen, M. et al., Curr. Opinions Biotech. 1994, 5, 516-520).

The promoters described herein can be functionally combined with one or more regulatory sequences to regulate (e.g., increase, decrease, optimize, repress, induce) the transcription activity in an expression cassette. For example, the promoter can be functionally linked to one or more enhancer sequences (e.g., a CMV or SV40 enhancer) to increase transcriptional activity, or one or more binding sites for transcription factors (e.g., Sp1, AP1) to up- or down-regulate transcriptional activity. In an embodiment, the regulatory sequence can be positioned in front of or behind the promoter.

### Transcription Factors

The expression of a gene which conditions the level of GDP-fucose can also be down regulated by reducing, e.g., eliminating, the expression of a transcription factor which positively controls expression of the gene. E.g., Arnt, ATF6, SREBP-1c, Lmo2, HNF-1A, GCNF-2, CUTL1, STAT3, POU2F1a or EsF-1 can be targeted to down regulate GDP-fucose synthetase. HFH-1, Gfi-1, c-Myb, POU2F2C, AREB6, AORalpha2, POU3F1, LUN-1, or PPAR-gamma2 can be targeted to down regulate fucose kinase. Evi-1, STAT1beta, GATA-3, POU2F1A, POU3F2 (N-Oct-5b), AREB6, N-Myc, CUTL1, HSFlshort, or C/EBPbeta can be targeted to down regulate GNDS.

### Chemical Inhibitors of GMD, FX, Fucokinase, GFPP or GDP-Fucose Synthetase

Enzyme inhibitors are molecules that bind to enzymes and decrease their activities. The binding of an inhibitor may stop a substrate from entering the enzyme active site and/or hinder the enzyme from catalyzing its reaction. Inhibitor binding may be either reversible or irreversible. Irreversible inhibitors usually react with the enzyme and change it chemically. These inhibitors modify key amino acid residues needed for enzyme activity. In contrast, reversible inhibitors bind non-covalently and different types of inhibition are produced depending on whether these inhibitors bind the enzyme, the enzyme-substrate complex, or both.

In some embodiments, the addition of particular chemical reagents or inhibitors may be used to lower the levels of the GDP-fucose. These reagents or inhibitors may inhibit GMD, FX, fucokinase, GFPP, GDP-fucose synthetase, or enzymes involved in the biosynthesis of GDP-mannose. Examples of these inhibitors include, but are not limited to, guanosine-5′-O-(2-thiodiphosphate)-fucose, guanosine-5′-O-(2-thiodiphosphate)-mannose, pyridoxal-5′-phosphate, GDP-4-dehydro-6-L-deoxygalactose, GDP-L-fucose, guanosine diphosphate (GDP), guanosine monophosphate (GMP), GDP-D-glucose, p-chloromercuriphenylsulfonate EDTA and fucose.

### Glycoproteins

Glycoproteins that can be made by methods described herein include those in Table 1 below.

TABLE 1

| Protein Product | Reference Listed Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| Laronidase | Aldurazyme ® |
| interferon alfa-N3, human leukocyte derived | Alferon N ® |
| human antihemophilic factor | Alphanate ® |
| virus-filtered human coagulation factor IX | AlphaNine ® SD |
| Alefacept; recombinant, dimeric fusion protein LFA3-Ig | Amevive ® |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| Interferon beta-1b | Betaseron ® |
| Tositumomab | BEXXAR ® |
| antihemophilic factor | Bioclate ™ |
| human growth hormone | BioTropin ™ |
| botulinum toxin type A | BOTOX ® |
| Alemtuzumab | Campath ® |
| acritumomab; technetium-99 labeled | CEA-Scan ® |
| alglucerase; modified form of beta-glucocerebrosidase | Ceredase ® |
| imiglucerase; recombinant form of beta-glucocerebrosidase | Cerezyme ® |
| crotalidae polyvalent immune Fab, ovine | CroFab ™ |
| digoxin immune fab [ovine] | DigiFab ™ |
| Rasburicase | Elitek ® |
| Etanercept | ENBREL ® |
| epoietin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Helixate ® |
| Antihemophilic Factor; Factor XIII | HEMOFIL |
| adefovir dipivoxil | Hepsera ™ |
| Trastuzumab | Herceptin ® |
| Insulin | Humalog ® |
| antihemophilic factor/von Willebrand factor complex-human | Humate-P ® |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| eptifibatide | Integrilin ™ |
| alpha-interferon | Intron A ® |
| Palifermin | Kepivance |
| Anakinra | Kineret ™ |
| antihemophilic factor | Kogenate ®FS |
| insulin glargine | Lantus ® |
| granulocyte macrophage colony-stimulating factor | Leukine ®/Leukine ® Liquid |
| lutropin alfa for injection | Luveris |
| OspA lipoprotein | LYMErix ™ |
| Ranibizumab | LUCENTIS ® |
| gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ (formerly LeuTech ®) |
| somatropin [rDNA] | Norditropin ®/Norditropin Nordiflex ® |
| Mitoxantrone | Novantrone ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human soluable fusion protein | Orencia ™ |
| muromomab-CD3 | Orthoclone OKT3 ® |
| high-molecular weight hyaluronan | Orthovisc ® |
| human chorionic gonadotropin | Ovidrel ® |
| live attenuated Bacillus Calmette-Guerin | Pacis ® |
| peginterferon alfa-2a | Pegasys ® |
| pegylated version of interferon alfa-2b | PEG-Intron ™ |
| Abarelix (injectable suspension); gonadotropin-releasing | Plenaxis ™ |
| hormone antagonist | |
| epoietin alfa | Procrit ® |
| Aldesleukin | Proleukin, IL-2 ® |
| Somatrem | Protropin ® |
| dornase alfa | Pulmozyme ® |
| Efalizumab; selective, reversible T-cell blocker | RAPTIVA ™ |
| combination of ribavirin and alpha interferon | Rebetron ™ |
| Interferon beta 1a | Rebif ® |
| antihemophilic factor | Recombinate ® rAHF/ |
| antihemophilic factor | ReFacto ® |
| Lepirudin | Refludan ® |
| Infliximab | REMICADE ® |
| Abciximab | ReoPro ™ |
| Reteplase | Retavase ™ |
| Rituxima | Rituxan ™ |
| interferon alfa-2^{a} | Roferon-A ® |
| Somatropin | Saizen ® |
| synthetic porcine secretin | SecreFlo ™ |
| Basiliximab | Simulect ® |
| Eculizumab | SOLIRIS (R) |
| Pegvisomant | SOMAVERT ® |
| Palivizumab; recombinantly produced, humanized mAb | Synagis ™ |
| thyrotropin alfa | Thyrogen ® |
| Tenecteplase | TNKase ™ |
| Natalizumab | TYSABRI ® |
| human immune globulin intravenous 5% and 10% solutions | Venoglobulin-S ® |
| interferon alfa-n1, lymphoblastoid | Wellferon ® |
| drotrecogin alfa | Xigris ™ |
| Omalizumab; recombinant DNA-derived humanized | Xolair ® |
| monoclonal antibody targeting immunoglobulin-E | |
| Daclizumab | Zenapax ® |
| ibritumomab tiuxetan | Zevalin ™ |
| Somatotropin | Zorbtive ™ (Serostim ®) |

### Analytical Methods

In general, a glycan preparation can be subjected to analysis to determine whether the glycan includes a particular type of structure (e.g., a glycan structure described herein). In some embodiments, the analysis comprises comparing the structure and/or function of glycans in one glycoprotein preparation to structure and/or function of glycans in at least one other glycoprotein preparation. In some embodiments, the analysis comprises comparing the structure and/or function of glycans in one or more of the samples to structure and/or function of glycans in a reference sample.

Structure and composition of glycans can be analyzed by any available method. In some embodiments, glycan structure and composition are analyzed by chromatographic methods, mass spectrometry (MS) methods, chromatographic methods followed by MS, electrophoretic methods, electrophoretic methods followed by MS, nuclear magnetic resonance (NMR) methods, and combinations thereof.

In some embodiments, glycan structure and composition can be analyzed by chromatographic methods, including but not limited to, liquid chromatography (LC), high performance liquid chromatography (HPLC), ultra performance liquid chromatography (HPLC), thin layer chromatography (TLC), amide column chromatography, and combinations thereof.

In some embodiments, glycan structure and composition can be analyzed by mass spectrometry (MS) and related methods, including but not limited to, tandem MS, LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), and combinations thereof.

In some embodiments, glycan structure and composition can be analyzed by electrophoretic methods, including but not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof.

In some embodiments, glycan structure and composition can be analyzed by nuclear magnetic resonance (NMR) and related methods, including but not limited to, one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof.

In some embodiments, techniques described herein may be combined with one or more other technologies for the detection, analysis, and or isolation of glycans or glycoproteins. For example, in certain embodiments, glycans are analyzed in accordance with the present disclosure using one or more available methods (to give but a few examples, see Anumula, Anal. Biochem. 350(1):1, 2006; Klein et al., Anal. Biochem., 179:162, 1989; and/or Townsend, R.R. Carbohydrate Analysis” High Performance Liquid Chromatography and Capillary Electrophoresis, Ed. Z. El Rassi, pp 181-209, 1995, each of which is incorporated herein by reference in its entirety). For example, in some embodiments, glycans are characterized using one or more of chromatographic methods, electrophoretic methods, nuclear magnetic resonance methods, and combinations thereof. Exemplary such methods include, for example, NMR, mass spectrometry, liquid chromatography, 2-dimensional chromatography, SDS-PAGE, antibody staining, lectin staining, monosaccharide quantitation, capillary electrophoresis, fluorophore-assisted carbohydrate electrophoresis (FACE), micellar electrokinetic chromatography (MEKC), exoglycosidase or endoglycosidase treatments, and combinations thereof. Those of ordinary skill in the art will be aware of other techniques that can be used to characterize glycans together with the methods described herein.

In some embodiments, methods described herein allow for detection of a glycan structure (such as a glycan structure described herein) that is present at low levels within a population of glycans. For example, the present methods allow for detection of glycan species that are present at levels less than 10%, less than 5%, less than 4%, less than 3%, less than 2%, less than 1.5%, less than 1%, less than 0.75%, less than 0.5%, less than 0.25%, less than 0.1%, less than 0.075%, less than 0.05%, less than 0.025%, or less than 0.01% within a population of glycans.

In some embodiments, methods described herein allow for detection of particular structures (e.g., a glycan structure described herein) that are present at low levels within a population of glycans. For example, the present methods allow for detection of particular structures that are present at levels less than 10%, less than 5%, less than 4%, less than 3%, less than 2%, less than 1.5%, less than 1%, less than 0.75%, less than 0.5%, less than 0.25%, less than 0.1%, less than 0.075%, less than 0.05%, less than 0.025%, or less than 0.01% within a population of glycans.

In some embodiments, methods described herein allow for detection of relative levels of individual glycan species within a population of glycans. For example, the area under each peak of a liquid chromatograph can be measured and expressed as a percentage of the total. Such an analysis provides a relative percent amount of each glycan species within a population of glycans. In another example, relative levels of individual glycan species are determined from areas of peaks in a 1D-NMR experiment, or from volumes of cross peaks from a ¹H-¹⁵N HSQC spectrum (e.g., with correction based on responses from standards), or by relative quantitation by comparing the same peak across samples.

In some embodiments, a biological activity of a glycoprotein preparation (e.g., a glycoprotein preparation) is assessed. Biological activity of glycoprotein preparations can be analyzed by any available method. In some embodiments, a binding activity of a glycoprotein is assessed (e.g., binding to a receptor). In some embodiments, a therapeutic activity of a glycoprotein is assessed (e.g., an activity of a glycoprotein in decreasing severity or symptom of a disease or condition, or in delaying appearance of a symptom of a disease or condition). In some embodiments, a pharmacologic activity of a glycoprotein is assessed (e.g., bioavailability, pharmacokinetics, pharmacodynamics). For methods of analyzing bioavailability, pharmacokinetics, and pharmacodynamics of glycoprotein therapeutics, see, e.g., Weiner et al., J. Pharm. Biomed. Anal. 15(5):571-9, 1997; Srinivas et al., J. Pharm. Sci. 85(1):1-4, 1996; and Srinivas et al., Pharm. Res. 14(7):911-6, 1997.

As would be understood to one of skill in the art, the particular biological activity or therapeutic activity that can be tested will vary depending on the particular glycoprotein or glycan structure.

The potential adverse activity or toxicity (e.g., propensity to cause hypertension, allergic reactions, thrombotic events, seizures, or other adverse events) of glycoprotein preparations can be analyzed by any available method. In some embodiments, immunogenicity of a glycoprotein preparation is assessed, e.g., by determining whether the preparation elicits an antibody response in a subject.

### Cells & Cell Lines

Methods described herein use cells to produce products having reduced fucosylation. Examples of cells useful in these and other methods described herein follow.

The cell useful in the methods described herein can be eukaryotic or prokaryotic, as long as the cell provides or has added to it the enzymes to activate and attach saccharides present in the cell or saccharides present in the cell culture medium or fed to the cells. Examples of eukaryotic cells include yeast, insect, fungi, plant and animal cells, especially mammalian cells. Suitable mammalian cells include any normal mortal or normal or abnormal immortal animal or human cell, including: monkey kidney CV1 line transformed by SV40 (COS-7, ATCC CRL 1651); human embryonic kidney line (293) (Graham et al., J. Gen. Virol. 36:59 (1977)); baby hamster kidney cells (BHK, ATCC CCL 10); Chinese Hamster Ovary (CHO), e.g., DG44, DUKX-V11, GS-CHO (ATCC CCL 61, CRL 9096, CRL 1793 and CRL 9618); mouse sertoli cells (TM4, Mather, Biol. Reprod. 23:243 251 (1980)); monkey kidney cells (CV1 ATCC CCL 70); African green monkey kidney cells (VERO-76, ATCC CRL 1587); human cervical carcinoma cells (HeLa, ATCC CCL 2); buffalo rat liver cells (BRL 3A, ATCC CRL 1442); human lung cells (W138, ATCC CCL 75); human liver cells (Hep G2, HB 8065); mouse melanoma cells (NSO); mouse mammary tumor (MMT 060562, ATCC CCL51), TR1 cells (Mather, et al., Annals N.Y. Acad. Sci. 383:44 46 (1982)); canine kidney cells (MDCK) (ATCC CCL 34 and CRL 6253), HEK 293 (ATCC CRL 1573), WI-38 cells (ATCC CCL 75) (ATCC: American Type Culture Collection, Rockville, Md.), MCF-7 cells, MDA-MB-438 cells, U87 cells, A127 cells, HL60 cells, A549 cells, SP10 cells, DOX cells, SHSY5Y cells, Jurkat cells, BCP-1 cells, GH3 cells, 9L cells, MC3T3 cells, C3H-10T1/2 cells, NIH-3T3 cells, C6/36 cells, human lymphoblast cell lines (e.g. GEX) and PER.C6® cells. The use of mammalian tissue cell culture to express polypeptides is discussed generally in Winnacker, FROM GENES TO CLONES (VCH Publishers, N.Y., N.Y., 1987).

Exemplary plant cells include, for example, Arabidopsis thaliana, rape seed, corn, wheat, rice, tobacco etc.) (Staub, et al. 2000 Nature Biotechnology 1(3): 333-338 and McGarvey, P. B., et al. 1995 Bio-Technology 13(13): 1484-1487; Bardor, M., et al. 1999 Trends in Plant Science 4(9): 376-380). Exemplary insect cells (for example, Spodoptera frugiperda Sf9, Sf21, Trichoplusia ni, etc. Exemplary bacteria cells include Escherichia coli. Various yeasts and fungi such as Pichia pastoris, Pichia methanolica, Hansenula polymorpha, and Saccharomyces cerevisiae can also be selected.

Culture Media and Processing

The methods described herein can include determining and/or selecting media components or culture conditions which result in the production of a desired glycan property or properties. Culture parameters that can be determined include media components, pH, feeding conditions, osmolarity, carbon dioxide levels, agitation rate, temperature, cell density, seeding density, timing and sparge rate.

Changes in production parameters such the speed of agitation of a cell culture, the temperature at which cells are cultures, the components in the culture medium, the times at which cultures are started and stopped, variation in the timing of nutrient supply can result in variation of a glycan properties of the produced glycoprotein product. Thus, methods described herein can include one or more of: increasing or decreasing the speed at which cells are agitated, increasing or decreasing the temperature at which cells are cultures, adding or removing media components, and altering the times at which cultures are started and/or stopped.

Sequentially selecting a production parameters or a combination thereof, as used herein, means a first parameter (or combination) is selected, and then a second parameter (or combination) is selected, e.g., based on a constraint imposed by the choice of the first production parameter.

Media

The methods described herein can include determining and/or selecting a media component and/or the concentration of a media component that has a positive correlation to a desired glycan property or properties. A media component can be added in or administered over the course of glycoprotein production or when there is a change in media, depending on culture conditions. Media components include components added directly to culture as well as components that are a byproduct of cell culture.

Media components include, e.g., buffer, amino acid content, vitamin content, salt content, mineral content, serum content, carbon source content, lipid content, nucleic acid content, hormone content, trace element content, ammonia content, co-factor content, indicator content, small molecule content, hydrolysate content and enzyme modulator content. Specific examples of media conditions that will lead to altered levels of GDP-fucose include but are not limited to altering the levels of cobalt, butyrate, fucose, guanosine, and manganese.

Table 2 provides examples of various media components that can be selected.

TABLE 2

| | |
|---|---|
| amino acids | sugar precursors |
| Vitamins | Indicators |
| Carbon source (natural and unnatural) | Nucleosides or nucleotides |
| Salts | butyrate or organics |
| Sugars | DMSO |
| Sera | Animal derived products |
| Plant derived hydrolysates | Gene inducers |
| sodium pyruvate | Non natural sugars |
| Surfactants | Regulators of intracellular pH |
| Ammonia | Betaine or osmoprotectant |
| Lipids | Trace elements |
| Hormones or growth factors | minerals |
| Buffers | Non natural amino acids |
| Non natural amino acids | Non natural vitamins |

Exemplary buffers include Tris, Tricine, HEPES, MOPS, PIPES, TAPS, bicine, BES, TES, cacodylate, MES, acetate, MKP, ADA, ACES, glycinamide and acetamidoglycine.

The media can be serum free or can include animal derived products such as, e.g., fetal bovine serum (FBS), fetal calf serum (FCS), horse serum (HS), human serum, animal derived serum substitutes (e.g., Ultroser G, SF and HY; non-fat dry milk; Bovine EX-CYTE), fetuin, bovine serum albumin (BSA), serum albumin, and transferrin. When serum free media is selected lipids such as, e.g., palmitic acid and/or steric acid, can be included.

Lipids components include oils, saturated fatty acids, unsaturated fatty acids, glycerides, steroids, phospholipids, sphingolipids and lipoproteins.

Exemplary amino acid that can be included or eliminated from the media include alanine, arginine, asparagine, aspartic acid, cysteine, glutamic acid, glutamine, glycine, histidine, proline, isoleucine, leucine, lysine, methionine, phenylalanine, proline, serine, threonine, tryptophan, tyrosine and valine.

Examples of vitamins that can be present in the media or eliminated from the media include vitamin A (retinoid), vitamin B1 (thiamine), vitamin B2 (riboflavin), vitamin B3 (niacin), vitamin B5 (pantothenic acid), vitamin B6 (pyroxidone), vitamin B7 (biotin), vitamin B9 (folic acid), vitamin B12 (cyanocobalamin), vitamin C (ascorbic acid), vitamin D, vitamin E, and vitamin K.

Minerals that can be present in the media or eliminated from the media include bismuth, boron, calcium, chlorine, chromium, cobalt, copper, fluorine, iodine, iron, magnesium, manganese, molybdenum, nickel, phosphorus, potassium, rubidium, selenium, silicon, sodium, strontium, sulfur, tellurium, titanium, tungsten, vanadium, and zinc. Exemplary salts and minerals include CaCl₂ (anhydrous), CuSO₄ 5H₂O, Fe(NO₃).9H₂O, KCl, KNO₃, KH₂PO₄, MgSO₄ (anhydrous), NaCl, NaH₂PO₄H₂O, NaHCO₃, Na₂SeO₃ (anhydrous), ZnSO₄.7H₂O; linoleic acid, lipoic acid, D-glucose, hypoxanthine 2Na, phenol red, putrescine 2HCl, sodium pyruvate, thymidine, pyruvic acid, sodium succinate, succinic acid, succinic acid.Na.hexahydrate, glutathione (reduced), para-aminobenzoic acid (PABA), methyl linoleate, bacto peptone G, adenosine, cytidine, guanosine, 2′-deoxyadenosine HCl, 2′-deoxycytidine HCl, 2′-deoxyguanosine and uridine. When the desired glycan characteristic is decreased fucosylation, the production parameters can include culturing a cell, e.g., CHO cell, e.g., dhfr deficient CHO cell, in the presence of manganese, e.g., manganese present at a concentration of about 0.1 μM to 50 μM. Decreased fucosylation can also be obtained, e.g., by culturing a cell (e.g., a CHO cell, e.g., a dhfr deficient CHO cell) at an osmolality of about 350 to 500 mOsm. Osmolality can be adjusted by adding salt to the media or having salt be produced as a byproduct as evaporation occurs during production.

Hormones include, for example, somatostatin, growth hormone-releasing factor (GRF), insulin, prolactin, human growth hormone (hGH), somatotropin, estradiol, and progesterone. Growth factors include, for example, bone morphogenic protein (BMP), epidermal growth factor (EGF), basic fibroblast growth factor (bFGF), nerve growth factor (NGF), bone derived growth factor (BDGF), transforming growth factor-beta1 (TGF-beta1), [Growth factors from U.S. Pat. No. 6,838,284 B2], hemin and NAD.

Examples of surfactants that can be present or eliminated from the media include Tween-80 and pluronic F-68.

Small molecules can include, e.g., butyrate, ammonia, non natural sugars, non natural amino acids, chloroquine, and betaine.

In some embodiments, ammonia content can be selected as a production parameter to produce a desired glycan characteristic or characteristics. For example, ammonia can be present in the media in a range from 0.001 to 50 mM. Ammonia can be directly added to the culture and/or can be produced as a by product of glutamine or glucosamine. When the desired glycan characteristic is one or more of an increased number of high mannose structures, decreased fucosylation and decreased galactosylation, the production parameters selected can include culturing a cell (e.g., a CHO cell, e.g., a dhfr deficient CHO cell) in the presence of ammonia, e.g., ammonia present at a concentration of about 0.01 to 50 mM. For example, if the desired glycan characteristic includes decreased galactosylation, production parameters selected can include culturing a cell (e.g., a CHO cell, e.g., a dhfr deficient CHO cell) in serum containing media and in the presence of ammonia, e.g., ammonia present at a concentration of about 0.01 to 50 mM.

Another production parameter is butyrate content. The presence of butyrate in culture media can result in increased galactose levels in the resulting glycoprotein preparation. Butyrate provides increased sialic acid content in the resulting glycoprotein preparation. Therefore, when increased galactosylation and/or sialylation is desired, the cell used to produce the glycoprotein (e.g., a CHO cell, e.g., a dhfr deficient CHO cell) can be cultured in the presence of butyrate. In some embodiments, butyrate can be present at a concentration of about 0.001 to 10 mM, e.g., about 2 mM to 10 mM. For example, if the desired glycan characteristic includes increased sialylation, production parameters selected can include culturing a cell (e.g., a CHO cell, e.g., a dhfr deficient CHO cell) in serum containing media and in the presence of butyrate, e.g., butyrate present at a concentration of about 2.0 to 10 mM. Such methods can further include selecting one or more of adherent culture conditions and culture in a T flask.

Physiochemical Parameters

Methods described herein can include selecting culture conditions that are correlated with a desired glycan property or properties. Such conditions can include temperature, pH, osmolality, shear force or agitation rate, oxidation, spurge rate, growth vessel, tangential flow, DO, CO₂, nitrogen, fed batch, redox, cell density and feed strategy. Examples of physiochemical parameters that can be selected are provided in Table 3.

TABLE 3

| | |
|---|---|
| Temperature | DO |
| pH | CO₂ |
| osmolality | Nitrogen |
| shear force, or agitation rate | Fed batch |
| oxidation | Redox |
| Spurge rate | Cell density |
| Growth vessel | Perfusion culture |
| Tangential flow | Feed strategy |
| Batch | |

For example, the production parameter can be culturing a cell under acidic, neutral or basic pH conditions. Temperatures can be selected from 10 to 42° C. For example, a temperature of about 28 to 36° C. does not significantly alter galactosylation, fucosylation, high mannose production, hybrid production or sialylation of glycoproteins produced by a cell (e.g., a CHO cell, e.g., a dhfr deficient CHO cell) cultured at these temperatures. In addition, any method that slows down the growth rate of a cell may also have this effect. Thus, temperatures in this range or methods that slow down growth rate can be selected when it is desirable not to have this parameter of production altering glycosynthesis.

In other embodiments, carbon dioxide levels can be selected which results in a desired glycan characteristic or characteristics. CO₂ levels can be, e.g., about 5%, 6%, 7%, 8%, 9%, 10%, 11%, 13%, 15%, 17%, 20%, 23% and 25% (and ranges in between). In one embodiment, when decreased fucosylation is desired, the cell can be cultured at CO₂ levels of about 11 to 25%, e.g., about 15%. CO₂ levels can be adjusted manually or can be a cell byproduct.

A wide array of flasks, bottles, reactors, and controllers allow the production and scale up of cell culture systems. The system can be chosen based, at least in part, upon its correlation with a desired glycan property or properties.

Cells can be grown, for example, as batch, fed-batch, perfusion, or continuous cultures.

Production parameters that can be selected include, e.g., addition or removal of media including when (early, middle or late during culture time) and how often media is harvested; increasing or decreasing speed at which cell cultures are agitated; increasing or decreasing temperature at which cells are cultured; adding or removing media such that culture density is adjusted; selecting a time at which cell cultures are started or stopped; and selecting a time at which cell culture parameters are changed. Such parameters can be selected for any of the batch, fed-batch, perfusion and continuous culture conditions.

### EXAMPLES

### Example 1

### Relationship Between Levels of GDP-Fucose and % Fucosylated Glycans

The levels of GDP-fucose levels and the degree of protein fucosylation on glycoproteins were analyzed for three different CHO cell lines expressing a representative secreted protein product (CTLA4Ig): CHO cells that are deficient in the enzyme GDP-mannose 4,6, dehydratase (Lec 13.6 A); CHO cells that have lowered levels of GDP-fucose (Lec 2); and wild-type CHO cells. Culture media did not contain free fucose except as indicated for Lec 13.6 A cells cultured in the presence of exogenous fucose supplemented at 0.01 and 1 mM in the culture media. Cells were harvested, and snap frozen, while culture supernatant was harvested and CTLA4Ig harvested by protein A purification for subsequent analysis. Cells were then subjected to nucleotide sugar extraction using standard methods. In short with chloroform:methanol:water (2:4:1), the pellets discarded and the resulting extraction dried down. The dried material was subsequently resuspended in 500 ul of 10% butanol in water and then extracted with 1 ml of 90% butanol in water. The butanol phase was discarded and the aqueous subjected to a second butanol extraction. The final aqueous phase was dried down and the sugar nucleotides further isolated by PGC chromatography eluting off with 25% acetonitrile (v/v) containing 50 mM triethylammonium acetate. For quantification, sugar-nucletides were resolved with RP chromatography.

Protein products were isolated from culture supernatant by protein A affinity, and subjected to PNGase F treatment to remove glycans. The resulting glycans were isolated by PGC chromatography and subsequently analyzed by MALDI mass spectrometry. The % fucosylation was determined by determining the ratio of the glycans with or without core fucosylation. Results are presented in Table 4. GDP-fucose levels are indicated in peak area as detected by UV.

TABLE 4

| | % of Parental | % Fucosylated |
|---|---|---|
| Cell Line | GDP-fucose | Glycans |
| Wild-type CHO | 100 | >90 |
| Lec 2 | 80 | >90 |
| Lec 13.6A | 61.6 | ≈20 |
| Lec 13.6A + 1 mM fucose | 270 | 100 |
| Lec 13.6A + 0.01 mM fucose | 62.5 | 45 |

What is claimed is:

1. A method of reducing fucosylation of a glycoprotein (or a preparation of glycoproteins), comprising:
providing a cell having or subject to a manipulation that results in a level of GDP-fucose in said cell that is below a first preselected level and, in embodiments, above a second preselected level;
culturing said cell, e.g., to provide a batch of cultured cells;
optionally, measuring the level of GDP-fucose in said cell or batch of cultured cells;
optionally, separating the glycoprotein from at least one component with which said cell or batch of cultured cells was cultured; and
optionally, evaluating the glycoprotein (or a glycoprotein on the surface of the cell) for a parameter related to fucosylation;
thereby providing a glycoprotein with reduced fucosylation, e.g., wherein the level of fucosylation is reduced by a predetermined level in comparison with a reference.

2. The method of claim 1, further comprising evaluating a glycan on the surface of said cell or batch of cultured cells in order to determine if the glycoprotein produced by said cell or batch of cultured cells has reduced fucosylation.

3. The method of claim 2, wherein said evaluation comprises evaluating a glycan on the surface of said cell or batch of cultured cells, to determine a property of said glycan, comparing the property to a reference, to thereby determine if said glycan structure is present on the product.

4. The method of claim 1, wherein said first preselected level of GDP-fucose is selected from:
i.a) approximately equal to or less than 80%, 70% or 60% of a reference level, e.g., the level in said cell or batch of cultured cells, e.g., a cell or batch of cultured cells which is otherwise similar, without the manipulation;
ii.a) approximately equal to, or less than, the point of maximum curvature above the inflection point (e.g., the inflection point in the second phase) on a graph of the amount of fucosylation vs. decrease in GDP-fucose;
ii.1.a) approximately equal to, or less than, the lowest level that results in a normal (e.g., that seen in an un-manipuated cell) level of fucosylation;
iii.a) approximately equal to or less than the point of maximum curvature below the inflection point on a graph of the amount of fucosylation vs. decrease in GDP-fucose;
iii.1.a) approximately equal to, or less than, the highest level that results in no further reduction in fucosylation;
iv.a) approximately equal to or less than point A on the curve in FIG. 1, or less than or equal to an analogous point on a plot of the amount of fucosylation (%) vs. the amount of GDP fucose as a % of control;
v.a) approximately equal to or less than that corresponding to an amount between points A and B on the curve in FIG. 1, or less than or equal to an analogous point on a plot of the amount of fucosylation (%) vs. the amount of GDP fucose as a % of control; or
vi.a) approximately equal to or less than point B on the curve in FIG. 1, or less than or equal to an analogous point on a plot of the amount of fucosylation (%) vs. the amount of GDP fucose as a % of control.

5. The method of claim 1, wherein said second preselected level of GDP-fucose is selected from:
i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;
ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;
iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level;
iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;
v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level; or
vi.b) approximately equal to or greater than point C on the curve in FIG. 1, or greater than or equal to an analogous point on a plot of the amount of fucosylation (%) vs. the amount of GDP fucose as a % of control.

6. The method of claim 1, wherein the level of GDP-fucose is selected to be outside the range between A and B on the curve in FIG. 1.

7. The method of claim 1, wherein the level of GDP-fucose is reduced by a predetermined level, e.g., in comparison with a reference.

8. The method of claim 7, wherein the reference is the amount present in a cell or batch of cultured cells, e.g., a CHO cell or batch of cultured cells, lacking the manipulation but otherwise the same or essentially the same as the cell having the manipulation.

9. The method of claim 1, wherein the level of GDP-fucose is reduced by, as much as, or more than, 10, 20, 30, 40, 50, 60, 70, 80 or 90%, as compared to the reference.

10. The method of claim 1, further comprising evaluating the glycoprotein for a parameter related to fucosylation, e.g., the amount of fucosylation in the glycan complement, the amount or fucosylation on a component of the glycan complement, or the amount of fucosylation on a glycan component, e.g., in a preparation of glycoproteins.

11. The method of claim 1, further comprising evaluating the glycoprotein for a parameter related to fucosylation, e.g., the proportion of a preselected glycan component which bears a fucosyl moiety, e.g., at a selected position on the glycan component, e.g., in a preparation of glycoproteins.

12. The method of claim 1, wherein the level of fucosylation at one, two, three, or more preselected amino acid residues is evaluated.

13. The method of claim 1, wherein the level of fucosylation is reduced by a predetermined level in comparison with a reference.

14. The method of claim 13, wherein the reference is the amount present in a cell or batch of cultured cells, e.g., a CHO cell or batch of cultured cells, lacking the manipulation but otherwise the same or essentially the same as the cell or batch of cultured cells having the manipulation.

15. The method of claim 1, wherein the level of fucosylation is reduced by, as much as, or more than, 10, 20, 30, 40, 50, 60, 70, 80 or 90%, as compared to the reference.

16. The method of claim 1, wherein X_{F} is greater than X_{G},
and wherein,
X_{F} is the % or proportion of reduction in the level of fucosylation (e.g., as compared to the level of fucosylation in a cell or batch of cultured cells lacking the manipulation); and
X_{G} is the % or proportion of reduction in the level of GDP fucose (as compared to the level of GDP fucose in a cell or batch of cultured cells lacking the manipulation).

17. The method of claim 1, wherein said manipulation is not a genetic lesion or the presence of an siRNA that reduces the level of an enzyme that promotes formation of GDP-fucose, or the attachment of a fucosyl moiety. For example, the manipulation is not a lesion that decreases the expression of GMD, FX, fucokinase, GFPP, GDP-synthetase, a fucosyltransferase or a GDP-Fucose transporter.

18. The method of claim 1, wherein the cell or batch of cultured cells is wild-type for one or all of GMD, FX, fucokinase, GFPP, GDP-synthetase, a fucosyltransferase or a GDP-Fucose transporter.

19. The method of claim 1, wherein the cell or batch of cultured cells does not include an siRNA that targets GMD, FX, fucokinase, GFPP, GDP-synthetase, a fucosyltransferase or a GDP-Fucose transporter.

20. The method of claim 1, wherein absent the manipulation, the level of fucosylation is substantially the same as the level in a wild-type cell.

21. The method of claim 1, wherein the manipulated cell carries no mutation that substantially lowers GDP-fucose levels.

22. The method of claim 1, wherein the manipulated cell has no siRNA that substantially lowers GDP-fucose levels.

23. The method of claim 1, wherein the cell has a mutation that decreases the level of GDP-fucose, e.g., a mutation in GMD, FX, fucokinase, GFPP, GDP-synthetase, a fucosyltransferase or a GDP-Fucose transporter, and fucose or another substance is present in the culture medium at a level that results in a level of GDP-fucose recited in claim 1.

24. The method of claim 1, wherein the manipulation is the presence of an siRNA that reduces the level of an enzyme that promotes formation of GDP-fucose, or the attachment of a fucosyl moiety, e.g., an siRNA that targets GMD, FX, fucokinase, GFPP, GDP-synthetase, a fucosyltransferase or a GDP-Fucose transporter, and fucose or another substance is present in the culture medium at a level that results in a level of GDP-fucose recited in claim 1.

25. The method of claim 1, wherein said culturing comprises culturing the cell in a medium that results in said level of GDP-fucose.

26. The method of claim 1, wherein the glycoprotein is an antibody.

27. The method of claim 26, wherein the antibody has reduced core fucosylation.

28. The method of claim 27, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

29. The method of claim 1, wherein the cell is a Chinese Hamster Ovary (CHO) cell.

30. The method of claim 29, wherein the glycoprotein is an antibody.

31. The method of claim 30, wherein the antibody has reduced core fucosylation.

32. The method of claim 31, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

33. The method of claim 1, wherein the glycoprotein is selected from Table 1.

34. The method of claim 1, further comprising culturing a plurality of the cells and separating as much as, or at least, 1, 10, 100, 1,000, or 10,000 grams of the glycoprotein from the cells.

35. The method of claim 1, further comprising combining the glycoprotein having reduced fucosylation with a pharmaceutically acceptable component and, e.g., formulating the glycoprotein having reduced fucosylation into a pharmaceutically acceptable formulation.

36. The method of claim 1, wherein the glycoprotein is analyzed by one or more of HPLC, CE, MALDI-MS and NMR.

37. The method of claim 1, where the manipulation is, or is the product of, a selection for reduced levels of GDP-fucose.

38. The method of claim 1, where the manipulation is, or is the product of, a selection for reduced fucosylation of a glycoprotein.

39. The method of claim 1, where the manipulation comprises contact with, or inclusion in or on the cell or batch of cultured cells, of an exogenous inhibitor of an enzyme involved in GDP-fucose biosynthesis, e.g., a specific or non-specific inhibitor.

40. The method of claim 1, wherein the level of fucosylation at one, two, three, or more preselected amino acid residues is evaluated.

41. The method of claim 1, wherein one or more of said cell or said batch of cultured cells, said manipulation, and said glycoprotein, is selected on the basis that it or the combination will provide a glycoprotein having reduced fucosylation.

42. The method of claim 1, further comprising, providing a value for a parameter associated with a compound other than GDP-fucose, wherein a parameter for the compound, e.g., the level of the compound, is correlated to the level of GDP-fucose.

43. The method of claim 42, further comprising providing a comparison of the value with a reference value, wherein optionally, a preselected relationship of the value to the reference value, e.g., greater than, equal to, or less than, is indicative of whether the level of GDP fucose is above, at or below the second level.

44. The method of claim 43, further comprising, responsive to the result of the comparison, increasing the level of GDP-fucose, decreasing the level of GDP-fucose or continuing cell culture without intervening to change the level of GDP-fucose.

45. The method of claim 42, wherein the compound other than GDP-fucose is GDP-mannose.

46. The method of claim 42, wherein the compound other than GDP-fucose is GDP-mannose and the parameter is the level of GDP-mannose.

47. The method of claim 1, further comprising, providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose.

48. The method of claim 47, comprising continuing to culture said cells, and repeating the steps of claim 47.

49. A method of reducing fucosylation of a glycoprotein or a preparation of glycoproteins, the method comprising:
providing a cell that expresses said glycoprotein and that is wild-type for one or more of GMD, FX, fucokinase, GFPP, GDP-Fucose synthetase, a fucosyltransferase or a GDP-Fucose transporter;
culturing said cell under conditions that result in a level of GDP-fucose in said cell that is below a first preselected level and, in embodiments, above a second preselected level, and results in a preselected level of fucosylation, which is less than in a reference cell cultured under reference conditions, e.g., to provide a batch of cultured cells;
optionally, measuring the level of GDP-fucose in said cell or batch of cultured cells; and
optionally, separating the glycoprotein from at least one component with which said cell or batch of cultured cells was cultured,
optionally, evaluating the glycoprotein (or a glycoprotein on the surface of the cell or batch of cultured cells) for a parameter related to fucosylation;
thereby providing a glycoprotein with reduced fucosylation, e.g., wherein the level of fucosylation is reduced by a predetermined level in comparison with a reference.

50. The method of claim 49, further comprising evaluating a glycan on the surface of said cell or batch of cultured cells in order to determine if the glycoprotein produced by said cell or batch of cultured cells has reduced fucosylation.

51. The method of claim 50, wherein said evaluation comprises evaluating a glycan on the surface of said cell or batch of cultured cells, to determine a property of said glycan, comparing the property to a reference, to thereby determine if said glycan structure is present on the product.

52. The method of claim 49, wherein said first preselected level of GDP-fucose is selected from:
i.a) approximately equal to or less than 80%, 70% or 60% of a reference level, e.g., the level in said cell or batch of cultured cells, e.g., a cell or batch of cultured cells which is otherwise similar, without the manipulation;
ii.a) approximately equal to, or less than, the point of maximum curvature above the inflection point (e.g., the inflection point in the second phase) on a graph of the amount of fucosylation vs. decrease in GDP-fucose;
ii.1.a) approximately equal to, or less than, the lowest level that results in a normal (e.g., that seen in an un-manipuated cell) level of fucosylation;
iii.a) approximately equal to or less than the point of maximum curvature below the inflection point on a graph of the amount of fucosylation vs. decrease in GDP-fucose;
iii.1.a) approximately equal to, or less than, the highest level that results in no further reduction in fucosylation;
iv.a) approximately equal to or less than point A on the curve in FIG. 1, or less than or equal to an analogous point on a plot of the amount of fucosylation (%) vs. the amount of GDP fucose as a % of control;
v.a) approximately equal to or less than that corresponding to an amount between points A and B on the curve in FIG. 1, or less than or equal to an analogous point on a plot of the amount of fucosylation (%) vs. the amount of GDP fucose as a % of control; or
vi.a) approximately equal to or less than point B on the curve in FIG. 1, or less than or equal to an analogous point on a plot of the amount of fucosylation (%) vs. the amount of GDP fucose as a % of control.

53. The method of claim 49, wherein said second preselected level of GDP-fucose is selected from:
i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;
ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;
iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level;
iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;
v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level; or
vi.b) approximately equal to or greater than point C on the curve in FIG. 1, or greater than or equal to an analogous point on a plot of the amount of fucosylation (%) vs. the amount of GDP fucose as a % of control.

54. The method of claim 49, wherein the level of GDP-fucose is selected to be outside the range between A and B on the curve in FIG. 1.

55. The method of claim 49, wherein the level of GDP-fucose is reduced by a predetermined level, e.g., in comparison with a reference.

56. The method of claim 55, wherein the reference is the amount present in a cell or batch of cultured cells, e.g., a CHO cell or batch of cultured cells, cultured under reference conditions but otherwise the same or essentially the same as the cell cultured under conditions that result in said level of GDP-fucose.

57. The method of claim 49, wherein the level of GDP-fucose is reduced by, as much as, or more than, 10, 20, 30, 40, 50, 60, 70, 80 or 90%, as compared to the reference.

58. The method of claim 49, further comprising evaluating the glycoprotein for a parameter related to fucosylation, e.g., the amount of fucosylation in the glycan complement, the amount or fucosylation on a component of the glycan complement, or the amount of fucosylation on a glycan component, e.g., in a preparation of glycoproteins.

59. The method of claim 49, further comprising evaluating the glycoprotein for a parameter related to fucosylation, e.g., the proportion of a preselected glycan component which bears a fucosyl moiety, e.g., at a selected position on the glycan component, e.g., in a preparation of glycoproteins.

60. The method of claim 49, wherein the level of fucosylation at one, two, three, or more preselected amino acid residues is evaluated.

61. The method of claim 49, wherein the level of fucosylation is reduced by a predetermined level in comparison with a reference.

62. The method of claim 61, wherein the reference is the amount present in a cell or batch of cultured cells, e.g., a CHO cell or batch of cultured cells, cultured under reference conditions but otherwise the same or essentially the same as the cell cultured under conditions that result in said level of GDP-fucose.

63. The method of claim 49, wherein the level of fucosylation is reduced by, as much as, or more than, 10, 20, 30, 40, 50, 60, 70, 80 or 90%, as compared to the reference.

64. The method of claim 49, wherein X_{F} is greater than X_{G},
and wherein,
X_{F} is the % or proportion of reduction in the level of fucosylation (e.g., as compared to the level of fucosylation in a cell or batch of cultured cells cultured under reference conditions); and
X_{G} is the % or proportion of reduction in the level of GDP fucose (as compared to the level of GDP fucose in a cell or batch of cultured cells cultured under reference conditions).

65. The method of claim 49, wherein the cell or batch of cultured cells does not include an siRNA that targets GMD, FX, fucokinase, GFPP, GDP-synthetase, a fucosyltransferase or a GDP-Fucose transporter.

66. The method of claim 49, wherein said culturing comprises culturing the cell in a medium that results in said level of GDP-fucose.

67. The method of claim 49, wherein the glycoprotein is an antibody.

68. The method of claim 67, wherein the antibody has reduced core fucosylation.

69. The method of claim 68, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

70. The method of claim 49, wherein the cell is a Chinese Hamster Ovary (CHO) cell.

71. The method of claim 70, wherein the glycoprotein is an antibody.

72. The method of claim 71, wherein the antibody has reduced core fucosylation.

73. The method of claim 72, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

74. The method of claim 49, wherein the glycoprotein is selected from Table 1.

75. The method of claim 49, further comprising culturing a plurality of the cells and separating as much as, or at least, 1, 10, 100, 1,000, or 10,000 grams of the glycoprotein from the cells.

76. The method of claim 49, further comprising combining the glycoprotein having reduced fucosylation with a pharmaceutically acceptable component and, e.g., formulating the glycoprotein having reduced fucosylation into a pharmaceutically acceptable formulation.

77. The method of claim 49, wherein the glycoprotein is analyzed by one or more of HPLC, CE, MALDI-MS and NMR.

78. The method of claim 49, wherein the level of fucosylation at one, two, three, or more preselected amino acid residues is evaluated.

79. The method of claim 49, further comprising, providing a value for a parameter associated with a compound other than GDP-fucose, wherein a parameter for the compound, e.g., the level of the compound, is correlated to the level of GDP-fucose.

80. The method of claim 79, further comprising providing a comparison of the value with a reference value, wherein optionally, a preselected relationship of the value to the reference value, e.g., greater than, equal to, or less than, is indicative of whether the level of GDP fucose is above, at or below the second level.

81. The method of claim 80, further comprising, responsive to the result of the comparison, increasing the level of GDP-fucose, decreasing the level of GDP-fucose or continuing cell culture without intervening to change the level of GDP-fucose.

82. The method of claim 79, wherein the compound other than GDP-fucose is GDP-mannose.

83. The method of claim 79, wherein the compound other than GDP-fucose is GDP-mannose and the parameter is the level of GDP-mannose.

84. The method of claim 49, further comprising, providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose.

85. The method of claim 84, comprising continuing to culture said cells, and repeating the steps of claim 84.

86. A method of providing a glycoprotein having fucosylation that is reduced compared to a reference glycoprotein, e.g., an FDA approved glycoprotein, the method comprising:
providing a cell that expresses said reference glycoprotein, which optionally, is wild-type for one or more of GMD, FX, fucokinase, GFPP, GDP-Fucose synthetase, a fucosyltransferase or a GDP-Fucose transporter;
culturing said cell (without inducing a mutation in, or adding an siRNA that targets one or more of GMD, FX, fucokinase, GFPP, GDP-Fuc synthetase, a fucosyltransferase or a GDP-Fucose transporter) under culture conditions that result in a level of GDP-fucose in said cell that is below a first preselected level and, in embodiments, above a second preselected level, and results in a preselected level of fucosylation, which is less than in a reference cell cultured under reference conditions, e.g., to provide a batch of cultured cells;
optionally, measuring the level of GDP-fucose in said cell or batch of cultured cells; and
optionally, separating the glycoprotein from at least one component with which said cell or batch of cultured cells was cultured;
optionally, evaluating the glycoprotein (or a glycoprotein on the surface of the cell or batch of cultured cells) for a parameter related to fucosylation;
thereby providing a glycoprotein having fucosylation that is reduced compared to a reference glycoprotein, e.g., an FDA approved glycoprotein.

87. The method of claim 86, further comprising evaluating a glycan on the surface of said cell or batch of cultured cells in order to determine if the glycoprotein produced by said cell or batch of cultured cells has reduced fucosylation.

88. The method of claim 87, wherein said evaluation comprises evaluating a glycan on the surface of said cell or batch of cultured cells, to determine a property of said glycan, comparing the property to a reference, to thereby determine if said glycan structure is present on the product.

89. The method of claim 86, wherein said first preselected level of GDP-fucose is selected from:
i.a) approximately equal to or less than 80%, 70% or 60% of a reference level, e.g., the level in said cell or batch of cultured cells, e.g., a cell or batch of cultured cells which is otherwise similar, without the manipulation;
ii.a) approximately equal to, or less than, the point of maximum curvature above the inflection point (e.g., the inflection point in the second phase) on a graph of the amount of fucosylation vs. decrease in GDP-fucose;
ii.1.a) approximately equal to, or less than, the lowest level that results in a normal (e.g., that seen in an un-manipuated cell) level of fucosylation;
iii.a) approximately equal to or less than the point of maximum curvature below the inflection point on a graph of the amount of fucosylation vs. decrease in GDP-fucose;
iii.1.a) approximately equal to, or less than, the highest level that results in no further reduction in fucosylation;
iv.a) approximately equal to or less than point A on the curve in FIG. 1, or less than or equal to an analogous point on a plot of the amount of fucosylation (%) vs. the amount of GDP fucose as a % of control;
v.a) approximately equal to or less than that corresponding to an amount between points A and B on the curve in FIG. 1, or less than or equal to an analogous point on a plot of the amount of fucosylation (%) vs. the amount of GDP fucose as a % of control; or
vi.a) approximately equal to or less than point B on the curve in FIG. 1, or less than or equal to an analogous point on a plot of the amount of fucosylation (%) vs. the amount of GDP fucose as a % of control.

90. The method of claim 86, wherein said second preselected level of GDP-fucose is selected from:
i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;
ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;
iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level;
iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;
v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level; or
vi.b) approximately equal to or greater than point C on the curve in FIG. 1, or greater than or equal to an analogous point on a plot of the amount of fucosylation (%) vs. the amount of GDP fucose as a % of control.

91. The method of claim 86, wherein the level of GDP-fucose is selected to be outside the range between A and B on the curve in FIG. 1.

92. The method of claim 86, wherein the level of GDP-fucose is reduced by a predetermined level, e.g., in comparison with a reference.

93. The method of claim 92, wherein the reference is the amount present in a cell or batch of cultured cells, e.g., a CHO cell or batch of cultured cells, cultured under reference conditions but otherwise the same or essentially the same as the cell cultured under conditions that result in said level of GDP-fucose.

94. The method of claim 86, wherein the level of GDP-fucose is reduced by, as much as, or more than, 10, 20, 30, 40, 50, 60, 70, 80 or 90%, as compared to the reference.

95. The method of claim 86, further comprising evaluating the glycoprotein for a parameter related to fucosylation, e.g., the amount of fucosylation in the glycan complement, the amount or fucosylation on a component of the glycan complement, or the amount of fucosylation on a glycan component, e.g., in a preparation of glycoproteins.

96. The method of claim 86, further comprising evaluating the glycoprotein for a parameter related to fucosylation, e.g., the proportion of a preselected glycan component which bears a fucosyl moiety, e.g., at a selected position on the glycan component, e.g., in a preparation of glycoproteins.

97. The method of claim 86, wherein the level of fucosylation at one, two, three, or more preselected amino acid residues is evaluated.

98. The method of claim 86, wherein the level of fucosylation is reduced by a predetermined level in comparison with a reference.

99. The method of claim 98, wherein the reference is the amount present in a cell or batch of cultured cells, e.g., a CHO cell or batch of cultured cells, cultured under reference conditions but otherwise the same or essentially the same as the cell cultured under conditions that result in said level of GDP-fucose.

100. The method of claim 86, wherein the level of fucosylation is reduced by, as much as, or more than, 10, 20, 30, 40, 50, 60, 70, 80 or 90%, as compared to the reference.

101. The method of claim 86, wherein X_{F} is greater than X_{G},
and wherein,
X_{F} is the % or proportion of reduction in the level of fucosylation (e.g., as compared to the level of fucosylation in a cell or batch of cultured cells cultured under reference conditions); and
X_{G} is the % or proportion of reduction in the level of GDP fucose (as compared to the level of GDP fucose in a cell or batch of cultured cells cultured under reference conditions).

102. The method of claim 86, wherein said culturing comprises culturing the cell in a medium that results in said level of GDP-fucose.

103. The method of claim 86, wherein the glycoprotein is an antibody.

104. The method of claim 103, wherein the antibody has reduced core fucosylation.

105. The method of claim 104, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

106. The method of claim 86, wherein the cell is a Chinese Hamster Ovary (CHO) cell.

107. The method of claim 106, wherein the glycoprotein is an antibody.

108. The method of claim 107 wherein the antibody has reduced core fucosylation.

109. The method of claim 108, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

110. The method of claim 86, wherein the glycoprotein is selected from Table 1.

111. The method of claim 86, further comprising culturing a plurality of the cells and separating as much as, or at least, 1, 10, 100, 1,000, or 10,000 grams of the glycoprotein from the cells.

112. The method of claim 86, further comprising combining the glycoprotein having reduced fucosylation with a pharmaceutically acceptable component and, e.g., formulating the glycoprotein having reduced fucosylation into a pharmaceutically acceptable formulation.

113. The method of claim 86, wherein the glycoprotein is analyzed by one or more of HPLC, CE, MALDI-MS and NMR.

114. The method of claim 86, wherein the level of fucosylation at one, two, three, or more preselected amino acid residues is evaluated.

115. The method of claim 86, further comprising, providing a value for a parameter associated with a compound other than GDP-fucose, wherein a parameter for the compound, e.g., the level of the compound, is correlated to the level of GDP-fucose.

116. The method of claim 115, further comprising providing a comparison of the value with a reference value, wherein optionally, a preselected relationship of the value to the reference value, e.g., greater than, equal to, or less than, is indicative of whether the level of GDP fucose is above, at or below the second level.

117. The method of claim 116, further comprising, responsive to the result of the comparison, increasing the level of GDP-fucose, decreasing the level of GDP-fucose or continuing cell culture without intervening to change the level of GDP-fucose.

118. The method of claim 115, wherein the compound other than GDP-fucose is GDP-mannose.

119. The method of claim 115, wherein the compound other than GDP-fucose is GDP-mannose and the parameter is the level of GDP-mannose.

120. The method of claim 86, further comprising, providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose.

121. The method of claim 120, comprising continuing to culture said cells, and repeating the steps of claim 120.

122. A reaction mixture containing one or more of a cell or batch of cultured cells having a manipulation, culture medium, and a glycoprotein having reduced fucosylation produced by the cell.

123. A device for the culture of cells comprising one or more of a cell having a manipulation, culture medium, and a glycoprotein having reduced fucosylation produced by the cell.

124. A method of making, or providing, a glycoprotein having a glycan structure having reduced fucosylation, comprising:
optionally, selecting a glycan structure having reduced fucosylation;
selecting a cell, preferably on the basis that it produces a protein having the primary amino acid sequence of said glycoprotein but which protein lacks said glycan structure having reduced fucosylation;
optionally, selecting a manipulation, e.g., selecting the manipulation on the basis that the manipulation decreases fucosylation and which manipulation thereby promotes the formation of said glycan structure having reduced fucosylation;
providing said manipulation to said cell to provide a cell having or subject to a manipulation that decreases the level of fucosylation and which manipulation thereby promotes the formation of said glycan structure having reduced fucosylation;
culturing said selected cell, e.g., to provide a batch of cultured cells;
optionally, separating the glycoprotein having a glycan structure from at least one component with which the cell or batch of cultured cells was cultured;
optionally, analyzing said glycoprotein to confirm the presence of the glycan structure having reduced fucosylation;
thereby making, or providing, a glycoprotein having a glycan structure having reduced fucosylation, e.g., by inhibiting or promoting the addition of a fucose moiety to a protein or glycoprotein.

125. The method of claim 124, further comprising evaluating a glycan on the surface of said cell or batch of cultured cells in order to determine if the glycoprotein produced by said cell or batch of cultured cells has reduced fucosylation.

126. The method of claim 125, wherein said evaluation comprises evaluating a glycan on the surface of said cell or batch of cultured cells, to determine a property of said glycan, comparing the property to a reference, to thereby determine if said glycan structure is present on the product.

127. The method of claim 124, further comprising evaluating the glycoprotein for a parameter related to fucosylation, e.g., the amount of fucosylation in the glycan complement, the amount or fucosylation on a component of the glycan complement, or the amount of fucosylation on a glycan component, e.g., in a preparation of glycoproteins.

128. The method of claim 124, further comprising evaluating the glycoprotein for a parameter related to fucosylation, e.g., the proportion of a preselected glycan component which bears a fucosyl moiety, e.g., at a selected position on the glycan component, e.g., in a preparation of glycoproteins.

129. The method of claim 124, wherein the level of fucosylation at one, two, three, or more preselected amino acid residues is evaluated.

130. The method of claim 124, wherein the level of fucosylation is reduced by a predetermined level in comparison with a reference.

131. The method of claim 130, wherein the reference is the amount present in a cell or batch of cultured cells, e.g., a CHO cell or batch of cultured cells, lacking the manipulation but otherwise the same or essentially the same as the cell or batch of cultured cells having the manipulation.

132. The method of claim 124, wherein the level of fucosylation is reduced by, as much as, or more than, 10, 20, 30, 40, 50, 60, 70, 80 or 90%, as compared to the reference.

133. The method of claim 124, wherein said manipulation is not a genetic lesion or the presence of an siRNA that reduces the level of an enzyme that promotes formation of GDP-fucose, or the attachment of a fucosyl moiety. For example, the manipulation is not a lesion that decreases the expression of GMD, FX, fucokinase, GFPP, GDP-synthetase, a fucosyltransferase or a GDP-Fucose transporter.

134. The method of claim 124, wherein the cell or batch of cultured cells is wild-type for one or all of GMD, FX, fucokinase, GFPP, GDP-synthetase, a fucosyltransferase or a GDP-Fucose transporter.

135. The method of claim 124, wherein the cell or batch of cultured cells does not include an siRNA that targets GMD, FX, fucokinase, GFPP, GDP-synthetase, a fucosyltransferase or a GDP-Fucose transporter.

136. The method of claim 124, wherein absent the manipulation, the level of fucosylation is substantially the same as the level in a wild-type cell.

137. The method of claim 124, wherein the manipulated cell carries no mutation that substantially lowers GDP-fucose levels.

138. The method of claim 124, wherein the manipulated cell has no siRNA that substantially lowers GDP-fucose levels.

139. The method of claim 124, wherein the cell has a mutation that decreases the level of GDP-fucose, e.g., a mutation in GMD, FX, fucokinase, GFPP, GDP-synthetase, a fucosyltransferase or a GDP-Fucose transporter, and fucose or another substance is present in the culture medium at a level that results in formation of said glycoprotein having a glycan structure having reduced fucosylation.

140. The method of claim 124, wherein the manipulation is the presence of an siRNA that reduces the level of an enzyme that promotes formation of GDP-fucose, or the attachment of a fucosyl moiety, e.g., an siRNA that targets GMD, FX, fucokinase, GFPP, GDP-synthetase, a fucosyltransferase or a GDP-Fucose transporter, and fucose or another substance is present in the culture medium at a level that results in formation of said glycoprotein having a glycan structure having reduced fucosylation.

141. The method of claim 124, wherein the glycoprotein is an antibody.

142. The method of claim 141, wherein the antibody has reduced core fucosylation.

143. The method of claim 142, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

144. The method of claim 124, wherein the cell is a Chinese Hamster Ovary (CHO) cell.

145. The method of claim 144, wherein the glycoprotein is an antibody.

146. The method of claim 145, wherein the antibody has reduced core fucosylation.

147. The method of claim 146, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

148. The method of claim 124, wherein the glycoprotein is selected from Table 1.

149. The method of claim 124, further comprising culturing a plurality of the cells and separating as much as, or at least, 1, 10, 100, 1,000, or 10,000 grams of the glycoprotein from the cells.

150. The method of claim 124, further comprising combining the glycoprotein having reduced fucosylation with a pharmaceutically acceptable component and, e.g., formulating the glycoprotein having reduced fucosylation into a pharmaceutically acceptable formulation.

151. The method of claim 124, wherein the glycoprotein is analyzed by one or more of HPLC, CE, MALDI-MS and NMR.

152. The method of claim 124, where the manipulation is, or is the product of, a selection for reduced levels of GDP-fucose.

153. The method of claim 124, where the manipulation is, or is the product of, a selection for reduced fucosylation of a glycoprotein.

154. The method of claim 124, where the manipulation comprises contact with, or inclusion in or on the cell or batch of cultured cells, of an exogenous inhibitor of an enzyme involved in GDP-fucose biosynthesis, e.g., a specific or non-specific inhibitor.

155. The method of claim 124, wherein the level of fucosylation at one, two, three, or more preselected amino acid residues is evaluated.

156. The method of claim 124, wherein one or more of said cell or said batch of cultured cells, said manipulation, and said glycoprotein, is selected on the basis that it or the combination will provide a glycoprotein having reduced fucosylation.

157. The method of claim 124, further comprising, providing a value for a parameter associated with a compound other than GDP-fucose, wherein a parameter for the compound, e.g., the level of the compound, is correlated to the level of GDP-fucose.

158. The method of claim 157, further comprising providing a comparison of the value with a reference value, wherein optionally, a preselected relationship of the value to the reference value, e.g., greater than, equal to, or less than, is indicative of whether the level of GDP fucose is above, at or below the second level.

159. The method of claim 158, further comprising, responsive to the result of the comparison, increasing the level of GDP-fucose, decreasing the level of GDP-fucose or continuing cell culture without intervening to change the level of GDP-fucose.

160. The method of claim 157, wherein the compound other than GDP-fucose is GDP-mannose.

161. The method of claim 157, wherein the compound other than GDP-fucose is GDP-mannose and the parameter is the level of GDP-mannose.

162. The method of claim 124, further comprising, providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose.

163. The method of claim 162, comprising continuing to culture said cells, and repeating the steps of claim 162.

164. A method of providing a cell that makes a glycoprotein having a glycan structure having reduced fucosylation, comprising:
optionally, selecting a glycan structure having reduced fucosylation;
selecting a cell, preferably on the basis that it produces a protein having the primary amino acid sequence of said glycoprotein but which protein lacks said glycan structure having reduced fucosylation;
optionally, selecting a manipulation, e.g., selecting the manipulation on the basis that the manipulation decreases the level of fucosylation, and which manipulation thereby promotes the formation of said glycan structure having reduced fucosylation;
providing said manipulation to said cell to provide a cell having or subject to a manipulation that decreases fucosylation, and which manipulation thereby promotes the formation of said glycan structure having reduced fucosylation;
optionally producing glycoprotein from said cell and determining if said glycoprotein has said glycan structure having reduced fucosylation, thereby providing a cell that makes a glycoprotein having a glycan structure.

165. The method of claim 164, further comprising evaluating a glycan on the surface of said cell in order to determine if the glycoprotein produced by said cell has reduced fucosylation.

166. The method of claim 165, wherein said evaluation comprises evaluating a glycan on the surface of said cell, to determine a property of said glycan, comparing the property to a reference, to thereby determine if said glycan structure is present on the product.

167. The method of claim 164, further comprising evaluating the glycoprotein for a parameter related to fucosylation, e.g., the amount of fucosylation in the glycan complement, the amount or fucosylation on a component of the glycan complement, or the amount of fucosylation on a glycan component, e.g., in a preparation of glycoproteins.

168. The method of claim 164, further comprising evaluating the glycoprotein for a parameter related to fucosylation, e.g., the proportion of a preselected glycan component which bears a fucosyl moiety, e.g., at a selected position on the glycan component, e.g., in a preparation of glycoproteins.

169. The method of claim 164, wherein the level of fucosylation at one, two, three, or more preselected amino acid residues is evaluated.

170. The method of claim 164, wherein the level of fucosylation is reduced by a predetermined level in comparison with a reference.

171. The method of claim 170, wherein the reference is the amount present in a cell, e.g., a CHO cell, lacking the manipulation but otherwise the same or essentially the same as the cell having the manipulation.

172. The method of claim 164, wherein the level of fucosylation is reduced by, as much as, or more than, 10, 20, 30, 40, 50, 60, 70, 80 or 90%, as compared to the reference.

173. The method of claim 164, wherein said manipulation is not a genetic lesion or the presence of an siRNA that reduces the level of an enzyme that promotes formation of GDP-fucose, or the attachment of a fucosyl moiety. For example, the manipulation is not a lesion that decreases the expression of GMD, FX, fucokinase, GFPP, GDP-synthetase, a fucosyltransferase or a GDP-Fucose transporter.

174. The method of claim 164, wherein the cell is wild-type for one or all of GMD, FX, fucokinase, GFPP, GDP-synthetase, a fucosyltransferase or a GDP-Fucose transporter.

175. The method of claim 164, wherein the cell does not include an siRNA that targets GMD, FX, fucokinase, GFPP, GDP-synthetase, a fucosyltransferase or a GDP-Fucose transporter.

176. The method of claim 164, wherein absent the manipulation, the level of fucosylation is substantially the same as the level in a wild-type cell.

177. The method of claim 164, wherein the manipulated cell carries no mutation that substantially lowers GDP-fucose levels.

178. The method of claim 164, wherein the manipulated cell has no siRNA that substantially lowers GDP-fucose levels.

179. The method of claim 164, wherein the cell has a mutation that decreases the level of GDP-fucose, e.g., a mutation in GMD, FX, fucokinase, GFPP, GDP-synthetase, a fucosyltransferase or a GDP-Fucose transporter, and fucose or another substance is present in the culture medium at a level that results in a level of GDP-fucose that results in formation of said glycan structure having reduced fucosylation.

180. The method of claim 164, wherein the manipulation is the presence of an siRNA that reduces the level of an enzyme that promotes formation of GDP-fucose, or the attachment of a fucosyl moiety, e.g., an siRNA that targets GMD, FX, fucokinase, GFPP, GDP-synthetase, a fucosyltransferase or a GDP-Fucose transporter, and fucose or another substance is present in the culture medium at a level that results in a level of GDP-fucose that results in formation of said glycan structure having reduced fucosylation.

181. The method of claim 164, wherein the glycoprotein is an antibody.

182. The method of claim 181, wherein the antibody has reduced core fucosylation.

183. The method of claim 182, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

184. The method of claim 164, wherein the cell is a Chinese Hamster Ovary (CHO) cell.

185. The method of claim 184, wherein the glycoprotein is an antibody.

186. The method of claim 185, wherein the antibody has reduced core fucosylation.

187. The method of claim 186, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

188. The method of claim 164, wherein the glycoprotein is selected from Table 1.

189. The method of claim 164, further comprising culturing a plurality of the cells and separating as much as, or at least, 1, 10, 100, 1,000, or 10,000 grams of the glycoprotein from the cells.

190. The method of claim 164, further comprising combining the glycoprotein having reduced fucosylation with a pharmaceutically acceptable component and, e.g., formulating the glycoprotein having reduced fucosylation into a pharmaceutically acceptable formulation.

191. The method of claim 164, wherein the glycoprotein is analyzed by one or more of HPLC, CE, MALDI-MS and NMR.

192. The method of claim 164, where the manipulation is, or is the product of, a selection for reduced levels of GDP-fucose.

193. The method of claim 164, where the manipulation is, or is the product of, a selection for reduced fucosylation of a glycoprotein.

194. The method of claim 164, where the manipulation comprises contact with, or inclusion in or on the cell, of an exogenous inhibitor of an enzyme involved in GDP-fucose biosynthesis, e.g., a specific or non-specific inhibitor.

195. The method of claim 164, wherein the level of fucosylation at one, two, three, or more preselected amino acid residues is evaluated.

196. The method of claim 164, wherein one or more of said cell, said manipulation, and said glycoprotein, is selected on the basis that it or the combination will provide a glycoprotein having reduced fucosylation.

197. The method of claim 164, further comprising, providing a value for a parameter associated with a compound other than GDP-fucose, wherein a parameter for the compound, e.g., the level of the compound, is correlated to the level of GDP-fucose.

198. The method of claim 197, further comprising providing a comparison of the value with a reference value, wherein optionally, a preselected relationship of the value to the reference value, e.g., greater than, equal to, or less than, is indicative of whether the level of GDP fucose is above, at or below the second level.

199. The method of claim 198, further comprising, responsive to the result of the comparison, increasing the level of GDP-fucose, decreasing the level of GDP-fucose or continuing cell culture without intervening to change the level of GDP-fucose.

200. The method of claim 199, wherein the compound other than GDP-fucose is GDP-mannose.

201. The method of claim 199, wherein the compound other than GDP-fucose is GDP-mannose and the parameter is the level of GDP-mannose.

202. The method of claim 164, further comprising, providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose.

203. The method of claim 202, comprising continuing to culture said cells, and repeating the steps of claim 202.

204. A method of monitoring a process, e.g., a process of culturing cells, e.g., of a selected type, to produce a product, comprising:
optionally, selecting a glycan structure having reduced fucosylation;
optionally, selecting a cell on the basis of the cell having or subject to a manipulation that decreases the level of fucosylation or GDP-fucose, and which manipulation decreases the level of fucosylation or GDP-fucose;
providing a cell having or subject to a manipulation that decreases the level of fucosylation or GDP-fucose;
culturing said cell, e.g., to provide a batch of cultured cells; and
evaluating (directly or indirectly) the level of GDP-fucose of, or a glycan complement, glycan component or glycan structure produced by, the cell or the batch of cultured cells,
to thereby monitor the process.

205. The method of claim 204, wherein the evaluating step comprises any of:
(a) isolating glycoproteins produced from the cell or the batch of cultured cells and evaluating the glycans containing on the glycoproteins,
(b) isolating a specific glycoprotein composition produced from the cell or the batch of cultured cells and evaluating the glycans from the isolated glycoprotein composition,
(c) obtaining a glycan preparation from a glycoprotein preparation or isolated glycoprotein produced from the cell or the batch of cultured cells and evaluating the glycans in the glycan preparation,
(d) cleaving monosaccharides from glycans present on a glycoprotein produced from the cell or the batch of cultured cells or from glycans on the surface of the cell or the batch of cultured cells, and detecting the cleaved monosaccharides,
(e) providing at least one peptide from a glycoprotein preparation produced from the cell or the batch of cultured cells, and evaluating the glycans on the at least one peptide, and
(f) evaluating glycans from glycans on the cell surface of the cell or the batch of cultured cells.

206. The method of claim 204, wherein the evaluating step comprises isolating glycoproteins produced from the cell or the batch of cultured cells and evaluating the glycans containing on the glycoproteins.

207. The method of claim 204, wherein the evaluating step comprises isolating a specific glycoprotein composition produced from the cell or the batch of cultured cells and evaluating the glycans from the isolated glycoprotein composition.

208. The method of claim 204, wherein the evaluating step comprises obtaining a glycan preparation from a glycoprotein preparation or isolated glycoprotein produced from the cell or the batch of cultured cells and evaluating the glycans in the glycan preparation.

209. The method of claim 204, wherein the evaluating step comprises cleaving monosaccharides from glycans present on a glycoprotein produced from the cell or the batch of cultured cells or from glycans on the surface of the cell or the batch of cultured cells, and detecting the cleaved monosaccharides.

210. The method of claim 204, wherein the evaluating step comprises providing at least one peptide from a glycoprotein preparation produced from the cell or the batch of cultured cells, and evaluating the glycans on the at least one peptide.

211. The method of claim 204, wherein the evaluating step comprises evaluating glycans from glycans on the cell surface of the cell or the batch of cultured cells.

212. The method of claim 204, further comprising:
if said observed value does not meet said reference, discarding said cell, continuing culture of said cell, or altering a culture condition and further culturing said cell.

213. The method of claim 204, further comprising, if said process value meets said reference value, continuing culture of said cell or said batch of cultured cells, altering a culture condition and further culturing said cell or said batch of cultured cells, or discarding said cell or said batch of cultured cells.

214. The method of claim 204, further comprising continuing culture of the cell or the batch of cultured cells.

215. The method of claim 204, further comprising altering a culture condition and further culturing said cell or said batch of cultured cells and optionally repeating the evaluation.

216. The method of claim 204, wherein said manipulation is not a genetic lesion or the presence of an siRNA that reduces the level of an enzyme that promotes formation of GDP-fucose, or the attachment of a fucosyl moiety. For example, the manipulation is not a lesion that decreases the expression of GMD, FX, fucokinase, GFPP, GDP-synthetase, a fucosyltransferase or a GDP-Fucose transporter.

217. The method of claim 204, wherein the cell or batch of cultured cells is wild-type for one or all of GMD, FX, fucokinase, GFPP, GDP-synthetase, a fucosyltransferase or a GDP-Fucose transporter.

218. The method of claim 204, wherein the cell or batch of cultured cells does not include an siRNA that targets GMD, FX, fucokinase, GFPP, GDP-synthetase, a fucosyltransferase or a GDP-Fucose transporter.

219. The method of claim 204, wherein absent the manipulation, the level of fucosylation is substantially the same as the level in a wild-type cell.

220. The method of claim 204, wherein the manipulated cell carries no mutation that substantially lowers GDP-fucose levels.

221. The method of claim 204, wherein the manipulated cell has no siRNA that substantially lowers GDP-fucose levels.

222. The method of claim 204, wherein the cell has a mutation that decreases the level of GDP-fucose, e.g., a mutation in GMD, FX, fucokinase, GFPP, GDP-synthetase, a fucosyltransferase or a GDP-Fucose transporter, and fucose or another substance is present in the culture medium at a level that results in formation of said glycan structure having reduced fucosylation.

223. The method of claim 204, wherein the manipulation is the presence of an siRNA that reduces the level of an enzyme that promotes formation of GDP-fucose, or the attachment of a fucosyl moiety, e.g., an siRNA that targets GMD, FX, fucokinase, GFPP, GDP-synthetase, a fucosyltransferase or a GDP-Fucose transporter, and fucose or another substance is present in the culture medium at a level that results in formation of said glycan structure having reduced fucosylation.

224. The method of claim 205, wherein the glycoprotein is an antibody.

225. The method of claim 224, wherein the antibody has reduced core fucosylation.

226. The method of claim 225, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

227. The method of claim 205, wherein the cell is a Chinese Hamster Ovary (CHO) cell.

228. The method of claim 227, wherein the glycoprotein is an antibody.

229. The method of claim 228, wherein the antibody has reduced core fucosylation.

230. The method of claim 229, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

231. The method of claim 205, wherein the glycoprotein is selected from Table 1.

232. The method of claim 205, wherein the glycoprotein is analyzed by one or more of HPLC, CE, MALDI-MS and NMR.

233. The method of claim 204, where the manipulation is, or is the product of, a selection for reduced levels of GDP-fucose.

234. The method of claim 204, where the manipulation is, or is the product of, a selection for reduced fucosylation of a glycoprotein.

235. The method of claim 204, where the manipulation comprises contact with, or inclusion in or on the cell or batch of cultured cells, of an exogenous inhibitor of an enzyme involved in GDP-fucose biosynthesis, e.g., a specific or non-specific inhibitor.

236. The method of claim 205, wherein the level of fucosylation at one, two, three, or more preselected amino acid residues is evaluated.

237. The method of claim 204, further comprising, providing a value for a parameter associated with a compound other than GDP-fucose, wherein a parameter for the compound, e.g., the level of the compound, is correlated to the level of GDP-fucose.

238. The method of claim 237, further comprising providing a comparison of the value with a reference value, wherein optionally, a preselected relationship of the value to the reference value, e.g., greater than, equal to, or less than, is indicative of whether the level of GDP fucose is above, at or below the second level.

239. The method of claim 238, further comprising, responsive to the result of the comparison, increasing the level of GDP-fucose, decreasing the level of GDP-fucose or continuing cell culture without intervening to change the level of GDP-fucose.

240. The method of claim 237, wherein the compound other than GDP-fucose is GDP-mannose.

241. The method of claim 237, wherein the compound other than GDP-fucose is GDP-mannose and the parameter is the level of GDP-mannose.

242. The method of claim 204, further comprising, providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose.

243. The method of claim 242, comprising continuing to culture said cells, and repeating the steps of claim 242.

244. A method of controlling a process for making a glycoprotein having a glycan structure with reduced fucosylation, comprising:
(1) providing a glycoprotein made by the process of
optionally, selecting a glycan structure having reduced fucosylation;
optionally, selecting a cell on the basis of the cell having or subject to a manipulation that decreases the level of fucosylation or GDP-fucose, and which manipulation decreases the level of fucosylation or GDP-fucose;
providing a cell having or subject to a manipulation that decreases the level of decreases the level of fucosylation or GDP-fucose; and
culturing the cell to provide a glycoprotein and, e.g., form a batch of cultured cells;
(2) evaluating (directly or indirectly) the level of GDP-fucose of the cell, or the glycan structure of the glycoprotein,
(3) responsive to said evaluation, selecting a production parameter, e.g., a culture
condition, e.g., a level of a nutrient or other component in the culture medium,
to thereby control the process for making a glycoprotein having a glycan structure.

245. The method of claim 244, comprising continuing culture of the cell or batch of cultured cells under conditions that differ from those used prior to the evaluation.

246. The method of claim 244, comprising continuing culture of the cell or batch of cultured cells under the same conditions used prior to the evaluation.

247. The method of claim 244, wherein said evaluation step comprises comparing the structure of said glycan structure having reduced fucosylation present on a glycoprotein from said cultured cell or batch of cultured cells to a reference, and determining if said glycan structure having reduced fucosylation present on a glycoprotein from said cultured cell or batch of cultured cells differs from the corresponding glycan structure formed by a cell or batch of cultured cells that lacks the manipulation.

248. The method of claim 244, further comprising evaluating the glycoprotein for a parameter related to fucosylation, e.g., the amount of fucosylation in the glycan complement, the amount or fucosylation on a component of the glycan complement, or the amount of fucosylation on a glycan component, e.g., in a preparation of glycoproteins.

249. The method of claim 244, further comprising evaluating the glycoprotein for a parameter related to fucosylation, e.g., the proportion of a preselected glycan component which bears a fucosyl moiety, e.g., at a selected position on the glycan component, e.g., in a preparation of glycoproteins.

250. The method of claim 244, wherein the level of fucosylation at one, two, three, or more preselected amino acid residues is evaluated.

251. The method of claim 244, wherein the level of fucosylation is reduced by a predetermined level in comparison with a reference.

252. The method of claim 251, wherein the reference is the amount present in a cell or batch of cultured cells, e.g., a CHO cell or batch of cultured cells, lacking the manipulation but otherwise the same or essentially the same as the cell or batch of cultured cells having the manipulation.

253. The method of claim 244, wherein the level of fucosylation is reduced by, as much as, or more than, 10, 20, 30, 40, 50, 60, 70, 80 or 90%, as compared to the reference.

254. The method of claim 244, wherein said manipulation is not a genetic lesion or the presence of an siRNA that reduces the level of an enzyme that promotes formation of GDP-fucose, or the attachment of a fucosyl moiety. For example, the manipulation is not a lesion that decreases the expression of GMD, FX, fucokinase, GFPP, GDP-synthetase, a fucosyltransferase or a GDP-Fucose transporter.

255. The method of claim 244, wherein the cell or batch of cultured cells is wild-type for one or all of GMD, FX, fucokinase, GFPP, GDP-synthetase, a fucosyltransferase or a GDP-Fucose transporter.

256. The method of claim 244, wherein the cell or batch of cultured cells does not include an siRNA that targets GMD, FX, fucokinase, GFPP, GDP-synthetase, a fucosyltransferase or a GDP-Fucose transporter.

257. The method of claim 244, wherein absent the manipulation, the level of fucosylation is substantially the same as the level in a wild-type cell.

258. The method of claim 244, wherein the manipulated cell carries no mutation that substantially lowers GDP-fucose levels.

259. The method of claim 244, wherein the manipulated cell has no siRNA that substantially lowers GDP-fucose levels.

260. The method of claim 244, wherein the cell has a mutation that decreases the level of GDP-fucose, e.g., a mutation in GMD, FX, fucokinase, GFPP, GDP-synthetase, a fucosyltransferase or a GDP-Fucose transporter, and fucose or another substance is present in the culture medium at a level that results in formation of said glycan structure having reduced fucosylation.

261. The method of claim 244, wherein the manipulation is the presence of an siRNA that reduces the level of an enzyme that promotes formation of GDP-fucose, or the attachment of a fucosyl moiety, e.g., an siRNA that targets GMD, FX, fucokinase, GFPP, GDP-synthetase, a fucosyltransferase or a GDP-Fucose transporter, and fucose or another substance is present in the culture medium at a level that results in formation of said glycan structure having reduced fucosylation.

262. The method of claim 244, wherein the glycoprotein is an antibody.

263. The method of claim 262, wherein the antibody has reduced core fucosylation.

264. The method of claim 263, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

265. The method of claim 244, wherein the cell is a Chinese Hamster Ovary (CHO) cell.

266. The method of claim 265, wherein the glycoprotein is an antibody.

267. The method of claim 266, wherein the antibody has reduced core fucosylation.

268. The method of claim 267, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

269. The method of claim 244, wherein the glycoprotein is selected from Table 1.

270. The method of claim 244, further comprising culturing a plurality of the cells and separating as much as, or at least, 1, 10, 100, 1,000, or 10,000 grams of the glycoprotein from the cells.

271. The method of claim 244, further comprising combining the glycoprotein having reduced fucosylation with a pharmaceutically acceptable component and, e.g., formulating the glycoprotein having reduced fucosylation into a pharmaceutically acceptable formulation.

272. The method of claim 244, wherein the glycoprotein is analyzed by one or more of HPLC, CE, MALDI-MS and NMR.

273. The method of claim 244, where the manipulation is, or is the product of, a selection for reduced levels of GDP-fucose.

274. The method of claim 244, where the manipulation is, or is the product of, a selection for reduced fucosylation of a glycoprotein.

275. The method of claim 244, where the manipulation comprises contact with, or inclusion in or on the cell or batch of cultured cells, of an exogenous inhibitor of an enzyme involved in GDP-fucose biosynthesis, e.g., a specific or non-specific inhibitor.

276. The method of claim 244, wherein the level of fucosylation at one, two, three, or more preselected amino acid residues is evaluated.

277. The method of claim 244, wherein one or more of said cell or said batch of cultured cells, said manipulation, and said glycoprotein, is selected on the basis that it or the combination will provide a glycoprotein having reduced fucosylation.

278. The method of claim 244, further comprising, providing a value for a parameter associated with a compound other than GDP-fucose, wherein a parameter for the compound, e.g., the level of the compound, is correlated to the level of GDP-fucose.

279. The method of claim 278, further comprising providing a comparison of the value with a reference value, wherein optionally, a preselected relationship of the value to the reference value, e.g., greater than, equal to, or less than, is indicative of whether the level of GDP fucose is above, at or below the second level.

280. The method of claim 279, further comprising, responsive to the result of the comparison, increasing the level of GDP-fucose, decreasing the level of GDP-fucose or continuing cell culture without intervening to change the level of GDP-fucose.

281. The method of claim 278, wherein the compound other than GDP-fucose is GDP-mannose.

282. The method of claim 278, wherein the compound other than GDP-fucose is GDP-mannose and the parameter is the level of GDP-mannose.

283. The method of claim 244, further comprising, providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose.

284. The method of claim 283, comprising continuing to culture said cells, and repeating the steps of claim 283.

285. A method of controlling a process for making a glycoprotein having a glycan structure with reduced fucosylation, comprising:
(1) providing a glycoprotein made by the process of:
optionally, selecting a glycan structure having reduced fucosylation;
optionally, selecting a cell on the basis of the cell having or subject to a manipulation that decreases the level of fucosylation or GDP-fucose, and which manipulation decreases the level of fucosylation or GDP-fucose;
providing a cell having or subject to a manipulation that decreases the level of decreases the level of fucosylation or GDP-fucose; and
culturing the cell to provide a glycoprotein and, e.g., form a batch of cultured cells;
(2) providing a value for a parameter associated with a compound other than GDP-fucose, wherein a parameter for the compound, e.g., the level of the compound, is correlated to the level of GDP-fucose,
(3) providing a comparison of the value with a reference value, wherein optionally, a preselected relationship of the value to the reference value, e.g., greater than, equal to, or less than, is indicative of whether the level of GDP fucose is above, at or below a preselected level
(4) responsive to said comparison, selecting a production parameter, e.g., a culture condition, e.g., a level of a nutrient or other component in the culture medium, to thereby control the process for making a glycoprotein having a glycan structure.

286. The method of claim 285, further comprising, responsive to the result of the comparison, increasing the level of GDP-fucose, decreasing the level of GDP-fucose or continuing cell culture without intervening to change the level of GDP-fucose.

287. The method of claim 285, wherein the compound other than GDP-fucose is GDP-mannose.

288. The method of claim 285, wherein the compound other than GDP-fucose is GDP-mannose and the parameter is the level of GDP-mannose.

289. The method of claim 285, further comprising, providing a value for the level of GDP-mannose, providing a comparison of the value with a reference value, and responsive to the result of the comparison, increasing the level of GDP-fucose, decreasing the level of GDP-fucose or continuing cell culture at without intervening to change the level of GDP-fucose.

290. The method of claim 285, comprising continuing to culture said cells, and repeating the steps of claim 285.

291. The method of claim 285, wherein said manipulation is not a genetic lesion or the presence of an siRNA that reduces the level of an enzyme that promotes formation of GDP-fucose, or the attachment of a fucosyl moiety. For example, the manipulation is not a lesion that decreases the expression of GMD, FX, fucokinase, GFPP, GDP-synthetase, a fucosyltransferase or a GDP-Fucose transporter.

292. The method of claim 285, wherein the cell or batch of cultured cells is wild-type for one or all of GMD, FX, fucokinase, GFPP, GDP-synthetase, a fucosyltransferase or a GDP-Fucose transporter.

293. The method of claim 285, wherein the cell or batch of cultured cells does not include an siRNA that targets GMD, FX, fucokinase, GFPP, GDP-synthetase, a fucosyltransferase or a GDP-Fucose transporter.

294. The method of claim 285, wherein absent the manipulation, the level of fucosylation is substantially the same as the level in a wild-type cell.

295. The method of claim 285, wherein the manipulated cell carries no mutation that substantially lowers GDP-fucose levels.

296. The method of claim 285, wherein the manipulated cell has no siRNA that substantially lowers GDP-fucose levels.

297. The method of claim 285, wherein the cell has a mutation that decreases the level of GDP-fucose, e.g., a mutation in GMD, FX, fucokinase, GFPP, GDP-synthetase, a fucosyltransferase or a GDP-Fucose transporter, and fucose or another substance is present in the culture medium at a level that results in formation of said glycan structure having reduced fucosylation.

298. The method of claim 285, wherein the manipulation is the presence of an siRNA that reduces the level of an enzyme that promotes formation of GDP-fucose, or the attachment of a fucosyl moiety, e.g., an siRNA that targets GMD, FX, fucokinase, GFPP, GDP-synthetase, a fucosyltransferase or a GDP-Fucose transporter, and fucose or another substance is present in the culture medium at a level that results in formation of said glycan structure having reduced fucosylation.

299. The method of claim 285, wherein the glycoprotein is an antibody.

300. The method of claim 299, wherein the antibody has reduced core fucosylation.

301. The method of claim 300, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

302. The method of claim 285, wherein the cell is a Chinese Hamster Ovary (CHO) cell.

303. The method of claim 302, wherein the glycoprotein is an antibody.

304. The method of claim 303, wherein the antibody has reduced core fucosylation.

305. The method of claim 304, wherein the antibody is selected from the group consisting of Rituximab, Trastuzamab, Bevacizumab, Tositumomab, Alemtuzumab, Arcitumomab, Cetuximab, Trastuzumab, Adalimumab, Ranibizumab, Gemtuzumab [ozogamicin], Fanolesomab, Efalizumab, Infliximab, Abciximab, Rituximab, Basiliximab, Eculizumab, Palivizumab, Natalizumab, Omalizumab, Daclizumab, and Ibritumomab.

306. The method of claim 285, wherein the glycoprotein is selected from Table 1.

307. The method of claim 285, further comprising culturing a plurality of the cells and separating as much as, or at least, 1, 10, 100, 1,000, or 10,000 grams of the glycoprotein from the cells.

308. The method of claim 285, further comprising combining the glycoprotein having reduced fucosylation with a pharmaceutically acceptable component and, e.g., formulating the glycoprotein having reduced fucosylation into a pharmaceutically acceptable formulation.

309. The method of claim 285, wherein the glycoprotein is analyzed by one or more of HPLC, CE, MALDI-MS and NMR.

310. The method of claim 285, where the manipulation is, or is the product of, a selection for reduced levels of GDP-fucose.

311. The method of claim 285, where the manipulation is, or is the product of, a selection for reduced fucosylation of a glycoprotein.

312. The method of claim 285, where the manipulation comprises contact with, or inclusion in or on the cell or batch of cultured cells, of an exogenous inhibitor of an enzyme involved in GDP-fucose biosynthesis, e.g., a specific or non-specific inhibitor.

313. A method of making a glycoprotein having reduced fucosylation, comprising:
(e) providing, acknowledging, selecting, accepting, or memorializing a defined, desired or preselected glycan structure having reduced fucosylation for the glycoprotein,
(f) optionally providing a cell manipulated to decrease the level of fucosylation or fucose-GDP,
(g) culturing a cell manipulated to decrease the level of fucosylation or fucose-GDP, e.g., to form a batch of cultured cells, and
(h) isolating from the cell or batch of cultured cells a glycoprotein having the desired glycan structure,
thereby making a glycoprotein.

314. A method of making a glycoprotein, comprising:
providing, acknowledging, selecting, accepting, or memorializing a defined, desired or preselected glycan structure having reduced fucosylation for the glycoprotein, chosen, e.g., from Table 1;
optionally, providing, acknowledging, selecting, accepting, or memorializing a manipulation described herein;
culturing a cell having the manipulation, e.g., to form a batch of cultured cells;
isolating from the cell or batch of cultured cells a glycoprotein having the desired glycan structure,
thereby making a glycoprotein.

315. A method of formulating a pharmaceutical composition comprising:
contacting a glycoprotein made by a method described herein with a pharmaceutically acceptable substance, e.g., an excipient or diluent.

316. A pharmaceutical preparation of a glycoprotein described herein or made by a method described herein, wherein the glycoprotein is selected from Table 1.

317. The method of claim 124, wherein said manipulation provides a first preselected level of GDP-fucose selected from:
i.a) approximately equal to or less than 80%, 70% or 60% of a reference level, e.g., the level in said cell or batch of cultured cells, e.g., a cell or batch of cultured cells which is otherwise similar, without the manipulation;
ii.a) approximately equal to, or less than, the point of maximum curvature above the inflection point (e.g., the inflection point in the second phase) on a graph of the amount of fucosylation vs. decrease in GDP-fucose;
ii.1.a) approximately equal to, or less than, the lowest level that results in a normal (e.g., that seen in an un-manipuated cell) level of fucosylation;
iii.a) approximately equal to or less than the point of maximum curvature below the inflection point on a graph of the amount of fucosylation vs. decrease in GDP-fucose;
iii.1.a) approximately equal to, or less than, the highest level that results in no further reduction in fucosylation;
iv.a) approximately equal to or less than point A on the curve in FIG. 1, or less than or equal to an analogous point on a plot of the amount of fucosylation (%) vs. the amount of GDP fucose as a % of control;
v.a) approximately equal to or less than that corresponding to an amount between points A and B on the curve in FIG. 1, or less than or equal to an analogous point on a plot of the amount of fucosylation (%) vs. the amount of GDP fucose as a % of control; or
vi.a) approximately equal to or less than point B on the curve in FIG. 1, or less than or equal to an analogous point on a plot of the amount of fucosylation (%) vs. the amount of GDP fucose as a % of control.

318. The method of claim 124, wherein said manipulation provides a second preselected level of GDP-fucose selected from:
i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;
ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;
iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level;
iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;
v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level; or
vi.b) approximately equal to or greater than point C on the curve in FIG. 1, or greater than or equal to an analogous point on a plot of the amount of fucosylation (%) vs. the amount of GDP fucose as a % of control.

319. The method of claim 164, wherein said manipulation provides a first preselected level of GDP-fucose selected from:
i.a) approximately equal to or less than 80%, 70% or 60% of a reference level, e.g., the level in said cell or batch of cultured cells, e.g., a cell or batch of cultured cells which is otherwise similar, without the manipulation;
ii.a) approximately equal to, or less than, the point of maximum curvature above the inflection point (e.g., the inflection point in the second phase) on a graph of the amount of fucosylation vs. decrease in GDP-fucose;
ii.1.a) approximately equal to, or less than, the lowest level that results in a normal (e.g., that seen in an un-manipuated cell) level of fucosylation;
iii.a) approximately equal to or less than the point of maximum curvature below the inflection point on a graph of the amount of fucosylation vs. decrease in GDP-fucose;
iii.1.a) approximately equal to, or less than, the highest level that results in no further reduction in fucosylation;
iv.a) approximately equal to or less than point A on the curve in FIG. 1, or less than or equal to an analogous point on a plot of the amount of fucosylation (%) vs. the amount of GDP fucose as a % of control;
v.a) approximately equal to or less than that corresponding to an amount between points A and B on the curve in FIG. 1, or less than or equal to an analogous point on a plot of the amount of fucosylation (%) vs. the amount of GDP fucose as a % of control; or
vi.a) approximately equal to or less than point B on the curve in FIG. 1, or less than or equal to an analogous point on a plot of the amount of fucosylation (%) vs. the amount of GDP fucose as a % of control.

320. The method of claim 164, wherein said manipulation provides a second preselected level of GDP-fucose selected from:
i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;
ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;
iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level;
iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;
v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level; or
vi.b) approximately equal to or greater than point C on the curve in FIG. 1, or greater than or equal to an analogous point on a plot of the amount of fucosylation (%) vs. the amount of GDP fucose as a % of control.

321. The method of claim 214, wherein said evaluation comprises determining if a first preselected level of GDP-fucose is:
i.a) approximately equal to or less than 80%, 70% or 60% of a reference level, e.g., the level in said cell or batch of cultured cells, e.g., a cell or batch of cultured cells which is otherwise similar, without the manipulation;
ii.a) approximately equal to, or less than, the point of maximum curvature above the inflection point (e.g., the inflection point in the second phase) on a graph of the amount of fucosylation vs. decrease in GDP-fucose;
ii.1.a) approximately equal to, or less than, the lowest level that results in a normal (e.g., that seen in an un-manipuated cell) level of fucosylation;
iii.a) approximately equal to or less than the point of maximum curvature below the inflection point on a graph of the amount of fucosylation vs. decrease in GDP-fucose;
iii.1.a) approximately equal to, or less than, the highest level that results in no further reduction in fucosylation;
iv.a) approximately equal to or less than point A on the curve in FIG. 1, or less than or equal to an analogous point on a plot of the amount of fucosylation (%) vs. the amount of GDP fucose as a % of control;
v.a) approximately equal to or less than that corresponding to an amount between points A and B on the curve in FIG. 1, or less than or equal to an analogous point on a plot of the amount of fucosylation (%) vs. the amount of GDP fucose as a % of control; or
vi.a) approximately equal to or less than point B on the curve in FIG. 1, or less than or equal to an analogous point on a plot of the amount of fucosylation (%) vs. the amount of GDP fucose as a % of control.

322. The method of claim 214, wherein said evaluation comprised determining if a second preselected level of GDP-fucose is:
i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;
ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;
iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level;
iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;
v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level; or
vi.b) approximately equal to or greater than point C on the curve in FIG. 1, or greater than or equal to an analogous point on a plot of the amount of fucosylation (%) vs. the amount of GDP fucose as a % of control.

323. The method of claim 244, wherein said evaluation comprises determining if a first preselected level of GDP-fucose is:
i.a) approximately equal to or less than 80%, 70% or 60% of a reference level, e.g., the level in said cell or batch of cultured cells, e.g., a cell or batch of cultured cells which is otherwise similar, without the manipulation;
ii.a) approximately equal to, or less than, the point of maximum curvature above the inflection point (e.g., the inflection point in the second phase) on a graph of the amount of fucosylation vs. decrease in GDP-fucose;
ii.1.a) approximately equal to, or less than, the lowest level that results in a normal (e.g., that seen in an un-manipuated cell) level of fucosylation;
iii.a) approximately equal to or less than the point of maximum curvature below the inflection point on a graph of the amount of fucosylation vs. decrease in GDP-fucose;
iii.1.a) approximately equal to, or less than, the highest level that results in no further reduction in fucosylation;
iv.a) approximately equal to or less than point A on the curve in FIG. 1, or less than or equal to an analogous point on a plot of the amount of fucosylation (%) vs. the amount of GDP fucose as a % of control;
v.a) approximately equal to or less than that corresponding to an amount between points A and B on the curve in FIG. 1, or less than or equal to an analogous point on a plot of the amount of fucosylation (%) vs. the amount of GDP fucose as a % of control; or
vi.a) approximately equal to or less than point B on the curve in FIG. 1, or less than or equal to an analogous point on a plot of the amount of fucosylation (%) vs. the amount of GDP fucose as a % of control.

324. The method of claim 244, wherein said evaluation comprised determining if a second preselected level of GDP-fucose is:
i.b) approximately equal to, or greater than, 10%, 15%, 20%, 25%, 30%, 35% or 40% of a reference level, e.g., the level in said cell or batch of cultured cells, e.g., a cell or batch of cultured cells which is otherwise similar, without the manipulation;
ii.b) an amount that provides an unacceptable level of fucose deprivation, e.g., an amount that results in decrease of GDP-mannose, e.g., a decrease in GDP-mannose that is equal to, greater than, 10%, 20%, 30%, 40% or 50% than a reference levee, e.g., the level of GDP-mannose in a cell or batch of cultured cells, e.g., a cell or batch of cultured cells which is otherwise similar, without the manipulation;
iii.b) an amount that provides an unacceptable level of fucose deprivation, e.g. an amount that results in a level of high mannose structures that are less than or equal to 10%, 20%, 30%, 40%, 50%, 60%, 70%, 80%, or 90% of a reference level;
iv.b) an amount that provides an unacceptable level of fucose deprivation, e.g., an amount that results in accumulation of GDP-mannose, e.g. an increase in GDP-mannose that is equal to or greater than 2×, 3×, 4×, 5×, 6×, 7×, 8×, 9×, or 10× of a reference level, e.g. the level of GDP-mannose in a cell or batch of cultured cells, e.g., a cell or batch of cultured cells which is otherwise similar, without the manipulation;
v.b) an amount that provides an unacceptable level of fucose deprivation, e.g., an amount that results in accumulation of high mannose structures that are more than or equal to 2×, 3×, 4×, 5×, 6×, 7×, 8×, 9×, or 10× of a reference level; or
vi.b) approximately equal to or greater than point C on the curve in FIG. 1, or greater than or equal to an analogous point on a plot of the amount of fucosylation (%) vs. the amount of GDP fucose as a % of control.


SEQUENCE LISTING (42 sequences)

SEQ ID NO: 1 - protein, 372 aa, Homo sapiens
MAHAPARCPSARGSGDGEMGKPRNVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAVKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFLHIGKTIVWEGKNENEVGRCKETGKVHVTVDLKYYRPTEVDFLQGDCTKAKQKLNWKPRVAFDELVREMVHADVELMRTNPNA

SEQ ID NO: 2 - DNA, 1119 bp, Homo sapiens
atggcacacgcaccggcacgctgccccagcgcccggggctccggggacggcgagatgggcaagcccaggaacgtggcgctcatcaccggtatcacaggccaggatggttcctacctggctgagttcctgctggagaaaggctatgaggtccatggaattgtacggcggtccagttcatttaatacgggtcgaattgagcatctgtataagaatccccaggctcacattgaaggaaacatgaagttgcactatggcgatctcactgacagtacctgccttgtgaagatcattaatgaagtaaagcccacagagatctacaaccttggagcccagagccacgtcaaaatttcctttgacctcgctgagtacactgcggacgttgacggagttggcactctacgacttctagatgcagttaagacttgtggccttatcaactctgtgaagttctaccaagcctcaacaagtgaactttatgggaaagtgcaggaaataccccagaaggagaccacccctttctatccccggtcaccctatggggcagcaaaactctatgcctattggattgtggtgaacttccgtgaggcgtataatctctttgcagtgaacggcattctcttcaatcatgagagtcccagaagaggagctaatttcgttactcgaaaaattagccggtcagtagctaagatttaccttggacaactggaatgtttcagtttgggaaatctggatgccaaacgagattggggccatgccaaggactatgtggaggctatgtggttgatgttgcagaatgatgagccggaggacttcgttatagctactggggaggtccatagtgtccgggaatttgtcgagaaatcattcttgcacattggaaaaaccattgtgtgggaaggaaagaatgaaaatgaagtgggcagatgtaaagagaccggcaaagttcacgtgactgtggatctcaagtactaccggccaactgaagtggactttctgcagggcgactgcaccaaagcgaaacagaagctgaactggaagccccgggtcgctttcgatgagctggtgagggagatggtgcacgccgacgtggagctcatgaggacaaaccccaatgcctga

SEQ ID NO: 3 - protein, 372 aa, Mus musculus
MAQAPAKCPSYPGSGDGEMGKLRKVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAIKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFMHIGKTIVWEGKNENEVGRCKETGKVHVTVDLKYYRPTEVDFLQGDCSKAQQKLNWKPRVAFDELVREMVQADVELMRTNPNA

SEQ ID NO: 4 - DNA, 1119 bp, Mus musculus
atggctcaagctcccgctaagtgcccgagctacccgggctccggggatggcgagatgggcaagctcaggaaggtggctctcatcactggcatcaccggacaggatggttcgtacttggcagaattcctgttggagaaagggtacgaggtccatggaatagtacggcgatctagttcatttaatacaggtcgaattgaacatttatataagaatcctcaggctcatattgaaggaaacatgaagttgcactatggtgacctcactgacagcacctgcctagtgaaaatcatcaatgaagtcaagcctacagagatctataatcttggagcccagagccatgtcaagatctcctttgacttagctgagtacaccgcagatgttgatggcgttggcaccttgcggcttctggatgcaattaaaacttgtggccttataaattctgtgaagttctaccaggcctcaacaagtgaactttatggaaaagtgcaggaaataccccagaaggagaccacacctttctatccgaggtcaccctatggagcagccaaactctatgcctattggattgtggtgaatttccgtgaagcttataatctctttgcagtgaatggaattctcttcaatcatgagagtcccagaagaggagctaattttgttactcgaaaaattagccggtcagtagctaagatttaccttggacaactggaatgtttcagcttgggaaatctggatgccaaacgagactggggccatgccaaggactatgtagaggctatgtggctcatgttgcagaatgatgagccagaggactttgtcatagctactggggaagttcacagtgtccgtgaatttgttgaaaagtcattcatgcacatcggaaaaaccattgtgtgggaaggaaagaatgaaaatgaagtgggcagatgtaaagagaccggcaaagttcacgtgactgtggatctgaaatactaccgaccgactgaagtggactttctgcagggagactgctccaaggctcagcagaagctaaactggaagccccgcgttgcctttgacgagctggtgagggagatggtgcaggccgacgtggagctcatgaggaccaaccccaacgcttga

SEQ ID NO: 5 - protein, 372 aa, Rattus norvegicus
MAHAPASCRRYPGSGDGEMGKLRKVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAIKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFMHIGKTIVWEGKNENEVGRCKETGKIHVTVDLKYYRPTEVDFLQGDCSKAQQKLNWKPRVAFDELVREMVQADVELMRTNPNA

SEQ ID NO: 6 - DNA, 1119 bp, Rattus norvegicus
atggcccacgctcccgctagctgccggagatacccgggctccggggatggcgagatgggcaagctcaggaaggtagctctcatcaccggcatcactggccaggatggttcatacttggcagaattcctgctggagaaaggatacgaggtccatggaatagtacggcgatctagttcatttaatacaggtcgaattgaacatttatataagaatcctcaggctcatattgaaggaaacatgaagttgcactatggcgacctgactgacagcacctgcctggtgaaaatcatcaatgaagtgaagcctacagagatctacaatcttggcgctcagagccatgtcaagatctcctttgacttagctgaatacaccgcagacgttgatggagttggcaccttgcggcttctggatgcaattaaaacttgcggccttataaattctgtgaagttctaccaggcctcgacaagtgaactttatggaaaagttcaggaaataccccagaaagagaccacacctttctatccgaggtcaccctatggagccgccaagctctatgcctattggattgtggtgaatttccgtgaagcttataatctctttgcagtgaatggcattctcttcaatcacgagagccccagaagaggagctaattttgttactcgaaaaattagccggtcagtagctaagatttaccttggacaactggaatgtttcagtttgggaaatctggatgccaaacgagactggggccatgccaaggactatgtagaggctatgtggctgatgttgcaaaatgatgagccggaggactttgtcatagctactggggaagttcacagtgtccgtgaatttgttgaaaaatcattcatgcacattggaaaaaccattgtgtgggaaggaaagaatgaaaatgaagtaggcagatgtaaggagaccggcaaaattcacgtgactgtggatctgaaatactaccgaccgactgaagtggactttctacagggagactgctccaaggctcagcagaaactgaactggaaaccccgcgttgccttcgatgagctggtgagagagatggtgcaggccgacgtggagctcatgaggaccaaccccaacgcttga

SEQ ID NO: 7 - protein, 372 aa, Cricetulus griseus; identical to SEQ ID NO: 1

SEQ ID NO: 8 - DNA, 1606 bp, Cricetulus griseus
agactgtggcggccgctgcagctccgtgaggcgactggcgcgcgcacccacgtctctgtcggcccgctgccggttccacggttccactcctccttccactcggctgcacgctcacccgcccgcggcgacatggctcacgctcccgctagctgcccgagctccaggaactctggggacggcgataagggcaagcccaggaaggtggcgctcatcacgggcatcaccggccaggatggctcatacttggcagaattcctgctggagaaaggatacgaggttcatggaattgtacggcgatccagttcatttaatacaggtcgaattgaacatttatataagaatccacaggctcatattgaaggaaacatgaagttgcactatggtgacctcaccgacagcacctgcctagtaaaaatcatcaatgaagtcaaacctacagagatctacaatcttggtgcccagagccatgtcaagatttcctttgacttagcagagtacactgcagatgttgatggagttggcaccttgcggcttctggatgcaattaagacttgtggccttataaattctgtgaagttctaccaggcctcaactagtgaactgtatggaaaagtgcaagaaataccccagaaagagaccacccctttctatccaaggtcgccctatggagcagccaaactttatgcctattggattgtagtgaactttcgagaggcttataatctctttgcggtgaacggcattctcttcaatcatgagagtcctagaagaggagctaattttgttactcgaaaaattagccggtcagtagctaagatttaccttggacaactggaatgtttcagtttgggaaatctggacgccaaacgagactggggccatgccaaggactatgtcgaggctatgtggctgatgttacaaaatgatgaaccagaggactttgtcatagctactggggaagttcatagtgtccgtgaatttgttgagaaatcattcatgcacattggaaagaccattgtgtgggaaggaaagaatgaaaatgaagtgggcagatgtaaagagaccggcaaaattcatgtgactgtggatctgaaatactaccgaccaactgaagtggacttcctgcagggagactgctccaaggcgcagcagaaactgaactggaagccccgcgttgcctttgacgagctggtgagggagatggtgcaagccgatgtggagctcatgagaaccaaccccaacgcctgagcacctctacaaaaaattcgcgagacatggactatggtgcagagccagccaaccagagtccagccactcctgagaccatcgaccataaaccctcgactgcctgtgtcgtccccacagctaagagctgggccacaggtttgtgggcaccaggacggggacactccagagctaaggccacttcgcttttgtcaaaggctcctctgaatgattttgggaaatcaagaagtttaaaatcacatactcattttacttgaaattatgtcactagacaacttaaatttttgagtcttgagattgtttttctcttttcttattaaatgatctttctatgaaccagcaaaaaaaaaaaaaaaaaaaa

SEQ ID NO: 9 - protein, 321 aa, Homo sapiens
MGEPQGSMRILVTGGSGLVGKAIQKVVADGAGLPGEDWVFVSSKDADLTDTAQTRALFEKVQPTHVIHLAAMVGGLFRNIKYNLDFWRKNVHMNDNVLHSAFEVGARKVVSCLSTCIFPDKTTYPIDETMIHNGPPHNSNFGYSYAKRMIDVQNRAYFQQYGCTFTAVIPTNVFGPHDNFNIEDGHVLPGLIHKVHLAKSSGSALTVWGTGNPRRQFIYSLDLAQLFIWVLREYNEVEPIILSVGEEDEVSIKEAAEAVVEAMDFHGEVTFDTTKSDGQFKKTASNSKLRTYLPDFRFTPFKQAVKETCAWFTDNYEQARK

SEQ ID NO: 10 - DNA, 966 bp, Homo sapiens
atgggtgaaccccagggatccatgcggattctagtgacagggggctctgggctggtaggcaaagccatccagaaggtggtagcagatggagctggacttcctggagaggactgggtgtttgtctcctctaaagacgccgatctcacggatacagcacagacccgcgccctgtttgagaaggtccaacccacacacgtcatccatcttgctgcaatggtggggggcctgttccggaatatcaaatacaatttggacttctggaggaaaaacgtgcacatgaacgacaacgtcctgcactcggcctttgaggtgggcgcccgcaaggtggtgtcctgcctgtccacctgtatcttccctgacaagacgacctacccgatagatgagaccatgatccacaatgggcctccccacaacagcaattttgggtactcgtatgccaagaggatgatcgacgtgcagaacagggcctacttccagcagtacggctgcaccttcaccgctgtcatccccaccaacgtcttcgggccccacgacaacttcaacatcgaggatggccacgtgctgcctggcctcatccacaaggtgcacctggccaagagcagcggctcggccctgacggtgtggggtacagggaatccgcggaggcagttcatatactcgctggacctggcccagctctttatctgggtcctgcgggagtacaatgaagtggagcccatcatcctctccgtgggcgaggaagatgaggtctccatcaaggaggcagccgaggcggtggtggaggccatggacttccatggggaagtcacctttgatacaaccaagtcggatgggcagtttaagaagacagccagtaacagcaagctgaggacctacctgcccgacttccggttcacacccttcaagcaggcggtgaaggagacctgtgcttggttcactgacaactacgagcaggcccggaagtga

SEQ ID NO: 11 - protein, 321 aa, Mus musculus
MGEPHGSMRILVTGGSGLVGRAIQKVVADGAGLPGEEWVFVSSKDADLTDAAQTQALFQKVQPTHVIHLAAMVGGLFRNIKYNLDFWRKNVHINDNVLHSAFEVGARKVVSCLSTCIFPDKTTYPIDETMIHNGPPHSSNFGYSYAKRMIDVQNRAYFQQHGCTFTAVIPTNVFGPYDNFNIEDGHVLPGLIHKVHLAKSSDSALTVWGTGKPRRQFIYSLDLARLFIWVLREYSEVEPIILSVGEEDEVSIKEAAEAVVEAMDFNGEVTFDSTKSDGQYKKTASNGKLRSYLPDFRFTPFKQAVKETCTWFTDNYEQARK

SEQ ID NO: 12 - DNA, 966 bp, Mus musculus
atgggcgaaccccatggatccatgaggatcctagtgacagggggctctggactggtgggtagagccatccagaaggtggttgcagatggggccggcttacctggagaggaatgggtgtttgtctcctccaaagatgcagatctgacggatgcagcccaaacccaagcactcttccagaaagtacagcccacccacgtcatccatctcgctgcaatggtaggcggccttttccggaatatcaaatacaacttggatttctggcggaaaaacgtgcacatcaatgacaacgtcctgcattcggccttcgaggtgggcgctcgcaaggtggtctcctgcctgtccacctgcatcttccctgacaagaccacctatcctattgacgagacaatgatccacaacgggccgcctcacagcagcaatttcgggtactcatacgccaagaggatgattgacgtgcagaacagagcctacttccagcagcacggctgtaccttcaccgccgtcatccctaccaatgtctttgggccttatgacaacttcaacatcgaagatggccacgtgctacccggcctcatccataaggtgcacctggccaagagtagtgactcggccctgacggtgtggggtacagggaagccgcggaggcagttcatctactcactggacctcgcccggctcttcatctgggtcctacgggagtacagtgaggtggagcccatcatcctctcagtgggtgaggaagatgaagtgtccatcaaggaggcagctgaggctgtagtggaggccatggacttcaatggggaagtcacttttgattcaacaaagtcagatgggcaatataagaagacagccagcaatggcaagttgcggtcctacttgcccgacttccgtttcacacccttcaagcaggctgtgaaggaaacctgcacttggttcaccgacaactatgagcaggcccggaagtaa

SEQ ID NO: 13 - protein, 321 aa, Rattus norvegicus
MGEPHGSMRILVTGGSGLVGRAIQKVVADGAGLPGEEWVFVSSKDADLTDAAQTQALFQKVQPTHVIHLAAMVGGLFRNIKYNLDFWRKNVHINDNVLHSAFEVGTRKVVSCLSTCIFPDKTTYPIDETMIHNGPPHSSNFGYSYAKRMIDVQNRAYFQQHGCTFTSVIPTNVFGPYDNFNIEDGHVLPGLIHKVHLAKSSGSALTVWGTGKPRRQFIYSLDLARLFIWVLREYNEVEPIILSVGEEDEVSIKEAAEAVVEAMDFSGEVTFDSTKSDGQYKKTASNGKLRSYLPDFCFTPFKQAVKETCAWFTENYEQARK

SEQ ID NO: 14 - DNA, 966 bp, Rattus norvegicus
atgggtgaaccccacggatccatgaggatcctagtaacagggggctctggactggtgggcagagccatccagaaggtggtcgcagatggggccggcttgcctggagaggaatgggtgtttgtctcctccaaagatgcagatctgacggatgcagcgcaaacccaagctctgttccagaaggtacagcccacccacgtcatccatcttgctgcaatggtaggcggccttttccggaatattaaatacaacttggatttctggaggaagaacgtgcacatcaatgacaacgtcctacattcagccttcgaggtgggcacacgcaaggtggtctcctgcctgtccacctgcatcttccctgacaagaccacctatcctattgatgagaccatgatccacaacgggccgcctcacagcagcaattttgggtactcatatgccaagaggatgattgacgtgcagaacagggcctacttccagcagcatggctgtaccttcacctctgtcatccctaccaatgtctttgggccttacgacaacttcaacatcgaagatggccacgtgctgccgggcctcatccataaggtgcacctggccaagagcagtggttcagccttgactgtgtggggtacggggaagccgcggagacagttcatctactcactggacctagcccggctcttcatctgggtccttcgggagtacaatgaggtggagcccatcatcctctcagtgggcgaggaagatgaagtgtctatcaaggaggcagctgaggctgtggtggaggccatggacttctctggggaagtcacttttgattcaacaaagtcagatgggcagtataagaagacagccagcaatggcaagttgcggtcctacttgcctgacttctgtttcacacccttcaagcaggctgtgaaggaaacttgtgcttggttcactgaaaactacgagcaggcccggaagtaa

SEQ ID NO: 15 - protein, 321 aa, Cricetulus griseus
MGEPQGSRRILVTGGSGLVGRAIQKVVADGAGLPGEEWVFVSSKDADLTDAAQTQALFQKVQPTHVIHLAAMVGGLFRNIKYNLDFWRKNVHINDNVLHSAFEVGTRKVVSCLSTCIFPDKTTYPIDETMIHNGPPHSSNFGYSYAKRMIDVQNRAYFQQHGCTFTAVIPTNVFGPHDNFNIEDGHVLPGLIHKVHLAKSNGSALTVWGTGKPRRQFIYSLDLARLFIWVLREYNEVEPIILSVGEEDEVSIKEAAEAVVEAMDFCGEVTFDSTKSDGQYKKTASNGKLRAYLPDFRFTPFKQAVKETCAWFTDNYEQARK

SEQ ID NO: 16 - DNA, 1311 bp, Cricetulus griseus
ccggaagtagctcttggactggtggaaccctgcgcaggtgcagcaacaatgggtgagccccagggatccaggaggatcctagtgacagggggctctggactggtgggcagagctatccagaaggtggtcgcagatggcgctggcttacccggagaggaatgggtgtttgtctcctccaaagatgcagatctgacggatgcagcacaaacccaagccctgttccagaaggtacagcccacccatgtcatccatcttgctgcaatggtaggaggccttttccggaatatcaaatacaacttggatttctggaggaagaatgtgcacatcaatgacaacgtcctgcactcagctttcgaggtgggcactcgcaaggtggtctcctgcctgtccacctgtatcttccctgacaagaccacctatcctattgatgaaacaatgatccacaatggtccaccccacagcagcaattttgggtactcgtatgccaagaggatgattgacgtgcagaacagggcctacttccagcagcatggctgcaccttcactgctgtcatccctaccaatgtctttggacctcatgacaacttcaacattgaagatggccatgtgctgcctggcctcatccataaggtgcatctggccaagagtaatggttcagccttgactgtttggggtacagggaaaccacggaggcagttcatctactcactggacctagcccggctcttcatctgggtcctgcgggagtacaatgaagttgagcccatcatcctctcagtgggcgaggaagatgaagtctccattaaggaggcagctgaggctgtagtggaggccatggacttctgtggggaagtcacttttgattcaacaaagtcagatgggcagtataagaagacagccagcaatggcaagcttcgggcctacttgcctgatttccgtttcacacccttcaagcaggctgtgaaggagacctgtgcctggttcaccgacaactatgagcaggcccggaagtgaagcatgggacaagcgggtgctcagctggcaatgcccagtcagtaggctgcagtctcatcatttgcttgtcaagaactgaggacagtatccagcaacctgagccacatgctggtctctctgccagggggcttcatgcagccatccagtagggcccatgtttgtccatcctcgggggaaggccagaccaacaccttgtttgtctgcttctgccccaacctcagtgcatccatgctggtcctgctgtcccttgtctagaaaccaataaaatggattttcataaaaaaaaaaaaaaaaaaa

SEQ ID NO: 17 - protein, 594 aa, Homo sapiens
MAAARDPPEVSLREATQRKLRRFSELRGKLVARGEFWDIVAITAADEKQELAYNQQLSEKLKRKELPLGVQYHVFVDPAGAKIGNGGSTLCALQCLEKLYGDKWNSFTILLIHSGGYSQRLPNASALGKIFTALPLGNPIYQMLELKLAMYIDFPLNMNPGILVTCADDIELYSIGEFEFIRFDKPGFTALAHPSSLTIGTTHGVFVLDPFDDLKHRDLEYRSCHRFLHKPSIEKMYQFNAVCRPGNFCQQDFAGGDIADLKLDSDYVYTDSLFYMDHKSAKMLLAFYEKIGTLSCEIDAYGDFLQALGPGATVEYTRNTSHVIKEESELVEMRQRIFHLLKGTSLNVVVLNNSKFYHIGTTEEYLFYFTSDNSLKSELGLQSITFSIFPDIPECSGKTSCIIQSILDSRCSVAPGSVVEYSRLGPDVSVGENCIISGSYILTKAALPAHSFVCSLSLKMNRCLKYATMAFGVQDNLKKSVKTLSDIKLLQFFGVCFLSCLDVWNLKVTEELFSGNKTCLSLWTARIFPVCSSLSDSVITSLKMLNAVKNKSAFSLNSYKLLSIEEMLIYKDVEDMITYREQIFLEISLKSSLM

SEQ ID NO: 18 - DNA, 1785 bp, Homo sapiens
atggcagctgctagggaccctccggaagtatcgctgcgagaagccacccagcgaaaattgcggaggttttccgagctaagaggcaaacttgtagcacgtggagaattctgggacatagttgcaataacagcggctgatgaaaaacaggaacttgcttacaaccaacagctgtcagaaaagctgaaaagaaaggagttaccccttggagttcaatatcacgtttttgtggatcctgctggagccaaaattggaaatggaggatcaacactttgtgcccttcaatgtttggaaaagctatatggagataaatggaattcttttaccatcttattaattcactctggtggctacagtcaacgacttccaaatgcaagtgctctgggaaaaattttcactgctttacctcttggtaaccccatttatcagatgctagaattaaagctagccatgtacattgatttccccttaaatatgaatcctggaattctggttacctgtgcagatgatattgaactttatagtattggagaatttgagtttattaggtttgacaaacctggctttactgctttagctcatccttctagtttgacgataggtaccacacatggagtatttgtcttagatccttttgatgatttaaaacatagagaccttgaatacaggtcttgccatcgtttccttcataagcccagcatagaaaagatgtatcagtttaatgctgtgtgtagacctggaaatttttgtcaacaggactttgctgggggtgacattgccgatcttaaattagactctgactatgtctacacagatagcctattttatatggatcataaatcagcaaaaatgttacttgctttttatgaaaaaataggcacactgagctgtgaaatagatgcctatggtgactttctgcaggctttgggacctggagcaactgtggagtacaccagaaacacatcacatgtcattaaagaagagtcagagttggtagaaatgaggcagagaatatttcatcttcttaaaggaacatcactaaatgttgttgttcttaataactccaaattttatcacattggaacaaccgaagaatatttgttttactttacctcagataacagtttaaagtcagagctcggcttacagtccataacttttagtatctttccagatataccagaatgctctggcaaaacatcctgtatcattcaaagcatactggattcaagatgttctgtggcacctggctcagttgtggagtattccagattggggcctgatgtttcagttggggaaaactgcattattagtggttcttacatcctaacaaaagctgccctccccgcacattcttttgtatgttccttaagcttaaagatgaatagatgcttaaagtatgcaactatggcatttggagtgcaagacaacttgaaaaagagtgtgaaaacattgtcagatataaagttacttcaattctttggagtctgtttcctgtcatgcttagatgtttggaatcttaaagttacagaggaactgttctctggtaacaagacatgtctgagtttgtggactgcacgcattttcccagtttgttcttctttgagtgactcagttataacatccctaaagatgttaaatgctgttaagaacaagtcagcattcagcctgaatagctataagttgctgtccattgaagaaatgcttatctacaaagatgtagaagatatgataacttacagggaacaaatttttctagaaatcagtttaaaaagcagtttgatgtag

SEQ ID NO: 19 - protein, 590 aa, Mus musculus
MASLREATLRKLRRFSELRGKPVAAGEFWDVVAITAADEKQELAYKQQLSEKLKKRELPLGVQYHVFPDPAGTKIGNGGSTLCSLECLESLCGDKWNSLKVLLIHSGGYSQRLPNASALGKIFTALPLGEPIYQMLELKLAMYVDFPSNMRPGVLVTCADDIELYSVGDSEYIAFDQPGFTALAHPSSLAVGTTHGVFVLHSDSSLQHGDLEYRQCYQFLHKPTIENMHRFNAVHRQRSFGQQNLSGGDTDCLPLHTEYVYTDSLFYMDHKSAKKLLDFYKSEGPLNCEIDAYGDFLQALGPGATAEYTRNTSHVTKEESQLLDMRQKIFHLLKGTPLNVVVLNNSRFYHIGTLQEYLLHFTSDSALKTELGLQSIAFSVSPSVPERSSGTACVIHSIVDSGCCVAPGSVVEYSRLGPEVSIGENCIISSSVIAKTVVPAYSFLCSLSVKINGHLKYSTMVFGMQDNLKNSVKTLEDIKALQFFGVCFLSCLDIWNLKATEKLFSGNKMNLSLWTACIFPVCSSLSESATASLGMLSAVRNHSPFNLSDFNLLSIQEMLVYKDVQDMLAYREHIFLEISSNKNQSDLEKS

SEQ ID NO: 20 - DNA, 3526 bp, Mus musculus
agtgtgctcccggaagtcggccatggcgtctctccgcgaagccaccctgcggaaactgcgcagattttctgagctgagaggcaaacccgtggcagctggagaattctgggatgtggttgcaataacagcagctgatgaaaagcaggagctcgcttacaagcaacagttgtccgagaagctgaagaaaagggaattgcctcttggagttcaataccatgtttttccagatcctgctgggaccaaaattggaaatggaggatcaacactttgttcccttgagtgtttggaaagcctctgtggagacaaatggaattctctgaaggtcctgctaatccactctggtggctacagccaacgccttcccaatgcgagtgctttaggaaagatcttcacagccttaccacttggtgaacccatttatcagatgttggagttaaaactagccatgtacgtggatttcccctcaaacatgaggcctggagtcttggtcacctgtgcagatgatatcgaactctacagtgttggggacagtgagtacattgcctttgaccagcctggctttactgccttagcccatccgtctagtctggctgtaggcactactcatggagtatttgtcttgcactctgacagttccttacaacatggtgaccttgagtacaggcaatgctaccaattcctccacaagcccaccattgaaaacatgcaccgctttaatgctgtgcatagacaacgaagctttggtcaacagaacttgtctggaggtgacactgactgtcttccattgcacactgagtatgtctacacagatagcctgttttacatggatcacaaatcagccaaaaagttacttgatttctataaaagtgaaggcccactgaactgtgaaatagatgcctatggagactttcttcaggcactggggcctggagcaactgcagagtacaccaggaacacatctcatgtcactaaagaagagtcccagttgttggacatgaggcagaaaatattccacctcctcaagggaacaccactgaatgttgttgttcttaataactccagattttatcacattggaacactgcaagagtatctgcttcatttcacctctgatagtgcattaaagacggagctgggcttacaatccatagctttcagtgtctctccaagtgttcctgagcgctccagtggaacagcctgtgtcattcacagtatagtggattcaggatgctgtgtggcccctggctcagtggtagagtattctagattggggcctgaggtgtccatcggggaaaactgcattatcagcagttctgtcatagcaaaaactgttgtgccagcatattcttttttgtgttctttaagtgtgaagataaatggacacttaaaatattctactatggtgtttggcatgcaagacaacttgaagaacagtgttaaaacactggaagacataaaggcacttcagttctttggagtctgttttctgtcttgtttagacatttggaatcttaaagctacagagaaactattctctggaaataagatgaatctgagcctgtggactgcatgcattttccctgtctgttcatctctgagtgagtcggctacagcatcccttgggatgttaagcgctgtaaggaaccattcaccattcaacctaagtgactttaaccttttgtccatccaggaaatgcttgtctacaaagatgtacaagacatgctagcttatagggaacacatttttctagaaattagttcaaataaaaatcaatctgatttagagaaatcttgaatatattttggccataaacaaaattgcaaatacaggcattttctatagacctctgacatttttgtttgttttaataaagtaatataataaaaattatgttaatataactgttgtagcttggtaatgagaatggtacaactgaccacttctgctagaagtacgttccaggactagagtcaggaaaggtcggctgttttagatgtttacaccatcttacaattgtgctctttggtaaagatccatttatgggacactgtttcattcacaaaataaatatttctgttttataggatgattttctaaacataacatatctttaaagcttttctatcttcttttgaaatttggaccaataaaattctaggtgatatggaggattgtattgctcaacttctcatagtgagacaacacgtaacaaaacattgttataaattcttagaagaaatgtcattatttgaggttttctttgaggactttgttctagttttattttatgtgtataaatgtgttacctgcatgtatgcatgtgcaccacttgcctgcggcacccatagaggctagaacagctgttctcaacatttgggttgggaccttttgtgggctcaaacaatcctttgaggggtaacctaagtccattggaaaacaaaatatttacattatgattcataacagtagggaaattacagttaagtagcaacaaaaataattttatatttggggtcactacagcatggggactgtattgaaaggatagcagcatcaggaaggttaaaaactgccggtctagaagaaagcattgggtctcttggaactagagttatagatgcttagaacctccgtgttgcttctgtaagtcaacctccttagtcctatgaaagtgctatataatgatgtttgtgcctcattggtcttgccaaaatgatataaaagtatgtatggatgattttgttcttatacactagaacatgtgttgccatatcttataaactatgtctactgatatattacactggtagctatgtacacacagaactcagttgtctgctcaggaggtggtagggatagttgagagccagtactcactcactatggaccttacttaatcctctcctagttaatccttctccaaatctcttaacttgacagtggacatttgccttgcatcattggtggtagtgatgctgtgaacaaacaataggcccaaagagaggaaattcaaataggcaatctgaagaactactcaaatcataaacaactgcagggaaatgaaatgggtggaattcctggttatgcgtacctattatgaaataaacacattagtggaatgtccttaggttgaactgtaatagagttaaattttatcatacttgtgtttaaaataccttaagtacattgtaatatctgctgtggcaactttaattctgtgtaagttttcataaaaatatatgataaacaagatatctgtcaaaactcctttatattatttatataagaatatttgcctttttgaggtactagataataaagcaaagaatgtacgatactatatgacaattattggtaaagttacagagaattcaatggatgttaaatgttattaaatactcaagactaaagtcctatcaacgatgagaattatgatttcatgttccaagaaaaaaatatcattaataaagaataccatcacttccttgtaaaaaaaaaaaaaaaaaaaaaaaaaaaaaaaaaaaa

SEQ ID NO: 21 - protein, 591 aa, Rattus norvegicus
METLREATLRKLRRFSELRGKPVAAGEFWDVVAITAADEKQELAYKQQLSEKLRRKELPLGVQYHVFPDPAGTKIGNGGSTLCSLQCLKSLYGDEWNSFKVLLIHSGGYSQRLPNASALGKIFTALPLGEPIYQMLELKLAMYVDFPSHMKPGVLVTCADDIELYSVGDCQYIAFDQPGFTALAHPSSLAVGTTHGVFVLHSASSLQHGDLQYRQCHRFLHKPTIENMHQFNAVQRQGSFAQQDFPGGDTACLPLHTEYVYTDSLFYMDHKSAKKLLDFYKNVNQLNCEIDAYGDFLQALGPGATAEYTRNTSHVTKEDSQLLDMRQKIFHLLKGTPLNVVVLNNSRFYHIGTTQEYLLHFTSDSTLRSRARLTVHSFQVSLQVSLNPPMKQPVSFTVYWDSGCCVAPGSVVEYSRLGPEVSIGENCIVSSSVLANTAVPAYSFVCSLSVRTNGLLEYSTMVFSVQDNLKGSVKTLEDIKALQFFGVCFLSCLDIWNLKATEKLFSGSKRNLSLWTARIFPVCPSLSESVTASLGMLSAVRSHSPFSLSNFKLMSIQEMLVYKDVQDMLAYREQIFLEINSNKKQSDLEKS

SEQ ID NO: 22 - DNA, 1776 bp, Rattus norvegicus
atggagactctccgggaagccaccctgcggaaactgcgcagattttcggagctgagaggcaaacctgtggcagctggagaattctgggatgtggttgcgataacagcagccgatgaaaagcaggagctcgcttacaagcagcagttgtcagaaaagctgagaagaaaggaattgcctcttggagttcaataccatgtttttcctgatcctgctgggaccaaaattggaaatggaggatcgacactttgttcccttcagtgcctaaaaagcctctatggagatgaatggaattctttcaaggtcctgttaattcactccggtggctacagtcaacgccttcccaatgcaagtgctttaggaaagatcttcacagccttaccacttggtgaacccatctatcagatgttggagttaaaactagccatgtacgtggatttcccctcacacatgaagcctggagtcttggtcacctgtgcagatgacattgaactgtacagtgttggggactgtcagtacattgcctttgaccagcctggctttactgccttagcccatccttccagtctggctgtaggcaccacacacggagtatttgtcttgcactctgccagttccttacaacatggtgaccttcagtacagacaatgccaccgtttcctccacaagcccaccattgaaaacatgcatcagtttaatgctgtgcaaagacaaggaagctttgctcaacaggacttccctggaggtgacaccgcgtgtcttccattgcacactgagtatgtctacacagatagcctgttttacatggaccacaaatcggccaaaaagttacttgatttctataaaaatgtaaaccaactgaactgtgaaatagatgcctatggtgactttctgcaggcactggggcctggagcaactgcagagtataccaggaacacatcacatgtcactaaagaagactcccagttgttggacatgaggcagaaaatattccacctcctcaaggggacaccactgaatgttgttgttcttaataactccagattttatcacattggaacaacacaagaatatctgcttcatttcacgtctgatagtacgttaaggtcaagagctaggcttacagtccatagctttcaagtgtctctccaagtatccctgaatcctccaatgaaacagcctgtatcattcacagtatactgggattcaggatgctgtgtggcacctggctcagttgtagagtattctagactggggcctgaggtgtccattggggaaaactgcattgtcagcagctctgtcctagcaaacactgctgtgccggcatattcttttgtgtgttctctaagtgtgaggacaaatggactcttggaatattctaccatggtgtttagtgtgcaggacaacttgaaaggcagtgttaaaaccctggaagatataaaggcacttcagttctttggagtctgtttcttgtcttgtttagacatctggaaccttaaagctacagagaaactgttctctggaagtaagaggaacctgagcctgtggactgcacggattttccctgtctgtccttctctgagtgagtcagttacagcatcccttgggatgttaagtgctgtaaggagccattcaccattcagcctaagcaactttaagctgatgtccatccaggaaatgcttgtctacaaagatgtacaagacatgctagcttatagggagcaaatttttctagaaattaattcaaataaaaaacaatctgatttagagaaatcttaa

SEQ ID NO: 23 - protein, 1084 aa, Homo sapiens
MEQPKGVDWTVIILTCQYKDSVQVFQRELEVRQKREQIPAGTLLLAVEDPEKRVGSGGATLNALLVAAEHLSARAGFTVVTSDVLHSAWILILHMGRDFPFDDCGRAFTCLPVENPEAPVEALVCNLDCLLDIMTYRLGPGSPPGVWVCSTDMLLSVPANPGISWDSFRGARVIALPGSPAYAQNHGVYLTDPQGLVLDIYYQGTEAEIQRCVRPDGRVPLVSGVVFFSVETAERLLATHVSPPLDACTYLGLDSGARPVQLSLFFDILHCMAENVTREDFLVGRPPELGQGDADVAGYLQSARAQLWRELRDQPLTMAYVSSGSYSYMTSSASEFLLSLTLPGAPGAQIVHSQVEEQQLLAAGSSVVSCLLEGPVQLGPGSVLQHCHLQGPIHIGAGCLVTGLDTAHSKALHGRELRDLVLQGHHTRLHGSPGHAFTLVGRLDSWERQGAGTYLNVPWSEFFKRTGVRAWDLWDPETLPAEYCLPSARLFPVLHPSRELGPQDLLWMLDHQEDGGEALRAWRASWRLSWEQLQPCLDRAATLASRRDLFFRQALHKARHVLEARQDLSLRPLIWAAVREGCPGPLLATLDQVAAGAGDPGVAARALACVADVLGCMAEGRGGLRSGPAANPEWMRPFSYLECGDLAAGVEALAQERDKWLSRPALLVRAARHYEGAGQILIRQAVMSAQHFVSTEQVELPGPGQWVVAECPARVDFSGGWSDTPPLAYELGGAVLGLAVRVDGRRPIGARARRIPEPELWLAVGPRQDEMTVKIVCRCLADLRDYCQPHAPGALLKAAFICAGIVHVHSELQLSEQLLRTFGGGFELHTWSELPHGSGLGTSSILAGTALAALQRAAGRVVGTEALIHAVLHLEQVLTTGGGWQDQVGGLMPGIKVGRSRAQLPLKVEVEEVTVPEGFVQKLNDHLLLVYTGKTRLARNLLQDVLRSWYARLPAVVQNAHSLVRQTEECAEGFRQGSLPLLGQCLTSYWEQKKLMAPGCEPLTVRRMMDVLAPHVHGQSLAGAGGGGFLYLLTKEPQQKEALEAVLAKTEGLGNYSIHLVEVDTQGLSLKLLGTEASTCCPFP

SEQ ID NO: 24 - DNA, 3255 bp, Homo sapiens
atggagcagccgaagggagttgattggacagtcatcatcctgacctgccagtacaaggacagtgtccaggtctttcagagagaactggaagtgcggcagaagcgggagcagatccctgctgggacgctgttactggccgtggaggacccagagaagcgtgtgggcagcggaggagccaccctcaacgccctgctggtggctgctgaacacctgagtgcccgggcaggcttcactgtggtcacatccgatgtcctgcactcggcctggatcctcattctgcacatgggtcgagacttcccctttgatgactgtggcagggctttcacctgcctccccgtggagaaccccgaggcccccgtggaagccttggtctgcaacctggactgcctgctggacatcatgacctatcggctgggcccgggctccccgccaggcgtgtgggtctgcagcaccgacatgctgctgtctgttcctgcaaatcctggtatcagctgggacagcttccggggagccagagtgatcgccctcccagggagcccggcctacgctcagaatcatggcgtctacctaactgacccccagggccttgttttggacatttactaccagggcactgaggcagagattcagcggtgtgtcaggcctgatgggcgggtgccactggtctctggggttgtcttcttctctgtggagactgccgagcgcctcctagccacccacgtgagcccgcccctggatgcctgcacctacctaggcttggactccggagcccggcctgtccagctgtctctgttttttgacattctccactgcatggctgagaacgtgaccagggaggacttcctggtggggaggcccccagagttggggcaaggcgatgcagatgtagcgggttatctgcagagcgcccgggcccagctgtggagggagcttcgcgatcagccccttaccatggcctatgtctccagcggcagctacagctacatgacctcctcagccagtgagttcctgctcagcctcacactccccggggctcctggggcccagattgtgcactcccaggtggaggagcagcagcttctggcggccgggagctctgtggtcagctgcctgctggagggccctgtccagctgggtcctgggagcgtcctgcagcactgccacctgcagggccccattcacataggcgctggctgcttggtgactggcctggatacagcccactccaaggccctgcatggccgggagctgcgtgaccttgtcctgcagggacaccacacgcggctacacggctccccgggccacgccttcaccctcgttggccgtctggacagctgggagagacagggggcaggcacatatctcaacgtgccctggagtgaattcttcaagaggacaggtgttcgagcctgggacctgtgggaccctgagacgctgcccgcagagtactgccttcccagcgcccgcctctttcctgtgctccacccctcgagggagctgggaccccaggacctgctgtggatgctggaccaccaggaggatgggggcgaggccctgcgagcctggcgggcctcctggcgcctgtcctgggagcagctgcagccgtgcctggatcgggctgccacgctggcctctcgccgggacctgttcttccgccaggccctgcataaggcgcggcacgtgctggaggcccggcaggacctcagcctgcgcccgctgatctgggctgctgtccgcgagggctgccccgggcccctgctggccacgctggaccaggttgcagctggggcaggagaccctggtgtggcggcacgggcactggcctgtgtggcggacgtcctgggctgcatggcagagggccgtgggggcttgcggagcgggccagctgccaaccctgagtggatgcggcccttctcatacctggagtgtggagacctggcagcgggcgtggaggcgcttgcccaggagagggacaagtggctaagcaggccagccttgctggtgcgagcggcccgccactatgagggggctggtcagatcctgatccgccaggctgtgatgtcagcccagcactttgtctccacagagcaggtggaactgccgggacctgggcagtgggtggtggctgagtgcccggcccgtgtggatttctctgggggctggagtgacacgccaccccttgcctatgagcttggcggggctgtgctgggcctggctgtgcgagtggacggccgccggcccatcggagccagggcacgccgcatcccggagcctgagctgtggctggcggtggggcctcggcaggatgagatgactgtgaagatagtgtgccggtgcctggctgacctgcgggactactgccagcctcatgccccaggggccctgctgaaggcggccttcatctgtgcagggatcgtgcatgtccactcggaactccagctgagtgagcagctgctccgcaccttcgggggcggctttgagctgcacacctggtctgagctgccccacggctctggcctgggcaccagcagcatcctggcaggcactgccctggctgccttgcagcgagccgcaggccgggtggtgggcacggaagccctgatccacgcagtgctgcacctggagcaggtgctcaccactggaggtggctggcaggaccaagtaggtggcctaatgcctggcatcaaggtggggcgctcccgggctcagctgccactgaaggtggaggtagaagaggtcacggtgcctgagggctttgtccagaagctcaatgaccacctgctcttggtgtacactggcaagacccgcctggctcggaacctgctgcaggatgtgctgaggagctggtatgcccgacttcctgctgtggtgcagaatgcccacagcctggtacggcaaactgaggagtgtgctgaaggcttccgccaaggaagcctgcctctgctgggccagtgcctgacctcgtactgggagcagaagaagctcatggctccaggctgtgagcccctgactgtgcggcgtatgatggatgtcctggccccccacgtgcatggccagagcctggctggggcaggcggtggaggctttctctatctgttgaccaaggagccacagcaaaaggaggccttggaggcggtgctggccaagaccgagggccttgggaattacagcatccacctggttgaagtggacactcagggcctgagcctgaagctgctggggaccgaggcctcaacctgttgccctttcccatga

SEQ ID NO: 25 - protein, 1090 aa, Mus musculus
MEQSEGVNWTVIILTCQYKDSVQVFQRELEVRQRREQIPAGTMLLAVEDPQTRVGSGGATLNALLVAAEHLSARAGFTVVTSDVLHSAWILILHMGRDFPFDDCGRAFTCLPVENPQAPVEALVCNLDCLLDIMTHRLGPGSPPGVWVCSTDMLLSVPPNPGISWDGFRGARVIAFPGSLAYALNHGVYLTDSQGLVLDIYYQGTKAEIQRCVGPDGLVPLVSGVVFFSVETAEHLLATHVSPPLDACTYMGLDSGAQPVQLSLFFDILLCMARNMSRENFLAGRPPELGQGDMDVASYLKGARAQLWRELRDQPLTMVYVPDGGYSYMTTDATEFLHRLTMPGVAVAQIVHSQVEEPQLLEATCSVVSCLLEGPVHLGPRSVLQHCHLRGPIRIGAGCFVSGLDTAHSEALHGLELHDVILQGHHVRLHGSLSRVFTLAGRLDSWERQGAGMYLNMSWNEFFKKTGIRDWDLWDPDTPPSDRCLLTARLFPVLHPTRALGPQDVLWMLHPRKHRGEALRAWRASWRLSWEQLQPCVDRAATLDFRRDLFFCQALQKARHVLEARQDLCLRPLIRAAVGEGCSGPLLATLDKVAAGAEDPGVAARALACVADVLGCMAEGRGGLRSGPAANPEWIQPFSYLECGDLMRGVEALAQEREKWLTRPALLVRAARHYEGAEQILIRQAVMTARHFVSTQPVELPAPGQWVVTECPARVDFSGGWSDTPPIAYELGGAVLGLAVRVDGRRPIGAKARRIPEPELWLAVGPRQDEMTMRIVCRSLDDLRDYCQPHAPGALLKAAFICAGIVHLHSELPLLEQLLHSFNGGFELHTWSELPHGSGLGTSSILAGAALAALQRAAGRAVGTEALIHAVLHLEQVLTTGGGWQDQVSGLMPGIKVGRSRAQLPLKVEVEEITVPEGFVQKINDHLLLVYTGKTRLARNLLQDVLRNWYARLPVVVQNARRLVRQTEKCAEAFRQGNLPLLGQYLTSYWEQKKLMAPGCEPLAVQRMMDVLAPYAYGQSLAGAGGGGFLYLLTKEPRQKETLEAVLAKAEGLGNYSVHLVEVDPQGLSLQLLGHDTRLCGAGPSEVGTT

SEQ ID NO: 26 - DNA, 3273 bp, Mus musculus
atggagcagtcagagggagtcaattggactgtcattatcctgacatgccagtacaaggacagtgtccaggtctttcagagagagctggaggtaaggcagagacgggagcagattcctgcggggacgatgttactggctgtggaggatccccagactcgagtcggcagcggaggagccaccctcaacgcactgctggtggctgctgaacacttgagtgcccgagctggcttcactgtggtcacgtccgatgtcctgcactctgcctggatcctcatcttgcacatgggccgagacttccccttcgatgactgtggcagggccttcacttgcctccctgtggagaacccacaggcccctgtggaggccttggtatgcaacctggactgcctgttggatatcatgacccaccggctgggtccaggttccccaccaggtgtgtgggtctgcagcaccgacatgcttctgtctgttcctccaaaccctgggatcagttgggatggcttccggggagccagagtgatcgcctttcctgggagcctggcctatgcgttgaaccacggtgtctacctcactgactcacagggcttggttttggacatttactaccagggcactaaggcggagatacaacgttgtgtcggacctgatgggctggtaccattggtctccggggtcgtcttcttctctgtggagactgctgagcacctcctagccacccatgtgagcccaccgctggatgcctgcacctatatgggcttggactctggagcccagcctgtgcagctgtctctgtttttcgacatcctgctctgcatggctcggaatatgagcagggagaacttcctggctgggcggcccccggagttggggcaaggtgacatggatgtagcaagttacctgaagggagcccgggcccagctgtggagggagcttcgagatcagcccctcacaatggtgtatgtccctgacggcggctacagctacatgacgactgatgccaccgagttcctgcacagactcacgatgcctggagtagctgtggcacagattgttcactcccaggtggaggagccacagctgctagaggctacgtgctcggtggtcagctgcctgctcgagggccctgtgcacctggggcctcgaagtgtcctgcagcactgtcacctgaggggccccattcgcatcggcgctggctgctttgtgagtggtctggatacagcccactcggaggcactgcatggcctggagctccatgatgtcatcctgcagggacaccatgtgcggctgcatggctccctgagccgtgtatttactcttgctggccgtctggacagctgggaaagacagggggcaggcatgtatctcaacatgtcctggaatgagttcttcaagaagacaggcattcgagactgggacctgtgggacccagatacacccccctcagatcgatgcctcctcactgcccgccttttccctgtgctccaccccacgagggccctggggccccaggatgtgctgtggatgctgcacccccgcaaacacagaggtgaggcccttcgggcctggcgagcctcctggcgtctgtcctgggagcagctgcaaccttgtgtggaccgggctgccacactggacttccgccgagatctgttcttctgccaggccttgcagaaggcaaggcatgtgttagaggcgcggcaggacctctgcctacgtccactgatccgggccgctgtcggggaaggttgctctgggcccctgctggccacacttgacaaggttgcagctggggcagaagatcctggcgtggcagcccgggctctggcttgtgtggccgatgtgctgggctgcatggcagagggccgaggaggcttgcgcagtgggccagctgccaaccctgagtggattcagcctttctcatacttggagtgtggagacctgatgaggggtgtggaggcgcttgcccaggagagagagaagtggctgaccaggcctgccttgctggttcgagctgcccgccattacgagggggccgagcagatcctgatccgccaggctgtgatgacagcccggcacttcgtctccacccagcccgtggagctgcccgcacccgggcagtgggtggtgactgagtgcccagcccgtgtggatttctctgggggctggagtgacacaccgcccattgcctatgagcttggtggagcagtgttgggcctggctgtgcgggtggatggccgccggcccatcggggccaaagcacgccgcatcccggagcctgagctctggctggcagtgggacctcggcaggatgagatgaccatgaggatagtgtgccggagcctggatgacctgcgggattactgccagcctcatgccccaggggccttgctgaaggcagcctttatctgtgctggcattgtgcatctccactcagagctccctctgcttgaacagttgttacactcctttaatggtggctttgagctgcacacgtggtcagagctgccgcacggctctggtcttggcaccagcagcatcctggcaggggctgccctggctgccttacagcgggctgcaggccgggcagtgggcacggaggctctcatccacgcagtgctgcacctggagcaggtgctcaccacaggaggtggctggcaggaccaagtcagtggcctaatgcctggcatcaaagtggggcgctcccgggcccagctgcccctcaaggtggaggtggaggaaatcactgtgcctgagggctttgtccagaagatcaatgaccatctgctcctggtttataccggcaagacccgattggcccggaatctgctgcaggacgtgctgaggaactggtacgctcggttgcccgttgtggtacagaatgcccgcagactggtgcgacagaccgagaagtgcgctgaagctttccgccaaggaaacctgcctctgctgggacagtacctgacctcatactgggagcagaagaagcttatggccccaggctgcgagccgctggccgtgcagcgaatgatggatgtcctggccccgtatgcgtatggccaaagcctggcaggggcaggtggtgggggctttctctatctattgaccaaggaaccccggcagaaagagactctggaagctgtcctggccaaggctgagggccttggcaactacagtgtccacctggtggaagtggatcctcagggcctgagcctgcagctgctgggacacgacacccgtctttgtggggccgggccctctgaagtgggcaccacctag

SEQ ID NO: 27 - protein, 1019 aa, Rattus norvegicus
MDQPKGVNWTVIILTCQYKDSVQVFQRELEVRQKREQIPAGTMLLAVEDPQTRVGSGGATLNALLVAAEHLSARAGFTVVTSDVLHSAWILILHMGRDFPFDDCGRAFTCLPVENPQAPVEALVCNLDCLLDIMTHRLGPGSPPGVWVCSTDMLLSVPPNPGISWDGFRGTRVIAFPGSLAYALNHGVYLTDSQGVVLDIYYQGTKAEIQRCVRPDGLVPLVSGVVFFSVETAEHLLATHVSPPLDACTYMGLDSGAQPVQLSLFFDILLCMARNMSRENFVAGRPPEMGQGDPDVARYLKGARAQLWRELRDQPLTMVYVPDGGYSYMTTDATEFLHRLTMPGVAVAQIVHSQVEEPQLLEATCSVVSCLLEGPVHLGPRSVLQHCHLRGPIHIGAGCFVSGLDTAHSEALHGLELHDLILQGHHIRLHGSQSRVFTLAGRLDSWERQGAGMYLNMSWNEFFKKTGIRDWDLWDPDTPLSDRCLLSARLFPVLHPTRALGPQDVLWMLHPHKDRGEALRAWRASWRLSWEQLQPRLDRAATLDFRRDLFFRQALQKARHVLEARQDLCLHPLIRAAVGEGCSGPLLATLDKVAAGAEDPGVAARALACVADVLGCMAEGQGGLRSGPAANPEWIQPFSYLERGDLMRGVEALAQEREKWLTRPALLVRAARHYEGAEQILIRQAVMTARHFVSTQPVELPAPGQWVVTECPARVDFSGGWSDTPPIAYELGGAVLGLAVRVDGRRPIGAKARRILEPELWLAVGPRQDEMTVKIVCRSLDDLQDYCQPHAPGALLKAAFICADIVHVNSEVPLHEQLLRSFNGGFELHTWSELPHGSGLGTSSILAGAALAALQRAAGRTVGTEALIHAVLHLEQVLTTGGGWQDQVSGLMPGIKVGRSRAQLPLKVEVEEITVPENFVQRKLMAPGCEPLAVHRMMDVLAPYAFGQSLAGAGGGGFLYLLTKEPRQKEVLEAVLAKVEGLGNYSVHLVQVDTQGLSLQLLGHDAHLCGAGPSEVGNT

SEQ ID NO: 28 - DNA, 3060 bp, Rattus norvegicus
atggaccagccaaagggggtcaattggacggtcattatcctgacatgccagtacaaggacagtgtccaggtctttcagagagagctggaggtaaggcagaagcgggagcagatccctgccgggacgatgttactggctgtggaggacccccagacccgagtaggcagtggaggagctactctcaatgcactgctggtggctgctgagcacctgagtgcccgagctggcttcaccgtggtcacgtcagatgtcctgcactcggcttggattctcatcttgcacatgggccgagacttcccctttgatgactgtggcagggccttcacttgcctccctgtggagaatccacaggcccctgtggaggccttggtatgcaacctggactgcctgttggatatcatgacccaccggctgggtccaggatccccaccaggtgtgtgggtctgcagcaccgacatgcttctgtctgttcctccaaaccctgggatcagttgggatggcttccggggaaccagagtgatcgcctttcctgggagcctggcctacgctctaaaccacggggtctacctcactgactcgcagggcgtggttttggacatttactaccagggcactaaggcagagatacaacggtgtgtcaggcctgatggactggtaccactggtctctggggttgtcttcttctctgtggagactgctgagcacctcctagccacccacgtgagcccaccgctggacgcctgcacctatatgggcttggactctggagcccagcctgtgcagctgtctctgtttttcgacatcctgctctgcatggctcggaatatgagcagggagaacttcgtggctgggcggcccccggagatggggcaaggtgacccggatgtagcacgttacctgaagggagcccgggcccagctgtggagggagcttcgagatcagcccctcactatggtgtatgtccctgatggcggttacagttacatgacaactgatgccacggagttcctgcacagactcacgatgcctggagtagctgtggcccagattgttcactctcaggtggaggagccacagctgctagaggctacgtgctccgtggtcagctgcctgctggagggtcccgtgcacctggggcctcgaagtgtcctgcagcactgtcacctgaggggccccattcatattggcgctggctgctttgtgagtggcctggataccgcccactccgaggcactgcatggcctggagcttcatgacctcatccttcagggacaccacatacggctgcatggctcccagagtcgtgtattcactcttgctggccgtctggacagctgggaaagacagggggcaggcatgtatctcaacatgtcctggaatgagttcttcaagaagacaggcattcgagactgggacctgtgggacccagatacacccctctcagatcgatgccttctcagtgcccgccttttccctgtgctccaccccacgagggctctggggccccaggatgtgctgtggatgctgcatcctcataaggacagaggcgaggccctgcgtgcctggagagcctcctggcgtctgtcctgggagcagctgcaacctcgcctggaccgggctgccacactggacttccgtcgggatctgttcttccgccaggccttgcagaaggcgaggcatgtgttagaggcccggcaggacctctgcctacatccactgatccgggctgctgtcggtgaaggttgctctgggcccctgctggccacacttgacaaggttgcagcaggggcagaagatcctggtgtggcagcccgggctctggcttgtgtggcagatgtactcggctgcatggcagagggccaaggaggcttgcgcagtgggccagctgccaaccctgagtggattcagcctttctcatacttggaacgtggagacctcatgaggggtgtggaggcacttgcccaggaaagagagaagtggctgaccaggcctgccttgttggttcgagctgcccgccattatgagggggctgagcagatcctgatccgacaggctgtgatgacagcccggcacttcgtctccacccagccagtggaattgccagcacctgggcagtgggtggtgactgagtgcccagcccgtgtggatttctctgggggctggagtgacacaccacccattgcctatgagcttggtggagcagtattgggcctggctgttcgggtggatggccgccggcccatcggggccaaggcacgccgcatcctagagcctgagctctggctggcagtgggacctcgacaggatgagatgaccgtgaagatagtgtgccggagccttgatgacctgcaggattactgccagcctcatgccccaggtgccttgctgaaggcagcctttatctgtgcggatattgtgcatgtcaactcagaggtccctctgcatgaacagttgctacgctcgtttaatggtggctttgagctgcacacatggtcagagctgccacacggctctggtcttggcactagcagcatcttggcaggggctgccctggctgctttgcagcgggctgcaggccggacagtgggcacagaggctctcatccatgcagtgttgcacctggagcaggtgctcaccacaggaggtggctggcaggaccaagtgagtggcctaatgcctggcatcaaggtggggcgctctcgggcacagctgcccctaaaggtggaggtggaggaaatcactgtgcctgagaactttgtccagaggaagcttatggccccaggctgtgagccgctggctgtgcatcggatgatggatgtcctggccccttatgccttcggccaaagtctggcaggggcaggcggtgggggctttctctatctgttgaccaaggaaccccggcagaaagaggtcctagaagctgtgctggccaaggtggagggcctcggcaactacagcgtccacctggtgcaagtggacactcagggcctgagcctgcagctgctaggacatgacgcccatctttgcggggctgggccctctgaagtgggcaacacctag

SEQ ID NO: 29 - protein, 446 aa, Homo sapiens
MAITVSLVNNKRKIVVLAQPTTVKRKRITPYKSIMTDLYYLSQTDGAGDWREKEAKDLTELVQRRITYLQNPKDCSKAKKLVCNINKGCGYGCQLHHVVYCFMIAYGTQRTLILESQNWRYATGGWETVFRPVSETCTDRSGISTGHWSGEVKDKNVQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPSLLKEAKTKYPNYEFISDNSISWSAGLHNRYTENSLRGVILDIHFLSQADFLVCTFSSQVCRVAYEIMQTLHPDASANFHSLDDIYYFGGQNAHNQIAIYAHQPRTADEIPMEPGDIIGVAGNHWDGYSKGVNRKLGRTGLYPSYKVREKIETVKYPTYPEAEK

SEQ ID NO: 30 - DNA, 2963 bp, Homo sapiens
ggccgacccgagcagccggttccctcctctccaggccccctccccatcccacccccgccgcctggccccagccgacccgtcccttcgtctccccgcggaatggggccggcactgctcagggtcgcgcgccctggacccagctcgctctcggtctcgcgctgtcagcgactgcccggctcgcgccgcctcgcgctctgcctcagtcagtggcgccgaaggctccgttaagcggcggcggcggttcctgtttccgtttcttcctctccgttcggtcgggagtagcatcctccactcagccacccttcccactcccccatcgtggggcagctgcggctgagggctgtggctttggcagctgcgacggggagcggcggagaccgcctctgctcccgcctggggttgctgcttttgctcagaggacatccatgaccctaatggtctttttgttcaagataaagtgattttttgcctttgttgattaactggacaaattcaggataccagaaggccctattgatcaggggccagctataggaagagtacgcgttttagaagagcagcttgttaaggccaaagaacagattgaaaattacaagaaacagaccagaaatggtctggggaaggatcatgaaatcctgaggaggaggattgaaaatggagctaaagagctctggtttttcctacagagtgaattgaagaaattaaagaacttagaaggaaatgaactccaaagacatgcagatgaatttcttttggatttaggacatcatgaaaggattctgatggcaattactgtctcattagtgaacaataaaagaaaaattgttgtattagcacaacctactactgtgaagaggaaaagaattaccccatacaagtctataatgacggatctatactacctcagtcagacagatggagcaggtgattggcgggaaaaagaggccaaagatctgacagaactggttcagcggagaataacatatcttcagaatcccaaggactgcagcaaagccaaaaagctggtgtgtaatatcaacaaaggctgtggctatggctgtcagctccatcatgtggtctactgcttcatgattgcatatggcacccagcgaacactcatcttggaatctcagaattggcgctatgctactggtggatgggagactgtatttaggcctgtaagtgagacatgcacagacagatctggcatctccactggacactggtcaggtgaagtgaaggacaaaaatgttcaagtggtcgagcttcccattgtagacagtcttcatccccgtcctccatatttacccttggctgtaccagaagacctcgcagatcgacttgtacgagtgcatggtgaccctgcagtgtggtgggtgtctcagtttgtcaaatacttgatccgcccacagccttggctagaaaaagaaatagaagaagccaccaagaagcttggcttcaaacatccagttattggagtccatgtcagacgcacagacaaagtgggaacagaagctgccttccatcccattgaagagtacatggtgcatgttgaagaacattttcagcttcttgcacgcagaatgcaagtggacaaaaaaagagtgtatttggccacagatgacccttctttattaaaggaggcaaaaacaaagtaccccaattatgaatttattagtgataactctatttcctggtcagctggactgcacaatcgatacacagaaaattcacttcgtggagtgatcctggatatacattttctctctcaggcagacttcctagtgtgtactttttcatcccaggtctgtcgagttgcttatgaaattatgcaaacactacatcctgatgcctctgcaaacttccattctttagatgacatctactattttgggggccagaatgcccacaatcaaattgccatttatgctcaccaaccccgaactgcagatgaaattcccatggaacctggagatatcattggtgtggctggaaatcattgggatggctattctaaaggtgtcaacaggaaattgggaaggacgggcctatatccctcctacaaagttcgagagaagatagaaacggtcaagtaccccacatatcctgaggctgagaaataaagctcagatggaagagataaacgaccaaactcagttcgaccaaactcagttcaaaccatttcagccaaactgtagatgaagagggctctgatctaacaaaataaggttatatgagtagatactctcagcaccaagagcagctgggaactgacataggcttcaattggtggaattcctctttaacaagggctgcaatgccctcatacccatgcacagtacaataatgtactcacatataacatgcaaacaggttgttttctactttgcccctttcagtatgtccccataagacaaacactgccatattgtgtaatttaagtgacacagacattttgtgtgagacttaaaacatggtgcctatatctgagagacctgtgtgaactattgagaagatcggaacagctccttactctgaggaagttgattcttatttgatggtggtattgtgaccactgaattcactccagtcaacagattcagaatgagaatggacgtttggtttttttttgtttttgtttttgttttttcctttataaggttgtctgtttttttttttttaaataattgcatcagttcattgacctcatcattaataagtgaagaatacatcagaaaataaaatattcactctccattagaaaattttgtaaaacaatgccatgaacaaattctttagtactcaatgtttctggacattctctttgataacaaaaaataaattttaaaaaggaattttgtaaagtttctagaattttatatcattggatgatatgttgatcagccttatgtggaagaactgtgataaaaagaggagctttttagtttttcagcttaaaaaaa

SEQ ID NO: 31 - protein, 575 aa, Rattus norvegicus
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHELLRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPALLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTDEEIPMEPGDIIGVAGNHWDGYSKGVNRKLGKTGLYPSYKVREKIETVKYPTYPEAEK

SEQ ID NO: 32 - DNA, 1728 bp, Rattus norvegicus
atgcgggcatggactggttcctggcgttggattatgctcattctttttgcctgggggaccttgttgttttatataggtggtcatttggttcgagataatgaccaccctgatcactctagcagagaactctccaagattcttgcaaagcttgaacgcttaaaacaacaaaatgaagacttgaggcgaatggctgagtctctacgaataccagaaggccccattgaccaggggacggctacgggaagagtccgtgttttagaagaacagcttgttaaggccaaagaacagattgaaaattacaagaaacaagccagaaatggtctggggaaggatcatgaactcttaaggaggaggattgaaaatggagctaaagagctctggttttttctacaaagtgaactgaagaaattaaagcatctagaaggaaatgaactccaaagacatgcagatgaaattcttttggatttaggacaccatgaaaggtctatcatgacggatctatactacctcagtcaaacagatggagcaggggattggcgtgaaaaagaggccaaagatctgacagagctggtccagcggagaataacttatctccagaatcccaaggactgcagcaaagccaggaagctggtgtgtaacatcaataagggctgtggctatggttgccaactccatcacgtggtctactgtttcatgattgcttatggcacccagcgaacactcatcttggaatctcagaattggcgctatgctactggtggatgggagactgtgtttagacctgtaagtgagacatgcacagacagatctggcctctccactggacactggtcaggtgaagtgaatgacaaaaatattcaagtggtggagctccccattgtagacagcctccatcctcggcctccttacttaccactggctgttccagaagaccttgcagatcgactcgtaagagtccatggtgatcctgcagtgtggtgggtgtcccagttcgtcaaatatttgattcgtccacaaccttggctagaaaaggaaatagaagaagccaccaagaagcttggcttcaaacatccagtcattggagtccatgtcagacgcacagacaaagtgggaacagaggcagccttccatcccatcgaagagtacatggtacatgttgaagaacattttcagcttctcgcacgcagaatgcaagtggataaaaaaagagtatatctggctaccgatgaccctgctttgttaaaggaggcaaagacaaagtactccaattatgaatttattagtgataactctatttcttggtcagctggattacacaatcggtacacagaaaattcacttcggggcgtgatcctggatatacactttctctctcaggctgacttcctagtgtgtactttttcatcccaggtctgtcgggttgcttatgaaatcatgcaaaccctgcatcctgatgcctctgcaaacttccactctttagatgacatctactattttggaggccaaaatgcccacaaccagattgccgtttatcctcacaaacctcgaactgatgaggaaattccaatggaacctggagatatcattggtgtggctggaaaccattgggatggttattctaaaggtgtcaacagaaaacttggaaaaacaggcttatatccctcctacaaagtccgagagaagatagaaacagtcaagtatcccacatatcctgaagctgaaaaatag

SEQ ID NO: 33 - protein, 575 aa, Mus musculus
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHEILRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLLRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPTLLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTEEEIPMEPGDIIGVAGNHWDGYSKGINRKLGKTGLYPSYKVREKIETVKYPTYPEAEK

SEQ ID NO: 34 - DNA, 1728 bp, Mus musculus
atgcgggcatggactggttcctggcgttggattatgctcattctttttgcctgggggaccttgttattttatataggtggtcatttggttcgagataatgaccaccctgatcactccagcagagaactctccaagattcttgcaaagcttgaacgcttaaaacagcaaaatgaagacttgaggcgaatggctgagtctctccgaataccagaaggccccattgaccaggggacagctacaggaagagtccgtgttttagaagaacagcttgttaaggccaaagaacagattgaaaattacaagaaacaagctagaaatggtctggggaaggatcatgaaatcttaagaaggaggattgaaaatggagctaaagagctctggttttttctacaaagcgaactgaagaaattaaagcatttagaaggaaatgaactccaaagacatgcagatgaaattcttttggatttaggacaccatgaaaggtctatcatgacagatctatactacctcagtcaaacagatggagcaggggattggcgtgaaaaagaggccaaagatctgacagagctggtccagcggagaataacatatctccagaatcctaaggactgcagcaaagccaggaagctggtgtgtaacatcaataaaggctgtggctatggttgtcaactccatcacgtggtctactgtttcatgattgcttatggcacccagcgaacactcatcttggaatctcagaattggcgctatgctactggtggatgggagactgtgtttagacctgtaagtgagacatgtacagacagatctggcctctccactggacactggtcaggtgaagtaaatgacaaaaacattcaagtggtcgagctccccattgtagacagcctccatcctcggcctccttacttaccactggctgttccagaagaccttgcagaccgactcctaagagtccatggtgaccctgcagtgtggtgggtgtcccagtttgtcaaatacttgattcgtccacaaccttggctggaaaaggaaatagaagaagccaccaagaagcttggcttcaaacatccagttattggagtccatgtcagacgcacagacaaagtgggaacagaagcagccttccaccccatcgaggagtacatggtacacgttgaagaacattttcagcttctcgcacgcagaatgcaagtggataaaaaaagagtatatctggctactgatgatcctactttgttaaaggaggcaaagacaaagtactccaattatgaatttattagtgataactctatttcttggtcagctggactacacaatcggtacacagaaaattcacttcggggtgtgatcctggatatacactttctctcacaggctgactttctagtgtgtactttttcatcccaggtctgtcgggttgcttatgaaatcatgcaaaccctgcatcctgatgcctctgcgaacttccattctttggatgacatctactattttggaggccaaaatgcccacaatcagattgctgtttatcctcacaaacctcgaactgaagaggaaattccaatggaacctggagatatcattggtgtggctggaaaccattgggatggttattctaaaggtatcaacagaaaacttggaaaaacaggcttatatccctcctacaaagtccgagagaagatagaaacagtcaagtatcccacatatcctgaagctgaaaaatag

SEQ ID NO: 35 - protein, 364 aa, Homo sapiens
MNRAPLKRSRILHMALTGASDPSAEAEANGEKPFLLRALQIALVVSLYWVTSISMVFLNKYLLDSPSLRLDTPIFVTFYQCLVTTLLCKGLSALAACCPGAVDFPSLRLDLRVARSVLPLSVVFIGMITFNNLCLKYVGVAFYNVGRSLTTVFNVLLSYLLLKQTTSFYALLTCGIIIGGFWLGVDQEGAEGTLSWLGTVFGVLASLCVSLNAIYTTKVLPAVDGSIWRLTFYNNVNACILFLPLLLLLGELQALRDFAQLGSAHFWGMMTLGGLFGFAIGYVTGLQIKFTSPLTHNVSGTAKACAQTVLAVLYYEETKSFLWWTSNMMVLGGSSAYTWVRGWEMKKTPEEPSPKDSEKSAMGV

SEQ ID NO: 36 - DNA, 1095 bp, Homo sapiens
atgaatagggcccctctgaagcggtccaggatcctgcacatggcgctgaccggggcctcagacccctctgcagaggcagaggccaacggggagaagccctttctgctgcgggcattgcagatcgcgctggtggtctccctctactgggtcacctccatctccatggtgttccttaataagtacctgctggacagcccctccctgcggctggacacccccatcttcgtcaccttctaccagtgcctggtgaccacgctgctgtgcaaaggcctcagcgctctggccgcctgctgccctggtgccgtggacttccccagcttgcgcctggacctcagggtggcccgcagcgtcctgcccctgtcggtggtcttcatcggcatgatcaccttcaataacctctgcctcaagtacgtcggtgtggccttctacaatgtgggccgctcactcaccaccgtcttcaacgtgctgctctcctacctgctgctcaagcagaccacctccttctatgccctgctcacctgcggtatcatcatcgggggcttctggcttggtgtggaccaggagggggcagaaggcaccctgtcgtggctgggcaccgtcttcggcgtgctggctagcctctgtgtctcgctcaacgccatctacaccacgaaggtgctcccggcggtggacggcagcatctggcgcctgactttctacaacaacgtcaacgcctgcatcctcttcctgcccctgctcctgctgctcggggagcttcaggccctgcgtgactttgcccagctgggcagtgcccacttctgggggatgatgacgctgggcggcctgtttggctttgccatcggctacgtgacaggactgcagatcaagttcaccagtccgctgacccacaatgtgtcgggcacggccaaggcctgtgcccagacagtgctggccgtgctctactacgaggagaccaagagcttcctctggtggacgagcaacatgatggtgctgggcggctcctccgcctacacctgggtcaggggctgggagatgaagaagactccggaggagcccagccccaaagacagcgagaagagcgccatgggggtgtga

SEQ ID NO: 37 - protein, 363 aa, Mus musculus
MNRAPLKRSRILRMALTGVSAVSEESESGNKPFLLRALQIALVVSLYWVTSISMVFLNKYLLDSPSLQLDTPIFVTFYQCLVTSLLCKGLSTLATCCPGMVDFPTLNLDLKVARSVLPLSVVFIGMITFNNLCLKYVGVPFYNVGRSLTTVFNVLLSYLLLKQTTSFYALLTCGVIIGGFWLGIDQEGAEGTLSLTGTIFGVLASLCVSLNAIYTKKVLPAVDHSIWRLTFYNNVNACVLFLPLMIVLGELRALLAFTHLSSAHFWLMMTLGGLFGFAIGYVTGLQIKFTSPLTHNVSGTAKACAQTVLAVLYYEEIKSFLWWTSNLMVLGGSSAYTWVRGWEMQKTQEDPSSKDGEKSAIRV

SEQ ID NO: 38 - DNA, 1092 bp, Mus musculus
atgaacagggcgcctctgaagcggtccaggatcctgcgcatggcgctgactggagtctctgctgtctccgaggagtcagagagcgggaacaagccatttctgctccgggctctgcagatcgcgctggtggtctctctctactgggtcacctccatttccatggtattcctcaacaagtacctgctggacagcccctccctgcagctggatacccccatttttgtcaccttctaccaatgcctggtgacctcactgctgtgcaagggcctcagcactctggccacctgctgccccggcatggtagacttccccaccctaaacctggacctcaaggtggcccgaagtgtgctgccgctgtcagtggtctttatcggcatgataaccttcaataacctctgcctcaagtacgtaggggtgcccttctacaacgtgggacgctcgctcaccaccgtgttcaacgttcttctctcctacctgctgctcaaacagaccacttccttctatgccctgctcacctgcggcgtcatcattggtggtttctggctgggtatagaccaagaaggagctgagggaaccttgtccctgacgggcaccatcttcggggtgctggccagcctctgcgtctccctcaatgccatctataccaagaaggtgctccctgcagtagaccacagtatctggcgcctaaccttctataacaatgtcaatgcctgcgtgctcttcttgcccctgatgatagtgctgggcgagctccgtgccctcctggccttcactcatctgagcagtgcccacttctggctcatgatgacgctgggtggcctgtttggctttgccatcggctatgtgacaggactgcagatcaaattcaccagtcccctgacccataacgtgtcaggcacggccaaggcctgtgcacagacagtgctggccgtgctctactacgaagagattaagagcttcctgtggtggacaagcaacctgatggtgctgggtggctcctccgcctacacctgggtcaggggctgggagatgcagaagacccaggaggaccccagctccaaagatggtgagaagagtgctatcagggtgtga

SEQ ID NO: 39 - protein, 363 aa, Rattus norvegicus
MNRVPLKRSRILRMALTGASAVSEEADSENKPFLLRALQIALVVSLYWVTSISMVFLNKYLLDSPSLQLDTPIFVTFYQCLVTSLLCKGLSTLATCCPGMVDFPTLNLDLKVARSVLPLSVVFIGMITFNNLCLKYVGVAFYNVGRSLTTVFNVLLSYLLLKQTTSFYALLTCAIIIGGFWLGIDQEGAEGTLSLTGTIFGVLASLCVSLNAIYTKKVLPAVDHSIWRLTFYNNVNACVLFLPLMVVLGELHALLAFAHLNSAHFWVMMTLGGLFGFAIGYVTGLQIKFTSPLTHNVSGTAKACAQTVLAVLYYEEIKSFLWWTSNLMVLGGSSAYTWVRGWEMQKTQEDPSSKEGEKSAIGV

SEQ ID NO: 40 - DNA, 1092 bp, Rattus norvegicus
atgaacagggtccctctgaagcggtccaggatcctgcgcatggcgctgactggagcctctgctgtctctgaggaggcagacagcgagaacaagccatttctgctacgggctctgcagatcgcgctggtggtttctctctactgggtcacctccatctccatggtattcctcaacaagtacctgctggacagcccctccctgcagctggatacccccatcttcgtcaccttctaccaatgcctggtgacctcactgctgtgcaagggcctcagcactctggccacctgctgccctggcatggtagacttccccaccctaaacctggacctcaaggtggcccgaagtgtgctgccgctgtccgtggtctttatcggcatgataaccttcaataacctctgcctcaagtacgtgggggtggccttctacaacgtgggacgctcgctcactaccgtgttcaatgtgcttctctcctacctgctgcttaaacagaccacttccttttatgccctgctcacctgtgccatcatcattggtggtttctggctgggaatagatcaagagggagctgagggcaccctgtccctgacgggcaccatcttcggggtgctggccagcctctgtgtctcactcaatgccatctacaccaagaaggtgctccctgccgtagaccacagtatctggcgcctaaccttctataacaacgtcaacgcctgtgtgctcttcttgcccctgatggtagtgctgggcgagctccatgctctcctggccttcgctcatctgaacagcgcccacttctgggtcatgatgacgctgggtggactcttcggctttgccattggctatgtgacaggactgcagatcaaattcaccagtcccctgacccataatgtgtcgggcacagccaaggcctgtgcacagacagtgctggctgtgctctactatgaagagattaagagcttcctgtggtggacaagcaacttgatggtgctgggtggctcctctgcctacacctgggtcaggggctgggagatgcagaagacccaggaggaccccagctccaaagagggtgagaagagtgctatcggggtgtga

SEQ ID NO: 41 - protein, 365 aa, Cricetulus griseus
MNRAPLKRSRILRMALTGGSTASEEADEDSRNKPFLLRALQIALVVSLYWVTSISMVFLNKYLLDSPSLQLDTPIFVTFYQCLVTSLLCKGLSTLATCCPGTVDFPTLNLDLKVARSVLPLSVVFIGMISFNNLCLKYVGVAFYNVGRSLTTVFNVLLSYLLLKQTTSFYALLTCGIIIGGFWLGIDQEGAEGTLSLIGTIFGVLASLCVSLNAIYTKKVLPAVDNSIWRLTFYNNVNACVLFLPLMVLLGELRALLDFAHLYSAHFWLMMTLGGLFGFAIGYVTGLQIKFTSPLTHNVSGTAKACAQTVLAVLYYEETKSFLWWTSNLMVLGGSSAYTWVRGWEMQKTQEDPSSKEGEKSAIRV

SEQ ID NO: 42 - DNA, 1098 bp, Cricetulus griseus
atgaacagggcgcctctgaagcggtccaggatcctgcgcatggcgctgactggaggctccactgcctctgaggaggcagatgaggacagcaggaacaagccgtttctgctgcgggcgctgcagatcgcgctggtcgtctctctctactgggtcacctccatctccatggtattcctcaacaagtacctgctggacagcccctccctgcagctggatacccctatcttcgtcactttctaccaatgcctggtgacctctctgctgtgcaagggcctcagcactctggccacctgctgccctggcaccgttgacttccccaccctgaacctggaccttaaggtggcccgcagcgtgctgccactgtcggtagtcttcattggcatgataagtttcaataacctctgcctcaagtacgtaggggtggccttctacaacgtggggcgctcgctcaccaccgtgttcaatgtgcttctgtcctacctgctgctcaaacagaccacttccttctatgccctgctcacatgtggcatcatcattggtggtttctggctgggtatagaccaagagggagctgagggcaccctgtccctcataggcaccatcttcggggtgctggccagcctctgcgtctccctcaatgccatctataccaagaaggtgctcccagcagtggacaacagcatctggcgcctaaccttctataacaatgtcaatgcctgtgtgctcttcttgcccctgatggttctgctgggtgagctccgtgccctccttgactttgctcatctgtacagtgcccacttctggctcatgatgacgctgggtggcctcttcggctttgccattggctatgtgacaggactgcagatcaaattcaccagtcccctgacccacaatgtatcaggcacagccaaggcctgtgcgcagacagtgctggccgtgctctactatgaagagactaagagcttcctgtggtggacaagcaacctgatggtgctgggtggctcctcagcctatacctgggtcaggggctgggagatgcagaagacccaagaggaccccagctccaaagagggtgagaagagtgctatcagggtgtga